SEQUENCE LISTING (81 sequences)

SEQ ID NO: 1 - DNA, 20 bp, Artificial Sequence
  Description of Artificial SequenceSynthetic
cacgccgcccacgtgaaggc

SEQ ID NO: 2 - DNA, 20 bp, Artificial Sequence
  Description of Artificial SequenceSynthetic
ttcgccaatgacaagacgct

SEQ ID NO: 3 - DNA, 1235 bp, Mouse
  CDS (161)..(796)
cgaggctcaagctccgggcggattctgcgtgccgctctcgctccttggggtctgttggccggcctgtgccacccggacgcccggctcactgcctctgtctcccccatcagcgcagccccggacgctatggcccacccctccagctggcccctcgagtaggatggtagcacgcaaccaggtggcagccgacaatgcgatctccccggcagcagagccccgacggcggtcagagccctcctcgtcctcgtcttcgtcctcgccagcggcccccgtgcgtccccggccctgcccggcggtcccagccccagcccctggcgacactcacttccgcaccttccgctcccactccgattaccggcgcatcacgcggaccagcgcgctcctggacgcctgcggcttctattggggacccctgagcgtgcacggggcgcacgagcggctgcgtgccgagcccgtgggcaccttcttggtgcgcgacagtcgtcaacggaactgcttcttcgcgctcagcgtgaagatggcttcgggccccacgagcatccgcgtgcacttccaggccggccgcttccacttggacggcagccgcgagaccttcgactgccttttcgagctgctggagcactacgtggcggcgccgcgccgcatgttgggggccccgctgcgccagcgccgcgtgcggccgctgcaggagctgtgtcgccagcgcatcgtggccgccgtgggtcgcgagaacctggcgcgcatccctcttaacccggtactccgtgactacctgagttccttccccttccagatctgaccggctgccgctgtgccgcagcattaagtgggggcgccttattatttcttattattaattattattatttttctggaaccacgtgggagccctccccgcctgggtcggagggagtggttgtggagggtgagatgcctcccacttctggctggagacctcatcccacctctcaggggtgggggtgctcccctcctggtgctccctccgggtcccccctggttgtagcagcttgtgtctggggccaggacctgaattccactcctacctctccatgtttacatattcccagtatctttgcacaaaccaggggtcggggagggtctctggcttcatttttctgctgtgcagaatatcctattttatatttttacagccagtttaggtaataaactttattatgaaagtttttttttaaaagaaaaaaaaaaaaaaaaaa

SEQ ID NO: 4 - protein, 212 aa, Mouse
MVARNQVAADNAISPAAEPRRRSEPSSSSSSSSPAAPVRPRPCPAVPAPAPGDTHFRTFRSHSDYRRITRTSALLDACGFYWGPLSVHGAHERLRAEPVGTFLVRDSRQRNCFFALSVKMASGPTSIRVHFQAGRFHLDGSRETFDCLFELLEHYVAAPRRMLGAPLRQRRVRPLQELCRQRIVAAVGRENLARIPLNPVLRDYLSSFPFQI

SEQ ID NO: 5 - DNA, 1121 bp, Mouse
  CDS (223)..(816)
gcgatctgtgggtgacagtgtctgcgagagactttgccacaccattctgccggaatttggagaaaaagaaccagccgcttccagtcccctccccctccgccaccatttcggacaccctgcacactctcgttttggggtaccctgtgacttccaggcagcacgcgaggtccactggccccagctcgggcgaccagctgtctgggacgtgttgactcatctcccatgaccctgcggtgcctggagccctccgggaatggagcggacaggacgcggagccagtgggggaccgcggggttgccggaggaacagtcccccgaggcggcgcgtctggcgaaagccctgcgcgagctcagtcaaacaggatggtactggggaagtatgactgttaatgaagccaaagagaaattaaaagaggctccagaaggaactttcttgattagagatagttcgcattcagactacctactaactatatccgttaagacgtcagctggaccgactaacctgcggattgagtaccaagatgggaaattcagattggattctatcatatgtgtcaagtccaagcttaaacagtttgacagtgtggttcatctgattgactactatgtccagatgtgcaaggataaacggacaggcccagaagccccacggaatgggactgttcacctgtacctgaccaaacctctgtatacatcagcacccactctgcagcatttctgtcgactcgccattaacaaatgtaccggtacgatctggggactgcctttaccaacaagactaaaagattacttggaagaatataaattccaggtataagtatttctctctctttttcgtttttttttaaaaaaaaaaaaacacatgcctcatatagactatctccgaatgcagctatgtgaaagagaacccagaggccctcctctggataactgcgcagaattctctcttaaggacagttgggctcagtctaacttaaaggtgtgaagatgtagctaggtattttaaagttccccttaggtagttttagctgaatgatgctttctttcctatggctgctcaagatcaaatggcccttttaaatgaaacaaaacaaaacaaaacaaaaaaaaaaaaaaaaa

SEQ ID NO: 6 - protein, 198 aa, Mouse
MTLRCLEPSGNGADRTRSQWGTAGLPEEQSPEAARLAKALRELSQTGWYWGSMTVNEAKEKLKEAPEGTFLIRDSSHSDYLLTISVKTSAGPTNLRIEYQDGKFRLDSIICVKSKLKQFDSVVHLIDYYVQMCKDKRTGPEAPRNGTVHLYLTKPLYTSAPTLQHFCRLAINKCTGTIWGLPLPTRLKDYLEEYKFQV

SEQ ID NO: 7 - DNA, 2187 bp, Mouse
  CDS (18)..(692)
cgctggctccgtgcgccatggtcacccacagcaagtttcccgccgccgggatgagccgccccctggacaccagcctgcgcctcaagaccttcagctccaaaagcgagtaccagctggtggtgaacgccgtgcgcaagctgcaggagagcggattctactggagcgccgtgaccggcggcgaggcgaacctgctgctcagcgccgagcccgcgggcacctttcttatccgcgacagctcggaccagcgccacttcttcacgttgagcgtcaagacccagtcggggaccaagaacctacgcatccagtgtgaggggggcagcttttcgctgcagagtgacccccgaagcacgcagccagttccccgcttcgactgtgtactcaagctggtgcaccactacatgccgcctccagggaccccctccttttctttgccacccacggaaccctcgtccgaagttccggagcagccacctgcccaggcactccccgggagtacccccaagagagcttactacatctattctgggggcgagaagattccgctggtactgagccgacctctctcctccaacgtggccaccctccagcatctttgtcggaagactgtcaacggccacctggactcctatgagaaagtgacccagctgcctggacccattcgggagttcctggatcagtatgatgctccactttaaggagcaaaagggtcagaggggggcctgggtcggtcggtcgcctctcctccgaggcacatggcacaagcacaaaaatccagccccaacggtcggtagctcccagtgagccaggggcagattggcttcttcctcaggccctccactcccgcagagtagagctggcaggacctggaattcgtctgaggggagggggagctgccacctgctttcccccctcccccagctccagcttctttcaagtggagccagccggcctggcctggtgggacaatacctttgacaagcggactctcccctccccttcctccacaccccctctgcttcccaagggaggtggggacacctccaagtgttgaacttagaactgcaaggggaatcttcaaactttcccgctggaacttgtttgcgctttgatttggtttgatcaagagcaggcacctgggggaaggatggaagagaaaagggtgtgtgaagggtttttatgctggccaaagaaataaccactcccactgcccaacctaggtgaggagtggtggctcctggctctggggagagtggcaaggggtgacctgaagagagctatactggtgccaggctcctctccatggggcagctaatgaaacctcgcagatcccttgcaccccagaaccctccccgttgtgaagaggcagtagcatttagaagggagacagatgaggctggtgagctggccgccttttccaacaccgaagggaggcagatcaacagatgagccatcttggagcccaggtttcccctggagcagatggagggttctgctttgtctctcctatgtggggctaggagactcgccttaaatgccctctgtcccagggatggggattggcacacaaggagccaaacacagccaataggcagagagttgagggattcacccaggtggctacaggccaggggaagtggctgcaggggagagacccagtcactccaggagactcctgagttaacactgggaagacattggccagtcctagtcatctctcggtcagtaggtccgagagcttccaggccctgcacagccctcctttctcacctggggggaggcaggaggtgatggagaagccttcccatgccgctcacaggggcctcacgggaatgcagcagccatgcaattacctggaactggtcctgtgttggggagaaacaagttttctgaagtcaggtatggggctgggtggggcagctgtgtgttggggtggcttttttctctctgttttgaataatgtttacaatttgcctcaatcacttttataaaaatccacctccagcccgcccctctccccactcaggccttcgaggctgtctgaagatgcttgaaaaactcaaccaaatcccagttcaactcagactttgcacatatatttatatttatactcagaaaagaaacatttcagtaatttataataaaagagcactattttttaatgaaaaaaaaaaaaaaaaaaaaaaaaaa

SEQ ID NO: 8 - protein, 225 aa, Mouse
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGTPSFSLPPTEPSSEVPEQPPAQALPGSTPKRAYYIYSGGEKIPLVLSRPLSSNVATLQHLCRKTVNGHLDSYEKVTQLPGPIREFLDQYDAPL

SEQ ID NO: 9 - DNA, 1094 bp, Human
ctccggctggccccttctgtaggatggtagcacacaaccaggtggcagccgacaatgcagtctccacagcagcagagccccgacggcggccagaaccttcctcctcttcctcctcctcgcccgcggcccccgcgcgcccgcggccgtgccccgcggtcccggccccggcccccggcgacacgcacttccgcacattccgttcgcacgccgattaccggcgcatcacgcgcgccagcgcgctcctggacgcctgcggattctactgggggcccctgagcgtgcacggggcgcacgagcggctgcgcgccgagcccgtgggcaccttcctggtgcgcgacagccgccagcggaactgctttttcgcccttagcgtgaagatggcctcgggacccacgagcatccgcgtgcactttcaggccggccgctttcacctggatggcagccgcgagagcttcgactgcctcttcgagctgctggagcactacgtggcggcgccgcgccgcatgctgggggccccgctgcgccagcgccgcgtgcggccgctgcaggagctgtgccgccagcgcatcgtggccaccgtgggccgcgagaacctggctcgcatccccctcaaccccgtcctccgcgactacctgagctccttccccttccagatttgaccggcagcgcccgccgtgcacgcagcattaactgggatgccgtgttattttgttattacttgcctggaaccatgtgggtaccctccccggcctgggttggagggagcggatgggtgtaggggcgaggcgcctcccgccctcggctggagacgaggccgcagaccccttctcacctcttgagggggtcctccccctcctggtgctccctctgggtccccctggttgttgtagcagcttaactgtatctggagccaggacctgaactcgcacctcctacctcttcatgtttacatatacccagtatctttgcacaaaccaggggttgggggagggtctctggctttatttttctgctgtgcagaatcctattttatattttttaaagtcagtttaggtaataaactttattatgaaagtttttttttttaaaaaaaa

SEQ ID NO: 10 - protein, 211 aa, Human
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 11 - DNA, 2807 bp, Rat
ggaaaccgaggcggggagaccaggaggccttggcctcagagcttcagagtcgcgtggcagcaaacagagaaacctgtagagggcagtgtgcgtcacttagctcagggaagctgcacgcgaaactcacccgccttcattcataaacatcgtcagctaggcacctactcctgggctttcaggacaaactgaatcacgaaaccacagtgtccttaaaataggtctgaccgcctgaatccctggccaaggtgtgtacggggcatgggagcccttgtgcagagatgcttgcaggagccttgaggggctctgtaagacagaggctaggaagacaaagttgggggctacagcttcttgtcctgcccggggcctcagtttcttcggttgcccacgtaggagtgcagagagtccagcccctggggacccaacccaaccccgcccagtttccgaggaactcgtccgggagcgggggcgcccctcccgcaccgccttaggcttcctttgaagcctctgcggtcaggccaccgcttcctgggaagcccaagccaaggccaggccgagtggccaacgggaggggcccgcgcgcgattctggaggagggcggcggccccacaggtctccagggctggctagccgggctcctagagcggagactgccaaggccttcgggtcctgggcaggaaggatcctggcagggaggagttgcttggggggtgggggggaaaggctccaggcgcggtggagctctgaccaggagaatgcacacactcggaggggaggaggcgtgtcagccccaagctagcatcccacccggggagcagcgatgtggggcgaaggtagccagagcaaaagagcaggcaccaggtgacacgaaacagaagattccgggtagagccagaaccccagaagtcccattcagggaaggtgcgaggcgagaacgagttaggtggaccctctccaggggcagccaaagaaatctaaagagaacccgaaggacttgccggaaagagaaaccgaaagcggcggtgggcgggatcggtgggcggggcctccctggtttaagagcttgatgcaggggcgggcagcagcagagagaactgcggccgtggcagcggcacggctcccggccccggagcatgcgcgacagcagccccggaacccccagccgcggcgccccgcgtcccgccgccaggtgagccgaggcagctgcgaaggagcaggcgggaggggatgggaggaaggggagcagagcctggcaggactatcctcgcagactgcatggcggggtcgtggatgctatgcctctggcgcccgccccaccggctggcccaggcggcccctcgcgcgcgcggggcgccgtcagcccctcctctccggccctgagcccggatcgtccgcccgggttccagttcccggcgtggccagtaggcggcaaccgcgaggcggcaagccacccagcggggacggcctggagtcgggcccctctccacgcccccttctccacgcgcgcggggaggcagggctccaccgccagtctggaagggttccacatacaggaacggcctacttcgcagatgagcccaccgaggctcaggctccgggcggattctgcgtgtcaccctcgctccttggggtccgctggccggcctgtgccacccggacgcccggttcactgcctctgtctcccccatcagcgcagccccggacgctatggcccacccctccagctggcccctcgagtaggatggtagcacgtaaccaggtggaagccgacaatgcgatctccccggcatcagagccccgacggcggccagagccatcctcgtcctcgtcttcgtcctcgccggcggccccggcgcgtccccggccctgcccggtggtcccggccccggctccgggcgacactcacttccgcaccttccgctcccactctgattaccggcgcatcacgcggaccagcgctctcctggacgcctgcggcttctactggggacccctgagcgtgcatggggcgcacgaacggctgcgttccgaacccgtgggcaccttcttggtgcgcgacagtcgccagcggaactgcttcttcgcgctcagcgtgaagatggcttcgggccccacgagcattcgtgtgcacttccaggccggccgcttccacctggacggcaaccgcgagaccttcgactgcctcttcgagctgctggagcactacgtggcggcgccgcgccgcatgttgggggccccactgcgccagcgccgcgtgcggccgctgcaggagctgtgtcgccagcgcatcgtggccgccgtgggtcgcgagaacctggcacgcatccctcttaacccggtactccgtgactacctgagttccttccccttccagatctgaccggctgccgccgtgcccgcagaattaagtgggagcgccttattatttcttattattaattattattatttttctggaaccacgtgggagccctccccgcctaggtcggagggagtgggtgtggagggtgagatccctcccacttctggctggagaccttatcccgcctctcggggggcctcccctcctggtgctccctcccggtccccctggttgtagcagcttgtgtctggggccaggacctgaactccacgcctacctctccatgtttacatgttcccagtatctttgcacaaaccaggggtgggggagggtctctggcttcatttttctgctgtgcagaatattctattttatatttttacatccagtttagataataaactttattatgaaagtttttttttttaaagaaacaaagatttctaga

SEQ ID NO: 12 - protein, 212 aa, Rat
MVARNQVEADNAISPASEPRRRPEPSSSSSSSSPAAPARPRPCPVVPAPAPGDTHFRTFRSHSDYRRITRTSALLDACGFYWGPLSVHGAHERLRSEPVGTFLVRDSRQRNCFFALSVKMASGPTSIRVHFQAGRFHLDGNRETFDCLFELLEHYVAAPRRMLGAPLRQRRVRPLQELCRQRIVAAVGRENLARIPLNPVLRDYLSSFPFQI

SEQ ID NO: 13 - DNA, 1611 bp, Mouse
  CDS (263)..(1525)
cgaattccgggcgggctgtgtgagtctgtgagtggaaggcgcgccggctcttttgtctgagtgtgacccggtggctttgttccaggcattccggtgatttcctccgggcagtccgcagaagccgcagcggccgcccgcgctctctctgcagtctccacacccgggagagcctgagcccgcgtcacgcccctcagcccccgctgagtcccttctctgttgtcgcgtccgaatcgagttcccggaatcagacggtgccccatagatggccagctttcccccgagggttaacgagaaagagatcgtgagatcacgtactataggggaactcttggctccagcagctccttttgacaagaaatgtggtggtgagaactggacggttgcttttgctcctgatggttcctactttgcgtggtcacaaggatatcgcatagtgaagcttgtcccgtggtcccagtgccgtaagaactttcttttgcatggttccaaaaatgttaccaattcaagctgtctaaaattggcaagacaaaacagtaatggtggtcagaaaaacaagcctcctgagcacgttatagactgtggagacatagtctggagtcttgcttttgggtcttcagttccagaaaaacagagtcgttgcgttaatatagaatggcatcggttccgatttggacaggatcagctactccttgccacaggattaaacaatggtcgcatcaaaatctgggatgtatatacaggaaaactcctccttaatttggtagaccacattgaaatggttagagatttaacttttgctccagatgggagcttactccttgtatcagcttcaagagacaaaactctaagagtgtgggacctgaaagatgatggaaacatggtgaaagtattgcgggcacatcagaattgggtgtacagttgtgcattctctcccgactgttctatgctgtgttcagtgggcgccagtaaagcagttttcctttggaatatggataaatacaccatgattaggaagctggaaggtcatcaccatgatgttgtagcttgtgacttttctcctgatggagcattgctagctactgcatcctatgacactcgtgtgtatgtctgggatccacacaatggagaccttctgatggagtttgggcacctgtttccctcgcccactccaatatttgctggaggagcaaatgaccgatgggtgagagctgtgtctttcagtcatgatggactgcatgttgccagccttgctgatgataaaatggtgaggttctggagaatcgatgaggattgtccggtacaagttgcacctttgagcaatggtctttgctgtgccttttctactgatggcagtgttttagctgctgggacacatgatggaagtgtgtatttttgggccactccaaggcaagtccctagccttcaacatatatgtcgcatgtcaatccgaagagtgatgtccacccaagaagtccaaaaactgcctgttccttccaaaatattggcgtttctctcctaccgcggttagactgaagactgcctttcctggtaggcctgccagacagagcgccctttacaagacacacctcaagctttacctcgtgccgaatt

SEQ ID NO: 14 - protein, 421 aa, Mouse
MASFPPRVNEKEIVRSRTIGELLAPAAPFDKKCGGENWTVAFAPDGSYFAWSQGYRIVKLVPWSQCRKNFLLHGSKNVTNSSCLKLARQNSNGGQKNKPPEHVIDCGDIVWSLAFGSSVPEKQSRCVNIEWHRFRFGQDQLLLATGLNNGRIKIWDVYTGKLLLNLVDHIEMVRDLTFAPDGSLLLVSASRDKTLRVWDLKDDGNMVKVLRAHQNWVYSCAFSPDCSMLCSVGASKAVFLWNMDKYTMIRKLEGHHHDVVACDFSPDGALLATASYDTRVYVWDPHNGDLLMEFGHLFPSPTPIFAGGANDRWVRAVSFSHDGLHVASLADDKMVRFWRIDEDCPVQVAPLSNGLCCAFSTDGSVLAAGTHDGSVYFWATPRQVPSLQHICRMSIRRVMSTQEVQKLPVPSKILAFLSYRG

SEQ ID NO: 15 - DNA, 783 bp, Human
ctgtcttcctccgcagcgcgaggctgggtacagggtctattgtctgtggttgactccgtactttggtctgaggccttcgggagctttcccgaggcagttagcagaagccgcagcgaccgcccccgcccgtctcctctgtccctgggcccgggagacaaacttggcgtcacgccctcagcggtcgccactctcttctctgttgttgggtccgcatcgtattcccggaatcagacggtgccccatagatggccagctttcccccgagggtcaacgagaaagagatcgtgagatcacgtactataggtgaacttttagctcctgcagctccttttgacaagaaatgtggtcgtgaaaattggactgttgcttttgctccagatggttcatactttgcttggtcacaaggacatcgcacagtaaagcttgttccgtggtcccagtgccttcagaactttctcttgcatggcaccaagaatgttaccaattcaagcagtttaagattgccaagacaaaatagtgatggtggtcagaaaaataagcctcgtgacatattatagactgtggagatatagtctggagtcttgcttttgggtcatcagttccagaaaaacagagtcgctgtgtaaatatagaatggcatcgcttcagatttggacaagatcagctacttcttgctacagggttgaacaatgggcgtatcaaaatatgggatgtatatcaggaaactcctccttaacttggtagatcatactgaagtggtcagagatttaacttttgctccag

SEQ ID NO: 16 - DNA, 1122 bp, Human
ctctgtatgtctgaatgaagctataacatttgcctttttattgcaggttttcctttggaatatggataaatacaccatgatacggaaactagaaggacatcaccatgatgtggtagcttgtgacttttctcctgatggagcattactggctactgcatcttatgatactcgagtatatatctgggatccacataatggagacattctgatggaatttgggcacctgtttcccccacctactccaatatttgctggaggagcaaatgaccggtgggtacgatctgtatcttttagccatgatggactgcatgttgcaagccttgctgatgataaaatggtgaggttctggagaattgatgaggattatccagtgcaagttgcacctttgagcaatggtctttgctgtgccttctctactgatggcagtgttttagctgctgggacacatgacggaagtgtgtatttttgggccactccacggcaggtccctagcctgcaacatttatgtcgcatgtcaatccgaagagtgatgcccacccaagaagttcaggagctgccgattccttccaagcttttggagtttctctcgtatcgtatttagaagattctgccttccctagtagtagggactgacagaatacacttaacacaaacctcaagctttactgacttcaattatctgtttttaaagacgtagaagatttatttaatttgatatgttcttgtactgcattttgatcagttgagcttttaaaatattatttatagacaatagaagtatttctgaacatatcaaatataaatttttttaaagatctaactgtgaaaacatacatacctgtacatatttagatataagctgctatatgttgaatggacccttttgcttttctgatttttagttctgacatgtatatattgcttcagtagagccacaatatgtatctttgctgtaaagtgcaaggaaattttaaattctgggacactgagttagatggtaaatactgacttacgaaagttgaattgggtgaggcgggcaaatcacctgaggtcagcagtttgagactagcctggcaaacatgatgaaaccctgtctctactaaaaatacaaaaaaaaaaaaa

SEQ ID NO: 17 - DNA, 2544 bp, Mouse
  CDS (423)..(2030)
  UNSURE (320): Xaa is unsure
cggcacgagccgggctccgtccggaggaagcgaggctgcgccgccggcccggcaggagcggaggacgggamgcgcgggcggtcgcgctcgccctgtcgctgactgcgctgccccggcccatccttgcctggccgcaggtgccctggatgaggccgccgcgcgtgtcccggccgctgagtgtcccccgcggtcgcccggcgcctgccctcaagcggccgcctctccttgcccgggtccccgttttcccccggcgcagtcctcctccggtgggcgcctccgcacctcggcgcaggcggcacggccctcgggccgggatggatccgccgggaagaggaagacaagccggggcgttgagcccctgcgcacggtgccgccgcgcgtagtgggagcttactcgcagtaggctctcgctcttctaatcaatggataaagtggggaaaatgtggaacaacttaaaatacagatgccagaatctcttcagccacgagggaggaagccgtaatgagaacgtggagatgaaccccaacagatgtccgtctgtcaaagagaaaagcatcagtctgggagaggcagctccccagcaagagagcagtcccttaagagaaaatgttgccttacagctgggactgagcccttccaagaccttttccaggcggaaccaaaactgtgccgcagagatccctcaagtggttgaaatcagcatcgagaaagacagtgactcgggtgccaccccaggaacgaggcttgcacggagagactcctactcgcggcacgccccgtggggaggaaagaagaaacattcctgttccacaaagacccagagttcattggataccgagaaaaagtttggtagaactcgaagcggccttcagaggcgagagcggcgctatggagtcagctccatgcaggacatggacagcgtttctagccgcgcggtcgggagccgctccctgaggcagaggctccaggacacggtgggtttgtgttttcccatgagaacttacagcaagcagtcaaagccactcttttccaataaaagaaaaatacatctttctgaattaatgctggagaaatgcccttttcctgctggctcggatttagcacaaaagtggcatttgattaaacagcataccgcccctgtgagcccacactcaacattttttgatacatttgatccatcactggtgtctacagaagatgaagaagataggcttcgcgagagaagacggcttagtatcgaagaaggggtggatccccctcccaacgcacaaatacacacctttgaagctactgcacaggtcaacccattgtataagctgggaccaaagttagctcctgggatgacagagataagtggagatggttctgcaattccacaagcsaattgtgactcagaagaggattcaaccaccctatgtctgcagtcacggaggcagaagcagcgccaggtgtccggggacagccacgcgcacgttagcagacagggagcttggaaagttcatacgcagatcgattacatacactgcctcgtgccagatttgcttcagatcacagggaatccctgttactggggcgtgatggaccgatacgaggccgaagcccttctagaagggaaaccggaaggcacgttcttgctcagggactctgcacaggaggactacctcttctctgtgagcttccgccgctacaacaggtctctgcacgcccggatcgagcagtggaaccacaacttcagcttcgatgcccatgacccctgcgtgtttcactcctccacwgtcacggggcttctcgaacactataaagaccccagctcttgcatgttttttgaaccgttgctaacgatatcactgaatagaactttccctttcagcctgcagtatatctgccgcgcagtgatctgcagatgcactacgtatgatgggattgacgggctcccgctaccgtcgatgttacaggattttttaaaagagtatcattataaacaaaaagttagggttcgctggttagaacgagarccagtcaaagcaaagtaactcctgtccccaaagggcactaactaagtctgctcctcccgtgcatcmgaactgcacccataggraggcagtcagctgctaggatttcccacccagaatgggagcttagtcattagcctctgccctatggggtccgctgttcctcagacaaaggtgcctagggacagcaagatggcttgcaggtgttcggtgggctgtgacaactgagggaggcaactctggggcatttgctatgaagaattctatttcttaccgaagaacaaattattaatattggatgggtatttcaatagtgtgactaatgtttgaaattattttttctaagaatttttctataaccttcagaaaaagtagtgatgtttgtagttactataaatcaagctttgaaagttcaaaacaaacaagttaaataaaagactaccttccttttagagaaaacaaatgcaagttttcccagccacaggcattgtgcactgttaatgttagcttgttatcagctcctttctcctcc

SEQ ID NO: 18 - protein, 536 aa, Mouse
  UNSURE (320): Xaa is unsure
MDKVGKMWNNLKYRCQNLFSHEGGSRNENVEMNPNRCPSVKEKSISLGEAAPQQESSPLRENVALQLGLSPSKTFSRRNQNCAAEIPQVVEISIEKDSDSGATPGTRLARRDSYSRHAPWGGKKKHSCSTKTQSSLDTEKKFGRTRSGLQRRERRYGVSSMQDMDSVSSRAVGSRSLRQRLQDTVGLCFPMRTYSKQSKPLFSNKRKIHLSELMLEKCPFPAGSDLAQKWHLIKQHTAPVSPHSTFFDTFDPSLVSTEDEEDRLRERRRLSIEEGVDPPPNAQIHTFEATAQVNPLYKLGPKLAPGMTEISGDGSAIPQXNCDSEEDSTTLCLQSRRQKQRQVSGDSHAHVSRQGAWKVHTQIDYIHCLVPDLLQITGNPCYWGVMDRYEAEALLEGKPEGTFLLRDSAQEDYLFSVSFRRYNRSLHARIEQWNHNFSFDAHDPCVFHSSXVTGLLEHYKDPSSCMFFEPLLTISLNRTFPFSLQYICRAVICRCTTYDGIDGLPLPSMLQDFLKEYHYKQKVRVRWLERXPVKAK

SEQ ID NO: 19 - DNA, 1221 bp, Human
gattaaacagcatacagctcctgtgagcccacattcaacattttttgatactttgatccatctttggtttctacagaagatgaagaagataggcttagagagagaaggcggcttagtattgaagaaggggttgatccccctcccaatgcacaaatacatacatttgaagctactgcacaggttaatccattattaaactgggaccaaaattagctcctggaatgactgaaataagtggggacagttctgcaattccacaagctaattgtgactcggaagaggatacaaccaccctgtgttgcagtcacggaggcagaagcagcgtcagatatctggagacagccatacccatgttagcagacagggagcttggaaagtccacacacagattgattacatacactgcttcgtgcctgatttgcttcaaattacagggaatccctgttactggggagtgatggaccgttatgaagcagaagcccttctcgaagggaaacctgaaggcacgtttttgctcagggactctgcgcaagaggactacttcttctctgtgagcttccgccgatacaacagatccctgcatgcccgaattgagcagtggaatcacaactttagtttcgacgcccatgacccgtgtgtatttcactcctccactgtaacgggacttttagaacattataaagatcccagttcgtgcatgttttttgaaccattgcttactatatcactaaataggactttcccttttagcctgcagtatatctgtcgcgcggtaatctgcaggtgcactacgtatgatggaattgatgggctccctctaccctcaatgttacaggattttttaaaagagtatcattataaacaaaaagttagagttcgctggttggaacgagaaccagtcaaggcaaagtaaactctccggtccccaaagggtgttaactaggtccgctttcatgtgcatcagacagtacacctatagcaagcacacgtagcagtgttaggctttttcatacagtatgtaagcttagtgttagtatctgtcagatgctacctgctgttacttattcagataaacatggtgcctattggaacaatagcggatagagctacaggtgttcagtaagactacaaaaacattttgcctatttcgctaacagtttggtttttaatggctgtggtatttgagtgaggcaactctggggcatttgttatgaagaaatg

SEQ ID NO: 20 - DNA, 2369 bp, Murine
  CDS (116)..(1327)
ggcacgaggcggtggtggcggcggcgggcgcggccgcggcggggcgggcgcggaatgaaggcccacggccctgggggctgaggcgcccgccgcctggggcgggccgcgcgtcctcatggaggccggagaggagccgctgctgctggctgaactcaagcctgggcgcccccaccagttcgactggaagtcaagctgcgagacctggagcgtggccttctcgccagacggttcctggttcgcctggtctcaaggacactgcgtggtcaagctggtcccctggcccttagaggaacagttcatccctaaaggattcgaagccaagagccgaagcagcaagaatgacccaaaaggacggggcagtctgaaggagaagacgctggactgtggccagattgtgtgggggctggccttcagcccgtggccctctccacccagcaggaaactctgggcacgtcaccatccccaggcgcctgatgtttcttgcctgatcctggccacaggtctcaacgatgggcagatcaagatttgggaggtacagacaggcctcctgcttctgaatctttctggccaccaagacgtcgtgagagatctgagcttcacgcccagcggcagtttgattttggtctctgcatcccgggataagacacttcgaatttgggacctgaataaacacggtaagcagatccaggtgttatccggccatctgcagtgggtttactgctgctccatctcccctgactgtagcatgctgtgctctgcagctggggagaagtcggtctttctgtggagcatgcggtcctacacactaatccggaaactagaaggccaccaaagcagtgttgtctcctgtgatttctctcctgattcagccttgcttgtcacagcttcgtatgacaccagtgtgattatgtgggacccctacaccggcgcgaggctgaggtcacttcatcacacacaacttgaacccaccatggatgacagtgacgtccacatgagctccctgaggtccgtgtgcttctcacctgaaggcttgtatctcgctacggtggcagatgacaggctgctcaggatctgggctctggaactgaaggctccggttgcctttgctccgatgaccaatggtctttgctgcacgttcttcccacacggtggaattattgccacagggacgagagatggccatgtccagttctggacagctccccgggtcctgtcctcactgaagcacttatgcaggaaagccctccgaagtttcctgacaacgtatcaagtcctagcactgccaatccccaagaagatgaaagagttcctcacatacaggactttctagcagtgccggctcccccacctcctgcagcagcagcagtacaagggactggctaggatggagtcaggcagctcacactggaccagtgtggaccttccttcctcccatggcatgtgcaagtaggtctgcgtgaccccacttctgtggtgccggccttacctcgtcttcatccgtggtgagcagccttcgtcagtctagttgtgttgaagccaagtgcagttgtggatgttgctggggtaataaaggcaagcgggctccagagcctctctggtggcggccaagccacactcccttaactgggaagtacctgccacgtagggcatttctgctgcctatttccagccagcggctgcatggtttgaagttcctccgttgtggtcagaagaactctggtgtttggttccctgctcagctgcgcgtggactgggctgagctcctcaccatacactagtgccggcttttgtttcctgtaaacagtggttgcatgtgtagagaagtaacaagcgagtattcagatcatacgaggaggcgttcctcggtgcatgacggtcagatggccatttatcagcatatttatttgtattttctcagcacatagtaaggtacaactgtgttttctcaattgtctcgaaaaaacagagttcttaagtggcccagttgtggagccaagtctaagtcgtgtggagtcagtgctgacatcactggcttgtgctgtctgtcacatgtgtttgtctctgctgcttgacctcatgggatgtaccctccagttcaactgcccaaaacagacagccccttccaagcaccgttctttgacagcggtagcagctacctattcaagacgcctcacacaaaatctgccttagaaagttaatatattttaaattattttaaaagaaactcaacatcttattctttggcctttcttaattgatgctttatggaggcagtgttaacattgtacagtgtatgcatagaggagtctcctctatttgaagaacaatgcaaaatgaggctttcattgaagggaaaaaaaaaaaaaaaa

SEQ ID NO: 21 - protein, 404 aa, Murine
MEAGEEPLLLAELKPGRPHQFDWKSSCETWSVAFSPDGSWFAWSQGHCVVKLVPWPLEEQFIPKGFEAKSRSSKNDPKGRGSLKEKTLDCGQIVWGLAFSPWPSPPSRKLWARHHPQAPDVSCLILATGLNDGQIKIWEVQTGLLLLNLSGHQDVVRDLSFTPSGSLILVSASRDKTLRIWDLNKHGKQIQVLSGHLQWVYCCSISPDCSMLCSAAGEKSVFLWSMRSYTLIRKLEGHQSSVVSCDFSPDSALLVTASYDTSVIMWDPYTGARLRSLHHTQLEPTMDDSDVHMSSLRSVCFSPEGLYLATVADDRLLRIWALELKAPVAFAPMTNGLCCTFFPHGGIIATGTRDGHVQFWTAPRVLSSLKHLCRKALRSFLTTYQVLALPIPKKMKEFLTYRTF

SEQ ID NO: 22 - DNA, 1246 bp, Human
gacactgcatcgtcaaactgatcccctggccgttggaggagcagttcatccctaaagggtttgaagccaaaagccgaagtagcaaaaatgagacgaaagggcggggcagcccaaaagagaagacgctggactgtggtcagattgtctgggggctggccttcagcctgtgctttccccacccagcaggaagctctgggcacgccaccacccccaagtgcccgatgtctcttgcctggttcttgctacgggactcaacgatgggcagatcaagatctgggaggtgcagacagggctcctgcttttgaatctttccggccaccaagatgtcgtgagagatctgagcttcacacccagtggcagtttgattttggtctccgcgtcacgggataagactcttcgcatctgggacctgaataaacacggtaaacagattcaagtgttatcgggccacctgcagtgggtttactgctgttccatctccccagactgcagcatgctgtgctctgcagctggagagaagtcggtctttctatggagcatgaggtcctacacgttaattcggaagctagagggccatcaaagcagtgttgtctcttgtgacttctcccccgactctgccctgcttgtcacggcttcttacgataccaatgtgattatgtgggacccctacaccggcgaaaggctgaggtcactccaccacacccaggttgaccccgccatggatgacagtgacgtccacattagctcactgagatctgtgtgcttctctccagaaggcttgtaccttgccacggtggcagatgacagactcctcaggatctgggccctggaactgaaaactcccattgcatttgctcctatgaccaatgggctttgctggcacattttttccacatggtggagtcattgccacagggacaagagatggccacgtccagttctggacagctcctagggtcctgtcctcactgaagcacttatgccggaaagcccttcgaagtttcctaacaacttaccaagtcctagcactgccaatccccaagaaaatgaaagagttcctcacatacaggactttttaagcaacaccacatcttgtgcttctttgtagcagggtaaatcgtcctgtcaaagggagttgctggaataatgggccaaacatctggtcttgcattgaaatagcatttctttgggattgtgaatagaatgtagcaaaaccagattccagtgtactagtcatggatttttc

SEQ ID NO: 23 - DNA, 422 bp, Human
accatggttccaagtcctctcccctgtggtcaagttgcccgaatgttgggcccaagtgccttttcctccttgggcctccccttctgacctgcaggacagttttccggagcccatttggtatgaggtattaattagccttaactaaattacaggggactcagaggccgtgctcctgaccgatccagacactattttttttttttttttttaacaatggtgtgcatgtgcaggaaatgacaaatttgtatgtcagattatacaaggatgtattcttaaaccgcatgactattcagatggctactgagttatcagtggccatttattagcatcatatttatttgtattttctcaacagatgttaaggtacaactgtgtttttctcgattatctaaaaaccatagtacttaaattgaaaaaaaaaa

SEQ ID NO: 24 - DNA, 2019 bp, Mouse
  UNSURE (2000): n is unsure
ggcacgaggcggggtcagggcggaggctgaggaccaagtaggcatggcggagggcgggaccggccccgatggacgggccggcccgggacccgcaggtcctaatctgaaggagtggctgagggagcagttctgtgaccatccactggagcactgtgacgatacaagactccatgatgcagcctatgtaggggacctccagaccctcaggaacctactgcaagaggagagctaccggagccgcatcaatgagaagtctgtctggtgctgcggctggcttccctgcacaccactgaggatcgcagccactgcaggccatgggaactgtgtggacttcctcatacgcaaaggggccgaggtggacctggtggatgtcaaggggcagactgccctgtatgtggctgtagtgaacgggcacttggagagcactgagatccttttggaagctggtgctgatcccaacggcagccggcaccaccgcagcactcctgtgtaccatgcctytcgtgtgggtagggacgacatcctgaaggctcttatcaggtatggggcagatgttgatgtcaaccatcatctgaattctgacacccggccccctttttcacggcggctaacctccttggtggtctgtcctctatacatcagtgctgcctaccataaccttcagtgcttcaggctgctcttgcaggctggggcaaatcctgacttcaattgcaatggccctgtcaacacccaggagttctacaggggatcccctgggtgtgtcatggatgctgtcctgcgccatggctgtgaagcagccttcgtgagtctgttggtagagtttggagccaacctgaacctggtgaagtgggaatccctgggcccagaggcaagaggcagaagaaagatggatcctgaggccttgcaggtctttaaagaggccagaagtattcccaggaccttgctgagtttgtgccgggtggctgtgagaagagctcttggcaaataccgactgcatctggttccctcgctgccgctgccagaccccataaagaagtttttgctttatgagtagcattcacatgcagtgctgactgcaatgtggaagccgatcacctgcagtgaaaactgacacagactctggcatcctgggaaccatggcctgtgctgccagcttgatccttggctgtcagtgaagaaaaaacggctgtgttctcttggactgtgattctatctcaggtgcttgggccatcgaacgctccttgagtcattgtcaactgagaggcacatacaaacttaattttgttcctcttcagtctctctgttttggattcttcctggcaatgtgtgcagcatgggctgagcctggtgattgccctagtggggaaggcttttttctccaggctatgcatctatttatgttcctactttgcaatttattgttcttttaaggcttgatatcaaaacagaaagaggtttgttaagaaaagatatagggagaaaggaattccggttccgtgcacttgctagcctgctttccttgcctgggtttgtctgtctatgctgcctggtgcacatcccttctctttgctgccactgttctattttgggagttgtcttccgtctaagatggcttctggggttctatcttattgcacagaggtcccagaacagtgttcatagggcaccatctgctctgccaagggttttctgatgtcttaccctggggatcttcagacagtggttacctttaggagacccacctggaactaaccattaagtgactgcccacattcagatcagggaccatcttaatagtactcactgccagtcctcacaagagaagatgacacgggtgctctcttcagacactcccatacaggaagttggaaaatgtcttggtcacctgggttgttcccaggctacaacttcttggtgttccactaaraccagratatcctagttttttgggttgactgttccctccccactttccttgaancccaatgcccntttgtktnggttgcttccctaaaaktt

SEQ ID NO: 25 - protein, 350 aa, Mouse
  UNSURE (167): Xaa is unsure
ARGGVRAEAEDQVGMAEGGTGPDGRAGPGPAGPNLKEWLREQFCDHPLEHCDDTRLHDAAYVGDLQTLRNLLQEESYRSRINEKSVWCCGWLPCTPLRIAATAGHGNCVDFLIRKGAEVDLVDVKGQTALYVAVVNGHLESTEILLEAGADPNGSRHHRSTPVYHAXRVGRDDILKALIRYGADVDVNHHLNSDTRPPFSRRLTSLVVCPLYISAAYHNLQCFRLLLQAGANPDFNCNGPVNTQEFYRGSPGCVMDAVLRHGCEAAFVSLLVEFGANLNLVKWESLGPEARGRRKMDPEALQVFKEARSIPRTLLSLCRVAVRRALGKYRLHLVPSLPLPDPIKKFLLYE

SEQ ID NO: 26 - DNA, 419 bp, Human
gcatccatggcggagggcggcagcacgacgggcgggcagggccgggctccgcaggtcgtaatctgaaggagtggctgagggagcaattttgtgatcatccgctggagcactgtgaggacacgaggctccatgatgcagcttacgtcggggacctccagaccctcaggagcctattgcaagaggagagctaccggagccgcatcaacgagaagtctgtctggtgctgtggctggctcccctgcacaccgttgcgaatcgcggccactgcaggccatgggagctgtgtggacttcctcatccggaagggggccgaggtggatctggtggacgtaaaaggacagacggccctgtatgtggctgtggtgaacgggcacctagagagtacccagatccttctcgaagctggcgcggaccccaac

SEQ ID NO: 27 - DNA, 595 bp, Human
gaggaagaagaaaagtggaccctgaggccttgcaggtctttaaagaggccagaagtgttcccagaaccttgctgtgtctgtgccgtgtggctgtgagaagagctcttggcaaaaccggcttcatctgattccttcgctgcctctgccagaccccataaagaagtttctactccatgagtagactccaagtgctgcggttgattccagtgagggagaaagtgatctgcagggaggtggacaccgagccctgagtgctgtgctgctgctggtctcctgatggctgttgctgcagaagatgtcctcgtagactgtcattgctcctcaggtgcctgggccgctgaacagtccttgggtcattgtcagctgagaggcttatactaaagttattattgtttttcccaagttctctgttctggattttcagttgcatattaatgtaacgggccatggggtatgtacatgtaggggctgaggttggaggcctactaatttcctgtagggaagactcccagcacttctggaactgtgcttctctttatttttctacttctcaatttgatggttcgattaaagccttctagtatctcaatgaaaa

SEQ ID NO: 28 - DNA, 896 bp, Mouse
  CDS (4)..(393)
  UNSURE (551): n is unsure
ctgatgtccgcaattctgaaggttggacaccactgctggctgcctgtgacatccgctgtcaatccccaaaggatgctgaggccaccaccaaccgctgttttcaactgtgccgcttgctgctgtctgtgggggcagatgctgatgaatacataccgtgtagttcagcttcctgaggaggccaagggcttggtgccaccagagattctacagaagtaccatggattctactcttccctctttgccttggtgaggcagcccaggtcgctgcagcatctctgccgttgtgcgctccgcagtcacctggagggctgtctgccccatgcactaccgcgccttcccctgccaccgcgcatgctccgctttctgcagctggactttgaggatctgctctactaggcttgctgccctgtgaacaaagcagaccccacccccaccccaagggcatctctcagcaatgaatgatgcaaggcggtctgtcttcaagtcaggagtggacgccttgatccacacttgagagaagaggccagatcagcaccyggctggtagtgatngcagagggcacctgtgcagatctgtgtgcgcactggaaatctctaggctgaaggcyagagcaaatggtgcargtgttagtccttgggangagagacaganggtgagaaagcaagacagaggtgagagtgcacatgtcaagtggtagattgccttaaaagaaagctaaaaaaagaaaaagattcgggcgaacttctttaggggtaatgctgcagcgtgttaaactgactgaccagcgtccatatctttggacccttcccgggtgaaaaagccccttcatcctccagcgctccccaagggtgcttagcaataccgggtgcttttctgccgcaaagtgagttaccaaa

SEQ ID NO: 29 - protein, 130 aa, Mouse
MSAILKVGHHCWLPVTSAVNPQRMLRPPPTAVFNCAACCCLWGQMLMNTYRVVQLPEEAKGLVPPEILQKYHGFYSSLFALVRQPRSLQHLCRCALRSHLEGCLPHALPRLPLPPRMLRFLQLDFEDLLY

SEQ ID NO: 30 - DNA, 436 bp, Mouse
gtgggggcgtcatcatgacctcctctagggctctgcaacatgactcctgtggtgcaaatcaacaaattgttcactgatgaatccacaaggatctctgggcctacaaccaggtcctggtccacatgactgtcgtcttcggagaaggcaccactcgcccccggcaggtacggctgacacctccatgggagaagacgtatccaggcagcagctgcgcggcccttcaagagggcacatcccgtcatctaaaggcacggtgtactgaaggtagtcctgagacatgagtccgattactacaggcacgtgttcctccaggtggaggctcaggtccccgggtgagctggggctgcagcgggactcagggcgcggctctggctgcaggtctcgcagctccctgggctgtagctcccgcagatccttgcgcacaccgttgactggt

SEQ ID NO: 31 - DNA, 2180 bp, Human
ttaatagtacctacatagtagaaaattataactccactttaaaacaatgttttctttctattcaaatcaatttaaaactttttataaacattaatgttgcaagagaatccagtccatttatgaaaattagttgacaatcaagttcacccaagaaaatgttgactaagctaaagaaatcacagataaaacattttaccaaaaggataggtaacacacaaaaaaatgctatcacaggaagctatgatcatctaatatttctttaataataattctagttccataggttttcatgttatgccaatttgtacccgagtttaattacagaaaaggcaacaatttctaaattggtggtatacatttctttacaattttttaatgtaaggccatttattaaaatagacaaactagaagatgaaaacgaaggcaacagaaaaattcaacttttcacaaccaaaagaattagcacaaccttagaaataatttagaaaaaagtgttgttaaaagatatgttgcagatctccgttccattacccaagattatgtcaattcacgattctaaataaatctttttaaagtaagagattaaaaactcatcttcagtgtatatgtaaattccgtggttttatcacacaggtatgtttattcaacactgctttggaaatggaccatttaaaaggacatggcaatttccattctgttaagtttcattcaacctttacttaggggttgattaccacatgaaatgtgcttttaatgcataaaaatcacagtggattagccagcaaaagggactgggcggggggggcattgaggagaatttgataattcacattgtgattattctgcacattgatgaaacataattcacacctctaaaacctcaagacttcccttttttaaagaaccaaaataaacccaagacaccttgctgacacttccccacccctaaacaaactgatgactcttttacacataaaactgaaatagttatggcagcaaaagattttgatggcaatgaaagtttgtaaactgtatttcaatctcttgttcttattcccaaagtgcaagatgcagggttctcaatctttcagtagtgcttctcctgtaaataatccttcattttgtttggcaaaggcagtttctgaattaagtctattctggtatactgacgtataacaaaacgacacaggtactgcaacgagcgcacctatgaaccccggaacactggttggcaagttctgacggaagtgcagattccaggcagcgagaccttgaataacaaaaagctcccattttcagagtccctgattgaatgctccaattagatcaactatggacgtatgtccttccacatcggctgttcataaaagctaaacctaccatttgagtgctcaattctagtgtgaagtgttttaccatgggagcgaaagtcacagcttaaaaggtaacggtcgtcagaactgtcccgaacaagaaaagaaccatctggcacgtttgctagcttcccttctgcctcccaacgtgtgattggtccccagtaccatccttgctttgcaagttttttcagctcctctgtaaggcttgtcacaaccatgggaccactactttgcactgagtcataaactcttgcaaccccaggagcagagttcggatcaaaattcaaatgacagcgcataactttcagccacgtggggctttctgtccagtgagtccactgaaagttcccctttgggatttggattattcctgcattggagtaaccaatggtgaagattggagggacatccatcgtgaacccgctctccggggttctgcaacatgactcccgtggtgccaatcaacaagccattcaccggactgatccacgaagatctctggggcgacaactaggtcctggtctacctgactctcatcctcggggaaagcgcgccctcccacttgaggaggaaccgcagagacttccatgggagaagagctgtccagacaatagctccgtgatccttccaaaggatacatcccctcatctaaaggcacagtatactgaatgtagtcctgaggcataagtccaataacgacaggcacatgttcatccaggtgaagatgcaggtctccattatgagaagccgagctcttcagtgaattggcttgctcctggcacgtggtctcagactggaggtcgt

SEQ ID NO: 32 - DNA, 2649 bp, Mouse
ggcacgaggctgtgtccagcacacagagagggcccggccatctgctttggttcagagccctgtgtctgtctgtcacttagactcttcctcccggctcgcagctcaccctccatcctccttactggctccagcatgactcgcttctcttatgcagagtactttgctctgtttcactctggctctgcaccttccaggtccccttcgtctcccgagaacccaccggcccgcgcacccctgggtctgttccaaggggtcatgcagaagtatagcagcaacctgttcaagacctcccagatggcggctatggaccccgtgctgaaggccatcaaggaaggggatgaagaggccttgaagatcatgatccaggatgggaagaatcttgcagagcccaacaaggagggctggctgccgctccacgaggctgcctactatggccagctgggctgcctgaaagtcctgcagcaagcctacccagggaccattgaccaacgcacactgcaggaagagacagcattatacctggccacatgcagagaacacctggattgcctcctgtcgctgctccaggcgggggcagagcctgacatctctaacaaatccagggagactccactttacaaagcctgtgagcgcaagaacgcggaggcggtgaggatattggtgcgatacaacgcagacgccaaccaccgctgtaacaggggctggaccgcactgcacgagtctgtctcccgcaatgacctggaggtcatggagatcctagtgagtggcggggccaaggtggaggccaagaatgtctacagcatcacccctttgtttgtggctgcccagagtgggcagctggaggccctgaggttcctggccaagcatggtgcagacatcaacacgcaggccagtgacagtgcatcagccctctacgaggccagcaagaatgagcatgaagacgtggtagagtttcttctctctcagggcgccgatgctaacaaagccaacaaggacggcctgctccccctgcatgttgcctccaagaagggcaactatagaatagtgcagatgctgctgcctgtgaccagccgcacgcgcgtgcgccgtagcggcatcagcccgctgcacctagcggccgagcgcaaccacgacgcggtgctggaggcgctgctggccgcgcgcttcgacgtgaacgcacctctggctcccgagcgcgcccgcctctacgaggaccgccgcagttctgcgctctacttcgctgtggtcaacaacaatgtgtacgccaccgagctgttgctgctggcgggcgcggaccccaaccgcgatgtcatcagccctctgctcgtggccatccgccacggctgcctgcgcaccatgcagctgctgttggaccatggcgccaacatcgacgcctacatcgccactcaccccaccgcctttccagccaccatcatgtttgccatgaagtgcctgtcgttactcaagttccttatggacctcggctgcgatggcgagccctgcttctcctgcctgtacggcaacgggccgcaccacccgccccgcgacctggccgcttccacgacgcacccgtggacgacaaggcacctagcgtggtgcagttctgtgagttcctgtcggccccggaagtgagccgctgggcgggacccatcatcgatgtcctcctggactatgtgggcaacgtgcagctgtgctcccggctgaaggagcacatcgacagctttgaggactgggctgtcatcaaggagaaggcagaacctccgagacctctggctcacctctgccggctgcgggttcggaaggccataggaaaataccggataaaactcctggacacactgccgcttcccggcaggctaatcagatacttgaaatatgagaatacacagtaaccagcctggagaggagatgtggccttcagactgtttccgggacgccccaggtggcctgcatccaggaccccctggggtcagaacaggtgtgaccttgctggttctttgctggagcttcacccaaagtgagaacctgatgtggggagtggacgtggaacctctgctttcacactgtcagcggatcgcagacccgctctgcttctggccatagccagagaccttcaacctggggccaggggagagctggtctgggcaaggtggcccaggcaggaatcctggccttaagctggagaacttgtaggaatccctcactggaccctcagctttcaggctgcgagggagacgcccagcccaagtattttatttccgtgacacaataacgttgtatcagaaaaaaaaaaaaacatgggcgcagcttattccttagtagggtatttacttgcatgcgcgcttaaagctactggaaacatgcgttccactatgcttgagaatccccttgcactggtaaacgagagccgacgtgcttcaaggttggatttttggttgcccctttggcgttccgcgggtttgtccgacgtaattgaccccgtgttttgtcactttcgagtgttccgactattggggggcttttggttgtccccaaaattgtgggtggtgtgcggacgccacgagaagtggttcatgggcgataatcattactggagaatgtagagcggcggttttacgaataaatattttttaagccgccttcccaaaa

SEQ ID NO: 33 - DNA, 495 bp, Human
cctcctgagagttcgccggcccgggcccaatgggttgttccaaggggtcatgcagaaatacagcagcagcttgttcaagacctcccagctggcgcctgcggaccccttgataaaggccatcaaggatgcgatgaagaggccttgaagaccatgatcaaggaagggaagaatctcgcagagcccaacaaggagggctggctgccgctgcacgaggccgcatactatggccaggtgggctgcctgaaagtcctgcagcgagcgtacccagggaccatcgaccagcgcaccctgcaggaggaaacagccgtttacttggcaacgtgcaggggccacctggactgtctcctgtcactgctccaagcaggggcagagcgggacatctccaacaaatcccgagagaaccgctctacaaagcctgtgagcgcaagaacgcggaagccgtgaagattcttggtgcagcacaacgcagacaccaacaacgctgcaaccgggctg

SEQ ID NO: 34 - DNA, 709 bp, Human
gtgcagctctgctcgcggctgaaggaacacatcgacagctttgaggactgggccgtcatcaaggagaaggcagaacctccaagacctctggctcacctttgccgactgcgggttcgaaaggccattgggaaataccgtataaaactcctagacaccttgccgctcccaggcaggctgattagatacctgaaatacgagaacacccagtaactggggccacggggagagaggagtagcccctcagactcttcttactaagtctcaggacgtcggtgttcccaactccaaggggacctggtgacagacgaggctgcaggctgcctccctctcagcctggacagctaccaggatctcactgggtctcagggcccagagctttggccagagcagagaacagaatgtgtcaaggagaagaatcatttgtttacaaactgatgagcagatcccagaccttctctaccttcaggaatggcagaaacctctattcctggggccagggcagagcttgaggtgttctggggaaggtggtgctcagagccttccctgtgcccctccacttgttctggaaaactcaccacttgacttcagagctttctctccaaagactaagatgaagacgtggcccaaggtagggggtagggggagcctgggtcttggagggctttgttaagtattaatataataaatgttacacatgtgaaaaaaaaaa

SEQ ID NO: 35 - DNA, 848 bp, Human
  CDS (1)..(621)
ttggagaagtgtggttggtattgggggccaatgaattgggaagatgcagagatgaagctgaaagggaaaccagatggttctttcctggtacgagacagttctgatcctcgttacatcctgagcctcagtttccgatcacagggtatcacccaccacactagaatggagcactacagaggaaccttcagcctgtggtgtcatcccaagtttgaggaccgctgtcaatctgttgtagagtttattaagagagccattatgcactccaagaatggaaagtttctctatttcttaagatccagggttccaggactgccaccaactcctgtccagctgctctatccagtgtcccgattcagcaatgtcaaatccctccagcacctttgcagattccggatacgacagctcgtcaggatagatcacatcccagatctcccactgcctaaacctctgatctcttatatccgaaagttctactactatgatcctcaggaagaggtatacctgtctctaaaggaagcgcagcgtcagtttccaaacagaagcaagaggtggaaccctccacgtagcgaggggctccctgctggtcaccaccaagggcatttggttgccaagctccagctttgaagaaccaaattaagctaccatgaaaagaagaggaaaagtgagggaacaggaaggttgggattctctgtgcagagactttggttccccacgcaagccctggggcttggaagaagcacatgaccgtactctgcgtggggctccacctcacacccacccctgggcatcttaggactggaggggctccttggaaaactggaagaagtctcaacactgtttctttttca

SEQ ID NO: 36 - protein, 207 aa, Human
LEKCGWYWGPMNWEDAEMKLKGKPDGSFLVRDSSDPRYILSLSFRSQGITHHTRMEHYRGTFSLWCHPKFEDRCQSVVEFIKRAIMHSKNGKFLYFLRSRVPGLPPTPVQLLYPVSRFSNVKSLQHLCRFRIRQLVRIDHIPDLPLPKPLISYIRKFYYYDPQEEVYLSLKEAQRQFPNRSKRWNPPRSEGLPAGHHQGHLVAKLQL

SEQ ID NO: 37 - DNA, 464 bp, Mouse
gttccaagcctaacccatctttgtcgtttggaaattcgggccagtctaaaagcagagcaccttcactctgacattttcatccatcagttgccacttcccagaagtctgcagaactatttgctctatgaagaggttttaagaatgaatgagattctagaaccagcagctaatcaggatggagaaaccagcaaggccacctgacacaggtcctttaattctgtttagtcacaaaagacggcttgtgtgactgtttggatttggtgatcaaatgtccatgtttacagttgcttttcccagtttgtgtctttcccaatattgtgaaccttatccatcttgccttactcagttttatttctagtgcactttgttgtgtattatttgtttacctgaccattttctactttattctgctaataaactgtaattctgaaaaaaaaaaaaaaaaaaaaaaaaaaaaaaaaaaa

SEQ ID NO: 38 - DNA, 747 bp, Human
ggggatcgaaagcgggggcttctgggacgcagctctggagacgcggcctcggaccagccatttcggtgtagaagtggcagcacggcagactggtcaaacaaatggattttacagaggcttacgcggacacgtgctctacagttggacttgctgccagggaaggcaatgttaaagtcttaaggaaactgctcaaaaagggccgaagtgtcgatgttgctgataacaggggatggatgccaattcatgaagcagcttatcacaactctgtagaatgtttgcaaatgttaattaatgcagattcatctgaaaactacattaagatgaagacctttgaaggtttctgtgctttgcatctcgctgcaagtcaaggacattggaaaatcgtacagattcttttagaagctggggcagatcctaatgcaactactttagaagaaacgacaccattgtttttagctgttgaaaatggacagatagatgtgttaaggctgttgcttcaacacggagcaaatgttaatggatcccattctatgtgtggatggaactccttgcaccaggcttcttttcaggaaaatgctgagatcataaaattgcttcttagaaaaggagcaaacaaggaatgccaggatgactttggaatcacacctttatttgtggctgctcagtatggccaagctagaaagctttgaagcatacttatttcatccgggtgcaaatgtcaattgtcaagccttggacaaagctacc

SEQ ID NO: 39 - DNA, 1018 bp, Human
cacaaatgggaccatacaaaaatcttggacttgttaataaccacttactaaccgggacctgtgacactgggctaaacaaagtaagtccctgtttactcagcagtgtttgggggacatgaaggattgcctagaaatattactccggaatggtctacagcccagacgcccaggcgtgccttgtttttggattcagttctcctgtgtgcatggctttccaaaaggaggtggagctgtagttctttggaattgtgaacattcttttgaaatatggagcccagataaatgaacttcatttggcatactgcctgaagtacgagaagttttcgatatttcgctactttttgaggaaaggttgctcattgggaccatggaaccatatatatgaatttgtaaatcatgcaattaaagcacaagcaaaatataaggagtggttgccacatcttctggttgctggatttgacccactgattctactgtgcaattcttggattgactcagtcagcattgacacccttatcttcactttggagtttactaattggaagacacttgcaccagctgttgaaaggatgctctctgctcgtgcctcaaacgcttggattctacagcaacatattgcccactgttccatccctgacccatctttgtcgtttggaaattcggtccagtctaaaatcagaacgtctacggtctgacagttatattagtcagctgccacttcccagaagcctacataattatttgctctatgaagacgttctgaggatgtatgaagttccagaactggcagctattcaagatggataaatcagtgaaactacttaacacagctaatttttttctctgaaaaatcatcgagacaaaagagccacagagtacaagtttttatgattttatagtcaaaagatgattattgattgtcagataggttaggttttggggggccagtagttcagtgagaatgtttatgtttacaactagccttcccagtaaaaaaaaaaaaaaaaaaaaaaaaaaa

SEQ ID NO: 40 - DNA, 1897 bp, Mouse
cggggggctgggacctggggcgtaaccgtctctaccacgacggcaagaaccagccaagtaaaacatacccagcctttctggagccggacgagacattcattgtccctgactcctttttcgtggccctggacatgratgatgggaccttaagtttcatcgtggatggacagtacatgggagtggctttccggggactcaagggtaaaaagctgtatcctgtagtgagtgccgtctggggccactgtgagatccgcatgcgctacttgaacggacttgatcctgagcccctgccactcatggacctgtgccggcgttcggtgcgcctagcgctgggaaaagagcgcctgggtgccatccccgctctgccgctacctgcctccctcaaagcctacctcctctaccagtgatccacatcccaggaccgccatacgacagccatctggtgccaartcactgagcccgttggggtccgccgacccctgcgcctgggatggaagcccacctcagccatgggcagacgtgccccctcatcctaccggctgcctctgctgggggaacctatgccaacggacttctcccttcccaacactggctgaagcagcagcacccaggcccttccctgaaccagatgcagagaataaactatgaaaacctctctcaggcgccttctgctctcaggtggagtgggctgccccccactctctgcagagagaggctacacccacctggggggtcctgggaggtaagactagtaggaggtgccagggctgartccaaaagcaggaatggccaggamcaggccatacagatgaagctcaggatgtcacataccatggacamtgagacagaaccccaggttggamttcccttgggccaacgagtgccagctttaatgtcagctgcmggtgctctgtggcctgtatttattctttaaacagtagcaaaggccatttatttattccacttagaaaggaaaccttggtgggtggyttccctcgatgtgctttcccccacctccctggaatgtgtgtgccacacctgtccttgtcccaggccaggactgtggcacatgagctggtgtgcacagatacacgtatgtcgtcgtgcatgacccctgactagttcctaagtagccctgcaccaagcaccagagcagaccccaagagaggcccgtgcaagtccccatgtccccaggtccctgcttctgttgccttgggactcatacaccggcacacgtgtttcagcctcttgacttccatgagcttcgaattttgcccccgattcttctgatatttcccattggcatcctccaaagctctgggcctggagggcattaggacacatggaatgagtggggtctccagcccctgggaaagccactggcaaggcaggattagaaagaccaagagcagggtggggcgccatgaagcctgtatgcctctcaggctcaagaccccgccacacacccactcaagcctcagaagtggtgtgtagggcagccccaggagaggaatgcctgtcctagcagcacgtacatggagcaccccacatgtgctccagccctctggctgtttctcttgctctagaatcaactccctacattgggaatgtagccatttggtagaggacttgcctagcctgcaggaagctcacgttccatcccctgcaccaaggagaatcaaagctcaggaggctgaggcaggaggattgctgtcagtggtgtacagaggtcatggccatcctgggctatattaaaccttgtcctttaagaaaaagaaaagaaatcaacttccattgaatctgagttctgctcatttctgcacaggtacaatagatgacttkatttgttgaaaaatgkttaatatatttacmtatatatatatttgtaagaagcatt

SEQ ID NO: 41 - protein, 134 aa, Mouse
  UNSURE (45): Xaa is unsure
GGWDLGRNRLYHDGKNQPSKTYPAFLEPDETFIVPDSFFVALDMXDGTLSFIVDGQYMGVAFRGLKGKKLYPVVSAVWGHCEIRMRYLNGLDPEPLPLMDLCRRSVRLALGKERLGAIPALPLPASLKAYLLYQ

SEQ ID NO: 42 - DNA, 265 bp, Human
aagggtaaaaaactgtatcctgtagtgagtgccgtctggggccactgtagatccgaatgcgctacttgaacggactcgatcccgagactgccgctcatggatttgtgccgtcgctcggtgcgcctggccctggggagggagcgcctgggggagaaccacacctgccgctgccggcttccctcaaggcctacctcctctaccagtgacgttcgccatcataccgccagcgcgacagccacctggtgccaactcactgagccgcctg

SEQ ID NO: 43 - DNA, 2438 bp, Mouse
aagtggcggcggtccctggagagcaggcggaggcagcggcaagtctgactctgggctgaccgtggagccggggcgggggctgacagccaggcctccgcctggcgggagccgcacgaggagcgggagtggccgggcctctcttccgcgcttgagcgagcgccgggtgatggcggtggtgatggcggcaggcgctcggacagctccgcttgagctgagctcggagagatccgtccagaaagtgcccagaagaaacttcctcttagaaaagctgaaaaacacartatttataacactggaaattgtaaagaatttgtttaaaatggctgaaaacaatagtaaaaatgtagatgtacggcctaaaacaagtcggagtcgaagtgctgacaggaaggatggttatgtgtggagtggaaagaagttgtcttggtccaaaaagagtgagagttgttctgaatctgaagccataggtactgttgagaatgttgaaattcctctaagaagccaagaaaggcagcttagctgttcgtccattgagttggacttagatcattcctgtgggcatagatttttaggccgatcccttaaacagaaactgcaagatgcggtggggcagtgttttccaataaagaattgtagtggccgacactctccagggcttccatctaaaagaaagattcatatcagtgaactcatgttagataagtgccctttcccacctcgctcagatttagcctttaggtggcattttattaaacgacacactgttcctatgagtcccaactcagatgaatgggtgagtgcagacctgtctgagaggaaactgagagatgctcagctgaaacgaagaaacacagaagatgacataccctgtttctcacataccaatggccagccttgtgtcataactgccaacagtgcttcgtgtacaggtggtcacataactggttctatgatgaacttggtcacaaacaacagcatagaagacagtgacatggattcagaggatgaaattataacgctgtgcacaagctccagaaaaaggaataagcccaggtgggaaatggaagaggagatcctgcagttggaggcacctcctaagttccacacccagatcgactacgtccactgccttgttccagacctccttcagatcagtaacaatccgtgctactggggtgtcatggacaaatatgcagccgaagctctgctggaaggaaagccagagggcacctttttacttcgagattcagcgcaggaagattatttattctctgttagttttagacgctacagtcgttctcttcatgctagaattgagcagtggaatcataactttagctttgatgcccatgatccttgtgtcttccattctcctgatattactgggctcctggaacactataaggaccccagtgcctgtatgttctttgagccgctcttgtccactcccttaatccggacgttccccttttccttgcagcatatttgcagaacggttatttgtaattgtacgacttacgatggcatcgatgcccttcccattccttcgcctatgaaattgtatctgaaggaataccattataaatcaaaagttaggttactcaggattgatgtgccagagcagcagtgatgcggagaggttagaatgtcgacctgcatacatattttcatttaatattttatttttcttatgcctctttgaatttttgtacaaaggcagttgaatcaaataaaactgtgccctaagttttaattccagatcaatttattttttttatgatacacttgttatatatttttaagcaggtgtttggttttgtttttaccatataaatttacatatggtccaggcatatttacaatttcaaggcattgcatatacatttgaatattctgtattttttaaataatcttttgttctttcctatgtgtgaaatattttgctaatctatgctatcagtattcttgtatgaccgaatagttacctattctcttttcatcttgaagattttcagtaaagagtgttgtaatcaatccattataatgtaattgacttttgtaatttgccaataggagtgttaaacaacaaaatgatttaaaatgaaacttaatgtattttcattttaaatattaactaaaccaagtttgtttgttagttattctagccaataagaaaagagaatgtagcatcctagaggtgtatttgttctgcagtttggcaggaccgtcagttagtccaaataaacatcccctcagcgtggaggcgaatggaacctgtgctcctttcttacgggaagctttgcaaagcaaaatagcagggttacaagcttggagttgttaaggcaactagagttttctctattaatttatagactgttgttgcacctacttagctcttttttgggaactctagttcccaggggaaaatacctcgtgcc

SEQ ID NO: 44 - protein, 542 aa, Mouse
  UNSURE (94): Xaa is unsure
SGGGPWRAGGGSGKSDSGLTVEPGRGLTARPPPGGSRTRSGSGRASLPRLSERRVMAVVMAAGARTAPLELSSERSVQKVPRRNFLLEKLKNTXFITLEIVKNLFKMAENNSKNVDVRPKTSRSRSADRKDGYVWSGKKLSWSKKSESCSESEAIGTVENVEIPLRSQERQLSCSSIELDLDHSCGHRFLGRSLKQKLQDAVGQCFPIKNCSGRHSPGLPSKRKIHISELMLDKCPFPPRSDLAFRWHFIKRHTVPMSPNSDEWVSADLSERKLRDAQLKRRNTEDDIPCFSHTNGQPCVITANSASCTGGHITGSMMNLVTNNSIEDSDMDSEDEIITLCTSSRKRNKPRWEMEEEILQLEAPPKFHTQIDYVHCLVPDLLQISNNPCYWGVMDKYAAEALLEGKPEGTFLLRDSAQEDYLFSVSFRRYSRSLHARIEQWNHNFSFDAHDPCVFHSPDITGLLEHYKDPSACMFFEPLLSTPLIRTFPFSLQHICRTVICNCTTYDGIDALPIPSPMKLYLKEYHYKSKVRLLRIDVPEQQ

SEQ ID NO: 45 - DNA, 5000 bp, Mouse
ccctctgggcaagccgccccccccccacccatctaccacacacacacacacacacacacacacacattcagaccttggggcaaaaacaaagcaaaataacaacaacaaaaacactgcctgtggaaagtccttacttcaggaaggttggcagatgaggagcaagggaacattttatcaggactgccacaaaggagtctttttttttaatggtttttcaagacagggtttctctgtatagccctggctgtcctggagctcactttgtagaccaggctggcctcgaactcagaaattcgcctgcctctgcctcctgagtgctgggattaaaggcgtgcagcaccatgtccaactggcattttctcaattaaggttcgttcctttcagataactctaggttctgggtcaagctgacacaaggctacacagcacagtttgtatgccacattcagttcagaagacacccaacctccctggaactggaacttatgcacatttgtgagcttccacttgggagtgggaacctgaactgggtcctctgcaagagcagccgtgctcttaactgctgagccatttcagcagcctcacatcagaattaagttagaaattagccgggtatgaatcatacccttagaatcctagcatctgaaagcagagctaagagaaacagggattcaagaccagctcttggctacagagcccgtcctgtcctaggatgggctacaagagactatttcaaagccatccaaacaacaataactacaacaacaacaaggttaaaattaggctgggcacagggtacacacctttaatgccaacactcaggaggcagaggcaggctgatcagtgtgagtttgagttcaacgtggtctacatagggagttctaggccagcagaggttacagtctctctctctctctctctctctctctctctctcacacacacacacacacacacacacacacacacacacacacacggtggcattatgggatttttttgggataaggtttctctgtctagccctggcatagattcactctgtagactaggctagccttgaactcagagatccgcctgcctctgcctcccaagtgctgggattataggtgttgcaccaccactgcccagccactttgggatttttgaactgttatcaagaggctttcgaggaggtcaaacttcaacagcaacctctccatgataatgtagctaatgatcaaacgacactcaaaacttaacccttaaagcacacatccaccagacagcgtgcccactcgtagttccattactcaggaggctgaagcaggaggatgaaggactaaggcttcagcaacctagggagccgcaggggacagtagtctcaatccctacattctcctgaacacaggagcaggagttcaggaagggtgtcaaggccgcttactgatcttagggcctcaggaatgactagctcaggcagagagagcaaaggtctccagtggagaagtctacacacacacacacacacacacacacacacacacacacacagaatccaaggcgatgacgtcatcaaagggttaattctagtctgggatgggggggagggtggggcacgcagctgtcaggtggctttggaaaaataaactgctgaagagtctgacgccagggagtcctgggagggacaagaggttacccactcaaagagtgtgctccacaaagcatgcgcgcttgtccacgtctggagtcgtcacttattttttgcctggattctttgtagccggtgggttctcaaggcggtaagtggtgtggccgccgtggtctgggaggtgacgatagggttaatcgtccacagagcccaggggcggagcgcgggcgggcgtccgcagccccgctggagccggaagcagtggctggtcaggggcgcttctagccttccctatctgtacttccacagaggtctctgcgagctagggggacagtgaggtgcggggtaggggcccggcgttagagccagcaaggggacggttcacggtaaggtctgagggagagagagctcctgagaaacttggggggcgcgacacagatagggtgaaagcagagtgatagacctgggatggttaggggaccaagggaagaccaggctggttggcatacaccggtgaacggatgggagtcctagggaaagatgatgcgcctaacagtcctttctgtctccacaccactccaggggacgatccggagctcaactttcaaaagcgagacgccccagcaagcctgttttgagaagttcttcagcggctctcctcatgggccagacggccctggcaaggggcagcagcagcacccctacctcgcaggctctgtactcggacttctctcctcccgagggcttggaggagctcctgtctgctccccctcctgacctggttgcccaacggcaccacggctggaaccccaaggattgctccgagaacatcgatgtcaaggaagggggtctgtgctttgagcggcgccctgtggcccagagcactgatggagtccgggggaaacggggctattcgagaggtctgcacgcctgggagatcagctggcccctggagcaaaggggcacacacgccgtggtgggcgtggccaccgccctcgccccgctgcaggctgaccactatgcggcgcttttgggcagcaacagcgagtcctggggctgggatattgggcggggaaaattgtatcatcagagtaagggcctcgaggccccccagtatccagctggacctcagggtgagcagctagtggtgccagagagactgctggtggttctggacatggaggaggggactcttggctactctattgggggcacgtacctgggaccagccttccgtggactgaaggggaggaccctctatccctctgtaagtgctgtttggggccagtgccaggtccgcatccgctacatgggcgaaagaagaggtgagatacggactaggtgtggggagatcactactcttggcaatggtttgggctggaaactcatggttggagcacaggaagtaggcttcttgtcactttggcctgtcacttagatggccttggatctagcttcactcccaatccctattggatgtgatgcacaaattcagagcctttgggtctccctcagctgaggtggcggtggaaatggaggaagaaggaagggtgcctgagcaggatctcaagttcaaggatgcctggagttgcttacttaccttgtcttccttctctctccgcagtggaggaaccacaatcccttctgcacctgagccgcctgtgtgtgcgccatgctctgggggacacccggctgggtcaaatatccactctgcctttgccccctgccatgaagcgctatctgctctacaaatgacccagtagtacagggtgtgctggcaccctaccgtggggacaggtggagaggcacccgctggcctagacaactttaaaaagctggtgaagctggggggggggggctggaccccttcacctccccttctcacaggagcaagacatatagaaatgatattaaacaccatggcagcctgggacaaagaggtttttgaagtaaaaaatgagatgtattgtcacaacctgtttcattattgttttttgttttgttttacactcccccaccccaggctagagccccatcactgtcttaaggaattatgacaacccacaaagctcaggcccaggtgtttatttcccttacatgtaggatggttcacaaacacaatacaggggctttggcaccgtgggggaggggactatcccaggcctcttagggtctcatgtataccgaattcagacccgaaagctctgaatttctgcatcagacatccagtagaacttgggagtgaagctagagccaaggccatctaagtgacaggccaaagtgacacgaagcccacttcctgtgctccaaccatgagtttccagcccaaaccaatggaaggtgatttcacttgtcagggcccaaagggacagtcagttctactccctcccctcactaggagccaccttggtgacagttgattctacccactgtaagtggtaaagggattggcctggtcccaaccataatagggcggtggaaacggctcaggagggtacagcgtggattaggccacaagatggggcagatgatgtcatcagaagcatgtgaccggtgggagcagttactaaacttctgggcaacctagtccatgctatgcaggcaggtagagggatgggcagtgctcattgtttggcattgatgatgtccacaaattcaggcttgagagatgcgccacccacaaggaagccgtccacgtcaggctggcttgccagctctttgcaggttgctccagtcacagaacctgtaccaggaacaagaagacagtttggtcaggtctatgatcagaacacttaagccccacctctctgtgcaaggcagcctcagtctgtcttagcccatttccgtcttagctagagccaaagccactcacctccataaatgatccgggtgctctgagccaccccatcattgacattggatttcagccatccccggagcttctcgtgtacttcctgtgcctagaaggaggaggcagagctactaagtaagctccttcctatctatcattcaaggagtaaaaaccactggttctcacatagagttgagtttccagaaaagccccgggaccagagagtggcaaggctccaatcccaccaggcttggaatgaacatttttggcaaagtcactctccttggtgagtttgggggccctctgtctctaaaggggcttggatgggctccatagctgtgtgagtctgttaaagccggacaggctgaggagctctgggtagttacctgctgaggggttgccgtcttgccagtcccaatggcccacacaggttcataggccaggaccaccttgctccagtctttcacattatctgtggggcagagaggagagtgagtaggaaggagctgacccgccaagc

SEQ ID NO: 46 - protein, 264 aa, Mouse
MGQTALARGSSSTPTSQALYSDFSPPEGLEELLSAPPPDLVAQRHHGWNPKDCSENIDVKEGGLCFERRPVAQSTDGVRGKRGYSRGLHAWEISWPLEQRGTHAVVGVATALAPLQADHYAALLGSNSESWGWDIGRGKLYHQSKGLEAPQYPAGPQGEQLVVPERLLVVLDMEEGTLGYSIGGTYLGPAFRGLKGRTLYPSVSAVWGQCQVRIRYMGERRVEEPQSLLHLSRLCVRHALGDTRLGQISTLPLPPAMKRYLLYK

SEQ ID NO: 47 - DNA, 5615 bp, Human
gtactttctttatatctccataattttatttactattactacatgatacattattttataaaagtctttgtaacctccttaaggattcactgcttaatctccagtgcttagcacaaatcattaaatgcgaaccagaaactcttccaaatgtgttacatctataacctcattggattctcactaccaaccccatgcaatagatactaatgtgatctctgtcttacagaggaagaaacaggcacagggaggttcagtaatttgcccaaggtcatacacacactggccttcaggtattcatgcccggggagtctggtcccacagctggcatgtttgccattatattatattgcctccttatagtgtcggcactcattaagcacattgacagctatgcttggtgagtgactactatgtacccagctctgtgctacatgctttacctggattatttcaactgcacaacaaccctgtgaggtaactaccatcattgctcctattttacataacagaaaactacagaaatctggggctgggcgtagtggctcatgcctgaaatcccagcactttgggagaccctgtctctaaaaaaaatttttttttggccggacgtggtggctcacacctgtaatctcagcactttgggaggctaaggcaggcagatcacaaggtcaggagttctagaccagcctggccaacatggcaaaaccctgtgtctactaaaaatacaaaaaatagctaggcgtggtggcaggtgcctgtaatcccagctactcaggaggctgaggcaggagaatcccctgaacctgggagatggaggttacagagagccgagatcgtgccgctgcactccagcctgggcaacaagagcaagactctgtctcgaaaaaaataaaaataaaaataaaaatatttttttaaaaattagctgggtgtggtagcacatgcctgtagtcccagctacttgggaggctgaggtaggaggatcacttgagcccaggaggtcaaggctgcagtgggctgtgatggcgccactgcactctagccttggtgacagcaagaccctgtctcaaaaaaaaaaaaaagagaaatcgggcaacttccccaagatcgcgcagttaactagtggcatagcttcactcaaactcgaagtcttaatcaggacactctaccaaatgagatcaacggctcagtaatggattggcatccagtatgaagactggaccagcagggagaactatgatgcgtacagcctagagcctgaagcagatttcacagcctcagaggtggcacaggctgactcacaacccggggcagaaagggaccagcccagaaacagtgacccagaatcacagggaagtagaaatgggattcggcacaatgaagcccctccttgaccccatgctccttaccctcaggggcgcaggagttagtcgctcaggcggctcaaaggtcttgacggtggagaacaccatccccagggattcccgacgcggtgatgccatcaaagcgttaattctgagatgggcctgcccgggtgcggactctgccgcagcaagagaagggttaactgccccgggccttcgccgtgggggcggggcctcggggagggtcacagcccgggactgagacccgaggttaaccgcccggggtgggctccacgggggcggggcatgctctccgcggctgctgccggtatagagcggtaactgcccaggagggggcggggccccacaggggcgtggcctcggagctgcacggccgtgggcggcgatgagagggttaagccccagagggccctggaggggcggggccgcgggacgggctcggcccaagggaggagctgggggcggaagcggccggcggtctgcgccctgcgcgcctcggcttctttccgcccggctccttcagaggcccggcgacctccagggctgggaagtcaaccgaggttcgggggcagcggcgagggctccgggcgagtaagggggatggtccatgctgaggcccaaatggggcgaactcgcgagagtctctggcgacctggatcagatggggcgagggcagatgaagggcccaggagctttggggcagcgaggagggaggagcgggcccgttggcaaacttgggtgaaaggatggggtacctgggtgacgagcccccgccaggattctgctcttcacgccccttttctcccagctcccttccaggtcaatccaaactggagctcaactttcagaagagaaagacgccccagcaagcctctttcggggagtcctctagctcctcacctccatgggccagacagctctggcagggggcagcagcagcacccccacgccacaggccctgtaccctgacctctcctgtcccgagggcttggaagagctgctgtctgcaccccctcctgacctgggggcccagcggcgccacggttggaaccccaaagactgttcagagaacatcgaggtcaaggaaggagggttgtactttgagcggcggcccgtggcccagagcactgatggggcccggggtaagaggggctattcaaggggcctgcacgcctgggagatcagctggcccctagagcagaggggcacgcatgccgtggtgggcgtggccacggccctcgccccgctgcagactgaccactacgcggcgctgctgggcagcaacagcgagtcgtggggctgggacatcgggcgggggaagctgtaccatcagagcaaggggcccggagccccccagtatccagcgggaactcagggtgagcagctggaggtgccagagagactgctggtggttctggacatggaggagggaactctgggctacgctattgggggcacctacctggggccagcattccgcggactgaagggcaggaccctctatccggcagtaagcgctgtctggggccagtgccaggtccgcatccgctacctgggcgaaaggagaggtgaggcctggggcagacgtggggagaactttctgtccctggtggcagtggtttgggatggaaactcttctgacaagagcagaggggatggaccttcatccagcctgcctcaacctctgttcagtgctgggaaaggctaggggtcttcacagctgttatttaatttaacccaacagcaatagaggtgaaacaggcttgagaaagcaactttctcaagttctcttggccagtaaatggtgaaccttcagaatggagggaggaactgcagggatgagagaattcaggagatatcaacccctgagcaagaggtgcaaagcgttaggtactgggtttgatgtacaggtccaaaagaaggatgggcagagccaggtacccaggctgtataccggattccctgggctctaacctgtctctgtgccacatacctacttccttcctcagccacacctctggatggagacactggggccctgggcaccagggaggagagcagtggaggaggcagggccttagggtggggcagcaggggaggagcctccccaggaactgactgggtccagggcttggagctgctctctgcagttgtgtgggctgtagagtggagggccatccctcctcacctcagccccagctcccaagcctctggagtcaaagcctgggccagctccaccactgtcagagccaccttggcctgttgtttagagggccttagccagctcttcacccccagctctgactagggatgtgtgaaatcttatctgggaggcagaacttccgggtatctcaaattcccctttcagccaggtgggcacactcgaagcaggaaagcagaaaggcatctgagtaggaccccgtagtttgaggacatctggctggtggctgcacccatacttacattcccctccttctctctcccagcggagccacactcccttctgcacctgagccgcctgtgtgtgcgccacaacctgggggatacccggctcggccaggtgtctgccctgcccttgccccctgccatgaagcgctacctgctctaccagtgagccctgtgataccacagactgtgctgaggtcttgccaccacccctccccttggggaggtggggaggcactgctggcctagaccagctgctgaaagctggtgaggctgagcccctaccccaacccaagctctgcggaaatcaacagccccagagccacttggagggaggaagaaagggagccggcgttcaaggctatgacagtctgctacgcaaaacattttttcaagtaaaaatagtaagagatgttgttatagaaacctgttcttgtttttttttttttcttgcacaaatgatcatttatatagctgcctcaaaaaggaagattatctgggcaagtccagtgaaggcagacaaaccacaagacctagtgccaggtttattccctcacatgggtggttcacatacacagcacagaggcacgggcaccatgggagagggcagcactcctgccttctgaggggatcttggcctcacggtgtaagaagggagaggatggtttctcttctgccctcactagggcctagggaacccaggagcaaatcccaccacgccttccatctctcagccaaggagaagccaccttggtgacgtttagttccaaccattatagtaagtggagaagggattggcctggtcccaaccattacagggtgaagatataaacagtaaaggaagatacagtttggatgaggccacaggaaggagcagatgacaccatcagaagcatatgcagggaaagggcagttactgggcttctgggctgcttagtccctggcttggcaggaagggtagggaagatggatggggctcattgtttggcattgatgatgtccacgaattcgggcttgagggaagcaccacccacaaggaagccatccacatcaggctggctggccagctccttgcaggttgccccagtcacagagcctgggaagggagcagaacaagggcttggtcaagaatgggatgagtctgccccatccccacctccatgtccgagggctcagtctagtcctcagcccactccacctcagccgggaaccaaagccactcacctccataaatgatacgggtgctctgagccaccgcatcagagacgttggacttcagccatcctcggagcttctcgtgtacttcctgggcctagaacaagaagctggcctaagtaagaccttttctgcctctctaagaggaaaaatcactggcaccagtggacacttagtgtggtttctgactgagtcagagtaccagggctctgatccaagccaggccctggactggatgcccttggacaagtcactgtctctgggttcaaggtctctgtgtctttgaaataaggggttgccccatgtgggctgtgtctgtccaaacctattgaggcaggctgggatgagggcagggctcctgggcccggttacctgttggggtgttgcagtcttgccagtaccaatggcccacacaggctcataggccaggacgaccttgctccagtccttcacgttatctgcagggcagagatacagatggagggaagggtgaacaagaaagagctctccagccaggttctccggagtacgaagaacggtggcctactgccccctagtggacattggggg

SEQ ID NO: 48 - protein, 263 aa, Human
MGQTALAGGSSSTPTPQALYPDLSCPEGLEELLSAPPPDLGAQRRHGWNPKDCSENIEVKEGGLYFERRPVAQSTDGARGKRGYSRGLHAWEISWPLEQRGTHAVVGVATALAPLQTDHYAALLGSNSESWGWDIGRGKLYHQSKGPGAPQYPAGTQGEQLEVPERLLVVLDMEEGTLGYAIGGTYLGPAFRGLKGRTLYPAVSAVWGQCQVRIRYLGERRAEPHSLLHLSRLCVRHNLGDTRLGQVSALPLPPAMKRYLLYQ

SEQ ID NO: 49 - DNA, 28 bp, Artificial Sequence
  Description of Artificial SequenceSynthetic
agctagatctggaccctacaatggcagc

SEQ ID NO: 50 - DNA, 36 bp, Artificial Sequence
  Description of Artificial SequenceSynthetic
agctagatctgccatcctactcgaggggccagctgg

SEQ ID NO: 51 - protein, 18 aa, Artificial Sequence
  Description of Artificial SequenceN-terminal GFP tag
MARQSKGEELFTELYKTR

SEQ ID NO: 52 - DNA, 30 bp, Artificial Sequence
  Description of Artificial Sequence3′ genomic oligonucleotide no.3243
aagtccgttcaagtagcgcatgcggatctc

SEQ ID NO: 53 - DNA, 30 bp, Artificial Sequence
  Description of Artificial Sequence5′ genomic oligonucleotide no.3244
gagatccgcatgcgctacttgaacggactt

SEQ ID NO: 54 - protein, 10 aa, Artificial Sequence
  Description of Artificial Sequence Amino acid sequence encoded by SEQ ID NO53
EIRMRYLNGL

SEQ ID NO: 55 - DNA, 34 bp, Artificial Sequence
  Description of Artificial Sequence3′ cDNA oligonucleotide no.3245
agctacgcgtctggtagaggaggtaggctttgag

SEQ ID NO: 56 - DNA, 822 bp, Mouse
  CDS (1)..(822)
atgggtcagaaggtcacgggagggatcaagactgtggacatgcgggaccccacataccgacctctgaagcaggaactccaggggctggattactgcaagcccacccggctggacctgctgctcgacatgccccccgtgtcctacgatgtgcagctgctccactcctggaacaataacgaccgttcgctcaacgtcttcgtgaaggaagatgacaagttgatctttcaccggcatccggtggcccagagcacggacgccatcaggggcaaagttgggtacacacgtggactgcacgtatggcagatcacatgggccatgaggcagcgaggcacgcatgccgtggtgggggtggccacagcagatgcccctttgcactccgttggggacacaacccttgtaggaaataaccatgaatcctggggctgggacctggggcgtaaccgtctctaccacgacggcaagaaccagccaagtaaaacatacccagcctttctggagccggacgagacattcattgtccctgactcctttctcgtggccctggacatggatgatgggaccttaagtttcatcgtggatggacagtacatgggagtggctttccggggactcaagggtaaaaagctgtatcctgtagtgagtgccgtctggggccactgtgagatccgcatgcgctacttgaacggacttgatcctgagcccctgccactcatggacctgtgccggcgttcggtgcgcctagcgctgggaaaggagcgcctgggtgccatccccgctctgccgctacctgcctccctcaaagcctacctcctctaccagtga

SEQ ID NO: 57 - protein, 273 aa, Mouse
MGQKVTGGIKTVDMRDPTYRPLKQELQGLDYCKPTRLDLLLDMPPVSYDVQLLHSWNNNDRSLNVFVKEDDKLIFHRHPVAQSTDAIRGKVGYTRGLHVWQITWAMRQRGTHAVVGVATADAPLHSVGYTTLVGNNHESWGWDLGRNRLYHDGKNQPSKTYPAFLEPDETFIVPDSFLVALDMDDGTLSFIVDGQYMGVAFRGLKGKKLYPVVSAVWGHCEIRMRYLNGLDPEPLPLMDLCRRSVRLALGKERLGAIPALPLPASLKAYLLYQ

SEQ ID NO: 58 - DNA, 35 bp, Artificial Sequence
  Description of Artificial Sequence Oligonucleotide no.3342
agctggcgcgccagggtcagaaggtcacgggaggg

SEQ ID NO: 59 - protein, 128 aa, Mus musculus
  UNSURE (1): Xaa is Leu, Ile, Val, Met, Ala or Pro
XXXXXXXXXXXXXXXXXXXXXXXXXXXXXXXXXXXXXXXXXXXXXXXXXXXXXXXXXXXXXXXXXXXXXXPXXXXXXXXXXXXXXXXXXXXXXXXXXXXXXXXXXXXXXXXXXXXXXXXXXXXXXXXX

SEQ ID NO: 60 - DNA, 1611 bp, Mouse
  CDS (1)..(1611)
atggataaagtggggaaaatgtggaacaacttaaaatacagatgccagaatctcttcagccacgagggaggaagccgtaatgagaacgtggagatgaaccccaacagatgtccgtctgtcaaagagaaaagcatcagtctgggagaggcagctccccagcaagagagcagtcccttaagagaaaatgttgccttacagctgggactgagcccttccaagaccttttccaggcggaaccaaaactgtgccgcagagatccctcaagtggttgaaatcagcatcgagaaagacagtgactcgggtgccaccccaggaacgaggcttgcacggagagactcctactcgcggcacgccccgtggggaggaaagaagaaacattcctgttccacaaagacccagagttcattggataccgagaaaaagtttggtagaactcgaagcggccttcagaggcgagagcggcgctatggagtcagctccatgcaggacatggacagcgtttctagccgcacggtcgggagccgctccctgaggcagaggctccaggacacggtgggtttgtgttttcccatgagaacttacagcaagcagtcaaagccactcttttccaataaaagaaaaatacatctttctgaattaatgctggagaaatgcccttttcctgctggctcggatttagcacaaaagtggcatttgattaaacagcataccgcccctgtgagcccacactcaacattttttgatacatttgatccatcactggtgtctacagaagatgaagaagataggcttcgcgagagaagacggcttagtatcgaagaaggggtggatccccctcccaacgcacaaatacacacctttgaagctactgcacaggtcaacccattgtataagctgggaccaaagttagctcctgggatgacagagataagtggagatggttctgcaattccacaaacgaattgtgactcagaagaggattcaaccaccctatgtctgcagtcacggaggcagaagcagcgccaggtgtccggggacagccacgcgcacgttagcagacagggagcttggaaagttcatacgcagatcgattacatacactgcctcgtgccagatttgcttcagatcacagggaatccctgttactggggcgtgatggaccgatacgaggccgaagcccttctagaagggaaaccggaaggcacgttcttgctcagggactctgcacaggaggactacctcttctctgtgagcttccgccgctacaacaggtctctgcacgcccggatcgagcagtggaaccacaacttcagcttcgatgcccatgacccctgcgtgtttcactcctccacagtcacggggcttctcgaacactataaagaccccagctcttgcatgttttttgaaccgttgctaacgatatcactgaatagaactttccctttcagcctgcagtatatctgccgcgcagtgatctgcagatgcactacgtatgatgggattgacgggctcccgctaccgtcgatgttacaggattttttaaaagagtatcattataaacaaaaagttagggttcgctggttagaacgagagccagtcaaagcaaagtaa

SEQ ID NO: 61 - protein, 536 aa, Mouse
MDKVGKMWNNLKYRCQNLFSHEGGSRNENVEMNPNRCPSVKEKSISLGEAAPQQESSPLRENVALQLGLSPSKTFSRRNQNCAAEIPQVVEISIEKDSDSGATPGTRLARRDSYSRHAPWGGKKKHSCSTKTQSSLDTEKKFGRTRSGLQRRERRYGVSSMQDMDSVSSRTVGSRSLRQRLQDTVGLCFPMRTYSKQSKPLFSNKRKIHLSELMLEKCPFPAGSDLAQKWHLIKQHTAPVSPHSTFFDTFDPSLVSTEDEEDRLRERRRLSIEEGVDPPPNAQIHTFEATAQVNPLYKLGPKLAPGMTEISGDGSAIPQTNCDSEEDSTTLCLQSRRQKQRQVSGDSHAHVSRQGAWKVHTQIDYIHCLVPDLLQITGNPCYWGVMDRYEAEALLEGKPEGTFLLRDSAQEDYLFSVSFRRYNRSLHARIEQWNHNFSFDAHDPCVFHSSTVTGLLEHYKDPSSCMFFEPLLTISLNRTFPFSLQYICRAVICRCTTYDGIDGLPLPSMLQDFLKEYHYKQKVRVRWLEREPVKAK

SEQ ID NO: 62 - DNA, 1601 bp, Mouse
  CDS (1)..(1599)
atgaagaaaatcagtctgaagaccttcaggaaatcttttaacctgagtaaaagcaaagacgaaactgagttcatggtggttcagccccagtcccttgctggtgacttcgtgaaagatgactctttattcgggagctgttatggcaaagacatggccagttgtgacattggcagcgaggatgagaaagggaagaacagatccaaaagcgagagcctgatgggcactttgaagaggcggttgtccgccaagcagaagaccaagggcaagggcggcactgcgtctacagatgaggacaccttctcctcagcttcagctcctggtgggctcaaggatgtgcgtgctccgcggcccatccgctccacatcactgagaagccaccattatagccccacgccctggccgctgcgtcccaccagctcggaggagacgtgcatcaagatggagatgcgagtgaaagcactggtgcatgctgccagcccaggaccagtcaacggtgtgcgcaaggatctgcgggagctacagcccagggagctgcgagacctgcagccagagccgcgccctgagtcccgctgcagccccagctcacccggggacctgagcctccacctggaggaacacgtgcctgtagtaatcggactcatgtctcaggactaccttcagtacaccgtgcctttagatgacgggatgtgccctcttgaagggccgcgcagctgctgcctggatacgtcttctcccatggaggtgtcagccgtacccctgccgggggcgagtggtgccttctccgaagacgacagtcatgtggaccaggacctggttgtaggcccagagatccttgtggattcatcagtgaacaatttgttgattggcaccacaggagtcatgttgcagagccctagaggaggtcatgatgacgcccctcccctctcaccattgctacctccaatgcagaataacccaatccaaaggaacttcagtggcctctcgggcccagacttgcacatggccgaaagtgttcgctgtcatttgaatttcgatcccaactctgcgcctggggttgctagagtttatgactcggtgcaaagtagtggccccatggttgttacaagtcttacggaggagctgaagaagcttgcaaaacaggggtggtattggggccccatcacacgctgggaggcagaggggaagttggcaaatgtgccagatggttcttttcttgtaagggatagttctgatgaccgttaccttttaagcctgagctttcgttcccatggtaaaacacttcacactagaattgagcactcaaatggtagattcagcttttatgaacagccagatgtggaagggcatacatctatagttgacttaatcgagcattcaatcagggactctgaaaatggagcattttgttattcaagatctcgattgcctggatcagcaacttacccagtcagactgaccaatccagtgtcacgattcatgcaggtgcgctcgctgcagtacctgtgccgctttgttatccgtcagtacaccagaatagacttaattcagaaactgcctttgccaaacaaaatgaaggattatttgcaggagaagcactactg

SEQ ID NO: 63 - protein, 533 aa, Mouse
MKKISLKTFRKSFNLSKSKDETEFMVVQPQSLAGDFVKDDSLFGSCYGKDMASCDIGSEDEKGKNRSKSESLMGTLKRRLSAKQKTKGKGGTASTDEDTFSSASAPGGLKDVRAPRPIRSTSLRSHHYSPTPWPLRPTSSEETCIKMEMRVKALVHAASPGPVNGVRKDLRELQPRELRDLQPEPRPESRCSPSSPGDLSLHLEEHVPVVIGLMSQDYLQYTVPLDDGMCPLEGPRSCCLDTSSPMEVSAVPLPGASGAFSEDDSHVDQDLVVGPEILVDSSVNNLLIGTTGVMLQSPRGGHDDAPPLSPLLPPMQNNPIQRNFSGLSGPDLHMAESVRCHLNFDPNSAPGVARVYDSVQSSGPMVVTSLTEELKKLAKQGWYWGPITRWEAEGKLANVPDGSFLVRDSSDDRYLLSLSFRSHGKTLHTRIEHSNGRFSFYEQPDVEGHTSIVDLIEHSIRDSENGAFCYSRSRLPGSATYPVRLTNPVSRFMQVRSLQYLCRFVIRQYTRIDLIQKLPLPNKMKDYLQEKHY

SEQ ID NO: 64 - protein, 34 aa, Mus musculus or Rattus norvegicus
VRPLQELCRQRIVAAVGRENLARIPLNPVLRDYL

SEQ ID NO: 65 - protein, 32 aa, Mus musculus
APTLQHFCRLAINKCTGTIWGLPLPTRLKDYL

SEQ ID NO: 66 - protein, 33 aa, Mus musculus
VATLQHLCRKTVNGHLDSYEKVTQLPGPIREFL

SEQ ID NO: 67 - protein, 34 aa, Homo sapiens
VRPLQELCRQRIVATVGRENLARIPLNPVLRDYL

SEQ ID NO: 68 - protein, 34 aa, Mus musculus
VPSLQHICRMSIRRVMSTQEVQKLPVPSKILAFL

SEQ ID NO: 69 - protein, 34 aa, Mus musculus
PFSLQYICRAVICRCTTYDGIDGLPLPSMLQDFL

SEQ ID NO: 70 - protein, 37 aa, Mus musculus
PRTLLSLCRVAVRRALGKYRLHLVPSLPLPDPIKKFL

SEQ ID NO: 71 - protein, 37 aa, Mus musculus
PRSLQHLCRCALRSHLEGCLPHALPRLPLPPRMLRFL

SEQ ID NO: 72 - protein, 34 aa, Homo sapiens
VRSLQYLCRFVICQYTRIDLIQKLPLPNKMKDYL

SEQ ID NO: 73 - protein, 37 aa, Mus musculus
PRPLAHLCRLRVRKAIGKYRIKLLDTLPLPGRLIRYL

SEQ ID NO: 74 - protein, 34 aa, Homo sapiens
VKSLQHLCRFRIRQYTRIDHIPDLPLPKPLISYI

SEQ ID NO: 75 - protein, 40 aa, Mus musculus
VPSLTHLCRLEIRASLKAEHLHSDIFIHQLPLPRSLQNYL

SEQ ID NO: 76 - protein, 37 aa, Mus musculus
PLPLMDLCRRSVRLALGKERLGAIPALPLPASLKAYL

SEQ ID NO: 77 - protein, 34 aa, Mus musculus
PFSLQHICRTVICNCTTYDGIDALPIPSPMKLYL

SEQ ID NO: 78 - protein, 37 aa, Mus musculus
PQSLLHLSRLCVRHALGDTRLGQISTLPLPPAMKRYL

SEQ ID NO: 79 - protein, 37 aa, Homo sapiens
PHSLLHLSRLCVRHNLGDTRLGQVSALPLPPAMKRYL

SEQ ID NO: 80 - protein, 34 aa, Mus musculus
LSSLKHLCRKALRSFLTTYQVLALPIPKKMKEFL

SEQ ID NO: 81 - protein, 34 aa, Mouse
VRSLQYLCRFVIRQYTRIDLIQKLPLPNKMKDYL

(19) United States
(12) Patent Application Publication
(10) Pub. No.: US 2008/0166730 A1
(43) Pub. Date: Jul. 10, 2008

(54) Therapeutic and diagnostic proteins comprising a SOCS box

(75) Inventors: Douglas J. Hilton, Warrandyte (AU); Warren S. Alexander, Moonee Ponds (AU); Elizabeth M. Viney, Bundoora (AU); Tracy A. Willson, North Balwyn (AU); Rachael T. Richardson, East Brighton (AU); Robyn Starr, Carlton (AU); Sandra E. Nicholson, Newport (AU); Donald Metcalf, Balwyn (AU); Nicos A. Nicola, Mont Albert (AU)

Correspondence Address: Scully, Scott, Murphy & Presser, 400 Garden City Plaza, Garden City, NY, 11530, US

(73) Assignee: The Walter and Eliza Hall Institute of Medical Research, Parkville (AU)

(21) Appl. No.: 11/977,132

(22) Filed: Oct. 23, 2007

Related U.S. Application Data
(63) Continuation of application No. 11/214,199, filed on Aug. 29, 2005, now abandoned.
Application No. 11/214,199 is a continuation of application No. 09/908,805, filed on Jul. 19, 2001, now Pat. No. 7,049,418.
Application No. 09/908,805 is a division of application No. 09/302,769, filed on Apr. 30, 1999, now Pat. No. 6,323,317.
Application No. 09/302,769 is a continuation-in-part of application No. 08/962,560, filed on Oct. 31, 1997, now Pat. No. 6,905,842.

(30) Foreign Application Priority Data
Nov. 1, 1996 (AU) PO3384/96
Feb. 14, 1997 (AU) PO5117/97
Oct. 31, 1997 (AU) PCT/AU97/00729

(51) Int. Cl. C12Q 1/68

(52) U.S. Cl. 435/6

(57) ABSTRACT

The present invention relates generally to therapeutic and diagnostic agents. More particularly, the present invention provides therapeutic molecules capable of modulating signal transduction such as but not limited to cytokine-mediated signal transduction. The molecules of the present invention are useful, therefore, in modulating cellular responsiveness to cytokines as well as other mediators of signal transduction such as endogenous or exogenous molecules, antigens, microbes and microbial products, viruses or components thereof, ions, hormones and parasites.

### CROSS-REFERENCE TO RELATED APPLICATIONS

This application is a continuation of application Ser. No. 09/908,805, filed Jul. 19, 2005, which is a divisional of application Ser. No. 09/302,769, filed Apr. 30, 1999, now U.S. Pat. No. 6,323,317, which is a continuation-in-part of Ser. No. 08/962,560, filed Oct. 31, 1997, now U.S. Pat. No. 6,905,842, which claims benefit to provisional Application Ser. No. 60/083,807, filed May 1, 1998. Foreign priority is claimed to Australian patent application PO5117/97 filed Feb. 14, 1997.

### FIELD OF THE INVENTION

The present invention relates generally to therapeutic and diagnostic agents. More particularly, the present invention provides therapeutic molecules capable of modulating signal transduction such as but not limited to cytokine-mediated signal transduction. The molecules of the present invention are useful, therefore, in modulating cellular responsiveness to cytokines as well as other mediators of signal transduction such as endogenous or exogenous molecules, antigens, microbes and microbial products, viruses or components thereof, ions, hormones and parasites.

Bibliographic details of the publications referred to in this specification by author are collected at the end of the description. The subject specification contains nucleotide and amino acid sequence information prepared using the programme PatentIn Version 2.0, presented herein after the bibliography. Each nucleotide or amino acid sequence is identified in the sequence listing by the numeric indicator <210> followed by the sequence identifier (e.g. <210>1, <210>2, etc). The length, type of sequence (DNA, protein (PRT), etc) and source organism for each nucleotide or amino acid sequence are indicated by information provided in the numeric indicator fields <211>, <212> and <213>, respectively. Nucleotide and amino acid sequences referred to in the specification are defined by the sequence identifier (eg. SEQ ID NO: 1, SEQ ID NO:2).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue. A summary of the sequence listing is given in Table 1.

### BACKGROUND OF THE INVENTION

Cells continually monitor their environment in order to modulate physiological and biochemical processes which in turn affects future behaviour. Frequently, a cell's initial interaction with its surroundings occurs via receptors expressed on the plasma membrane. Activation of these receptors, whether through binding endogenous ligands (such as cytokines) or exogenous ligands (such as antigens), triggers a biochemical cascade from the membrane through the cytoplasm to the nucleus.

Of the endogenous ligands, cytokines represent a particularly important and versatile group. Cytokines are proteins which regulate the survival, proliferation, differentiation and function of a variety of cells within the body [Nicola, 1994]. The haemopoietic cytokines have in common a four-alpha helical bundle structure and the vast majority interact with a structurally related family of cell surface receptors, the type I and type II cytokine receptors [Bazan, 1990; Sprang, 1993]. In all cases, ligand-induced receptor aggregation appears to be a critical event in initiating intracellular signal transduction cascades. Some cytokines, for example growth hormone, erythropoietin (Epo) and granulocyte-colony-stimulating factor (G-CSF), trigger receptor homodimerisation, while for other cytokines, receptor heterodimerisation or heterotrimerisation is crucial. In the latter cases, several cytokines share common receptor subunits and on this basis can be grouped into three subfamilies with similar patterns of intracellular activation and similar biological effects [Hilton, 1994]. Interleukin-3 (IL-3), IL-5 and granulocyte-macrophage colony-stimulating factor (GM-CSF) use the common β-receptor subunit (βc) and each cytokine stimulates the production and functional activity of granulocytes and macrophages. IL-2, IL-4, IL-7, IL-9, and IL-15 each use the common γ-chain (γc), while IL-4 and IL-13 share an alternative γ-chain (γ′c or IL-13 receptor α-chain). Each of these cytokines plays an important role in regulating acquired immunity in the lymphoid system. Finally, IL-6, IL-1, leukaemia inhibitory factor (LIF), oncostatin-M (OSM), ciliary neurotrophic factor (CNTF) and cardiotrophin (CT) share the receptor subunit gp 130. Each of these cytokines appears to be highly pleiotropic, having effects both within and outside the haemopoietic system [Nicola, 1994].

In all of the above cases at least one subunit of each receptor complex contains the conserved sequence elements, termed box1 and box2, in their cytoplasmic tails [Murakami, 1991]. Box1 is a proline-rich motif which is located more proximal to the transmembrane domain than the acidic box 2 element. The box-1 region serves as the binding site for a class of cytoplasmic tyrosine kinases termed JAKs (Janus kinases). Ligand-induced receptor dimerisation serves to increase the catalytic activity of the associated JAKs through cross-phosphorylation. Activated JAKs then tyrosine phosphorylate several substrates, including the receptors themselves. Specific phosphotyrosine residues on the receptor then serve as docking sites for SH2-containing proteins, the best characterised of which are the signal transducers and activators on transcription (STATs) and the adaptor protein, shc. The STATs are then phosphorylated on tyrosines, probably by JAKs, dissociate from the receptor and form either homodimers or heterodimers through the interaction of the SH2 domain of one STAT with the phosphotyrosine residue of the other. STAT dimers then translocate to the nucleus where they bind to specific cytokine-responsive promoters and activate transcription [Darnell, 1994; Ihle, 1995; Ihle, 1995]. In a separate pathway, tyrosine phosphorylated shc interacts with another SH2 domain-containing protein, Grb-2, leading ultimately to activation of members of the MAP kinase family and in turn transcription factors such as fos and jun [Sato, 1993; Cutler, 1993]. These pathways are not unique to members of the cytokine receptor family since cytokines that bind receptor tyrosine kinases also being able to activate STATs and members of the MAP kinase family [David, 1996; Leaman, 1996; Shual, 1993; Sato, 1993; Cutler, 1993].

Four members of the JAK family of cytoplasmic tyrosine kinases have been described, JAK1, JAK2, JAK3 and TYK2, each of which binds to a specific subset of cytokine receptor subunits. Six STATs have been described (STAT1 through STAT6), and these too are activated by distinct cytokine/receptor complexes. For example, STAT1 appears to be functionally specific to the interferon system, STAT4 appears to be specific to IL-12, while STAT6 appears to be specific for IL-4 and IL-13. Thus, despite common activation mechanisms some degree of cytokine specificity may be achieved through the use of specific JAKs and STATs [Thierfelder, 1996; Kaplan, 1996; Takeda, 1996; Shimoda, 1996; Meraz, 1996; Durbin, 1996].

In addition to those described above, there are clearly other mechanisms of activation of these pathways. For example, the JAK/STAT pathway appears to be able to activate MAP kinases independent of the shc-induced pathway [David, 1995] and the STATs themselves can be activated without binding to the receptor, possibly by direct interaction with JAKs [Gupta, 1996]. Conversely, full activation of STATS may require the action of MAP kinase in addition to that of JAKs [David, 1995; Wen, 1995].

While the activation of these signalling pathways is becoming better understood, little is known of the regulation of these pathways, including employment of negative or positive feedback loops. This is important since once a cell has begun to respond to a stimulus, it is critical that the intensity and duration of the response is regulated and that signal transduction is switched off. It is likewise desirable to increase the intensity of a response systemically or even locally as the situation requires.

In work leading up to the present invention, the inventors sought to isolate negative regulators of signal transduction. The inventors have now identified a new family of proteins which are capable of acting as regulators of signalling. The new family of proteins is defined as the suppressor of cytokine signalling (SOCS) family based on the ability of the initially identified SOCS molecules to suppress cytokine-mediated signalling. It should be noted, however, that not all members of the SOCS family need necessarily share suppressor function nor target solely cytokine mediated signalling. The SOCS family comprises at least three classes of protein molecules based on amino acid sequence motifs located N-terminal of a C-terminal motif called the SOCS box. The identification of this new family of regulatory molecules permits the generation of a range of effector or modulator molecules capable of modulating signal transduction and, hence, cellular responsiveness to a range of molecules including cytokines. The present invention, therefore, provides therapeutic and diagnostic agents based on SOCS proteins, derivatives, homologues, analogues and mimetics thereof as well as agonists and antagonists of SOCS proteins.

### SUMMARY OF THE INVENTION

Throughout this specification and the claims which follow, unless the context requires otherwise the word “comprise”, or variations such as “comprises” or “comprising”, will be understood to imply the inclusion of a stated integer or group of integers but not the exclusion of any other integer or group of integers.

The present invention provides inter alia nucleic acid molecules encoding members of the SOCS family of proteins as well as the proteins themselves. Reference hereinafter to “SOCS” encompasses any or all members of the SOCS family. Specific SOCS molecules are defined numerically such as, for example, SOCS1, SOCS2 and SOCS3. The species from which the SOCS has been obtained may be indicated by a preface of a single letter abbreviation where “h” is human, “m” is murine and “r” is rat. Accordingly, “mSOCS1” is a specific SOCS from a murine animal. Reference herein to “SOCS” is not to imply that the protein solely suppresses cytokine-mediated signal transduction, as the molecule may modulate other effector-mediated signal transductions such as by hormones or other endogenous or exogenous molecules, antigens, microbes and microbial products, viruses or components thereof, ions, hormones and parasites. The term “modulates” encompasses up-regulation, down-regulation as well as maintenance of particular levels.

One aspect of the present invention provides a nucleic acid molecule comprising a sequence of nucleotides encoding or complementary to a sequence encoding a protein or a derivative, homologue, analogue or mimetic thereof or a nucleotide sequence capable of hybridizing thereto under low stringency conditions at 42° C. wherein said protein comprises a SOCS box in its C-terminal region

Another aspect of the present invention provides a nucleic acid molecule comprising a sequence of nucleotides encoding or complementary to a sequence encoding a protein or a derivative, homologue, analogue or mimetic thereof or a nucleotide sequence capable of hybridizing thereto under low stringency conditions at 42° C. wherein said protein comprises a SOCS box in its C-terminal region and a protein:molecule interacting region.

Yet another aspect of the present invention is directed to a nucleic acid molecule comprising a sequence of nucleotides encoding or complementary to a sequence encoding a protein or a derivative, homologue, analogue or mimetic thereof or a nucleotide sequence capable of hybridizing thereto under low stringency conditions at 42° C. wherein said protein comprises a C-terminal region and a protein:molecule interacting region located in a region N-terminal of the SOCS box.

Preferably, the protein:molecule interacting region is a protein:DNA or protein:protein binding region.

Still a further aspect of the present invention provides a nucleic acid molecule comprising a sequence of nucleotides encoding or complementary to a sequence encoding a protein or a derivative, homologue, analogue or mimetic thereof or a nucleotide sequence capable of hybridizing thereto under low stringency conditions at 42° C. wherein said protein comprises a SOCS box in its C-terminal region and one or more of an SH2 domain, WD-40 repeats or ankyrin repeats N-terminal of the SOCS box.

Even still a further aspect of the present invention is directed to a nucleic acid molecule comprising a sequence of nucleotides encoding or complementary to a sequence encoding a protein or a derivative, homologue, analogue or mimetic thereof or a nucleotide sequence capable of hybridizing thereto under low stringency conditions at 42° C. wherein said protein comprises a SOCS box in its C-terminal region wherein the SOCS box comprises the amino acid sequence:

X₁X₂X₃X₄X₅X₆X₇X₈X₈X₁₀X₁₁X₁₂X₁₃X₁₄X₁₅X₁₆[Xᵢ]ₙX₁₇X₁₈
X₁₉X₂₀X₂₁X₂₂X₂₃[Xj]ₙX₂₄X₂₅X₂₆X₂₇X₂₈

wherein: X₁ is L, I, V, M, A or P;
   X₂ is any amino acid residue;
   X₃ is P, T or S;
   X₄ is L, I, V, M, A or P;
   X₅ is any amino acid;
   X₆ is any amino acid;
   X₇ is L, I, V, M, A, F, Y or W;
   X₈ is C, T or S;
   X₉ is R, K or H;
   X₁₀ is any amino acid;
   X₁₁ is any amino acid;
   X₁₂ is L, I, V, M, A or P;
   X₁₃ is any amino acid;
   X₁₄ is any amino acid;
   X₁₅ is any amino acid,
   X₁₆ is L, I, V, M, A, P, G, C, T or S;
   [Xᵢ]ₙ is a sequence of n amino acids wherein n is from 1 to 50 amino acids and wherein the sequence Xᵢ may comprise the same or different amino acids selected from any amino acid residue;
   X₁₇ is L, I, V, M, A or P;
   X₁₈ is any amino acid;
   X₁₉ is any amino acid;
   X₂₀ L, I, V, M, A or P;
   X₂₁ is P;
   X₂₂ is L, I, V, M, A, P or G;
   X₂₃ is P or N;
   [Xⱼ]ₙ is a sequence of n amino acids wherein n is from 0 to 50 amino acids and wherein the sequence Xⱼ may comprise the same or different amino acids selected from any amino acid residue;
   X₂₄ is L, I, V, M, A or P;
   X₂₅ is any amino acid;
   X₂₆ is any amino acid;
   X₂₇ is Y or F;
   X₂₈ is L, I, V, M, A or P;
      and a protein:molecule interacting region such as but not limited to one or more of an SH2 domain, WD-40 repeats and/or ankyrin repeats N-terminal of the SOCS box.

Another aspect of the present invention is directed to a nucleic acid molecule comprising a sequence of nucleotides encoding or complementary to a sequence encoding a protein or a derivative, homologue, analogue or mimetic thereof or a nucleotide sequence capable of hybridizing thereto under low stringency conditions at 42° C. wherein said protein exhibits the following characteristics:

(i) comprises a SOCS box in its C-terminal region having the amino acid sequence:

X₁X₂X₃X₄X₅X₆X₇X₈X₈X₁₀X₁₁X₁₂X₁₃X₁₄X₁₅X₁₆[Xᵢ]ₙX₁₇X₁₈
X₁₉X₂₀X₂₁X₂₂X₂₃[Xj]ₙX₂₄X₂₅X₂₆X₂₇X₂₈

wherein: X₁ is L, I, V, M, A or P;
   X₂ is any amino acid residue;
   X₃ is P, T or S;
   X₄ is L, I, V, M, A or P;
   X₅ is any amino acid;
   X₆ is any amino acid;
   X₇ is L, I, V, M, A, F, Y or W;
   X₈ is C, T or S;
   X₉ is R, K or H;
   X₁₀ is any amino acid;
   X₁₁ is any amino acid;
   X₁₂ is L, I, V, M, A or P;
   X₁₃ is any amino acid;
   X₄ is any amino acid;
   X₁₅ is any amino acid;
   X₁₆ is L, I, V, M, A, P, G, C, T or S;
   [Xᵢ]ₙ is a sequence of n amino acids wherein n is from 1 to 50 amino acids and wherein the sequence Xᵢ may comprise the same or different amino acids selected from any amino acid residue;
   X₇ is L, I, V, M, A or P;
   X₁₈ is any amino acid;
   X₁₉ is any amino acid;
   X₂₀ L, I, V, M, A or P;
   X₂₁ is P;
   X₂₂ is L, I, V, M, A, P or G;
   X₂₃ is P or N;
   [Xⱼ]ₙ is a sequence of n amino acids wherein n is from 0 to 50 amino acids and wherein the sequence Xⱼ may comprise the same or different amino acids selected from any amino acid residue;
   X₂₄ is L, I, V, M, A or P;
   X₂₅ is any amino acid;
   X₂₆ is any amino acid;
   X₂₇ is Y or F;
   X₂₈ is L, I, V, M, A or P; and
      (ii) comprises at least one of a SH2 domain, WD-40 repeats and/or ankyrin repeats or other protein:molecule interacting domain in a region N-terminal of the SOCS box.

Preferably, the SOCS molecules modulate signal transduction such as from a cytokine or hormone or other endogenous or exogenous molecule, a microbe or microbial product, an antigen or a parasite.

More preferably, the SOCS molecule modulate cytokine mediated signal transduction.

Still another aspect of the present invention comprises a nucleic acid molecule comprising a sequence of nucleotides encoding or complementary to a sequence encoding a protein or a derivative, homologue, analogue or mimetic thereof or comprises a nucleotide sequence capable of hybridizing thereto under low stringency conditions at 42° C. wherein said protein exhibits the following characteristics;

(i) is capable of modulating signal transduction;
(ii) comprises a SOCS box in its C-terminal region having the amino acid sequence:

X₁X₂X₃X₄X₅X₆X₇X₈X₈X₁₀X₁₁X₁₂X₁₃X₁₄X₁₅X₁₆[Xᵢ]ₙX₁₇X₁₈
X₁₉X₂₀X₂₁X₂₂X₂₃[Xj]ₙX₂₄X₂₅X₂₆X₂₇X₂₈

wherein: X₁ is L, I, V, M, A or P;
   X₂ is any amino acid residue;
   X₃ is P, T or S;
   X₄ is L, I, V, M, A or P;
   X₅ is any amino acid;
   X₆ is any amino acid;
   X₇ is L, I, V, M, A, F, Y or W;
   X₈ is C, T or S;
   X₉ is R, K or H;
   X₁₀ is any amino acid;
   X₁₁ is any amino acid;
   X₁₂ is L, I, V, M, A or P;
   X₁₃ is any amino acid;
   X₁₄ is any amino acid;
   X₁₅ is any amino acid;
   X₁₆ is L, I, V, M, A, P, G, C, T or S;
   [Xᵢ]ₙ is a sequence of n amino acids wherein n is from 1 to 50 amino acids and wherein the sequence Xᵢ may comprise the same or different amino acids selected from any amino acid residue;
   X₁₇ is L, I, V, M, A or P;
   X₁₈ is any amino acid;
   X₁₉ is any amino acid;
   X₂₀ L, I, V, M, A or P;
   X₂₁ is P;
   X₂₂ is L, I, V, M, A, P or G;
   X₂₃ is P or N;
   [Xⱼ]ₙ is a sequence of n amino acids wherein n is from 0 to 50 amino acids and wherein the sequence Xⱼ may comprise the same or different amino acids selected from any amino acid residue;
   X₂₄ is L, I, V, M, A or P;
   X₂₅ is any amino acid;
   X₂₆ is any amino acid;
   X₂₇ is Y or F;
   X₂₈ is L, I, V, M, A or P; and
      (iii) comprises at least one of a SH2 domain, WD-40 repeats and/or ankyrin repeats or other protein:molecule interacting domain in a region N-terminal of the SOCS box.

Preferably, the signal transduction is mediated by a cytokine such as one or more of EPO, TPO, G-CSF, GM-CSF, IL-3, IL-2, IL-4, IL-7, IL-13, IL-6, LIF, IL-12, IFNα, TNFα, IL-1 and/or M-CSF.

Preferably, the signal transduction is mediated by one or more of Interleukin 6 (IL-6), Leukaemia Inhibitory Factor (LIF), Oncostatin M (OSM), Interferon (IFN)-α and/or thrombopoietin.

Preferably, the signal transduction is mediated by IL-6.

Particularly preferred nucleic acid molecules comprise nucleotide sequences substantially set forth in SEQ ID NO: 3 (mSOCS1), SEQ ID NO: 5 (mSOCS2), SEQ ID NO: 7 (mSOCS3), SEQ ID NO: 9 (hSOCS1), SEQ ID NO: 11 (rSOCS1), SEQ ID NO: 13 (mSOCS4), SEQ ID NOS: 15 and 16 (hSOCS4), SEQ ID NO: 17 (mSOCS5), SEQ ID NO: 19 (hSOCS5), SEQ ID NO: 20 (mSOCS6), SEQ ID NOS: 22 and 23 (hSOCS6), SEQ ID NO: 24 (mSOCS7), SEQ ID NOS: 26 and 27 (hSOCS7), SEQ ID NO: 28 (mSOCS8), SEQ ID NO: 30 (mSOCS9), SEQ ID NO: 31 (hSOCS9), SEQ ID NO: 32 (mSOCS10), SEQ ID NOS: 33 and 34 (hSOCS10), SEQ ID NO: (hSOCS11), SEQ ID NO: 37 (mSOCS12), SEQ ID NOS: 38 and 39 (hSOCS12), SEQ ID NO: 40 (mSOCS13), SEQ ID NO: 42 (hSOCS13), SEQ ID NO: 43 (mSOCS14), SEQ ID NO: 45 (mSOCS15) and SEQ ID NO: 47 (hSOCS15) or a nucleotide sequence having at least about 15% similarity to all or a region of any of the listed sequences or a nucleotide acid molecule capable of hybridizing to any one of the listed sequences under low stringency conditions at 42° C.

Another aspect of the present invention relates to a protein or a derivative, homologue, analogue or mimetic thereof comprising a SOCS box in its C-terminal region.

Yet another aspect of the present invention is directed to a protein or a derivative, homologue, analogue or mimetic thereof comprising a SOCS box in its C-terminal region and a protein:molecule interacting region.

Even yet another aspect of the present invention provides a protein or a derivative, homologue, analogue or mimetic thereof comprising an interacting region located in a region N-terminal of the SOCS box.

Preferably, the protein:molecule interacting region is a protein:DNA or a protein:protein binding region.

Another aspect of the present invention contemplates a protein or a derivative, homologue, analogue or mimetic thereof comprising a SOCS box in its C-terminal region and a SH2 domain, D-40 repeats or ankyrin repeats N-terminal of the SOCS box.

Still yet another aspect of the present invention provides a protein or a derivative, homologue, analogue or mimetic thereof exhibiting the following characteristics:

(i) comprises a SOCS box in its C-terminal region having the amino acid sequence:

X₁X₂X₃X₄X₅X₆X₇X₈X₈X₁₀X₁₁X₁₂X₁₃X₁₄X₁₅X₁₆[Xᵢ]ₙX₁₇X₁₈
X₁₉X₂₀X₂₁X₂₂X₂₃[Xj]ₙX₂₄X₂₅X₂₆X₂₇X₂₈

wherein: X₁ is L, I, V, M, A or P;
   X₂ is any amino acid residue;
   X₃ is P, T or S;
   X₄ is L, I, V, M, A or P;
   X₅ is any amino acid;
   X₆ is any amino acid;
   X₇ is L, I, V, M, A, F, Y or W;
   X₈ is C, T or S;
   X₉ is R, K or H;
   X₁₀ is any amino acid;
   X₁₁ is any amino acid;
   X₁₂ is L, I, V, M, A or P;
   X₁₃ is any amino acid;
   X₁₄ is any amino acid;
   X₁₅ is any amino acid;
   X₁₆ is L, I, V, M, A, P, G, C, T or S;
   [Xᵢ]ₙ is a sequence of n amino acids wherein n is from 1 to 50 amino acids and wherein the sequence Xᵢ may comprise the same or different amino acids selected from any amino acid residue;
   X₁₇ is L, I, V, M, A or P;
   X₁₈ is any amino acid;
   X₁₉ is any amino acid;
   X₂₀ L, I, V, M, A or P;
   X₂₁ is P;
   X₂₂ is L, I, V, M, A, P or G;
   X₂₃ is P or N;
   [Xⱼ]ₙ is a sequence of n amino acids wherein n is from 0 to 50 amino acids and wherein the sequence Xⱼ may comprise the same or different amino acids selected from any amino acid residue;
   X₂₄ is L, I, V, M, A or P;
   X₂₅ is any amino acid;
   X₂₆ is any amino acid;
   X₂₇ is Y or F;
   X₂₈ is L, I, V, M, A or P; and
      (ii) comprises at least one of a SH2 domain, WD-40 repeats and/or ankyrin repeats or other protein:molecule interacting domain in a region N-terminal of the SOCS box.

Preferably, the proteins modulate signal transduction such as cytokine-mediated signal transduction.

Preferred cytokines are EPO, TPO, G-CSF, GM-CSF, IL-3, IL-2, IL-4, IL-7, IL-13, IL-6, LIF, L-12, IFNγ, TNFα, IL-1 and/or M-CSF.

A particularly preferred cytokine is IL-6.

Even yet another aspect of the present invention provides a protein or derivative, homologue, analogue or mimetic thereof exhibiting the following characteristics:

(i) is capable of modulating signal transduction such as cytokine-mediated signal transduction;
(ii) comprises a SOCS box in its C-terminal region having the amino acid sequence:

X₁X₂X₃X₄X₅X₆X₇X₈X₈X₁₀X₁₁X₁₂X₁₃X₁₄X₁₅X₁₆[Xᵢ]ₙX₁₇X₁₈
X₁₉X₂₀X₂₁X₂₂X₂₃[Xj]ₙX₂₄X₂₅X₂₆X₂₇X₂₈

wherein: X₁ is L, I, V, M, A or P;
   X₂ is any amino acid residue;
   X₃ is P, T or S;
   X₄ is L, I, V, M, A or P;
   X₅ is any amino acid;
   X₆ is any amino acid;
   X₇ is L, I, V, M, A, F, Y or W;
   X₈ is C, T or S;
   X₉ is R, K or H;
   X₁₀ is any amino acid;
   X₁₁ is any amino acid;
   X₁₂ is L, I, V, M, A or P;
   X₁₃ is any amino acid;
   X₁₄ is any amino acid;
   X₁₅ is any amino acid;
   X₁₆ is L, I, V, M, A, P, G, C, T or S;
   [Xᵢ]ₙ is a sequence of n amino acids wherein n is from 1 to 50 amino acids and wherein the sequence Xⱼ may comprise the same or different amino acids selected from any amino acid residue;
   X₁₇ is L, I, V, M, A or P;
   X₁₈ is any amino acid;
   X₁₉ is any amino acid;
   X₂₀ L, I, V, M, A or P;
   X₂₁ is P;
   X₂₂ is L, I, V, M, A, P or G;
   X₂₃ is P or N;
   [Xⱼ]ₙ is a sequence of n amino acids wherein n is from 0 to 50 amino acids and wherein the sequence Xⱼ may comprise the same or different amino acids selected from any amino acid residue;
   X₂₄ is L, I, V, M, A or P;
   X₂₅ is any amino acid;
   X₂₆ is any amino acid;
   X₂₇ is Y or F;
   X₂₈ is L, I, V, M, A or P; and
      (iii) comprises at least one of a SH2 domain, WD-40 repeats and/or ankyrin repeats or other protein-molecule interacting domain in a region N-terminal of the SOCS box.

Particularly preferred SOCS proteins comprise an amino acid sequence substantially as set forth in SEQ ID NO: 4 (mSOCS1), SEQ ID NO:6 (mSOCS2), SEQ ID NO:8 (mSOCS3), SEQ ID NO:10 (hSOCS1), SEQ ID NO:12 (rSOCS1), SEQ ID NO:14 (mSOCS4), SEQ ID NO:18 (mSOCS5), SEQ ID NO:21 (mSOCS6), SEQ ID NO:25 (mSOCS7), SEQ ID NO:29 (mSOCS8), SEQ ID NO:36 (hSOCS11), SEQ ID NO:41 (mSOCS13), SEQ ID NO:44 (mSOCS14), SEQ ID NO:46 (mSOCS15) and SEQ ID NO:48 (hSOCS15) or an amino acid sequence having at least 15% similarity to all or a region of any one of the listed sequences.

Another aspect of the present invention contemplates a method of modulating levels of a SOCS protein in a cell said method comprising contacting a cell containing a SOCS gene with an effective amount of a modulator of SOCS gene expression or SOCS protein activity for a time and under conditions sufficient to modulate levels of said SOCS protein.

A related aspect of the present invention provides a method of modulating signal transduction in a cell containing a SOCS gene comprising contacting said cell with an effective amount of a modulator of SOCS gene expression or SOCS protein activity for a time sufficient to modulate signal transduction.

Yet a further related aspect of the present invention is directed to a method of influencing interaction between cells wherein at least one cell carries a SOCS gene, said method comprising contacting the cell carrying the SOCS gene with an effective amount of a modulator of SOCS gene expression or SOCS protein activity for a time sufficient to modulate signal transduction.

In accordance with the present invention, n in [Xᵢ]ₙ and [Xⱼ]ₙ may, in addition from being 1-50, be from 1-30, 1-20, 1-10 and 1-5.

A summary of the sequence listing referred to in the subject specification is given in Table 1.

TABLE 1
SUMMARY OF SEQUENCE IDENTIFYING NUMBERS

| SEQUENCE | SEQ ID NO |
|---|---|
| PCR Primer | 1 |
| PCR Primer | 2 |
| Mouse SOCS1 (nucleotide) | 3 |
| Mouse SOCS1 (amino acid) | 4 |
| Mouse SOCS2 (nucleotide) | 5 |
| Mouse SOCS2 (amino acid) | 6 |
| Mouse SOCS3 (nucleotide) | 7 |
| Mouse SOCS3 (amino acid) | 8 |
| Human SOCS1 (nucleotide) | 9 |
| Human SOCS1 (amino acid) | 10 |
| Rat SOCS1 (nucleotide) | 11 |
| Rat SOCS1 (amino acid) | 12 |
| nucleotide sequence of murine SOCS4 | 13 |
| amino acid sequence of murine SOCS4 | 14 |
| nucleotide sequence of SOCS4 cDNA human contig 4.1 | 15 |
| nucleotide sequence of SOCS4 cDNA human contig 4.2 | 16 |
| nucleotide sequence of murine SOCS5 | 17 |
| amino acid sequence of murine SOCS5 | 18 |
| nucleotide sequence of human SOCS5 | 19 |
| nucleotide sequence of murine SOCS6 | 20 |
| amino acid of murine SOCS6 | 21 |
| nucleotide sequence of human SOCS6 contig h6.1 | 22 |
| nucleotide sequence of human SOCS6 contig h6.2 | 23 |
| nucleotide sequence of murine SOCS7 | 24 |
| amino acid sequence of murine SOCS7 | 25 |
| nucleotide sequence of human SOCS7 contig h7.1 | 26 |
| nucleotide sequence of human SOCS7 contig 17.2 | 27 |
| nucleotide sequence of murine SOCS8 | 28 |
| amino acid sequence of murine SOCS 8 | 29 |
| nucleotide sequence of murine SOCS9 | 30 |
| nucleotide sequence of human SOCS9 | 31 |
| nucleotide sequence of murine SOCS10 | 32 |
| nucleotide sequence of human SOCS10 contig h10.1 | 33 |
| nucleotide sequence of human SOCS10 contig h10.2 | 34 |
| nucleotide sequence of human SOCS11 | 35 |
| amino acid sequence of human SOCS11 | 36 |
| nucleotide sequence of mouse SOCS12 | 37 |
| nucleotide sequence of human SOCS12 contig h12.1 | 38 |
| nucleotide sequence of human SOCS12 contig h12.2 | 39 |
| nucleotide sequence of murine SOCS13 | 40 |
| amino acid sequence of murine SOCS13 | 41 |
| nucleotide sequence of human SOCS13 cDNA contig h13.1 | 42 |
| nucleotide sequence of murine SOCS14 cDNA | 43 |
| amino acid sequence of murine SOCS14 | 44 |
| nucleotide sequence of murine SOCS15 cDNA | 45 |
| amino acid sequence of murine SOCS15 | 46 |
| nucleotide sequence of human SOCS15 | 47 |
| amino acid sequence of human SOCS15 | 48 |
| 5′ oliognucleotide sequence (2465) | 49 |
| 3′ oligonucleotide sequence (2466) | 50 |
| N-terminal GFP tag | 51 |
| 3′ genomic oligonucleotide no. 3243 | 52 |
| 5′ genomic oligonucleotide no. 3244 | 53 |
| Amino acid sequence of SEQ ID NO: 53 | 54 |
| 3′ cDNA oligonucleotide no. 3245 | 55 |
| Nucleotide sequence of murine SOCS13 | 56 |
| Amino acid sequence of murine SOCS13 | 57 |
| Oligonucleotide no. 3342 | 58 |
| Amino acid sequence of SOCS box | 59 |
| Nucleotide sequence of murine SOCS5 | 60 |
| Amino acid sequence of murine SOCS5 | 61 |
| Nucleotide sequence of murine SOCS9 | 62 |
| Amino acid sequence of murine SOCS9 | 63 |
| SOCS Box Motif in mSOCS-1 and rSOCS-1 | 64 |
| SOCS Box Motif in mSOCS-2 | 65 |
| SOCS Box Motif in mSOCS-3 | 66 |
| SOCS Box Motif in hSOCS-1 | 67 |
| SOCS Box Motif in mSOCS-4 | 68 |
| SOCS Box Motif in mSOCS-5 | 69 |
| SOCS Box Motif in mSOCS-7 | 70 |
| SOCS Box Motif in mSOCS-8 | 71 |
| SOCS Box Motif in hSOCS-9 | 72 |
| SOCS Box Motif in mSOCS-10 | 73 |
| SOCS Box Motif in hSOCS-11 | 74 |
| SOCS Box Motif in mSOCS-12 | 75 |
| SOCS Box Motif in mSOCS-13 | 76 |
| SOCS Box Motif in mSOCS-14 | 77 |
| SOCS Box Motif in mSOCS-15 | 78 |
| SOCS Box Motif in hSOCS-15 | 79 |
| SOCS Box Motif in mSOCS-6 | 80 |
| SOCS Box Motif in mSOCS-9 | 81 |

Single and three letter abbreviations are used to denote amino acid residues and these are summarized in Table 2.

TABLE 2

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue | Xaa | X |

### BRIEF DESCRIPTION OF THE DRAWINGS

In some of the Figures, abbreviations are used to denote SOCS proteins with certain binding motifs. SOCS proteins which contain WD-40 repeats are referred to as WSB1-WSB4. SOCS proteins with ankyrin repeats are referred to as ASB1-ASB3. Deletion mutants are “Δ”. For example, ΔC is a deletion in the carboxy terminal region and ΔN is a deletion in the amino terminal region.

FIG. 1 is a diagrammatic representation showing generation of an IL-6-unresponsive M1 clone by retroviral infection. The RUFneo retrovirus, showing the position of landmark restriction endonuclease cleavage sites, the 4A2 cDNA insert and the position of PCR primer sequences.

FIGS. 2A-2B are representations of the nucleotide sequence and structure of the SOCS1 gene. 2A. The genomic context of SOCS1 in relation to the protamine gene cluster on murine chromosome 16. The accession number of this locus is MMPRMGNS (direct submission; G. Schlueter, 1995) for the mouse and BTPRMTNP2 for the rat (direct submission; G. Schlueter, 1996). 2B. The nucleotide sequence of the SOCS1 cDNA and deduced amino acid sequence. Conventional three letter abbreviations are used for the amino acid sequence and the asterisk indicates the stop codon. The polyadenylation signal sequence is underlined. The coding region is shown in uppercase and the untranslated region is shown in lower case.

FIGS. 3(I)-3(III) are representations of a comparison of the amino acid sequences of SOCS1, SOCS2, SOCS3 and CIS. Alignment of the predicted amino acid sequence of mouse (mm), human (hs) and rat (rr) SOCS1, SOCS2, SOCS3 and CIS. Those residues shaded are conserved in three or four mouse SOCS family members. The SH2 domain is boxed in dotted lines, while the SOCS box is bounded by double lines.

FIGS. 4A(I)-4F(II) are representations of a comparison of the amino acid sequence of the SOCS proteins. Schematic representation of structures of SOCS proteins including proteins which contain WD-40 repeats (WSB) and ankyrin repeats (ASB). 4A(I)-4A(II): Alignment of N-terminal regions of SOCS proteins. 4B(I)-4B(II): Alignment of the SH2 domains of CIS, SOCS1, 2, 3, 5, 9, 11 and 14. 4C(I)-4C(III): Alignment of the WD-40 repeats of SOCS4, SOCS6, SOCS13 and SOCS15. 4D: Alignment of the ankyrin repeats of SOCS7 and SOCS10. 4E(I)-4E(II): Alignment of the regions between SH2, WD-40 and ankyrin repeats and the SOCS box. 4F(I)-4F(II): Alignment of the SOCS box. In each case the conventional one letter abbreviations for amino acids are used, with X denoting residues of uncertain identity and [symbol] denoting the beginning and the end of contigs. Amino acid sequence obtained from conceptual translation of nucleic acid sequence derived from isolated cDNAs is shown in upper case while amino acid sequence obtained by conceptual translation of ESTs is shown in lower case and is approximate only. Conserved residues, defined as (LIVMA), (FYW), (DE), (QN), (C, S, T), (KRH), (PG) are shaded in the SH2 domain, WD-40 repeats, ankyrin repeats and the SOCS box. For the alignment of SH2 domains, WD-40 repeats and ankyrin repeats a consensus sequence is shown above. In each case this has been derived from examination of a large and diverse set of domains (Neer et al, 1994; Bork, 1993).

FIG. 5 is a representation showing the nucleotide sequence of the mouse SOCS4 cDNA. The nucleotides encoding the mature coding region from the predicted ATG “start” codon to the stop codon is shown in upper case, while the predicted 5′ and 3′ untranslated regions are shown in lower case.

FIG. 6 is a representation showing the predicted amino acid sequence of the mouse SOCS4 protein, derived from the nucleotide sequence in FIG. 5. The SOCS box, which also shown in FIG. 4, is underlined.

FIG. 7 is a representation showing the nucleotide sequence of human SOCS4 cDNA contigs h4.1 and h4.2, derived from analysis of ESTs listed in Table 4.1.

FIG. 8 is a diagrammatic representation showing the relationship of mouse SOCS5 genomic (57-2) and cDNA (5-3-2) clones to contigs derived from analysis of mouse ESTs (Table 5.1) and human cDNA clone (5-94-2) and ESTs (Table 5.2). The nucleotide sequence of the mouse SOCS5 contig is shown in FIG. 9A, with the sequence of human SOCS5 contig (h5.1) being shown in FIG. 10. The deduced amino acid sequence of mouse SOCS5 is shown in FIG. 9B. The structure of the protein is shown schematically, with the SH2 domain indicated by the open white wave box and the SOCS box by the open hatched box. The putative 5′ and 3′ translated regions are shown by the thin solid line.

FIG. 9A is a representation showing the nucleotide sequence of the mouse SOCS5 derived from analysis of genomic and cDNA clones. The nucleotides encoding the mature coding region from the predicted ATG “start” codon to the stop codon is shown in upper case, while the predicted 5′ and 3′ untranslated regions are shown in lower case. The relationship of mouse cDNA sequence to mouse and human EST contigs is illustrated in FIG. 8.

FIG. 9B is a representation of the predicted amino acid sequence of mouse SOCS5 protein, derived from the nucleotide sequence in FIG. 9A. The SOCS box, which also shown in FIG. 4 is underlined.

FIG. 10 is a representation showing the nucleotide sequence of human SOCS5 cDNA contig h5.1, derived from analysis of cDNA clone 5-94-2 and the ESTs listed in Table 5.2. The relationship of these contigs to the mouse cDNA sequence is illustrated in FIG. 8.

FIG. 11 is a diagrammatic representation showing the relationship of mouse SOCS6 cDNA clones (6-1A, 6-2A, 6-5B, 6-4N, 6-18, 6-29, 6-3N and 6-5N) to contigs derived from analysis of mouse ESTs (Table 6.1) and human ESTs (Table 6.2). The nucleotide sequence of the mouse SOCS-6 contig is shown in FIG. 12A, with the sequence of human SOCS6 contigs (h6.1 and h6.2) being shown in FIG. 13. The deduced amino acid sequence of mouse SOCS6 is shown in FIG. 12B. The structure of the protein is shown schematically, while the WD-40 repeats indicated by the open hatched boxes and the SOCS box by the open dotted box. The putative 5′ and 3′ untranslated regions are shown by the thin solid line.

FIG. 12A is a representation showing the nucleotide sequence of the mouse SOCS6 derived from analysis of cDNA clone 64-10A-11. The nucleotides encoding the part of the predicted coding region, ending in the stop codon are shown in upper case, while the predicted, 3′ untranslated regions are shown in lower case. The relationship of mouse cDNA sequence to mouse and human EST contigs is illustrated in FIG. 11.

FIG. 12B is a representation showing the predicted amino acid sequence of mouse SOCS6 protein, derived from the nucleotide sequence in FIG. 12A. The SOCS box, which also shown in FIG. 4 is underlined.

FIG. 13 is a representation showing the nucleotide sequence of human SOCS6 cDNA contig h6.1 and contig h6.2, derived from analysis of cDNA clone 5-94-2 and the ESTs listed in Table 6.2. The relationship of these contigs to the mouse cDNA sequence is illustrated in FIG. 11.

FIG. 14 is a diagrammatic representation showing the relationship of mouse SOCS7 cDNA clone (74-10A-11) to contigs derived from analysis of mouse ESTs (Table 7.1), and human ESTs (Table 7.2). The nucleotide sequence of the mouse SOCS7 contig is shown in FIG. 15A with the sequence of human SOCS7 contigs (h7.1 and h7.2) being shown in FIG. 16. The deduced amino acid sequence of mouse SOCS7 is shown in FIG. 15B. The structure of the protein is shown schematically, with the ankyrin repeats indicated by the cross hatched box and the SOCS box by the open dotted box. The putative 5′ and 3′ untranslated regions are shown by the thin solid line in the mouse and by the wavy line in h7.2. Based on analysis of clones isolated to date and ESTs the 3′ untranslated regions of mSOCS7 and hSOCS7 share little similarity.

FIG. 15A is a representation showing the nucleotide sequence of the mouse SOCS7 derived from analysis of cDNA clone 74-10A-11. The nucleotides encoding the part of the predicted coding region, ending in the stop codon are shown in upper case, while the predicted 3′ untranslated regions are shown in lower case. The relationship of mouse cDNA sequence to mouse and human EST contigs is illustrated in FIG. 14.

FIG. 15B is a representation showing the predicted amino acid sequence of mouse SOCS7 protein, derived from the nucleotide sequence in FIG. 15A. The SOCS box, which also shown in FIG. 4 is underlined.

FIG. 16 is a representation showing the nucleotide sequence of human SOCS7 cDNA contig h7.1 and h7.2 derived from analysis of the ESTs listed in Table 7.2. The relationship of these contigs to the mouse cDNA sequence is illustrated in FIG. 14.

FIG. 17 is a diagrammatic representation of the relationship of sequence derived from analysis of mouse SOCS8 ESTs (Table 8.1) to the predicted protein structure of mouse SOCS8. The deduced partial amino acid sequence of mouse SOCS8 is shown in FIG. 18B. The structure of the protein is shown schematically with the SOCS box indicated by the cross hatched box. The predicted 3′ untranslated region is shown by the thin line.

FIG. 18A is a representation showing the partial nucleotide sequence of mouse SOCS8 cDNA (contig 8.1) derived from analysis of ESTs. The nucleotides encoding the part of the predicted coding region, ending in the STOP codon are shown in upper case, while the predicted 3′ untranslated regions are shown in lower case.

FIG. 18B is a representation showing the partial predicted amino acid sequence of the mouse SOCS8 protein, derived from the nucleotide sequence in FIG. 18A. The SOCS box, which also shown in FIG. 4 is underlined.

FIG. 19 is a diagrammatic representation showing the relationship of mouse SOCS9 ESTs (Table 9.1) and human SOCS9 ESTs (Table 9.2). The nucleotide sequence of the mouse SOCS9 contig (m9.1) is shown in FIG. 20, with the sequence of human SOCS9 contig (h9.1) being shown in FIG. 21. The deduced amino acid sequence of human SOCS9 is shown schematically, with the SH2 domain indicated by the open white wave box and the SOCS box by the open hatched box. The putative 3′ untranslated region is shown by the thin solid line.

FIG. 20 is a representation showing the partial nucleotide sequence of mouse SOCS9 cDNA (contig m9.1), derived from analysis of the ESTs listed in Table 9.1. The relationship of these contigs to the mouse cDNA sequence is illustrated in FIG. 19.

FIG. 21 is a representation showing the partial nucleotide sequence of human SOCS9 cDNA contig h9.1), derived from analysis of the ESTs listed in Table 9.2. Although it is clear that contig h9.1 encodes a protein with an SH2 domain and a SOCS box, the quality of the sequence is not high enough to derive a single unambiguous open reading frame. The relationship of these contigs to the mouse cDNA sequence is illustrated in FIG. 19.

FIG. 22 is a representation showing the relationship of mouse SOCS10 cDNA clones (10-9, 10-12, 10-23 and 10-24) to contigs derived from analysis of mouse ESTs (Table 10.1) and human ESTs (Table 10.2). The nucleotide sequence of the mouse SOCS10 contig is shown in FIG. 23, with the sequence of human SOCS10 contigs (h10.1 and h10.2) being shown in FIG. 24. The predicted structure of the protein is shown schematically, with the ankyrin repeats indicated by the cross hatched box and the SOCS box by open hatched box. The putative 3′ untranslated regions is shown by the thin line solid line in the mouse and by the wavy line in h10.2. Based on analysis of clones isolated to date and ESTs the 3′ untranslated regions of mSOCS-10 and hSOCS-10 share little similarity.

FIG. 23 is a representation showing the nucleotide sequence of the mouse SOCS10 derived from analysis of cDNA clone 10-9, 10-12, 10-23 and 10-24. The nucleotides encoding the part of the predicted coding region, ending in the stop codon are shown in upper case, while the predicted 3′ untranslated regions are shown in lower case. Although it is clear that contig m10.1 encodes a protein with a series of ankyrin repeats and a SOCS box, the quality of the sequence is not high enough to derive a single unambiguous open reading frame. The relationship of mouse cDNA sequence to mouse and human EST contigs is illustrated in FIG. 22.

FIG. 24 is a representation showing the nucleotide sequence of human SOCS10 cDNA contig h10.2 and h10.2 derived from analysis of the ESTs listed in Table 10.2. The relationship of these contigs to the mouse cDNA sequence is illustrated in FIG. 22.

FIG. 25A is a representation showing the partial nucleotide sequence of the human SOCS11 cDNA derived from analysis of ESTs listed in Table 11.1 The nucleotides encoding the mature coding region from the predicted ATG “start” codon to the stop codon is shown in upper case, while the predicted 5′ and 3′ untranslated regions are shown in lower case. The relationship of the partial cDNA sequence, derived from ESTs, to the predicted protein is shown in FIG. 26.

FIG. 25B is a representation showing the partial predicted amino acid sequence of human SOCS11 protein, derived from the nucleotide sequence in FIG. 25A. The SOCS box, which also shown in FIG. 4, is underlined.

FIG. 26 is a diagrammatic representation showing the relationship of sequence derived from analysis of human SOCS-11 ESTs (Table 11.1) to the predicted protein structure of human SOCS11. The deduced partial amino acid sequence of human SOCS11 is shown in FIG. 25B. The structure of the protein is shown schematically with the SH2 domain shown by the open hatched box and the SOCS box shown by the open white box. The predicted 3′ untranslated region is shown by the thin line.

FIG. 27 is a diagrammatic representation showing the relationship of mouse SOCS12 cDNA clones (12-1) to contigs derived from analysis of mouse ESTs (Table 12.1) and human ESTs (Table 12.2). The nucleotide sequence of the mouse SOCS12 contig is shown in FIG. 28, with the sequence of human SOCS12 contigs (h12.1 and h12.2) being shown in FIG. 29. The structure of the protein is shown schematically, with the ankyrin repeats indicated by the cross hatched box and the SOCS box by the open hatched box. The putative 3′ untranslated region is shown by the thin line solid line in the mouse and by the wavy line in h12.2. Based on analysis of clones isolated to date and ESTs the 3′ untranslated regions of mSOCS12 and hSOCS12 share little similarity.

FIG. 28 is a representation showing the nucleotide sequence of the mouse SOCS12 derived from analysis of cDNA clone 12-1 and the ESTs listed in Table 12.1. The nucleotides encoding the part of the predicted coding region, including the stop codon are shown in upper case, while the predicted 3′ untranslated region is shown in lower case. By homology with human SOCS12 it is clear that contig m12.1 encodes a protein with a series of ankyrin repeats and a SOCS box, the quality of the sequence is not high enough to derive a single unambiguous open reading frame. The relationship of mouse cDNA sequence to mouse and human EST contigs is illustrated in FIG. 27.

FIG. 29 is a representation showing the nucleotide sequence of human SOCS12 cDNA contig h12.1 and h12.2 derived from analysis of the ESTs listed in Table 12.2. The relationship of these contigs to the mouse cDNA sequence is illustrated in FIG. 27.

FIG. 30 is a diagrammatic representation showing the relationship of contig m13.1 derived from analysis of mouse SOCS13 cDNA clones (62-1, 62-6-7, 62-14) and mouse ESTs (Table 13.1) to contig h13.1 derived from analysis of human ESTs (Table 13.2). The nucleotide sequence of the mouse SOCS13 contig is shown in FIG. 31A, with the sequence of human SOCS13 contig (h13.1) being shown in FIG. 32. The deduced amino acid sequence of mouse SOCS13 is shown in FIG. 31B. The structure of the protein is shown schematically, with the WD-40 repeats highlighted by the open hatched box and the SOCS box shown by the densely hatched box. The 3′ untranslated region is shown by the thin line solid line.

FIG. 31A is a representation showing the nucleotide sequence of the mouse SOCS13 derived from analysis of cDNA clones 62-1, 62-6-7 and 62-14. The nucleotides encoding part of the predicted coding region, ending in the stop codon are shown in upper case, while those encoding the predicted 3′ untranslated regions are shown in lower case. The relationship of mouse cDNA sequence to mouse and human EST contigs is illustrated in FIG. 30.

FIG. 31B is a representation showing the predicted amino acid sequence of mouse SOCS13 protein, derived from the nucleotide sequence in FIG. 31A. The SOCS box, which also shown in FIG. 4 is underlined.

FIG. 32 is a representation showing the nucleotide sequence of human SOCS13 cDNA contig h13.1 derived from analysis of the ESTs listed in Table 13.2. The relationship of these contigs to the mouse cDNA sequence is illustrated in FIG. 30.

FIG. 33 is a diagrammatic representation showing the relationship of a partial mouse SOCS14 cDNA clone (14-1) to contigs derived from analysis of mouse ESTs (Table 14.1). The nucleotide sequence of the mouse SOCS14 contig is shown in FIG. 34A. The deduced partial amino acid sequence of mouse SOCS14 is shown in FIG. 34B. The structure of the protein is shown schematically, with the SH2 domain indicated by the open wave box and the SOCS box by the densely hatched box. The putative 3′ untranslated region is shown by the thin line.

FIG. 34A is a representation showing the nucleotide sequence of the mouse SOCS14 derived from analysis of genomic and cDNA clones. The nucleotides encoding the mature coding region from the predicted ATG “start” codon to the stop codon is shown in upper case, while the predicted 5′ and 3′ untranslated regions are shown in lower case. The relationship of mouse cDNA sequence to mouse and human EST contigs is illustrated in FIG. 33.

FIG. 34B is a representation showing the predicted amino acid sequence of mouse SOCS14 protein, derived from the nucleotide sequence in FIG. 34A. The SOCS box, which also shown in FIG. 4 is underlined.

FIG. 35 is a diagrammatic representation showing the relationship of contig m15.1 derived from analysis of mouse BAC and mouse ESTs (Table 15.1) to contig h15.1 derived from analysis of the human BAC and human ESTs (Table 15.2). The nucleotide sequence of the mouse SOCS15 contig is shown in FIG. 36A, with the sequence of human SOCS15 contig (h15.1) being shown in FIG. 38A. The deduced amino acid sequence of mouse SOCS15 is shown in FIG. 36B. The structure of the protein is shown schematically, with the WD-40 repeats highlighted by the open hatched box on the left and the SOCS box highlighted by the hatched box on the right. The 5′ and 3′ untranslated region are shown by the thin line solid line. The introns which interrupt the coding region are shown by ̂.

FIGS. 36A(i)-(iii) are representations showing the nucleotide sequence covering the mouse SOCS15 gene derived from analysis the mouse BAC listed in Table 15.1. The nucleotides encoding the predicted coding region, beginning with the ATG and ending in the stop codon are shown in upper case, while those encoding the predicted 5′ untranslated region, the introns and the 3′ untranslated region are shown in lower case. The relationship of mouse BAC to mouse and human ESTs contigs is illustrated in FIG. 35.

FIG. 36B is a representation showing the predicted amino acid sequence of mouse SOCS15 protein, derived from the nucleotide sequence in FIG. 36A. The SOCS box, which also shown in FIG. 4 is underlined.

FIGS. 37A(i)-(iv) are representations showing the nucleotide sequence covering the human SOCS15 gene derived from analysis the human BAC listed in Table 15.2. The nucleotides encoding the predicted coding region, beginning with the ATG and ending in the stop codon are shown in upper case, while those encoding the predicted 5′ untranslated region, the introns and the 3′ untranslated region are shown in lower case. The relationship of the human BAC to mouse and human ESTs contigs is illustrated in FIG. 35.

FIG. 37B is a representation showing the predicted amino acid sequence of human SOCS15 protein, derived from the nucleotide sequence in FIG. 37A. The SOCS box, which also shown in FIG. 4 is underlined.

FIG. 38 is a diagrammatic representation of pβgalpAloxneo.

FIG. 39 is a diagrammatic representation of pβgalpAloxneoTK.

FIG. 40 is a diagrammatic representation of SOCS1 knockout construct.

FIGS. 41A-41C are the nucleotide sequence and predicted amino acid sequence of the coding region mouse SOCS1mutXHO.

FIG. 42 is the alignment of the nucleotide and predicted amino acid sequence of the first 28 amino acids of the SH2 domain of CIS, SOCS1, SOCS2 and SOCS3, introduced nucleotide changes that lead to the R>K amino acid substitution are underlined.

FIG. 43A is a schematic representation of SOCS-1 genomic map in mice.

FIG. 43B is a schematic representation of the genomic map in mice.

FIG. 43C is a schematic representation of the SOCS-3 genomic map in mice.

FIG. 43D is a schematic representation of the SOCS-5 genomic map in mice.

FIG. 43E is a schematic representation of SOCS-9 genomic map in mice.

FIG. 43F is a schematic representation of the SOCS-11 genomic map in mice.

FIGS. 44A and 44B are diagrammatic representation of SOCS-1 targeting.

FIGS. 45A and 45B are diagrammatic representation of SOCS-2 targeting.

FIGS. 46A and 46B are diagrammatic representation of SOCS-3 targeting.

FIGS. 47A and 47B are diagrammatic representation of CIS targeting.

FIG. 48 is a diagrammatic representation of pEF-GFP-I/m SOCS-1.

FIG. 49 is a representation showing nucleotide sequences of oligonucleotides and full length coding sequence of SOCS-1.

FIG. 50 is a representation of amino acid sequence of SOCS-13.

FIGS. 51A and 51B are (A) nucleotide of mouse SOCS-5. The predicted translational start and stop sites underlined; (B) predicted amino acid sequence of mouse SOCS-5. The conventional three-letter code for amino acids is used.

FIGS. 52A and 52B are (A) nucleotide of mouse SOCS-9. The predicted translational start and stop sites underlined; (B) predicted amino acid sequence of mouse SOCS-9. The conventional three-letter code for amino acids is used.

### DETAILED DESCRIPTION OF PREFERRED EMBODIMENTS

The present invention provides a new family of modulators of signal transduction. As the initial members of this family suppressed cytokine signalling, the family is referred to as the “suppressors of cytokine signalling” family of “SOCS”. The SOCS family is defined by the presence of a C-terminal domain referred to as a “SOCS box”. Different classes of SOCS molecules are defined by a motif generally but not exclusively located N-terminal to the SOCS box and which is involved by protein:molecule interaction such as protein:DNA, or protein:protein interaction. Particularly preferred motifs are selected from an SH2 domain, WD-40 repeats and ankyrin repeats.

WD-40 repeats were originally recognised in the β-subunit of G-proteins. WD-40 repeats appear to form a β-propeller-like structure and may be involved in protein-protein interactions. Ankyrin repeats were originally recognised in the cytoskeletal protein ankryin.

Members of the SOCS family may be identified by any number of means. For example, SOCS1 to SOCS3 were identified by their ability to suppress cytokine-mediated signal transduction and, hence, were identified based on activity. SOCS4 to SOCS15 were identified as nucleotide sequences exhibiting similarity at the level of the SOCS box.

The SOCS box is a conserved motif located in the C-terminal region of the SOCS molecule. In accordance with the present invention, the amino acid sequence of the SOCS box is:

X₁X₂X₃X₄X₅X₆X₇X₈X₈X₁₀X₁₁X₁₂X₁₃X₁₄X₁₅X₁₆[Xᵢ]ₙX₁₇X₁₈
X₁₉X₂₀X₂₁X₂₂X₂₃[Xj]ₙX₂₄X₂₅X₂₆X₂₇X₂₈

wherein: X₁ is L, I, V, M, A or P;
   X₂ is any amino acid residue;
   X₃ is P, T or S;
   X₄ is L, I, V, M, A or P;
   X₅ is any amino acid;
   X₆ is any amino acid;
   X₇ is L, I, V, M, A, F, Y or W;
   X₈ is C, T or S;
   X₉ is R, K or H;
   X₁₀ is any amino acid;
   X₁₁, is any amino acid;
   X₁₂ is L, I, V, M, A or P;
   X₁₃ is any amino acid;
   X₁₄ is any amino acid;
   X₁₅ is any amino acid;
   X₁₆ is L, I, V, M, A, P, G, C, T or S;
   [Xᵢ]ₙ is a sequence of n amino acids wherein n is from 1 to 50 amino acids and wherein the sequence Xᵢ may comprise the same or different amino acids selected from any amino acid residue;
   X₁₇ is L, I, V, M, A or P;
   X₁₈ is any amino acid;
   X₁₉ is any amino acid;
   X₂₀ L, I, V, M, A or P;
   X₂₁ is P;
   X₂₂ is L, I, V, M, A, P or G;
   X₂₃ is P or N;
   [Xⱼ]ₙ is a sequence of n amino acids wherein n is from 0 to 50 amino acids and wherein the sequence Xⱼ may comprise the same or different amino acids selected from any amino acid residue;
   X₂₄ is L, I, V, M, A or P;
   X₂₅ is any amino acid;
   X₂₆ is any amino acid;
   X₂₇ is Y or F; and
   X₂₈ is L, I, V, M, A or P.

As stated above and in accordance with the present invention, SOCS proteins are divided into separate classes based on the presence of a protein:molecule interacting region such as but not limited to an SH2 domain, WD-40 repeats and ankyrin repeats located N-terminal of the SOCS box. The latter three domains are protein:protein interacting domains.

Examples of SH2 containing SOCS proteins include SOCS1, SOCS2, SOCS3, SOCS5, SOCS9, SOCS11 and SOCS14. Examples of SOCS containing WD-40 repeats include SOCS4, SOCS6 and SOCS15. Examples of SOCS containing ankyrin repeats include SOCS7, SOCS10 and SOCS12.

The present invention provides inter alia nucleic acid molecules encoding SOCS proteins, purified naturally occurring SOCS proteins as well as recombinant forms of SOCS proteins and methods of modulating signal transduction by modulating activity of SOCS proteins or expression of SOCS genes. Preferably, signal transduction is mediated by a cytokine, examples of which include EPO, TPO, G-CSF, GM-CSF, IL-3, IL-2, IL-4, IL-7, IL-13, IL-6, LIF, IL-12, IFNγ, TNFα, IL-1 and/or M-CSF. Particularly preferred cytokines include IL-6, LIF, OSM, IFN-γ and/or thrombopoietin.

Accordingly, one aspect of the present invention provides an isolated nucleic acid molecule comprising a sequence of nucleotides encoding or complementary to a sequence encoding a protein or a derivative, homologue, analogue or mimetic thereof or comprises a nucleotide sequence capable of hybridizing thereto under low stringency conditions at 42° C. wherein said protein comprises a SOCS box in its C-terminal region and optionally a protein:molecule interacting domain N-terminal of the SOCS box.

Preferably, the protein:molecule interacting domain is a protein:DNA or protein:protein interacting domain. Most preferably, the protein:molecule interacting domain is one of an SH2 domain, WD-40 repeats and/or ankyrin repeats.

As stated above, preferably the subject SOCS modulate cytokine-mediated signal transduction. The present invention extends, however, to SOCS molecules modulating other effector-mediated signal transduction such as mediated by other endogenous or exogenous molecules, antigens, microbes and microbial products, viruses or components thereof, ions, hormones and parasites. Endogenous molecules in this context are molecules produced within the cell carrying the SOCS molecule. Exogenous molecules are produced by other cells or are introduced to the body.

Preferably, the nucleic acid molecule or SOCS protein is in isolated or purified form. The terms “isolated” and “purified” mean that a molecule has undergone at least one purification step away from other material.

Preferably, the nucleic acid molecule is in isolated form and is DNA such as cDNA or genomic DNA. The DNA may encode the same amino acid sequence as the naturally occurring SOCS or the SOCS may contain one or more amino acid substitutions, deletions and/or additions. The nucleotide sequence may correspond to the genomic coding sequence (including exons and introns) or to the nucleotide sequence in cDNA from mRNA transcribed from the genomic gene or it may carry one or more nucleotide substitutions, deletions and/or additions thereto.

In a preferred embodiment, the nucleic acid molecule comprises a sequence of nucleotide encoding or complementary to a sequence encoding a SOCS protein or a derivative, homologue, analogue or mimetic thereof wherein the amino acid sequence of said SOCS protein is selected from SEQ ID NO:4 (mSOCS1), SEQ ID NO:6 (mSOCS2), SEQ ID NO:8 (mSOCS3), SEQ, ID NO:10 (hSOCS1), SEQ ID NO:12 (rSOCS1), SEQ ID NO:14 (mSOCS4), SEQ ID NO:18 (mSOCS5), SEQ ID NO:21 (mSOCS6), SEQ ID NO:25 (mSOCS27), SEQ ID NO:29 (mSOCS8), SEQ ID NO:36 (hSOCS11), SEQ ID NO:41 (mSOCS13), SEQ ID NO:44 (mSOCS14), SEQ ID NO:46 (mSOCS15) and SEQ ID NO:48 (mSOCS15) or encodes an amino acid sequence with a single or multiple amino acid substitution, deletion and/or addition to the listed sequences or is a nucleotide sequence capable of hybridizing to the nucleic acid molecule under low stringency conditions at 42° C.

In an even more preferred embodiment, the present invention provides a nucleic acid molecule comprising a sequence of nucleotides encoding or complementary to a sequence encoding a SOCS protein or a derivative, homologue, analogue or mimetic thereof wherein the nucleotide sequence is selected from a nucleotide sequence substantially set forth in SEQ ID NO:3 (mSOCS1), SEQ ID NO:5 (mSOCS2), SEQ ID NO:7 (mSOCS3), SEQ ID NO:9 (hSOCS11), SEQ ID NO:11 (rSOCS1), SEQ ID NO:13 (mSOCS4), SEQ ID NO:15 and SEQ ID NO:16 (hSOCS4), SEQ ID NO: 17 (mSOCS5), SEQ ID NO: 19 (hSOCS5), SEQ ID NO:20 (mSOCS6), SEQ ID NO:22 and SEQ ID NO:23 (hSOCS6), SEQ ID NO:24 (mSOCS7), SEQ ID NO:26 and SEQ ID NO:27 (hSOCS7), SEQ ID NO:28 (mSOCS8), SEQ ID NO:30 (mSOCS9), SEQ ID NO:31 (hSOCS9), SEQ ID NO:32 (mSOCS10), SEQ ID NO:33 and SEQ ID NO:34 (hSOCS10), SEQ ID NO:35 (hSOCS11), SEQ ID NO:37 (mSOCS12), SEQ ID NO:38 and SEQ ID NO:39 (hSOCS12), SEQ ID NO:40 (mSOCS13), SEQ ID NO:42 (hSOCS13), SEQ ID NO:43 (mSOCS14), SEQ ID NO:45 (mSOCS15) and SEQ ID NO:47 (hSOCS15) or a nucleotide sequence having at least about 15% similarity to all or a region of any of the listed sequences or a nucleic acid molecule capable of hybridizing to any of the listed sequences under low stringency conditions at 42° C.

Reference herein to a low stringency at 42° C. includes and encompasses from at least about 1% v/v to at least about 15% v/v formamide and from at least about 1M to at least about 2M salt for hybridisation, and at least about 1M to at least about 2M salt for washing conditions. Alternative stringency conditions may be applied where necessary, such as medium stringency, which includes and encompasses from at least about 16% v/v to at least about 30% v/v formamide and from at least about 0.5M to at least about 0.9M salt for hybridisation, and at least about 0.5M to at least about 0.9M salt for washing conditions, or high stringency, which includes and encompasses from at least about 31% v/v to at least about 50% v/v formamide and from at least about 0.01M to at least about 0.15M salt for hybridisation, and at least about 0.01M to at least about 0.15M salt for washing conditions.

In another embodiment, the present invention is directed to a SOCS protein or a derivative, homologue, analogue or mimetic thereof wherein said SOCS protein is identified as follows:
human SOCS4 characterised by EST81149, EST180909, EST182619, ya99H09, ye70co4, yh53c09, yh77g11, yh87h05, yi45h07, yj04e06, yq12h06, yq56a06, yq60e02, yq92g03, yq97h06, yr90f01, yt69c03, yv30a08, yv55f07, yv57h09, yv87h02, yv98e11, yw68d10, yw82a03, yx08a07, yx72h06, yx76b09, yy37h08, yy66b02, za81f08, zb18f07, zc06e08, zd14g06, zd51h12, zd52b09, ze25 g11, ze69f02, zf54f03, zh96e07, zv66h12, zs83a08 and zs83g08;
mouse SOCS-4 characterised by mc65f04, mf42e06, mp10c10, mr81 g09, and mt19h12;
human SOCS-5 characterised by EST15B103, EST15B105, EST27530 and zf50f01;
mouse SOCS-5 characterised by mc55a01, mh98f09, my26h12 and ve24e06;
human SOCS-6 characterised by yf61e08, yf93a09, yg05f12, yg41f04, yg45c02, yh11f10, yh13b05, zc35a12, ze02h08, zl09a03, zl69e10, zn39d08 and zo39e06;
mouse SOCS-6 characterised by mc04c05, md48a03, mf31d03, mh26b07, mh78e11, mh88h09, mh94h07, mi27h04 and mj29c05, mp66g04, mw75g03, va53b05, vb34h02, vc55d07, vc59e05, vc67d03, vc68d10, vc97h01, vc99c08, vd07h03, vd08c01, vd09b12, vd19b02, vd29a04 and vd46d06;
human SOCS-7 characterised by STS WI30171, EST00939, EST12913, yc29b05, yp49f10, zt10f03 and zx73g04;
mouse SOCS-7 characterised by mj39a01 and vi52h07;
mouse SOCS-8 characterised by mj6e09 and vj27a029;
human SOCS-9 characterised by CSRL-82f2-u, EST 114054, yy06b07, yy06g06, zr40c09, zr72h01, yx92c08, yx93b08 and hfe0662;
mouse SOCS-9 characterised by me65d05;
human SOCS-10 characterised by aa48h10, zp35h01, zp97h12, zq08h01, zr34g05, EST73000 and HSDHEI005;
mouse SOCS-10 characterised by mb14d12, mb40f06, mg89b11, mq89e12, mp03g12 and vh53c11;
human SOCS-11 characterised by zt24h06 and zr43b02;
human SOCS-13 characterised by EST59161;
mouse SOCS-13 characterised by ma39a09, me60c05, mi78g05, mk10c11, mo48g12, mp94a01, vb57c07 and vh07c11; and
human SOCS-14 characterised by mi75e03, vd29h11 and vd53g07; or a derivative or homologue of the above ESTs characterised by a nucleic acid molecule being capable of hybridizing to any of the listed ESTs under low stringency conditions at 42° C.

In another embodiment, the nucleotide sequence encodes the following amino acid sequence:

X₁X₂X₃X₄X₅X₆X₇X₈X₈X₁₀X₁₁X₁₂X₁₃X₁₄X₁₅X₁₆[Xᵢ]ₙX₁₇X₁₈
X₁₉X₂₀X₂₁X₂₂X₂₃[Xj]ₙX₂₄X₂₅X₂₆X₂₇X₂₈

wherein: X₁ is L, I, V, M, A or P;
   X₂ is any amino acid residue;
   X₃ is P, T or S;
   X₄ is L, I, V, M, A or P;
   Xs is any amino acid;
   X₆ is any amino acid;
   X₇ is L, I, V, M, A, F, Y or W;
   X₈ is C, T or S;
   X₉ is R, K or H;
   X₁₀ is any amino acid;
   X₁₁ is any amino acid;
   X₁₂ is L, I, V, M, A or P;
   X₁₃ is any amino acid;
   X₁₄ is any amino acid;
   X₁₅ is any amino acid;
   X₁₆ is L, I, V, M, A, P, G, C, T or S;
   [Xᵢ]ₙ is a sequence of n amino acids wherein n is from 1 to 50 amino acids and wherein the sequence Xᵢ may comprise the same or different amino acids selected from any amino acid residue;
   X₁₇ is L, I, V, M, A or P;
   X₁₈ is any amino acid;
   X₁₉ is any amino acid;
   X₂₀ L, I, V, M, A or P;
   X₂₁ is P;
   X₂₂ is L, I, V, M, A, P or G;
   X₂₃ is P or N;
   [Xⱼ]ₙ is a sequence of n amino acids wherein n is from 0 to 50 amino acids and wherein the sequence Xⱼ may comprise the same or different amino acids selected from any amino acid residue;
   X₂₄ is L, I, V, M, A or P;
   X₂₅ is any amino acid;
   X₂₆ is any amino acid;
   X₂₇ is Y or F; and
   X₂₈ is L, I, V, M, A or P.

The above sequence comparisons are preferably to the whole molecule but may also be to part thereof. Preferably, the comparisons are made to a contiguous series of at least about 21 nucleotides or at least about 5 amino acids. More preferably, the comparisons are made against at least about 21 contiguous nucleotides or at least 7 contiguous amino acids. Comparisons may also only be made to the SOCS box region or a region encompassing the protein:molecule interacting region such as the SH2 domain WD-40 repeats and/or ankyrin repeats.

Still another embodiment of the present invention contemplates an isolated polypeptide or a derivative, homologue, analogue or mimetic thereof comprising a SOCS box in its C-terminal region.

Preferably the polypeptide further comprises a protein:molecule interacting domain such as a protein:DNA or protein:protein interacting domain. Preferably, this domain is located N-terminal of the SOCS box. It is particularly preferred for the protein:molecule interacting domain to be at least one of an SH2 domain, WD-40 repeats and/or ankyrin repeats.

Preferably, the signal transduction is mediated by a cytokine selected from EPO, TPO, G-CSF, GM-CSF, IL-3, IL-2, IL-4, IL-7, IL-13, IL-6, LIF, IL-12, IFNγ, TNFα, IL-1 and/or M-CSF. Preferred cytokines are IL-6, LIF, OSM, IFN-γ or thrombopoietin.

More preferably, the protein comprises a SOCS box having the amino acid sequence:

X₁X₂X₃X₄X₅X₆X₇X₈X₈X₁₀X₁₁X₁₂X₁₃X₁₄X₁₅X₁₆[Xᵢ]ₙX₁₇X₁₈
X₁₉X₂₀X₂₁X₂₂X₂₃[Xj]ₙX₂₄X₂₅X₂₆X₂₇X₂₈

wherein: X₁ is L, I, V, M, A or P;
   X₂ is any amino acid residue;
   X₃ is P, T or S;
   X₄ is L, I, V, M, A or P;
   X₅ is any amino acid;
   X₆ is any amino acid;
   X₇ is L, I, V, M, A, F, Y or W;
   X₈ is C, T or S;
   X₉ is R, K or H;
   X₁₀ is any amino acid;
   X₁₁ is any amino acid;
   X₁₂ is L, I, V, M, A or P;
   X₁₃ is any amino acid;
   X₁₄ is any amino acid;
   X₁₅ is any amino acid;
   X₁₆ is L, I, V, M, A, P, G, C, T or S;
   [Xᵢ]ₙ is a sequence of n amino acids wherein n is from 1 to 50 amino acids and wherein the sequence Xᵢ may comprise the same or different amino acids selected from any amino acid residue;
   X₁₇ is L, I, V, M, A or P;
   X₁₈ is any amino acid;
   X₁₉ is any amino acid;
   X₂₀ L, I, V, M, A or P;
   X₂₁ is P;
   X₂₂ is L, I, V, M, A, P or G;
   X₂₃ is P or N;
   [Xⱼ]ₙ is a sequence of n amino acids wherein n is from 0 to 50 amino acids and wherein the sequence Xⱼ may comprise the same or different amino acids selected from any amino acid residue;
   X₂₄ is L, I, V, M, A or P;
   X₂₅ is any amino acid;
   X₂₆ is any amino acid;
   X₂₇ is Y or F; and
   X₂₈ is L, V, M, A or P.

Still another embodiment provides an isolated polypeptide or a derivative, homologue, analogue or mimetic thereof comprising a sequence of amino acids substantially as set forth in SEQ ID NO:4 (mSOCS1), SEQ ID NO:6 (mSOCS2), SEQ ID NO:8 (mSOCS3), SEQ ID NO:10 (hSOCS1), SEQ ID NO:12 (rSOCS1), SEQ ID NO:14 (mSOCS4), SEQ ID NO:18 (mSOCS5), SEQ ID NO:21 (mSOCS6), SEQ ID NO:25 (mSOCS7), SEQ ID NO:29 (mSOCS8), SEQ ID NO:36 (hSOCS11), SEQ ID NO:41 (mSOCS13), SEQ ID NO:44 (mSOCS14), SEQ ID NO:46 (mSOCS15) and SEQ ID NO:48 (hSOCS15) or an amino acid sequence having at least 15% similarity to all or a part of the listed sequences.

Preferred nucleotide percentage similarities include at least about 20%, at least about 40%, at least about 50%, at least about 60%, at least about 70%, at least about 80%, at least about 90% or above such as 93%, 95%, 98% or 99%.

Preferred amino acid similarities include at least about 20%, at least about 30%, at least about 40%, at least about 50%, at least about 60%, at least about 70%, at least about 80%, at least about 90%, at least about 95%, at least about 97% or 98% or above.

As stated above, similarity may be measured against an entire molecule or a region comprising at least 21 nucleotides or at least 7 amino acids. Preferably, similarity is measured in a conserved region such as SH2 domain, WD-40 repeats, ankyrin repeats or other protein:molecule interacting domains or a SOCS box.

The term “similarity” includes exact identity between sequences or, where the sequence differs, different amino acids are related to each other at the structural, functional, biochemical and/or conformational levels.

The nucleic acid molecule may be isolated from any animal such as humans, primates, livestock animals (e.g. horses, cows, sheep, donkeys, pigs), laboratory test animals (e.g. mice, rats, rabbits, hamsters, guinea pigs), companion animals (e.g. dogs, cats) or captive wild animals (e.g. deer, foxes, kangaroos).

The terms “derivatives” or its singular form “derivative” whether in relation to a nucleic acid molecule or a protein includes parts, mutants, fragments and analogues as well as hybrid or fusion molecules and glycosylation variants. Particularly useful derivatives comprise single or multiple amino acid substitutions, deletions and/or additions to the SOCS amino acid sequence.

Preferably, the derivatives have functional activity or alternatively act as antagonists or agonists. The present invention further extends to homologues of SOCS which include the functionally or structurally related molecule from different animal species. The present invention also encompasses analogues and mimetics. Mimetics include a class of molecule generally but not necessarily having a non-amino acid structure and which functionally are capable of acting in an analogous manner to the protein for which it is a mimic, in this case, a SOCS. Mimetics may comprise a carbohydrate, aromatic ring, lipid or other complex chemical structure or may also be proteinaceous in composition. Mimetics as well as agonists and antagonists contemplated herein are conveniently located through systematic searching of environments, such as coral, marine and freshwater river beds, flora and microorganisms. This is sometimes referred to as natural product screening. Alternatively, libraries of synthetic chemical compounds may be screened for potentially useful molecules.

The present invention further extends to a range of deletion mutants such as SOCS molecules carrying deletion in the carboxy terminal region, the amino terminal region and in both the carboxy and amino terminal regions. Molecules are also contemplated by the present invention which encompasses only the carboxy terminal region or amino terminal region or fused to another peptide, polypeptide or protein. Molecules comprising the amino terminal portion of the SOCS molecules are particularly useful as molecules capable of interacting with cytokines. For example, the N-terminal region of SOCS-1 is critical for inhibition of M1 macrophage differentiation and LIF and IL-6 signalling.

As stated above, the present invention contemplates agonists and antagonists of the SOCS. One example of an antagonist is an antisense oligonucleotide sequence. Useful oligonucleotides are those which have a nucleotide sequence complementary to at least a portion of the protein-coding or “sense” sequence of the nucleotide sequence. These anti-sense nucleotides can be used to effect the specific inhibition of gene expression. The antisense approach can cause inhibition of gene expression apparently by forming an anti-parallel duplex by complementary base pairing between the antisense construct and the targeted mRNA, presumably resulting in hybridisation arrest of translation. Ribozymes and co-suppression molecules may also be used. Antisense and other nucleic acid molecules may first need to be chemically modified to permit penetration of cell membranes and/or to increase their serum half life or otherwise make them more stable for in vivo administration. Antibodies may also act as either antagonists or agonists although are more useful in diagnostic applications or in the purification of SOCS proteins. Antagonists and agonists may also be identified following natural product screening or screening of libraries of chemical compounds or may be derivatives or analogues of the SOCS molecules. Agonists and antagonists of SOCS proteins contemplated by the present invention include carboxy-terminal and N-terminal portions of the SOCS molecule. For example, the N-terminal portion of SOCS-1 is useful in inhibiting LIF and IL-6 signalling.

Accordingly, the present invention extends to analogues of the SOCS proteins of the present invention. Analogues may be used, for example, in the treatment or prophylaxis of cytokine mediated dysfunction such as autoimmunity, immune suppression or hyperactive immunity or other condition including but not limited to dysfunctions in the haemopoietic, endocrine, hepatic and neural systems. Dysfunctions mediated by other signal transducing elements such as hormones or endogenous or exogenous molecules, antigens, microbes and microbial products, viruses or components thereof, ions, hormones and parasites are also contemplated by the present invention.

Analogues of the proteins contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecule or their analogues.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 3.

TABLE 3

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

These types of modifications may be important to stabilise the cytokines if administered to an individual or for use as a diagnostic reagent.

Other derivatives contemplated by the present invention include a range of glycosylation variants from a completely unglycosylated molecule to a modified glycosylated molecule. Altered glycosylation patterns may result from expression of recombinant molecules in different host cells.

Another embodiment of the present invention contemplates a method for modulating expression of a SOCS protein in a mammal, said method comprising contacting a gene encoding a SOCS or a factor/element involved in controlling expression of the SOCS gene with an effective amount of a modulator of SOCS expression for a time and under conditions sufficient to up-regulate or down-regulate or otherwise modulate expression of SOCS. An example of a modulator is a cytokine such as IL-6 or other transcription regulators of SOCS expression.

Expression includes transcription or translation or both.

Another aspect of the present invention contemplates a method of modulating activity of SOCS in a human, said method comprising administering to said mammal a modulating effective amount of a molecule for a time and under conditions sufficient to increase or decrease SOCS activity. The molecule may be a proteinaceous molecule or a chemical entity and may also be a derivative of SOCS or a chemical analogue or truncation mutant of SOCS.

A further aspect of the present invention provides a method of inducing synthesis of a SOCS or transcription/translation of a SOCS comprising contacting a cell containing a SOCS gene with an effective amount of a cytokine capable of inducing said SOCS for a time and under conditions sufficient for said SOCS to be produced. For example, SOCS1 may be induced by IL-6.

Still a further aspect of the present invention contemplates a method of modulating levels of a SOCS protein in a cell said method comprising contacting a cell containing a SOCS gene with an effective amount of a modulator of SOCS gene expression or SOCS protein activity for a time and under conditions sufficient to modulate levels of said SOCS protein.

Yet a further aspect of the present invention contemplates a method of modulating signal transduction in a cell containing a SOCS gene comprising contacting said cell with an effective amount of a modulator of SOCS gene expression or SOCS protein activity for a time sufficient to modulate signal transduction.

Even yet a further aspect of the present invention contemplates a method of influencing interaction between cells wherein at least one cell carries a SOCS gene, said method comprising contacting the cell carrying the SOCS gene with an effective amount of a modulator of SOCS gene expression or SOCS protein activity for a time sufficient to modulate signal transduction.

As stated above, of the present invention contemplates a range of mimetics or small molecules capable of acting as agonists or antagonists of the SOCS. Such molecules may be obtained from natural product screening such as from coral, soil, plants or the ocean or antarctic environments. Alternatively, peptide, polypeptide or protein libraries or chemical libraries may be readily screened. For example, M1 cells expressing a SOCS do not undergo differentiation in the presence of IL-6. This system can be used to screen molecules which permit differentiation in the presence of IL-6 and a SOCS. A range of test cells may be prepared to screen for antagonists and agonists for a range of cytokines. Such molecules are preferably small molecules and may be of amino acid origin or of chemical origin. SOCS molecules interacting with signalling proteins (eg. JAKS) provide molecular screens to detect molecules which interfere or promote this interaction. Once such screening protocol involves natural product screening.

Accordingly, the present invention contemplates a pharmaceutical composition comprising SOCS or a derivative thereof or a modulator of SOCS expression or SOCS activity and one or more pharmaceutically acceptable carriers and/or diluents. These components are referred to as the “active ingredients”. These and other aspects of the present invention apply to any SOCS molecules such as but not limited to SOCS1 to SOCS15.

The pharmaceutical forms containing active ingredients suitable for injectable use include sterile aqueous solutions (where water soluble) sterile powders for the extemporaneous preparation of sterile injectable solutions. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as licithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets. For oral therapeutic administration, the active compound may be incorporated with excipients and used in the form of ingestible tablets, buccal tablets, troches, capsules, elixirs, suspensions, syrups, wafers and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter. A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The present invention also extends to forms suitable for topical application such as creams, lotions and gels.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form as hereinbefore disclosed. A unit dosage form can, for example, contain the principal active compound in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients. The effective amount may also be conveniently expressed in terms of an amount per kg of body weight. For example, from about 0.01 ng to about 10,000 mg/kg body weight may be administered.

The pharmaceutical composition may also comprise genetic molecules such as a vector capable of transfecting target cells where the vector carries a nucleic acid molecule capable of modulating SOCS expression or SOCS activity. The vector may, for example, be a viral vector. In this regard, a range of gene therapies are contemplated by the present invention including isolating certain cells, genetically manipulating and returning the cell to the same subject or to a genetically related or similar subject.

Still another aspect of the present invention is directed to antibodies to SOCS and its derivatives. Such antibodies may be monoclonal or polyclonal and may be selected from naturally occurring antibodies to SOCS or may be specifically raised to SOCS or derivatives thereof. In the case of the latter, SOCS or its derivatives may first need to be associated with a carrier molecule. The antibodies and/or recombinant SOCS or its derivatives of the present invention are particularly useful as therapeutic or diagnostic agents.

For example, SOCS and its derivatives can be used to screen for naturally occurring antibodies to SOCS. These may occur, for example in some autoimmune diseases. Alternatively, specific antibodies can be used to screen for SOCS. Techniques for such assays are well known in the art and include, for example, sandwich assays and ELISA. Knowledge of SOCS levels may be important for diagnosis of certain cancers or a predisposition to cancers or monitoring cytokine mediated cellular responsiveness or for monitoring certain therapeutic protocols.

Antibodies to SOCS of the present invention may be monoclonal or polyclonal. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool for assessing apoptosis or monitoring the program of a therapeutic regimen.

For example, specific antibodies can be used to screen for SOCS proteins. The latter would be important, for example, as a means for screening for levels of SOCS in a cell extract or other biological fluid or purifying SOCS made by recombinant means from culture supernatant fluid. Techniques for the assays contemplated herein are known in the art and include, for example, sandwich assays and ELISA.

It is within the scope of this invention to include any second antibodies (monoclonal, polyclonal or fragments of antibodies or synthetic antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of SOCS.

Both polyclonal and monoclonal antibodies are obtainable by immunization with the enzyme or protein and either type is utilizable for immunoassays. The methods of obtaining both types of sera are well known in the art. Polyclonal sera are less preferred but are relatively easily prepared by injection of a suitable laboratory animal with an effective amount of SOCS, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art.

Another aspect of the present invention contemplates a method for detecting SOCS in a biological sample from a subject said method comprising contacting said biological sample with an antibody specific for SOCS or its derivatives or homologues for a time and under conditions sufficient for an antibody-SOCS complex to form and then detecting said complex.

The presence of SOCS may be accomplished in a number of ways such as by Western blotting and ELISA procedures. A wide range of immunoassay techniques are available as can be seen by reference to U.S. Pat. Nos. 4,016,043, 4,424,279 and 4,018,653. These, of course, include both single-site and two-site or “sandwich” assays of the non-competitive types, as well as in the traditional competitive binding assays. These assays also include direct binding of a labelled antibody to a target.

Sandwich assays are among the most useful and commonly used assays and are favoured for use in the present invention. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention the sample is one which might contain SOCS including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

In the typical forward sandwich assay, a first antibody having specificity for the SOCS or antigenic parts thereof, is either covalently or passively bound to a solid surface. The solid surface Is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. room temperature to 37° C.) to allow binding of any subunit present in the antibody. Following the incubation period, the antibody subunit solid phase is washed and dried and incubated with a second antibody specific for a portion of the hapten. The second antibody is linked to a reporter molecule which is used to indicate the binding of the second antibody to the hapten.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescene and EIA techniques are both very well established in the art and are particularly preferred for the present method. However, other reporter molecules, such as radioisotope, chemiluminescent or bioluminescent molecules, may also be employed.

The present invention also contemplates genetic assays such as involving PCR analysis to detect SOCS gene or its derivatives. Alternative methods or methods used in conjunction include direct nucleotide sequencing or mutation scanning such as single stranded conformation polymorphisms analysis (SSCP) as specific oligonucleotide hybridisation, as methods such as direct protein truncation tests.

Since cytokines are involved in transcription of some SOCS molecules, the detection of SOCS provides surrogate markers for cytokines or cytokine activity. This may be useful in assessing subjects with a range of conditions such as those will autoimmune diseases, for example, rheumatoid arthritis, diabetes and stiff man syndrome amongst others.

The nucleic acid molecules of the present invention may be DNA or RNA. When the nucleic acid molecule is in DNA form, it may be genomic DNA or cDNA. RNA forms of the nucleic acid molecules of the present invention are generally mRNA.

Although the nucleic acid molecules of the present invention are generally in isolated form, they may be integrated into or ligated to or otherwise fused or associated with other genetic molecules such as vector molecules and in particular expression vector molecules. Vectors and expression vectors are generally capable of replication and, if applicable, expression in one or both of a prokaryotic cell or a eukaryotic cell. Preferably, prokaryotic cells include E. coli, Bacillus sp and Pseudomonas sp. Preferred eukaryotic cells include yeast, fungal, mammalian and insect cells.

Accordingly, another aspect of the present invention contemplates a genetic construct comprising a vector portion and a mammalian and more particularly a human SOCS gene portion, which SOCS gene portion is capable of encoding a SOCS polypeptide or a functional or immunologically interactive derivative thereof.

Preferably, the SOCS gene portion of the genetic construct is operably linked to a promoter on the vector such that said promoter is capable of directing expression of said SOCS gene portion in an appropriate cell.

In addition, the SOCS gene portion of the genetic construct may comprise all or part of the gene fused to another genetic sequence such as a nucleotide sequence encoding glutathione-S-transferase or part thereof.

The present invention extends to such genetic constructs and to prokaryotic or eukaryotic cells comprising same.

The present invention also extends to any or all derivatives of SOCS including mutants, part, fragments, portions, homologues and analogues or their encoding genetic sequence including single or multiple nucleotide or amino acid substitutions, additions and/or deletions to the naturally occurring nucleotide or amino acid sequence. The present invention also extends to mimetics and agonists and antagonists of SOCS.

The SOCS and its genetic sequence of the present invention will be useful in the generation of a range of therapeutic and diagnostic reagents and will be especially useful in the detection of a cytokine involved in a particular cellular response or a receptor for that cytokine. For example, cells expressing SOCS gene such as M1 cells expressing the SOCS1 gene, will no longer be responsive to a particular cytokine such as, in the case of SOCS1, IL-6. Clearly, the present invention further contemplates cells such as M1 cells expressing any SOCS gene such as from SOCS1 to SOCS15. Furthermore, the present invention provides the use of molecules that regulate or potentiate the ability of therapeutic cytokines. For example, molecules which block some SOCS activity, may act to potential therapeutic cytokine activity (eg. G-CSF).

Soluble SOCS polypeptides are also contemplated to be particularly useful in the treatment of disease, injury or abnormality involving cytokine mediated cellular responsiveness such as hyperimmunity, immunosuppression, allergies, hypertension and the like.

A further aspect of the present invention contemplates the use of SOCS or its functional derivatives in the manufacture of a medicament for the treatment of conditions involving cytokine mediated cellular responsiveness.

The present invention further contemplates transgenic mammalian cells expressing a SOCS gene. Such cells are useful indicator cell lines for assaying for suppression of cytokine function. One example is M1 cells expressing a SOCS gene. Such cell lines may be useful for screening for cytokines or screening molecules such as naturally occurring molecules from plants, coral, microorganisms or bio-organically active soil or water capable of acting as cytokine antagonists or agonists. The present invention further contemplates transgenic animals such as mice, rats, sheep, pigs, rabbits and guinea pigs which are homozygous or heterozygous knockout animals for the SOCS genes or parts thereof.

The present invention further contemplates hybrids between different SOCS from the same or different animal species. For example, a hybrid may be formed between all or a functional part of mouse SOCS1 and human SOCS1. Alternatively, the hybrid may be between all or part of mouse SOCS1 and mouse SOCS2. All such hybrids are contemplated herein and are particularly useful in developing pleiotropic molecules.

The present invention further contemplates a range of genetic based diagnostic assays screening for individuals with defective SOCS genes. Such mutations may result in cell types not being responsive to a particular cytokine or resulting in over responsiveness leading to a range of conditions. The SOCS genetic sequence can be readily verified using a range of PCR or other techniques to determine whether a mutation is resident in the gene. Appropriate gene therapy or other interventionist therapy may then be adopted.

The present invention is further described by the following non-limiting Examples.

Examples 1-16 relate to SOCS1, SOCS2 and SOCS3 which were identified on the basis of activity. Examples 17-24 relate to various aspects of SOCS4 to SOCS15 which were cloned initially on the basis of sequence similarity. Examples 25-36 relate to specific aspects of SOCS4 to SOCS15, respectively.

### Example 1

### Cell Culture and Cytokines

The M1 cell line was derived from a spontaneously arising leukaemia in SL mice [Ichikawa, 1969]. Parental M1 cells used in this study have been in passage at the Walter and Eliza Hall Institute for Medical Research, Melbourne, Victoria, Australia, for approximately 10 years. M1 cells were maintained by weekly passage in Dulbecco's modified Eagle's medium (DME) containing 10% (v/v) foetal bovine serum (FCS). Recombinant cytokines are generally available from commercial sources or were prepared by published methods. Recombinant murine LIF was produced in Escherichia coli and purified, as previously described [Gearing, 1989]. Purified human oncostatin M was purchased from PeproTech Inc (Rocky Hill, N.J., USA), and purified mouse IFN-γ was obtained from Genzyme Diagnostics (Cambridge, Mass., USA). Recombinant murine thrombopoietin was produced as a FLAGTM-tagged fusion protein in CHO cells and then purified.

### Example 2

### Agar Colony Assays

In order to assay the differentiation of M1 cells in response to cytokines, 300 cells were cultured in 35 mm Petri dishes containing 1 ml of DME supplemented with 20% (v/v) fÏtal calf serum (FCS), 0.3% (w/v) agar and 0.1 ml of serial dilutions of IL-6, LIF, OSM, IFN-γ, tpo or dexamethasone (Sigma Chemical Company, St Louis, Mich.). After 7 days culture at 37° C. in a fully humidified atmosphere, containing 10% (v/v) CO₂ in air, colonies of M1 cells were counted and classified as differentiated if they were composed of dispersed cells or had a corona of dispersed cells around a tightly packed centre.

### Example 3

### Generation of Retroviral Library

A cDNA expression library was constructed from the factor-dependent haemopoietic cell line FDC-P1, essentially as described [Rayner, 1994]. Briefly, cDNA was cloned into the retroviral vector pRUFneo and then transfected into an amphotropic packaging cell line (PA317). Transiently generated virus was harvested from the cell supernatant at 48 hr posttransfection, and used to infect Y2 ecotropic packaging cells, to generate a high titre virus-producing cell line.

### Example 4

### Retroviral Infection of M1 Cells

Pools of 10⁶ infected Ψ2 cells were irradiated (3000 rad) and cocultivated with 10⁶ M1 cells in DME supplemented with 10% (v/v) FCS and 4 μg/ml Polybrene, for 2 days at 37° C. To select for IL-6-unresponsive clones, retrovirally-infected M1 cells were washed once in DME, and cultured at approximately 2×10⁴ cells/ml in 1 ml agar cultures containing 400 μg/ml geneticin (GibcoBRL, Grand Island, N.Y.) and 100 ng/ml IL-6. The efficiency of infection of M1 cells was 1-2%, as estimated by agar plating the infected cells in the presence of geneticin only.

### Example 5

### PCR

Genomic DNA from retrovirally-infected M1 cells was digested with Sac I and 1 μg of phenol/chloroform extracted DNA was then amplified by polymerase chain reaction (PCR). Primers used for amplification of cDNA inserts from the integrated retrovirus were GAG3 (5′ CACGCCGCCCACGTGAAGGC 3′ [SEQ ID NO:1]), which corresponds to the vector gag sequence approximately 30 bp 5′ of the multiple cloning site, and HSVTK (5′ TTCGCCAATGACAAGACGCT 3′ [SEQ ID NO:2]), which corresponds to the pMC1neo sequence approximately 200 bp 3, of the multiple cloning site. The PCR entailed an initial denaturation at 94° C. for 5 min, 35 cycles of denaturation at 94° C. for 1 min, annealing at 56° C. for 2 min, and extension at 72° C. for 3 min, followed by a final 10 min extension. PCR products were gel purified and then ligated into the pGEM-T plasmid (Promega, Madison, Wis.), and sequenced using an ABI PRISM Dye Terminator Cycle Sequencing Kit and a Model 373 Automated DNA Sequencer (Applied Biosystems Inc., Foster City, Calif.).

### Example 6

### Cloning of cDNAs

Independent cDNA clones encoding mouse SOCS1 were isolated from a murine thymus cDNA library essentially as described (Hilton et al, 1994). The nucleotide and predicted amino acid sequences of mouse SOCS1 cDNA were compared to databases using the BLASTN and TFASTA algorithms (Pearson and Lipman, 1988; Pearson, 1990; Altshcul et al, 1990). Oligonucleotides were designed from the ESTs encoding human SOCS1 and mouse SOC-1 and SOCS3 and used to probe commercially available mouse thymus and spleen cDNA libraries. Sequencing was performed using an ABI automated sequencer according to the manufacturer's instructions.

### Example 7

### Southern and Northern Blot Analyses and RT-PCR

³²P-labelled probes were generated using a random decanucleotide labelling kit (Bresatec, Adelaide, South Australia) from a 600 bp Pst I fragment encoding neomycin phophotransfease from the plasmid pPGKneo, 1070 bp fragment of the SOCS1 gene obtained by digestion of the 1.4 kbp PCR product with Xho I, SOCS2, SOCS3, CIS and a 1.2 kbp fragment of the chicken glyceraldehyde 3-phosphate dehydrogenase gene [Dugaiczyk, 1983].

Genomic DNA was isolated from cells using a proteinase K-sodium dodecyl sulfate procedure essentially as described. Fifteen micrograms of DNA was digested with either BamH I or Sac I, fractionated on a 0.8% (w/v) agarose gel, transferred to GeneScreenPlus membrane (Du Pont NEN, Boston Mass.), prehybridised, hybridised with random-primed ³²P-labelled DNA fragments and washed essentially as described [Sambrook, 1989].

Total RNA was isolated from cells and tissues using Trizol Reagent, as recommended by the manufacturer (GibcoBRL, Grand Island, N.Y.). When required polyA+ mRNA was purified essentially as described [Alexander, 1995]. Northern blots were prehybridised, hybridized with random-primed ³²P-labelled DNA fragments and washed as described [Alexander, 1995].

To assess the induction of SOCS genes by IL-6, mice (C57BL6) were injected intravenously with 5 μg IL-6 followed by harvest of the liver at the indicated timepoints after injection. M1 cells were cultured in the presence of 20 ng/ml IL-6 and harvested at the indicated times. For RT-PCR analysis, bone marrow cells were harvested as described (Metacalf et al, 1995) and stimulated for 1 hr at 37° C. with 100 ng/ml of a range of cytokines. RT-PCR was performed on total RNA as described (Metcalf et al, 1995). PCR products were resolved on an agarose gel and Southern blots were hybridised with probes specific for each SOCS family member. Expression of β-actin was assessed to ensure uniformity of amplification.

### Example 8

### DNA Constructs and Transfection

A cDNA encoding epitope-tagged SOCS1 was generated by subcloning the entire SOCS1 coding region into the pEF-BOS expression vector [Mizushima, 1990], engineered to encode an inframe FLAG epitope downstream of an initiation methionine (pF-SOCS1). Using electroporation as described previously [Hilton, 1994], M1 cells expressing the thrombopoietin receptor (M1.mpl) were transfected with the 20 μg of Aat II-digested pF-SOCS1 expression plasmid and 2 μg of a Sca I-digested plasmid in which transcription of a cDNA encoding puromycin N-acetyl transferase was driven from the mouse phosphoglycerokinase promoter (pPGKPuropA). After 48 hours in culture, transfected cells were selected with 20 μg/ml puromycin (Sigma Chemical Company, St Louis Mo.), and screened for expression of SOCS1 by Western blotting, using the M2 anti-FLAG monoclonal antibody according to the manafacturer's instructions (Eastman Kodak, Rochester N.Y.). In other experiments M1 cells were transfected with only the pF-SOCS1 plasmid or a control and selected by their ability to grow in agar in the presence of 100 ng/ml of IL-6.

### Example 9

### Immunoprecipitation and Western Blotting

Prior to either immunoprecipitation or Western blotting, 10⁷ M1 cells or their derivatives were washed twice, resuspended in 1 ml of DME, and incubated at 37° C. for 30 min. The cells were then stimulated for 4 min at 37° C. with either saline or 100 ng/ml IL-6, after which sodium vanadate (Sigma Chemical Co., St Louis, Mich.) was added to a concentration of 1 mM. Cells were placed on ice, washed once with saline containing 1 mM sodium vanadate, and then solubilised for 5 min on ice with 300 μl 1% (v/v) Triton X-100, 150 mM NaCl, 2 mM EDTA, 50 mM Tris-HCl pH 7.4, containing Complete protease inhibitors (Boehringer Mannheim, Mannheim, Germany) and 1 mM sodium vanadate. Lysates were cleared by centrifugation and quantitated using a Coomassie Protein Assay Reagent (Pierce, Rockford Ill.).

For immunoprecipitations, equal concentrations of protein extracts (1-2 mg) were incubated for 1 hr or overnight at 4° C. with either 4 μg of anti-gp130 antibody (M20; Santa Cruz Biotechnology Inc., Santa Cruz, Calif.) or 4 μg of anti-phosphotyrosine antibody (4G10; Upstate Biotechnology Inc., Lake Placid N.Y.), and 15 μl packed volume of Protein G Sepharose Pharmacia, Uppsala, Sweden) [Hilton et al, 1996]. Immunoprecipitates were washed twice in 1% (v/v) NP40, 150 mM NaCl, 50 mM Tris-HCl pH 8.0, containing Complete protease inhibitors (Boehringer Mannheim, Mannheim, Germany and 1 mM sodium vanadate. The samples were heated for 5 min at 95° C. in SDS sample buffer (625 mM Tris-HCl pH 6.8, 0.05% (w/v) SDS, 0.1% (v/v) glycerol, bromophenol blue, 0.125% (v/v) 2-mercaptoethanol), fractionated by SDS-PAGE and immunoblotted as described above.

For Western blotting, 10 μg of protein from a cellular extract or material from an immunoprecipitation reaction was loaded onto 4-15% Ready gels (Bio-Rad Laboratories, Hercules Calif.), and resolved by sodium dodecyl sulfate polyacrylamide gel electrophoresis (SDS-PAGE). Proteins were transferred to PVDF membrane (Micron Separations Inc., Westborough Mass.) for 1 hr at 100 V. The membranes were probed with the following primary antibodies; anti-tyrosine phosphorylated STAT3 (1:1000 dilution; New England Biolabs, Beverly, Mass.); anti-STAT3 (C-20; 1:100 dilution; Santa Cruz Biotechnology Inc., Santa Cruz Calif.); anti-gp130 (M20, 1:100 dilution; Santa Cruz Biotechnology Inc., Santa Cruz Calif.); anti-phosphotyrosine (horseradish peroxidase-conjugated RC20, 1:5000 dilution; Transduction Laboratories, Lexington Ky.); anti-tyrosine phosphorylated MAP kinase and anti-MAP kinase antibodies (1:1000 dilution; New England Biolabs, Beverly, Mass.). Blots were visualised using peroxidase-conjugated secondary antibodies and Enhanced Chemiluminescence (ECL) reagents according to the manafacturer's instructions (Pierce, Rockford Ill.).

### Example 10

### Electrophoretic Mobility Shift Assays

Assays were performed as described [Novak, 1995], using the high affinity SIF (c-sis-inducible factor) binding site m67 [Wakao, 1994]. Protein extracts were prepared from M1 cells incubated for 4-10 min at 37° C. in 10 ml serum-free DME containing either saline, 100 ng/ml IL-6 or 100 ng/ml IFN-γ. The binding reactions contained 4-6 μg protein (constant within a given experiment), 5 ng ³²P-labelled m67 oligonucleotide, and 800 ng sonicated salmon sperm DNA. For certain experiments, protein samples were preincubated with an excess of unlabelled m67 oligonucleotide, or antibodies specific for either STAT1 (Transduction Laboratories, Lexington, Ky.) or STAT3 (Santa Cruz Biotechnology Inc., Santa Cruz Calif.), as described [Novak, 1995].

Western blots were performed using anti-tyrosine phosphorylated STAT3 or anti-STAT3 (New England Biolabs, Beverly, Mass.) or anti-gp130 (Santa Cruz Biotechnology Inc.) as described (Nicola et al, 1996). EMSA were performed using the m67 oligonucleotide probe, as described (Novak et al, 1995).

### Example 11

### Expression Cloning of a Novel Suppressor of Cytokine Signal Transduction

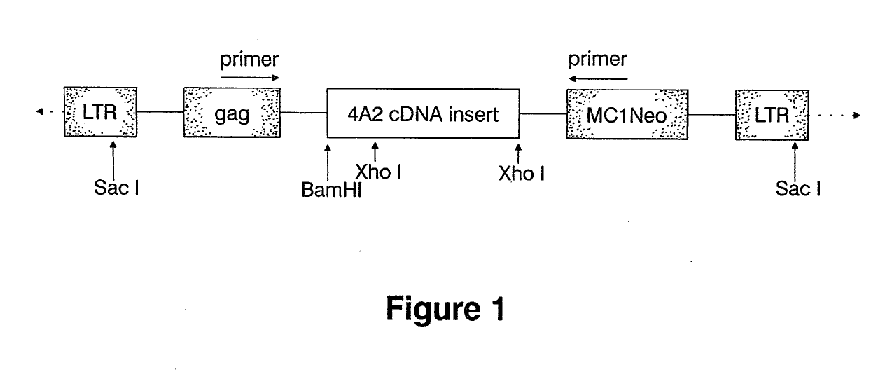

In order to identify cDNAs capable of suppressing cytokine signal transduction, an expression cloning approach was adopted. This strategy centred on M1 cells, a monocytic leukaemia cell line that differentiates into mature macrophages and ceases proliferation in response to the cytokines IL-6, LIF, OSM and IFN-γ, and the steroid dexamethasone. Parental M1 cells were infected with the RUFneo retrovirus, into which cDNAs from the factor-dependent haemopoietic cell line FDC-P1 had been cloned. In this retrovirus, transcription of both the neomycin resistance gene and the cloned cDNA was driven off the powerful constitutive promoter present in the retroviral LTR (FIG. 1). When cultured in semi-solid agar, parental M1 cells form large tightly packed colonies. Upon stimulation with IL-6, M1 cells undergo rapid differentiation, resulting in the formation in agar of only single macrophages or small dispersed clusters of cells Retrovirally-infected M1 cells that were unresponsive to IL-6 were selected in semi-solid agar culture by their ability to form large, tightly packed colonies in the presence of IL-6 and geneticin. A single stable IL-6-unresponsive clone, 4A2, was obtained after examining 10⁴ infected cells.

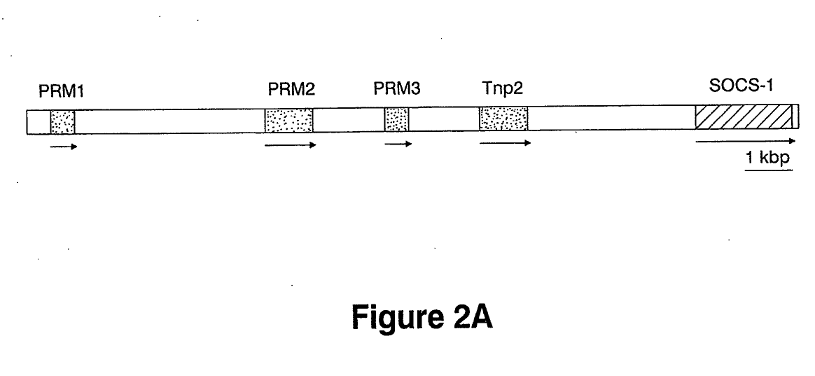

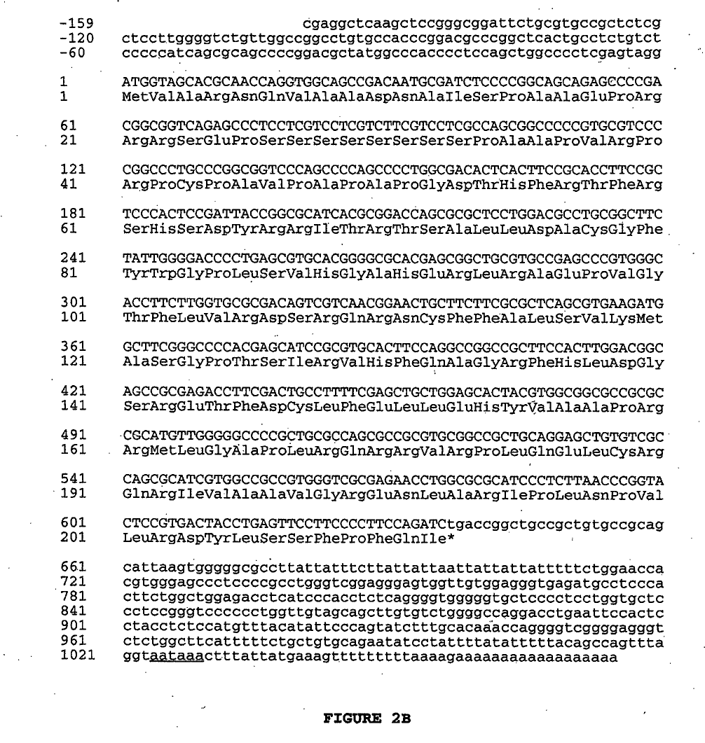

A fragment of the neomycin phosphotransferase (neo) gene was used to probe a Southern blot of genomic DNA from clone 4A2 and this revealed that the cell line was infected with a single retrovirus containing a cDNA approximately 1.4 kbp in length (FIG. 2). PCR amplification using primers from the retroviral vector which flanked the cDNA cloning site enabled recovery of a 1.4 kbp cDNA insert, which we have named suppressor of cytokine signalling-1, or SOCS1. This PCR product was used to probe a similar Southern blot of 4A2 genomic DNA and hybridised to two fragments, one which corresponded to the endogenous SOCS1 gene and the other, which matched the size of the band seen using the neo probe, corresponded to the SOCS1 cDNA cloned into the integrated retrovirus. The latter was not observed in an M1 cell clone infected with a retrovirus containing an irrelevant cDNA. Similarly, Northern blot analysis revealed that SOCS1 mRNA was abundant in the cell line 4A2, but not in the control infected M1 cell clone.

### Example 12

### SOCS1, SOCS2, SOCS3 and CIS Define a New Family of SH2-Containing Proteins

The SOCS1 PCR product was used as a probe to isolate homologous cDNAs from a mouse thymus cDNA library. The sequence of the cDNAs proved to be identical to the PCR product, suggesting that constitutive or over expression, rather than mutation, of the SOCS1 protein was sufficient for generating an IL-6-unresponsive phenotype. Comparison of the sequence of SOCS1 cDNA with nucleotide sequence databases revealed that it was present on mouse and rat genomic DNA clones containing the protamine gene cluster found on mouse chromosome 16. Closer inspection revealed that the 1.4 kb SOCS1 sequence was not homologous to any of the protamine genes, but rather represented a previously unidentified open reading frame located at the extreme 3′ end of these clones (FIG. 2). There were no regions of discontinuity between the sequences of the SOCS1 cDNA and genomic locus, suggesting that SOCS1 is encoded by a single exon. In addition to the genomic clone containing the protamine genes, a series of murine and human expressed sequenced tags (ESTs) also revealed large blocks of nucleotide sequence identity to mouse SOCS1. The sequence information provided by the human ESTs allowed the rapid cloning of cDNAs encoding human SOCS1.

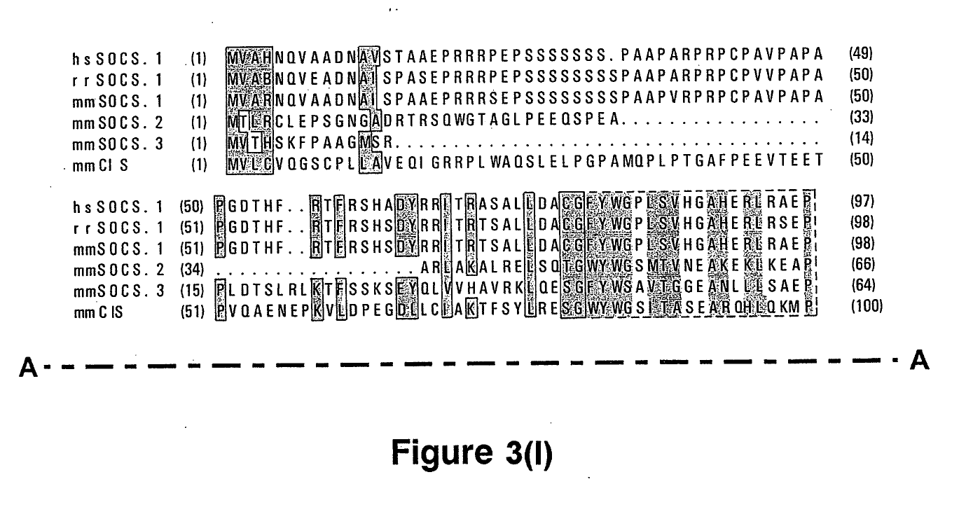

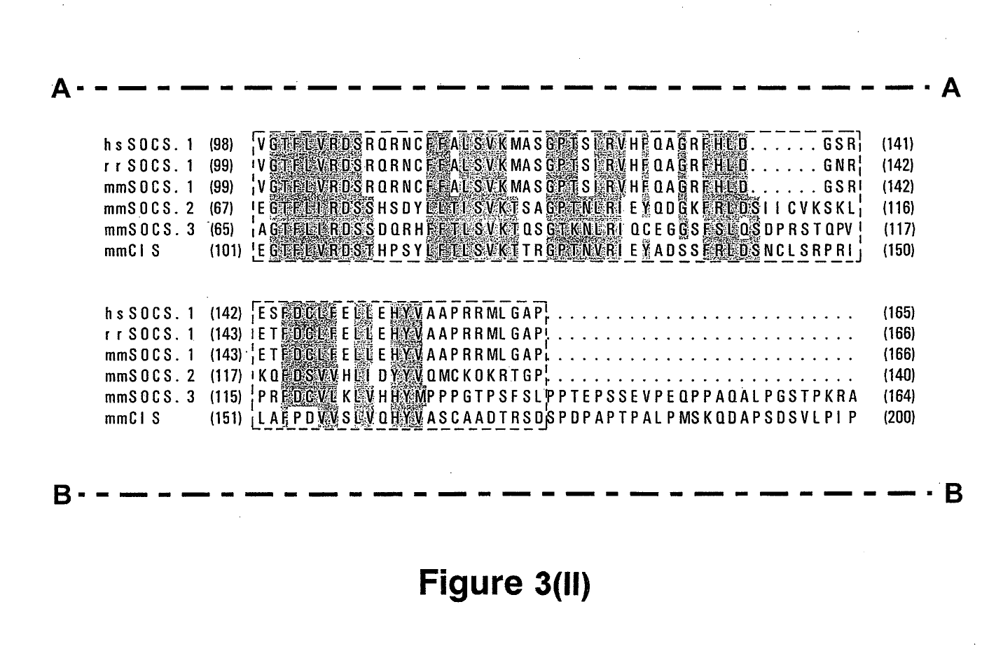

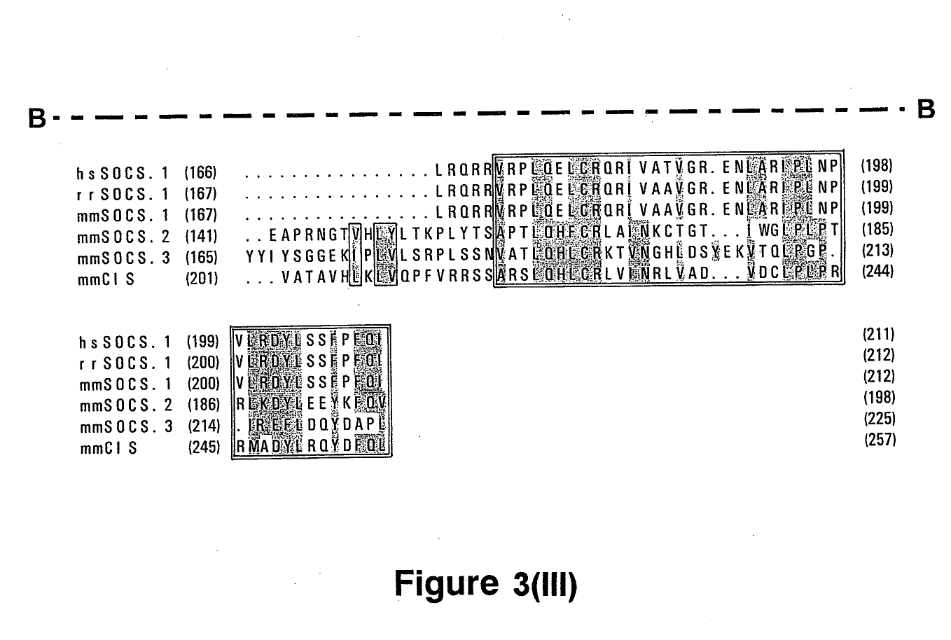

The mouse and rat SOCS1 gene encodes a 212 amino acid protein whereas the human SOCS1 gene encodes a 211 amino acid protein. Mouse, rat and human SOCS1 proteins share 95-99% amino acid identity (FIG. 3). A search of translated nucleic acid databases with the predicted amino acid sequence of SOCS1 showed that it was most related to a recently cloned cytokine-inducible immediate early gene product, CIS, and two classes of ESTs. Full length cDNAs from the two classes of ESTs were isolated and found to encode proteins of similar length and overall structure to SOCS1 and CIS. These clones were given the names SOCS2 and SOCS3. Each of the four proteins contains a central SH2 domain and a C-terminal region termed the SOCS motif. The SOCS1 proteins exhibit an extremely high level of amino acid sequence similarity (95-99% identity) amongst different species. However, the forms of the SOCS1, SOCS2, SOCS3 and CIS from the same animal, while clearly defining a new family of SH2-containing proteins, exhibited a lower amino acid identity. SOCS2 and CIS exhibit approximately 38% amino acid identity, while the remaining members of the family share approximately 25% amino acid identity (FIG. 3). The coding region of the genes for SOCS1 and SOC3 appear to contain no introns while the coding region of the genes for SOCS2 and CIS contain one and two introns, respectively.

The Genbank Accession Numbers for the sequences referred to herein are mouse SOCS1 cDNA U88325), human SOCS1 cDNA (U88326), mouse SOCS2 cDNA (U88327), mouse SOCS3 DNA (U88328).

### Example 13

### Constitutive Expression of SOCS1 Suppresses the Action of a Range of Cytokines

To formally establish that the phenotype of the 4A2 cell line was directly related to expression of SOCS1, and not to unrelated genetic changes which may have occurred independently in these cells, a cDNA encoding an epitope-tagged version of SOCS1 under the control of the EF1α promoter was transfected into parental M1 cells, and M1 cells expressing the receptor for thrombopoietin, c-mpl (M1.mpl). Transfection of the SOCS1 expression vector into both cell lines resulted in an increase in the frequency of IL-6 unresponsive M1 cells.

Multiple independent clones of M1 cells expression SOCS1, as detected by Western blot, displayed a cytokine-unresponsive phenotype that was indistinguishable from 4A2. Further, if transfectants were not maintained in puromycin, expression of SOCS1 was lost over time and cells regained their cytokine responsiveness. In the absence of cytokine, colonies derived from 4A2 and other SOCS1 expressing clones characteristically grew to a smaller size than clones formed by control M1 cells.

The effect of constitutive SOCS1 expression on the response of M1 cells to a range of cytokines was investigated using the 4A2 cell line and a clone of M1.mpl cells expressing SOCS1 (M1.mpl.SOCS1). Unlike parental M1 cells and M1.mpl cells, the two cell lines expressing SOCS1 continued to proliferate and failed to form differentiated colonies in response to either IL-6, LIF, OSM, IFN-γ or, in the case of the M1.mpl.SOCS1 cell line, thrombopoietin. For both cell lines, however, a normal response to dexamethasone was observed, suggesting that SOCS1 specifically affected cytokine signal transduction rather than differentiation per se. Consistent with these data, while parental M1 cells and M1.mpl cells became large and vacuolated in response to IL-6, 4A2 and M1.mpl.SOCS1 cells showed no evidence of morphological differentiation in response to IL-6 or other cytokines.

### Example 14

### SOCS1 Inhibits a Range of IL-6 Signal Transduction Processes, Including Stat3 Phosphorylation and Activation

Phosphorylation of the cell surface receptor component gp130, the cytoplasmic tyrosine kinase JAK1 and the transcription factor STAT3 is thought to play a central role in IL-6 signal transduction. These events were compared in the parental M1 and M1.mpl cell lines and their SOCS1-expressing counterparts. As expected, gp130 was phosphorylated rapidly in response to IL-6 in both parental lines, however, this was reduced five- to ten-fold in the cell lines expressing SOCS1. Likewise, STAT3 phosphorylation was also reduced by approximately ten-fold in response to IL-6 in those cell lines expressing SOCS1. Consistent with a reduction in STAT3 phosphorylation, activation of specific STAT DNA binding complexes, as determined by electrophoretic mobility shift assay, was also reduced. Notably, there was a reduction in the formation of SIF-α (containing STAT3), SIF-B (STAT1/STAT3 heterodimer) and SIF-C (containing STAT1), the three STAT complexes induced in M1 cells stimulated with IL-6. Similarly, constitutive expression of SOCS1 also inhibited IFN-γ-stimulated formation of p91 homodimers. STAT phosphorylation and activation were not the only cytoplasmic processes to be effected by SOCS1 expression, as the phosphorylation of other proteins, including shc and MAP kinase, was reduced to a similar extent.

### Example 15

### Transcription of the SOCS1 Gene is Stimulated by IL-6 In Vitro and In Vivo

Although SOCS1 can inhibit cytokine signal transduction when constitutively expressed in M1 cells, this does not necessarily indicate that SOCS1 normally functions to negatively regulate an IL-6 response. In order to investigate this possibility the inventors determined whether transcription of the SOCS1 gene is regulated in the response of M1 cells to IL-6 and, because of the critical role IL-6 plays in regulating the acute phase response to injury and infection, the response of the liver to intravenous injection of 5 mg IL-6. In the absence of IL-6, SOCS1 mRNA was undetectable in either M1 cells or in the liver. However, for both cell types, a 1.4 kb SOCS1 transcript was induced within 20 to 40 minutes by IL-6. For M1 cells, where the IL-6 was present throughout the experiment, the level of SOCS1 mRNA remained elevated. In contrast, IL-6 was administered in vivo by a single intravenous injection and was rapidly cleared from the circulation, resulting in a pulse of IL-6 stimulation to the liver. Consistent with this, transient expression of SOCS1 mRNA was detectable in the liver, peaking approximately 40 minutes after injection and declining to basal levels within 4 hours.

### Example 16

### Regulation of SOCS Genes

Since CIS was cloned as a cytokine-inducible immediate early gene the inventors examined whether SOCS1, SOCS2 and SOCS3 were similarly regulated. The basal pattern of expression of the four SOCS genes was examined by Northern blot analysis of mRNA from a variety of tissues from male and female C57B1/6 mice. Constitutive expression of SOCS1 was observed in the thymus and to a lesser extend in the spleen and the lung. SOCS2 expression was restricted primarily to the testis and in some animals the liver and lung; for SOCS3 a low level of expression was observed in the lung, spleen and thymus, while CIS expression was more widespread, including the testis, heart, lung, kidney and, in some animals, the liver.

The inventors sought to determine whether expression of the four SOCS genes was regulated by IL-6. Northern blots of mRNA prepared from the livers of untreated and IL-6-injected mice, or from unstimulated and IL-6-stimulated M1 cells, were hybridised with labelled fragments of SOCS1, SOCS2, SOCS3 and CIS cDNAs. Expression of all four SOCS genes was increased in the liver following IL-6 injection, however the kinetics of induction appeared to differ. Expression of SOCS1 and SOCS3 was transient in the liver, with mRNA detectable after 20 minutes of IL-6 injection and declining to basal levels within 4 hours for SOCS and 8 hours for SOCS3. Induction of SOCS2 and CIS mRNA in the liver followed similar initial kinetics to that of SOCS1, but was maintained at an elevated level for at least 24 hours. A similar induction of SOCS gene mRNA was observed in other organs, notably the lung and the spleen. In contrast, in M1 cells, while SOCS1 and CIS mRNA were induced by IL-6, no induction of either SOCS2 or SOCS3 expression was detected. This result highlights cell type-specific differences in the expression of the genes of SOCS family members in response to the same cytokine.

In order to examine the spectrum of cytokines that was capable of inducing transcription of the various members of the SOCS gene family, bone marrow cells were stimulated for an hour with a range of cytokines, after which mRNA was extracted and cDNA was synthesised. PCR was then used to assess the expression of SOCS1, SOCS2, SOCS3 and CIS. In the absence of stimulation, little or no expression of any of the SOCS genes was detectable in bone marrow by PCR. Stimulation of bone marrow cells with a broad array of cytokines appeared capable of up regulating mRNA for one or more members of the SOCS family. IFNγ, for example, induced expression of all four SOCS genes, while erythropoietin, granulocyte colony-stimulating factor, granulocyte-macrophage colony stimulating factor and interleukin-3 induced expression of SOCS2, SOCS3 and CIS. Interestingly, tumor necrosis factor alpha, macrophage colony-stimulating factor and interleukin-1, which act through receptors that do not fall into the type I cytokine receptor class also appeared capable of inducing expression of SOCS3 and CIS, suggesting that SOCS proteins may play a broader role in regulating signal transduction.

As constitutive expression of SOCS1 inhibited the response of M1 cells to a range of cytokines, the inventors examined whether phosphorylation of the cell surface receptor component gp 130 and the transcription factor STAT3, which are though to play a central role in IL-6 signal transduction, were affected. These events were compared in the parental M1 and M1.mpl cell lines and their SOCS1-expressing counterparts. As expected, gp130 was phosphorylated rapidly in response to IL-6 in both parental lines, however, this was reduced in the cell lines expressing SOCS1. Likewise, STAT3 phosphorylation was also reduced in response to IL-6 in those cell lines expressing SOCS1. Consistent with a reduction in STAT3 phosphorylation, activation of specific STAT/DNA binding complexes, as determined by electrophoretic mobility shift assay, was also reduced. Notably, there was a failure to form SIF-α (containing STAT3) and SIF-B(STAT1/STAT3 heterodimer), the major STAT complexes induced in M1 cells stimulated with IL-6. Similarly, constitutive expression of SOCS1 also inhibited IFNγ-stimulating formation of SIF-C(STAT1 homodimer; FIG. 12B). These experiments are consistent with the proposal that SOCS1 inhibits signal transduction upstream of receptor and STAT phosphorylation, potentially at the level of the JAK kinases.

The ability of SOCS1 to inhibit signal transduction and ultimately the biological response to cytokines suggest that, like the SH2-containing phosphatase SHP-1 [Ihle et al, 1994; Yi et al, 1993], the SOCS proteins may play a central role in controlling the intensity and/or duration of a cell's response to a diverse range of extracellular stimuli by suppressing the signal transduction process. The evidence provided here indicates that the SOCS family acts in a classical negative feedback loop for cytokine signal transduction. Like other genes such as OSM, expression of genes encoding the SOCS proteins is induced by cytokines through the activation of STATs. Once expressed, it is proposed that the SOCS proteins inhibit the activity of JAKs and so reduce the phosphorylation of receptors and STATs, thereby suppressing signal transduction and any ensuing biological response. Importantly, inhibition of STAT activation will, over time, lead to a reduction in SOCS gene expression, allowing cells to regain responsiveness to cytokines.

### Example 17

### Database Searches

The NCBI genetic sequence database (Genbank), which encompasses the major database of expressed sequence tags (ESTs) and TIGR database of human expressed sequence tags, were searched for sequences with similarity to a consensus SOCS box sequence using the TFASTA and MOTIF/PATTERN algorithms [Pearson, 1990; Cockwell and Giles, 1989]. Using the software package SRS [Etzold et al, 1996], ESTs that exhibited similarity to the SOCS box (and their partners derived from sequencing the other end of cDNAs) were retrieved and assembled into contigs using Autoassembler (Applied Biosystems, Foster City, Calif.). Consensus nucleotide sequences derived from overlapping ESTs were then used to search the various databases using BLASTN [Altschul et al, 1990]. Again, positive ESTs were retrieved and added to the contig. This process was repeated until no additional ESTs could be recovered. Final consensus nucleotide sequences were then translated using Sequence Navigator (Applied Biosystems, Foster City, Calif.).

The ESTs encoding the new SOCS proteins are as follows: human SOCS4 (EST81149, EST180909, EST182619, ya99H09, ye70co4, yh53c09, yh77g11, yh87h05, yi45h07, yj04e06, yq12h06, yq56a06, yq60e02, yq92g03, yq97h06, yr90f01, yt69c03, yv30a08, yv55f07, yv57h09, yv87h02, yv98e11, yw68d10, yw82a03, yx08a07, yx72h06, yx76b09, yy37h08, yy66b02, za81f08, zb18f07, zc06e08, zd14g06, zd51h12, zd52b09, ze25g11, ze69f02, zf54f03, zh96e07, zv66h12, zs83a08 and zs83g08). mouse SOCS4 (mc65f04, mf42e06, mp10c10, mr81 g09, and mt19h12). human SOCS-5 (EST15B103, EST15B105, EST27530 and zf50f01). mouse SOCS-5 (mc55a01, mh98f09, my26h12 and ve24e06). human SOCS-6 (yf61e08, yf93a09, yg05f12, yg41f04, yg45c02, yh11f10, yh13b05, zc35a12, ze02h08, zl09a03, zl69e10, zn39d08 and zo39e06). mouse SOCS-6 (mc04c05, md48a03, mf31 d03, mh26b07, mh78e11, mh88h09, mh94h07, mi27h04 and mj29c05, mp 66g04, mw75g03, va53b05, vb34h02, vc55d07, vc59e05, vc67d03, vc68d10, vc97h01, vc99c08, vd07h03, vd08c01, vd09b12, vd19b02, vd29a04 and vd46d06). human SOCS-7 (STS WI30171, EST00939, EST12913, yc29b05, yp49f10, zt10f03 and zx73g04). mouse SOCS-7 (mj39a01 and vi52h07). mouse SOCS-8 (mj6e09 and vj27a029). human SOCS-9 (CSRL-82f2-u, EST114054, yy06b07, yy06g06, zr40c09, zr72h01, yx92c08, yx93b08 and hfe0662). mouse SOCS-9 (me65d05). human SOCS-10 (aa48h10, zp35h01, zp97h12, zq08h01, zr34g05, EST73000 and HSDHEI005). mouse SOCS-10 (mb14d12, mb40f06, mg89b11, mq89e12, mp03g12 and vh53c11). human SOCS-11 (zt24h06 and zr43b02). human SOCS-13 (EST59161). mouse SOCS-13 (ma39a09, me60c05, mi78g05, mk10c11, mo48g12, mp 94a01, vb57c07 and vh07c11). human SOCS-14 (mi75e03, vd29h11 and vd53g07).

### Example 18

### cDNA Cloning

Based on the consensus sequences derived from overlapping ESTs, oligonucleotides were designed that were specific to various members of the SOCS family. As described above, oligonucleotides were labelled and used to screen commercially available genomic and cDNA libraries cloned with λ bacteriophage. Genomic and/or cDNA clones covering the entire coding region of mouse SOCS4, mouse SOCS5 and mouse SOCS6 were isolated. The entire gene for SOCS15 is on the human 12p13 BAC (Genbank Accession Number HSU47924) and the mouse chromosome 6 BAC (Genbank Accession Number AC002393). Partial cDNAs for mouse SOCS7, SOCS9, SOCS10, SOCS11, SOCS12, SOCS13 and SOCS14 were also isolated.

### Example 19

### Northern Blots and rtPCR

Northern blots were performed as described above. The sources of hybridisation probes were as follows; (i) the entire coding region of the mouse SOCS1 cDNA, (ii) a 1059 bp PCR product derived from coding region of SOCS5 upstream of the SH2 domain, (iii) the entire coding region of the mouse SOCS6 cDNA, (iv) a 790 bp PCR product derived from the coding region of a partial SOCS7 cDNA and (v) a 1200 bp Pst I fragment of the chicken glyceraldehyde 3-phosphate dehydrogenase (GAPDH) cDNA.

### Example 20

### Additional Members of SOCS Family

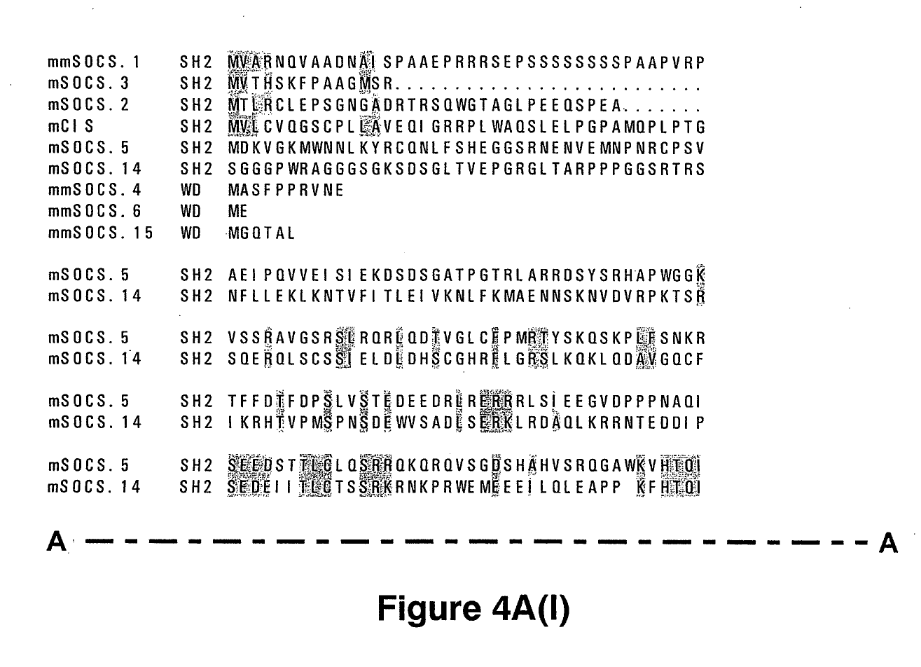

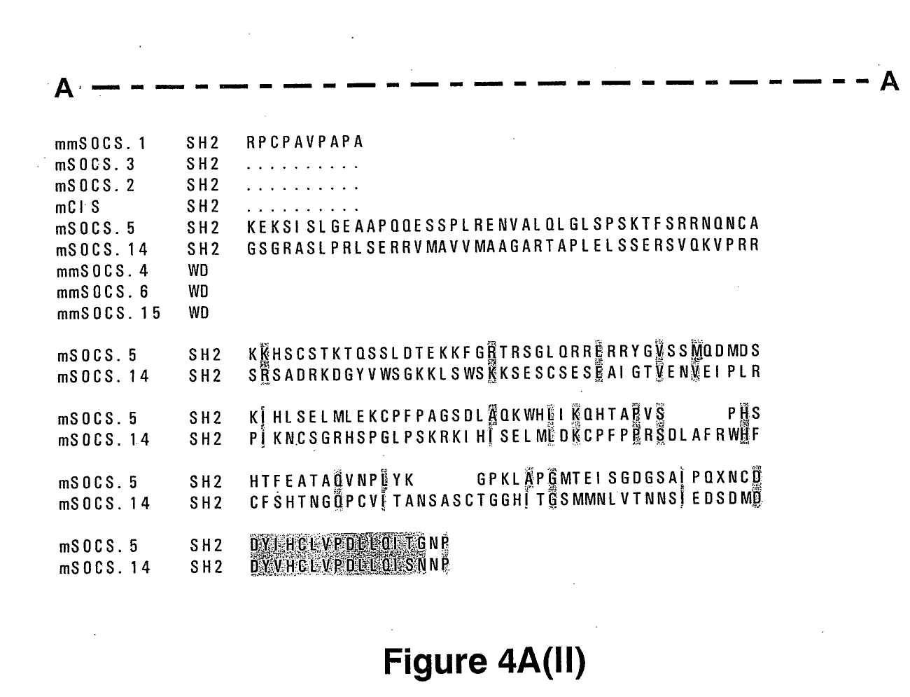

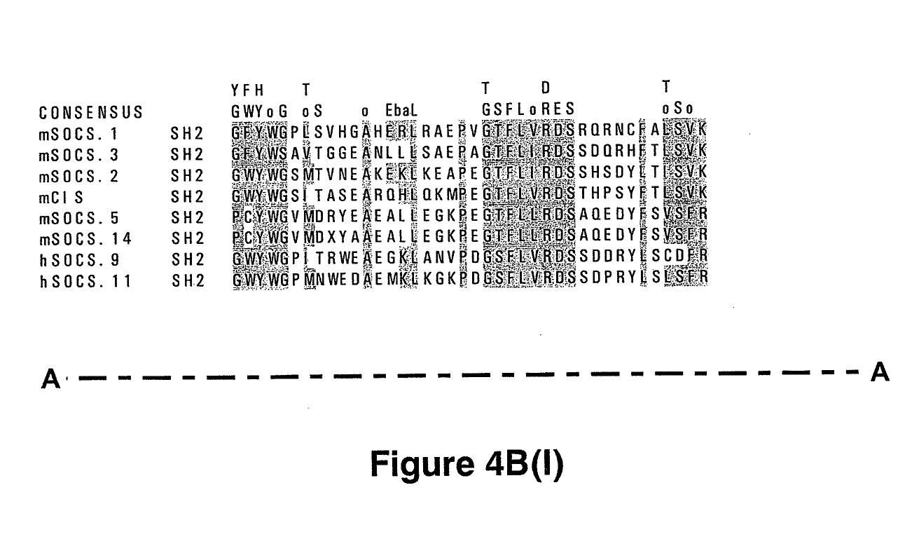

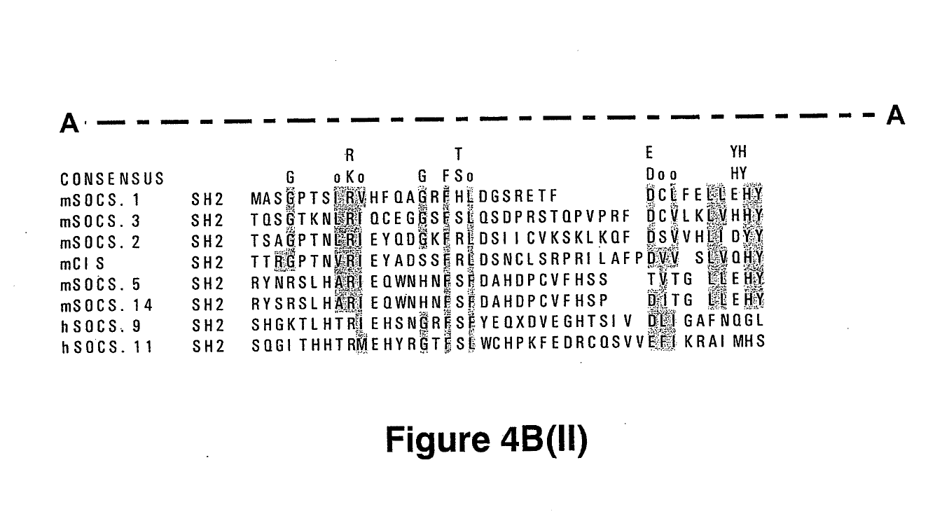

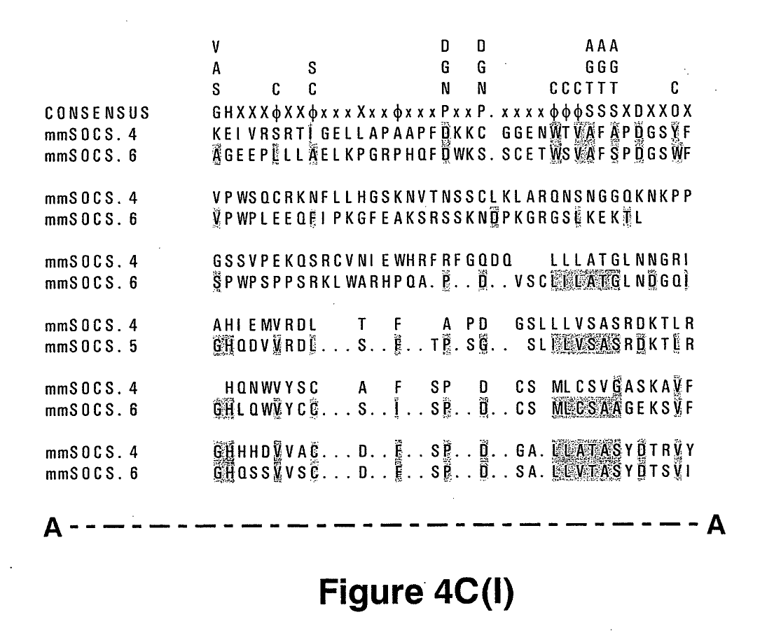

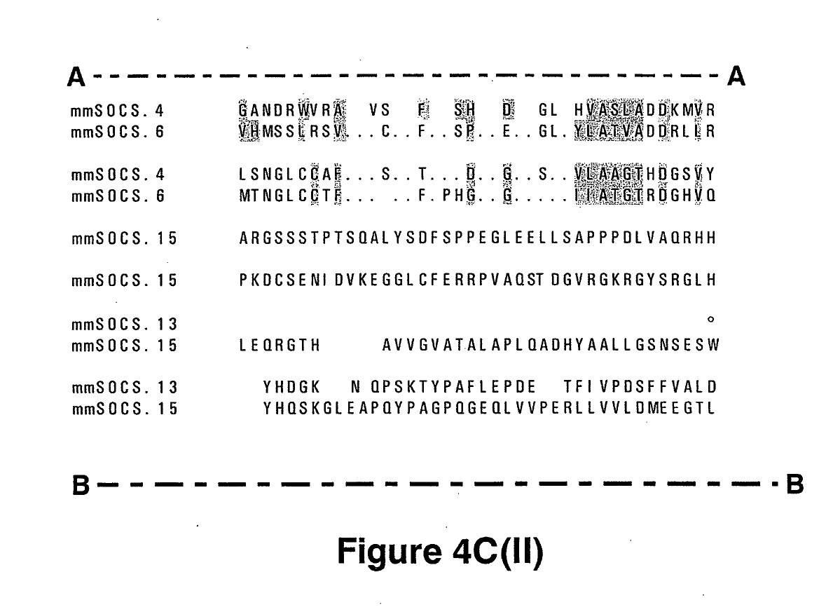

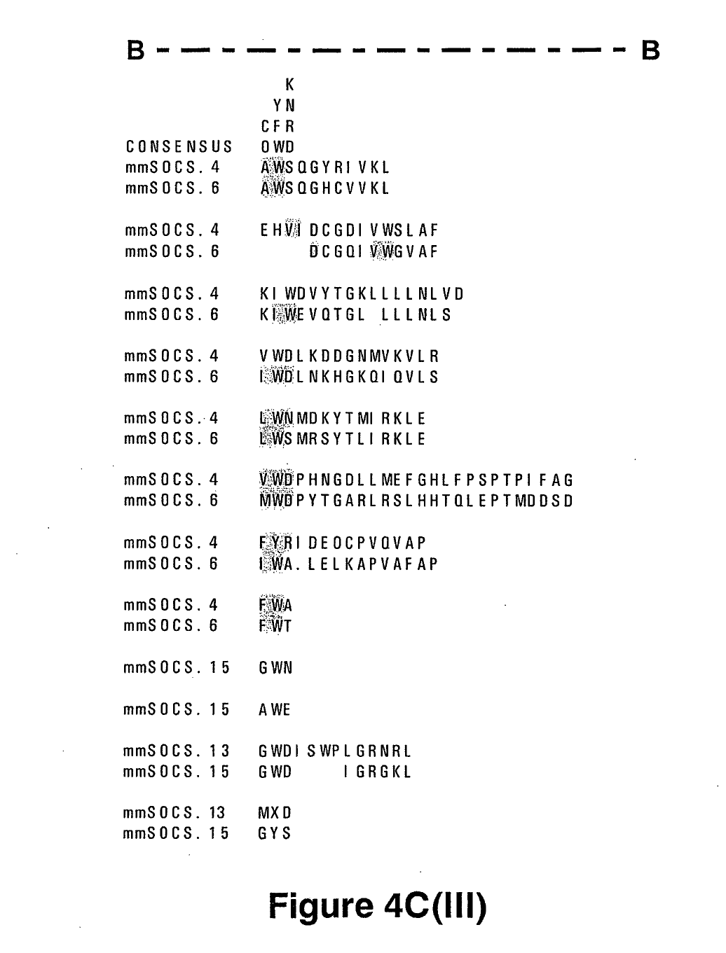

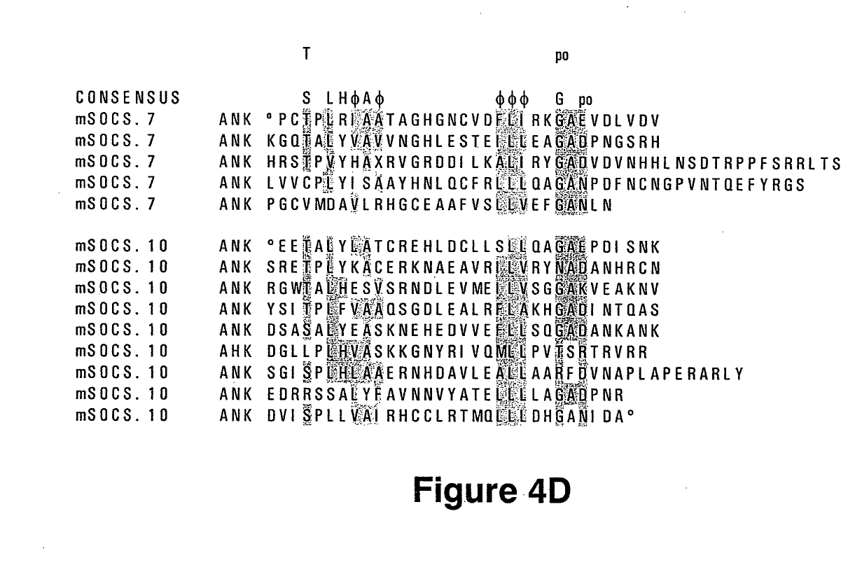

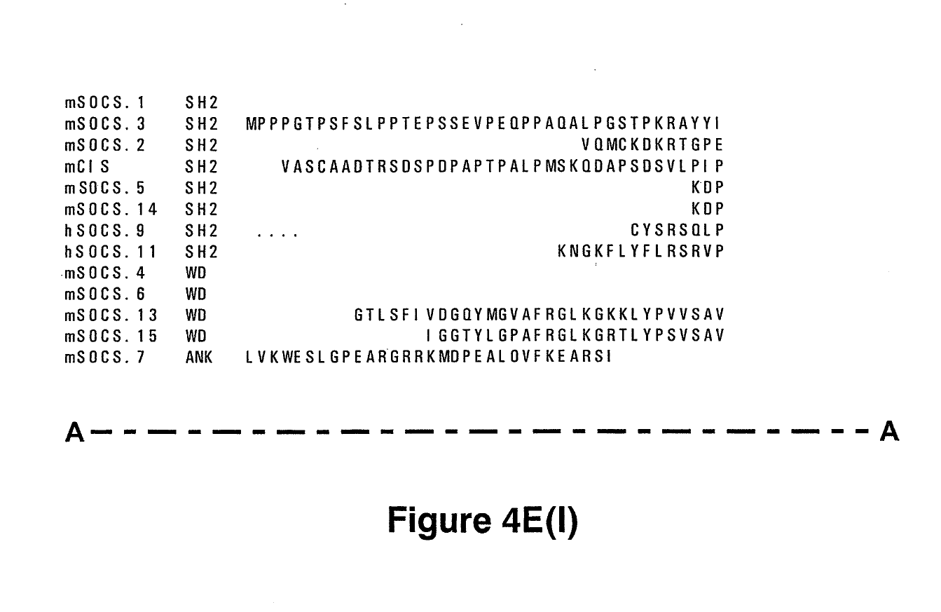

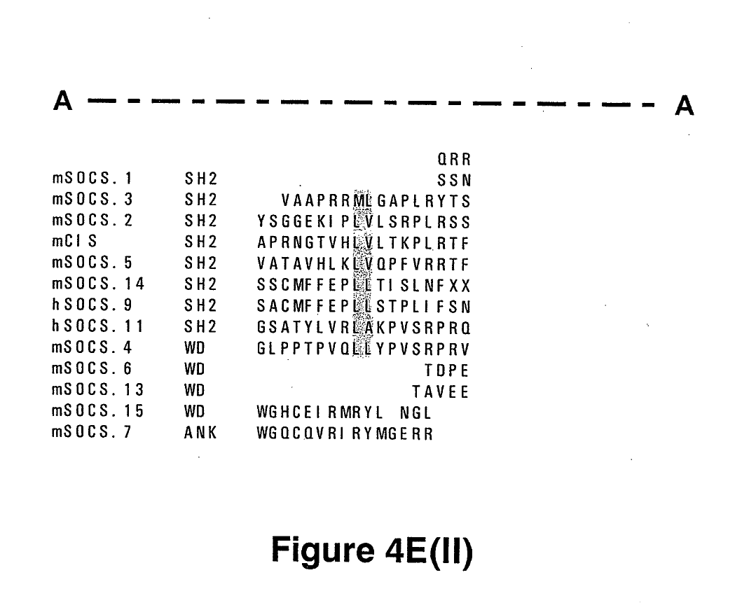

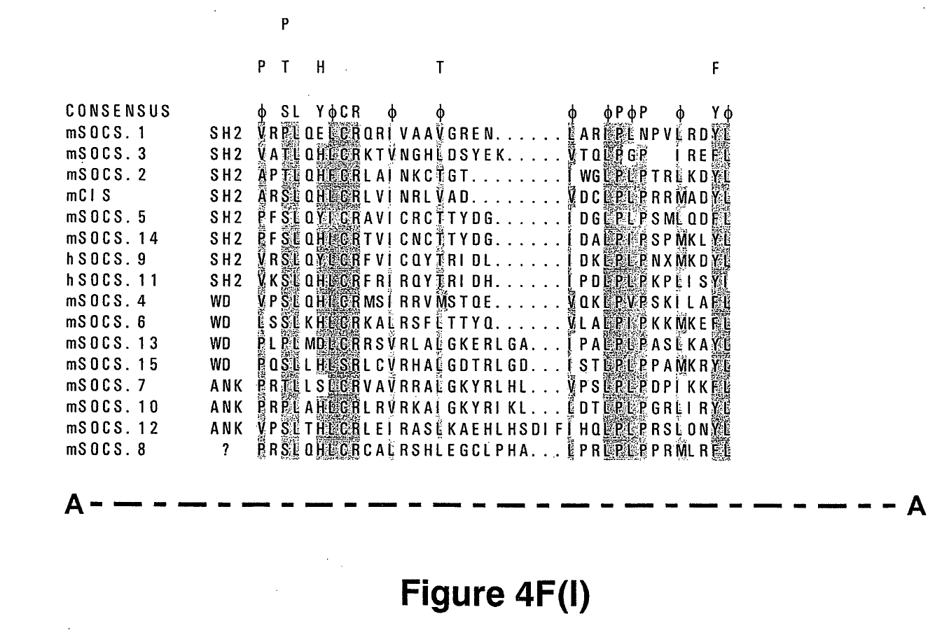

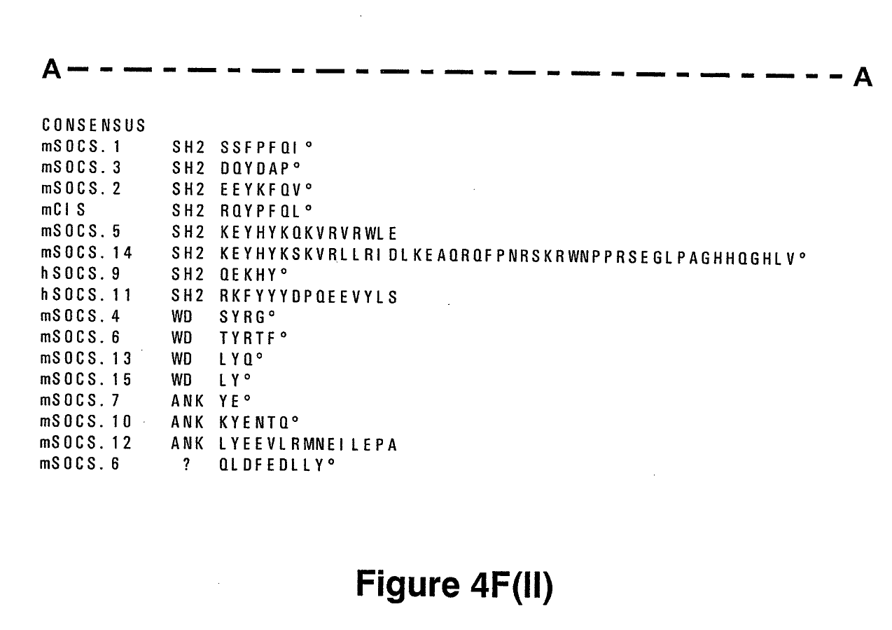

SOCS1, SOCS2 and SOCS3 are members of the SOCS protein family identified in Examples 1-16. Each contains a central SH2 domain and a conserved motif at the C-terminus, named the SOCS box. In order to isolate further members of this protein family, various DNA databases were searched with the amino acid sequence corresponding to conserved residues of the SOCS box. This search revealed the presence of human and mouse ESTs encoding twelve further members of the SOCS protein family (FIG. 4). Using this sequence information cDNAs encoding SOCS4, SOCS5, SOCS6, SOCS7, SOCS9, SOCS10, SOCS11, SOCS12, SOCS13, SOCS14 and SOCS15 have been isolated. Further analysis of contigs derived from ESTs and cDNAs revealed that the SOCS proteins could be placed into three groups according to their predicted structure N-terminal of the SOCS box. The three groups are those with (i) SH2 domains, (ii) WD-40 repeats and (iii) ankyrin repeats.

### Example 21

### SOCS Protein with SH2 Domains

Eight SOCS proteins with SH2 domains have been identified. These include SOCS1, SOCS2 and SOCS3, SOCS5, SOCS9, SOCS11 and SOCS14 (FIG. 4). Full length cDNAs were isolated for mouse SOCS5 and SOCS14 and partial clones encoding mouse SOCS9 and SOCS14. Analysis of primary amino acid sequence and genomic structure suggest that pairs of these proteins (SOCS1 and SOCS3, SOCS2 and CIS, SOCS5 and SOCS14 and SOCS9 and SOCS11) are most closely related (FIG. 4). Indeed, the SH2 domains of SOCS5 and SOCS14 are almost identical (FIG. 4B), and unlike CIS, SOCS1, SOCS2 and SOCS3, SOCS5 and SOCS14 have an extensive, though less well conserved, N-terminal region preceding their SH2 domains (FIG. 4A).

### Example 22

### SOCS Proteins with WD-40 Repeats

Four SOCS proteins with WD-40 repeats were identified. As with the SOCS proteins with SH2 domains, pairs of these proteins appeared to be closely related. Full length cDNAs of mouse SOCS4 and SOCS6 were isolated and shown to encode proteins containing eight WD-40 repeats N-terminal of the SOCS box (FIG. 4) and SOCS4 and SOCS6 share 65% amino acid similarity. SOCS15 was recognised as an open reading frame upon sequencing BACs from human chromosome 12p13 and the synthetic region of mouse chromosome 6 [Ansari-Lari et al, 1997]. In the human, chimp and mouse, SOCS15 is encoded by a gene with two coding exons that lies within a few hundred base pairs of the 3′ end of the triose phosphate isomerase (TPI) gene, but which is encoded on the opposite strand to TPI (9). In addition to a C-terminal SOCS box, the SOCS15 protein contains four WD-40 repeats. Interestingly, within the EST databases, there is a sequence of a nematode, an insect and a fish relative of SOCS15. SOCS15 appears most closely related to SOCS13.

### Example 23

### SOCS Proteins with Ankyrin Repeats

Three SOCS proteins with ankyrin repeats were identified. Analysis of partial cDNAs of mouse SOCS7, SOCS10 and SOCS12 demonstrated the presence of multiple ankyrin repeats.

### Example 24

### Expression Pattern of SOCS Proteins

The expression of mRNA from representative members of each class of SOCS proteins —SOCS1 and SOCS5 from the SH2 domain group, SOCS6 from the WD-40 repeat group and SOCS7 from the ankyrin repeat group was examined. As shown above, SOCS1 mRNA is found in abundance in the thymus and at lower levels in other adult tissues.

Since transcription of the SOCS1 gene is induced by cytokines, the inventors sought to determine whether levels of SOCS5, SOCS6 and SOCS7 mRNA increased upon cytokine stimulation. In the livers of mice injected with IL-6, SOCS1 mRNA is detectable after 20 min and decreases to background levels within 2 hours. In contrast, the kinetics of SOCS5 mRNA expression are quite different, being only detectable 12 to 24 hours after IL-6 injection. SOCS6 mRNA appears to be expressed constitutively while SOCS7 mRNA was not detected in the liver either before injection of IL-6 or at any time after injection.

Expression of these genes was also examined after cytokine stimulation of the factor-dependent cell line FDCP-1 engineered to express bcl-w. Again, SOCS6 mRNA was expressed constitutively.

### Example 25

### SOCS4

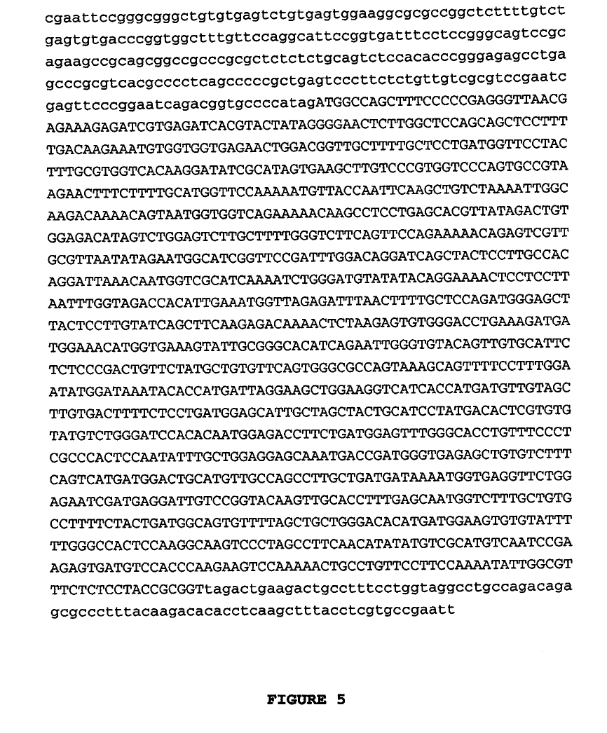

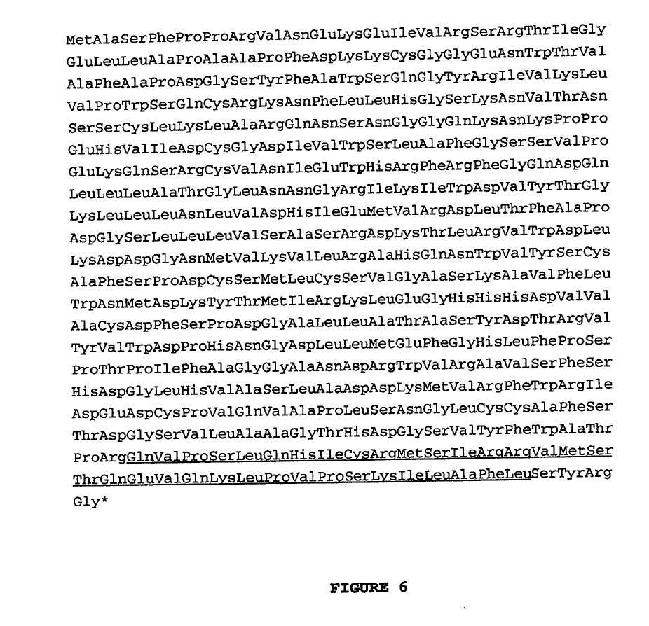

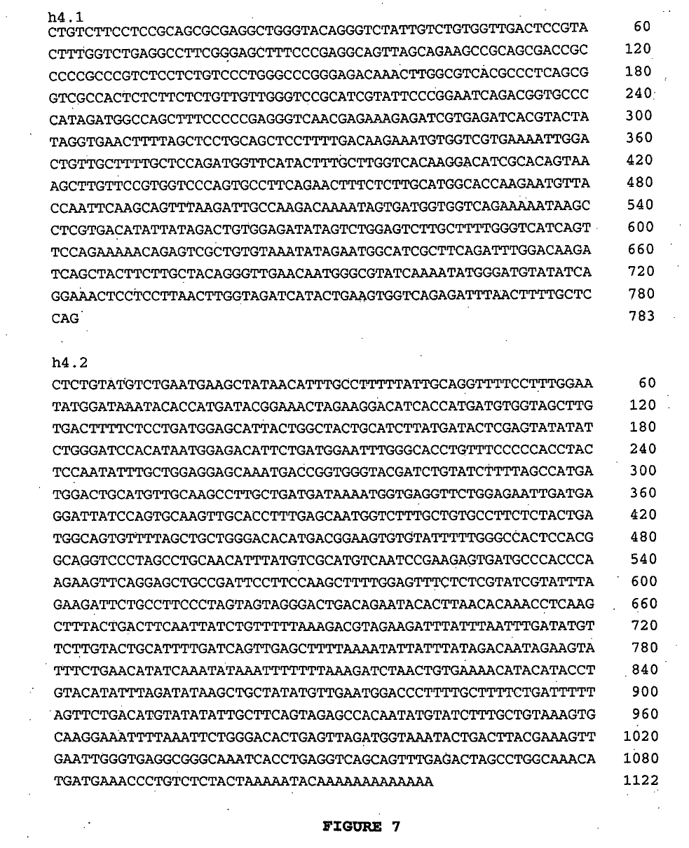

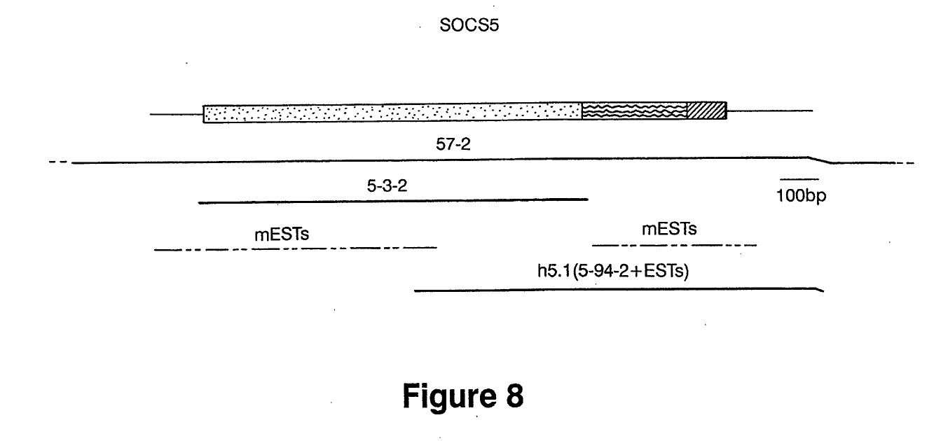

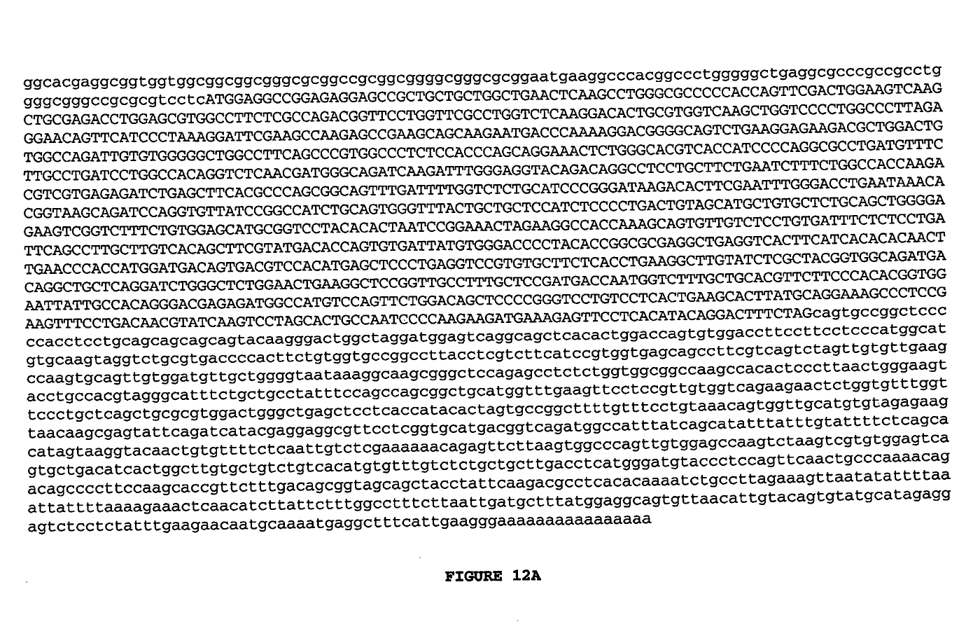

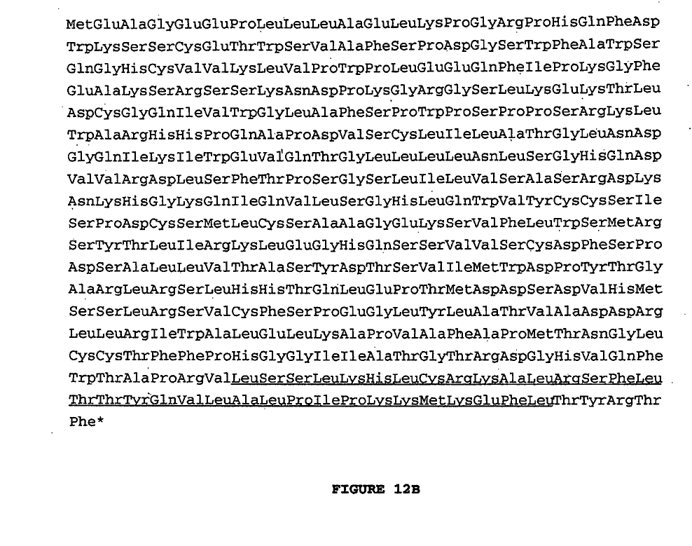

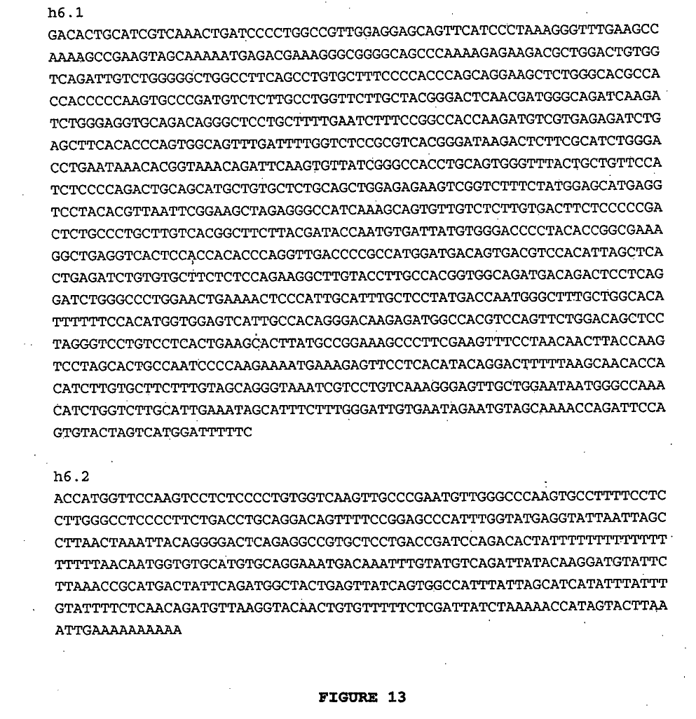

Mouse and human SOCS4 were recognized through searching EST databases using the SOCS box consensus (FIG. 13). Those ESTs derived from mouse and human SOCS4 cDNAs are tabulated below (Tables 4.1 and 4.2). Using sequence information derived from mouse ESTs several oligonucleotides were designed and used to screen, in the conventional manner, a mouse thymus cDNA library cloned into 1-bacteriophage. Two cDNAs encoding mouse SOCS4 were isolated and sequenced in their entirety (FIG. 5) and shown to overlap the mouse ESTs identified in the database (Table 4.1). These cDNAs include a region of 5′ untranslated region, the entire mouse SOCS4 coding region and a region of 3′ untranslated region (FIG. 7). Analysis of the sequence confirms that the SOCS4 cDNA encodes a SOCS Box at its C-terminus and a series of 8 WD-40 repeats before the SOCS Box (FIGS. 6 and 7). The relationship of the two sequence contigs of human SOCS4 (h4.1 and h4.2) to the experimentally determined mouse SOCS4 cDNA sequence is shown in FIG. 7. The nucleotide sequence of the two human contigs is listed in FIG. 8.

SEQ ID NOS:13 and 14 represent the nucleotide sequence of murine SOCS4 and the corresponding amino acid sequence. SEQ ID NOS:15 and 16 are SOCS4 cDNA human contigs h4.1 and h4.2, respectively.

### Example 26

### SOCS5

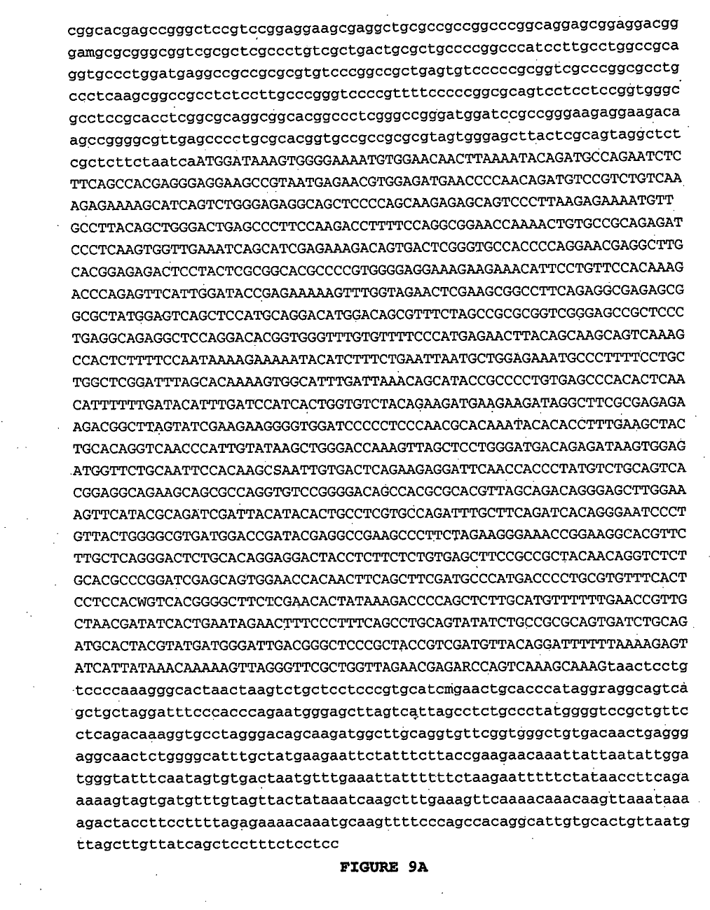

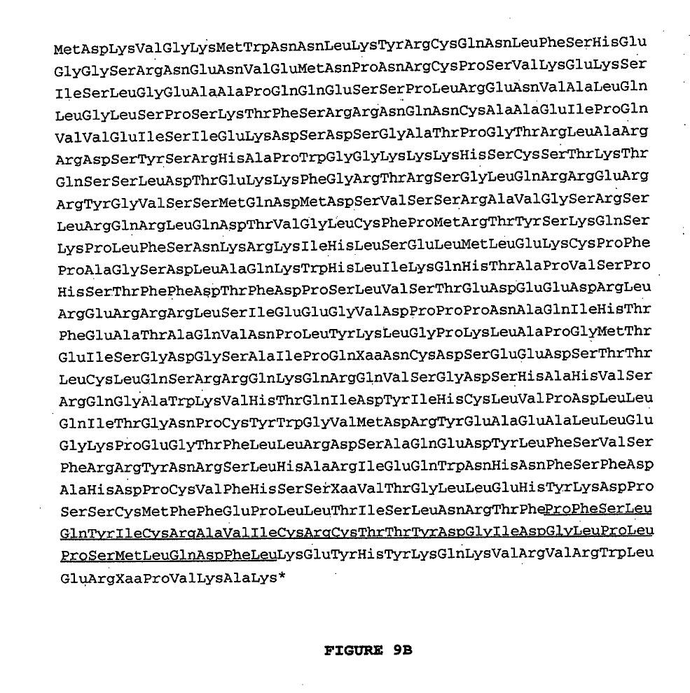

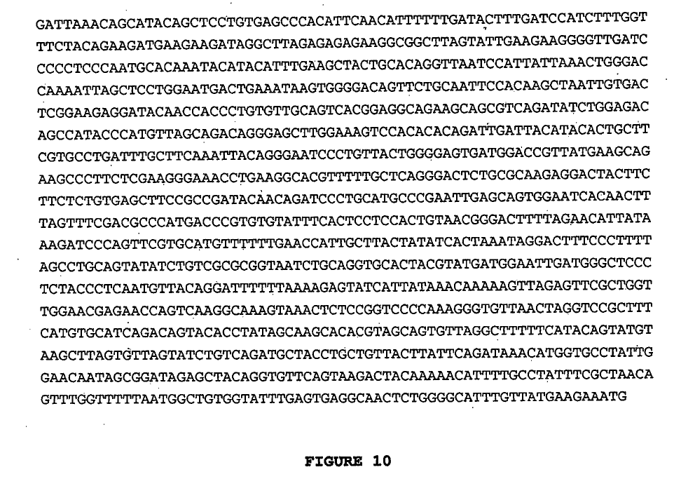

Mouse and human SOCS5 were recognized through searching EST databases using the SOCS box consensus (FIG. 4). Those ESTs derived from mouse and human SOCS5 cDNAs are tabulated below (Tables 5.1 and 5.2). Using sequence information derived from mouse and human ESTs, several oligonucleotides were designed and used to screen, in the conventional manner, a mouse thymus cDNA library, a mouse genomic DNA library and a human thymus cDNA library cloned into 1-bacteriophage. A single genomic DNA clone (57-2) and (5-3-2) cDNA clone encoding mouse SOCS5 were isolated and sequenced in their entirety and shown to overlap with the mouse ESTs identified in the database (FIGS. 8 and 9A). The entire coding region, in addition to a region of 5′ and 3′ untranslated regions of mouse SOCS5 appears to be encoded on a single exon (FIG. 8). Analysis of the sequence (FIG. 9) confirms that SOCS5 genomic and cDNA clones encode a protein with a SOCS box at its C-terminus in addition to an SH2 domain (FIGS. 8 and 9B). The relationship of the human SOCS5 contig (h 5.1; FIG. 10) derived from analysis of cDNA clone 5-94-2 and the human SOCS5 ESTs (Table 5.2) to the mouse SOCS5 DNA sequence is shown in FIG. 8. The nucleotide sequence and corresponding amino acid sequence of murine SOCS5 are shown in SEQ ID NOS:17 and 18, respectively. The human SOCS5 nucleotide sequence is shown in SEQ ID NO: 19.

### Example 27

### SOCS6

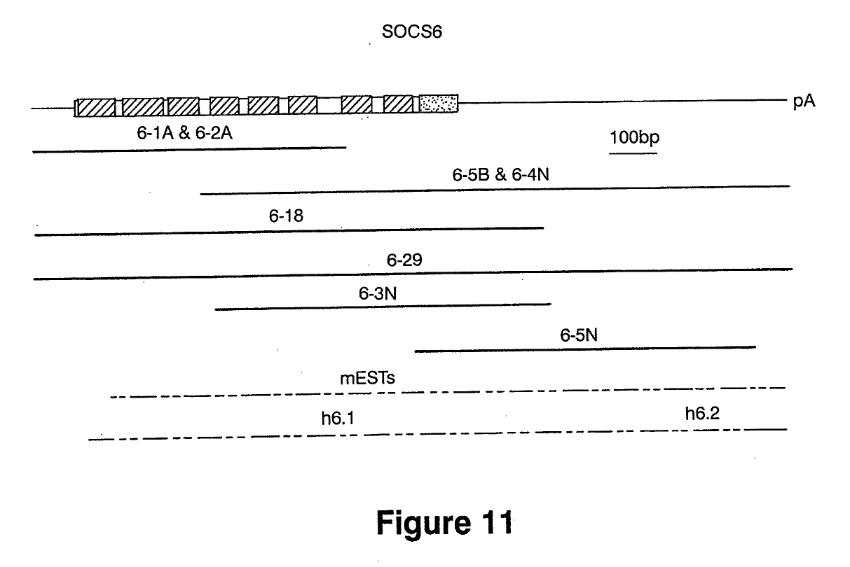

Mouse and human SOCS6 were recognized through searching EST databases using the SOCS box consensus (FIG. 4). Those ESTs derived from mouse and human SOCS6 cDNAs are tabulated below (Tables 6.1 and 6.2). Using sequence information derived from mouse ESTs, several oligonucleotides were designed and use to screen, in the conventional manner, a mouse thymus cDNA library. Eight cDNA clones (6-1A, 6-2A, 6-5B, 6-4N, 6-18, 6-29, 6-3N, 6-5N) cDNA clone encoding mouse SOCS6 were isolated and sequenced in their entirety and shown to overlap with the mouse ESTs identified in the database (FIGS. 11 and 12A). Analysis of the sequence (FIG. 12) confirms that the mouse SOCS6 cDNA clones encode a protein with a SOCS box at its C-terminus in addition to a eight WD-40 repeats (FIGS. 11 and 12B). The relationship of the human SOCS-6 contigs (h6.1 and h6.2; FIG. 4) derived from analysis of human SOCS6 ESTs (Table 6.2) to the mouse SOCS6 DNA sequence is shown in FIG. 11. The nucleotide and corresponding amino acid sequences of murine SOCS6 are shown in SEQ ID NOS:20 and 21, respectively. SOCS6 human contigs h6.1 and h6.2 are shown in SEQ ID NOS:22 and 23, respectively.

### Example 28

### SOCS7

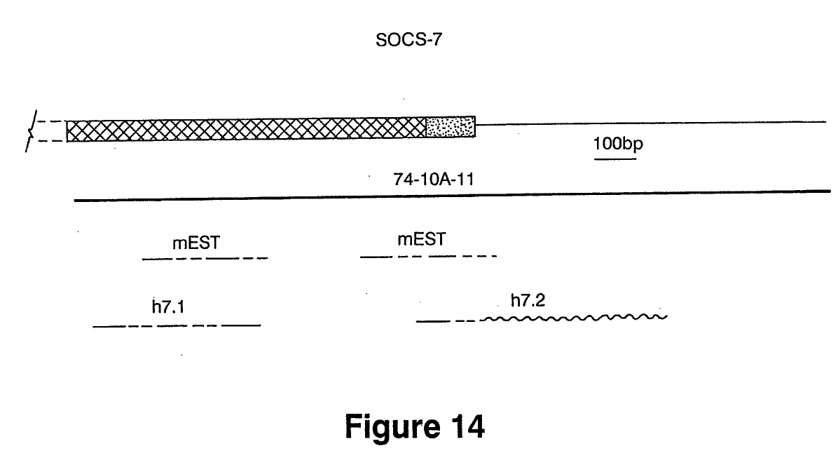

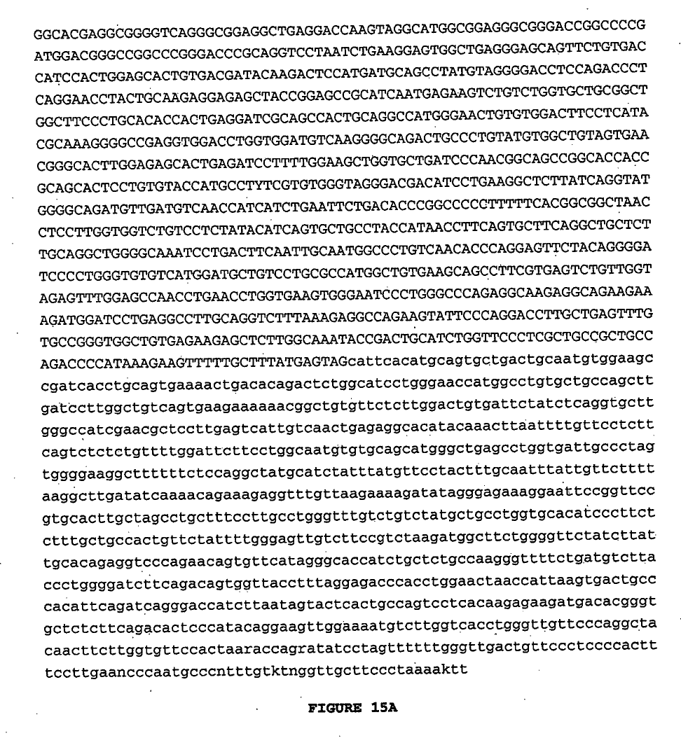

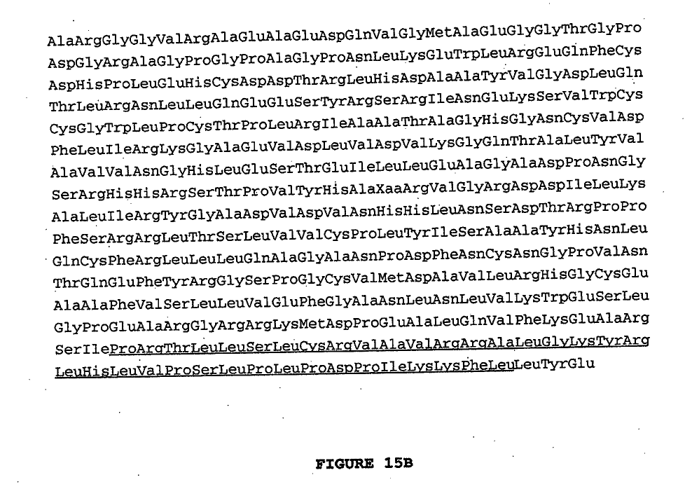

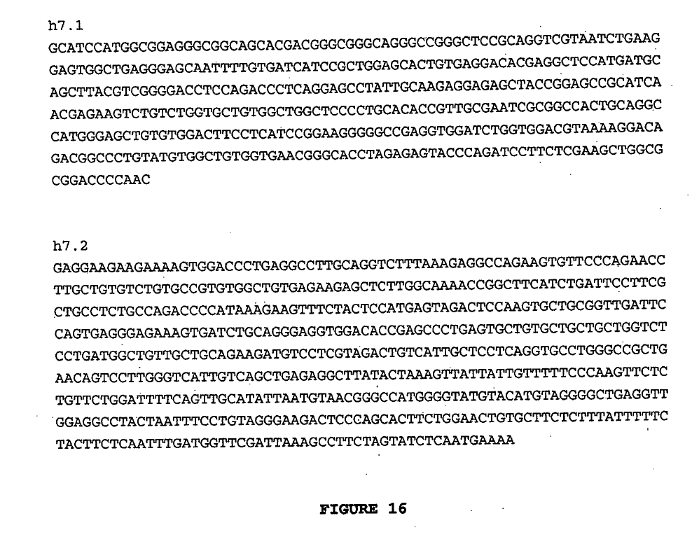

Mouse and human SOCS7 were recognized through searching EST databases using the SOCS box consensus (FIG. 4). Those ESTs derived from mouse and human SOCS-7 cDNAs are tabulated below (Tables 7.1 and 7.2). Using sequence information derived from mouse ESTs, several oligonucleotides were designed and use to screen, in the conventional manner, a mouse thymus cDNA library. One cDNA clone (74-10A-11) cDNA clone encoding mouse SOCS7 was isolated and sequenced in its entirety and shown to overlap with the mouse ESTs identified in the database (FIGS. 14 and 15A). Analysis of the sequence (FIG. 15) suggests that mouse SOCS7 encodes a protein with a SOCS box at its C-terminus, in addition to several ankyrin repeats (FIGS. 14 and 15B). The relationship of the human SOCS7 contigs (h7.1 and h7.2; FIG. 16) derived from analysis of human SOCS7 ESTs (Table 7.2) to the mouse SOCS7 DNA sequence is shown in FIG. 14. The nucleotide and corresponding amino acid sequences of murine SOCS7 are shown in SEQ ID NOS:24 and 25, respectively. The nucleotide sequence of SOCS7 human contigs h7.1 and h7.2 are shown in SEQ ID NOS:26 and 27, respectively.

### Example 29

### SOCS8

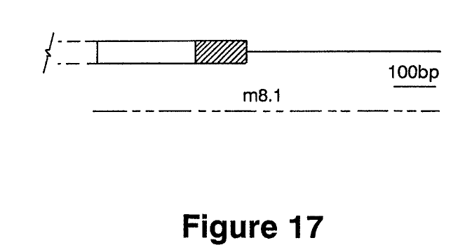

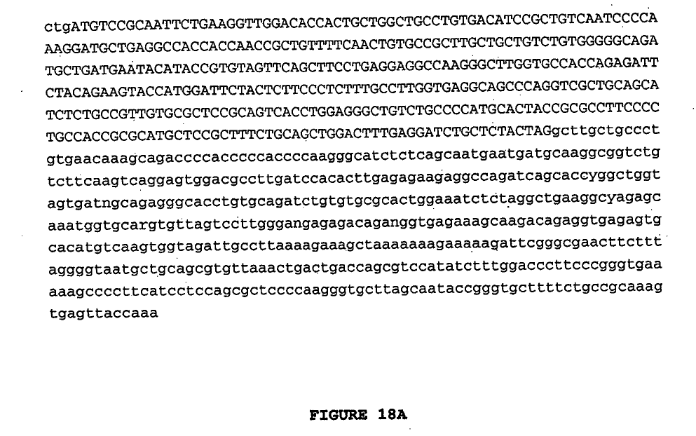

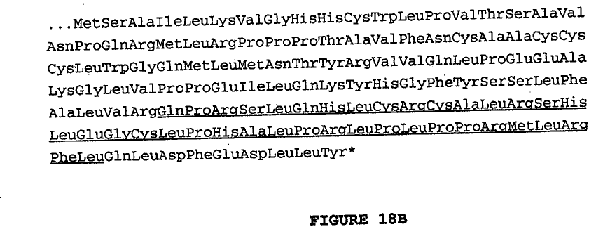

ESTs derived from mouse SOCS8 cDNAs are tabulated below (Table 8.1). As described for other members of the SOCS family, it is possible to isolate cDNAs for mouse SOCS8 using sequence information derived from mouse ESTs. The relationship of the ESTs to the predicted coding region of SOCS8 is shown in FIG. 17. With the nucleotide sequence obtained from the ESTs shown in FIG. 18A and the partial amino acid sequence of SOCS8 shown in FIG. 18B. The nucleotide sequence and corresponding amino acid sequences for murine SOCS8 are shown in SEQ ID NOS:28 and 29, respectively.

### Example 30

### SOCS9

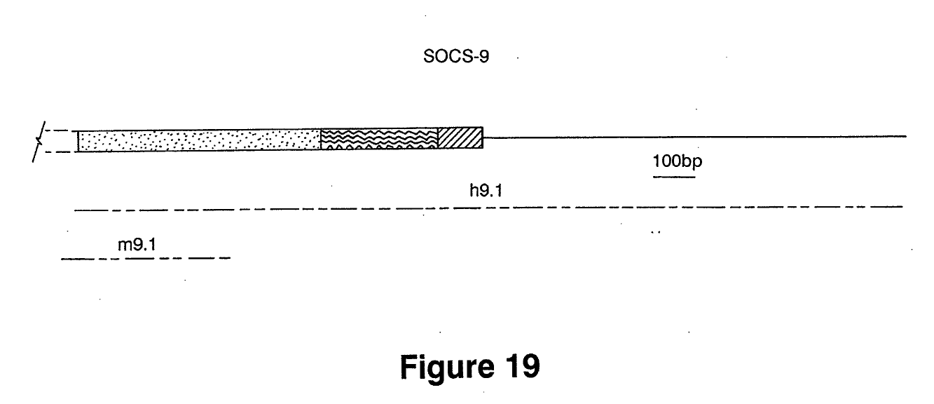

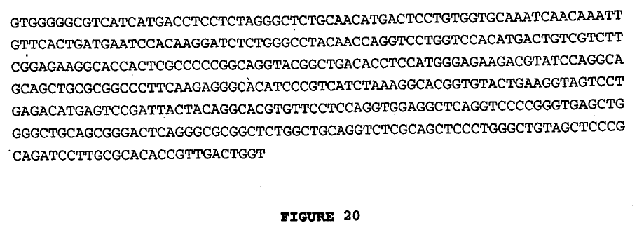

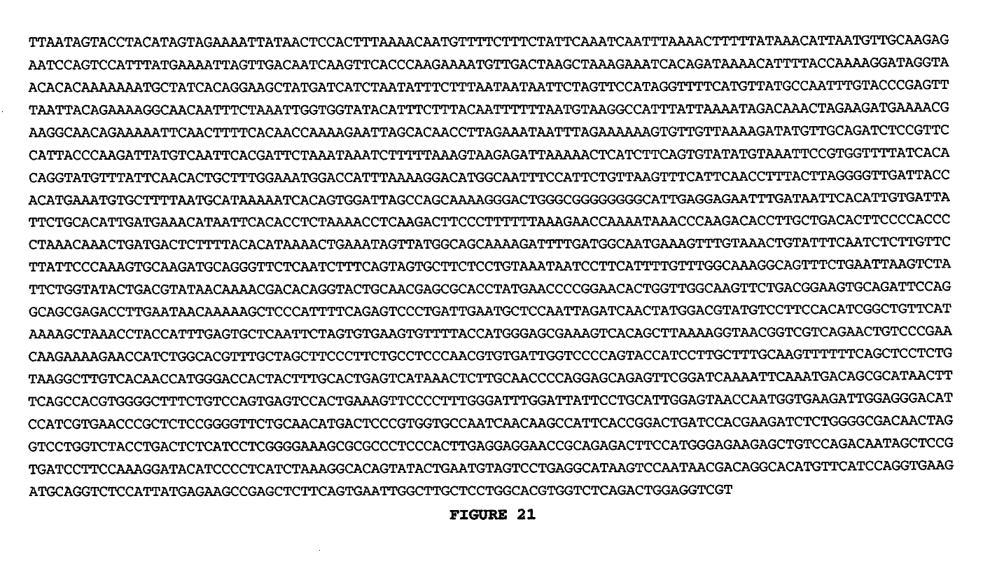

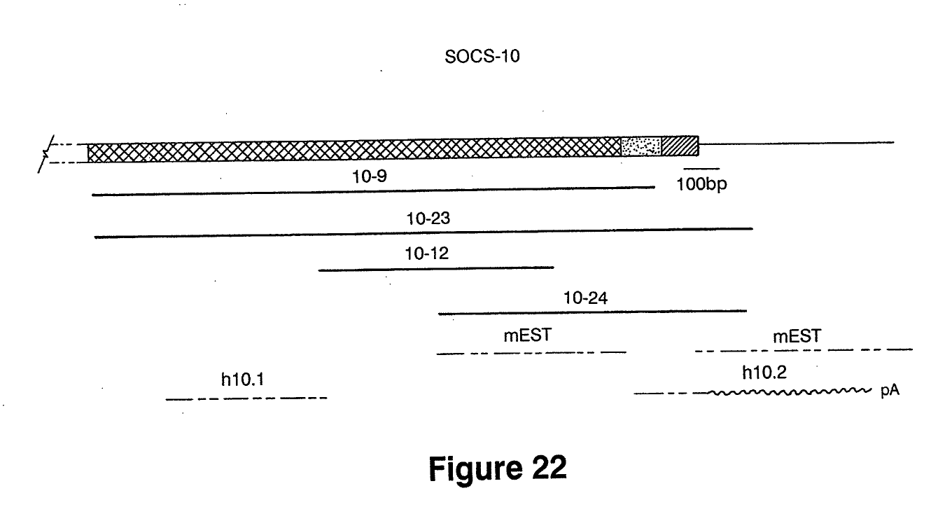

Mouse and human SOCS-9 were recognized through searching EST databases using the SOCS box consensus (FIG. 4). Those ESTs derived from mouse and human SOCS9 cDNAs are tabulated below (Tables 9.1 and 9.2). The relationship of the mouse SOCS9 contigs derived from analysis of the mouse SOCS9 EST (Table 9.1) to the human SOCS-9 DNA contig (h9.1; FIG. 21) derived from analysis of human SOCS9 ESTs (Table 9.2) is shown in FIG. 20. Analysis of the sequence (FIG. 22) indicates that the human SOCS9 cDNA encodes a protein with a SOCS box at its C-terminus, in addition to an SH2 domain (FIG. 19). The nucleotide sequence of muring SOCS9 cDNA is shown in SEQ ID NO:30. The nucleotide sequence of human SOCS9 cDNA is shown in SEQ ID NO:31.

### Example 31

### SOCS10

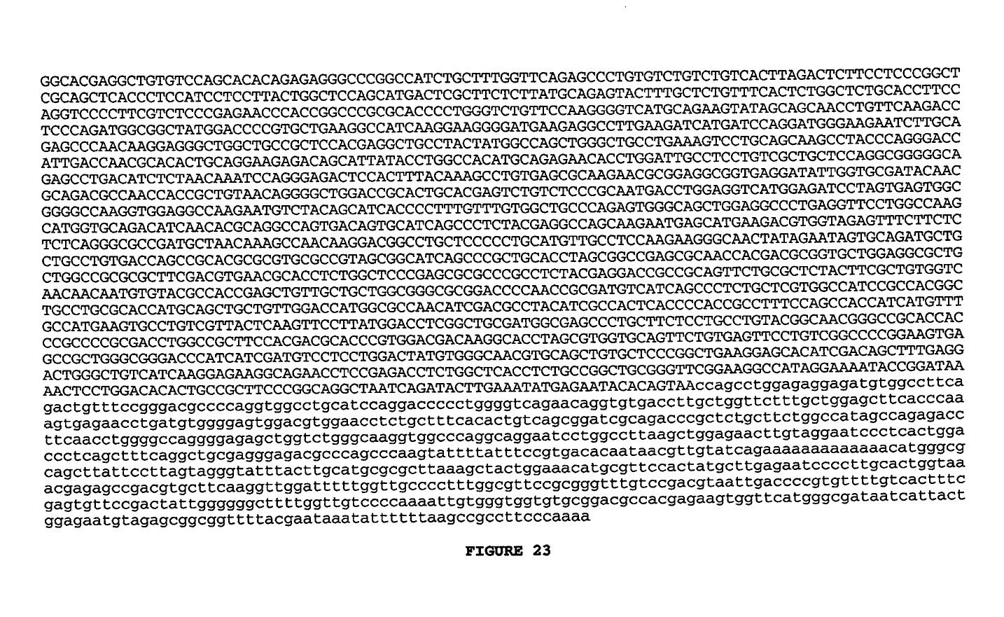

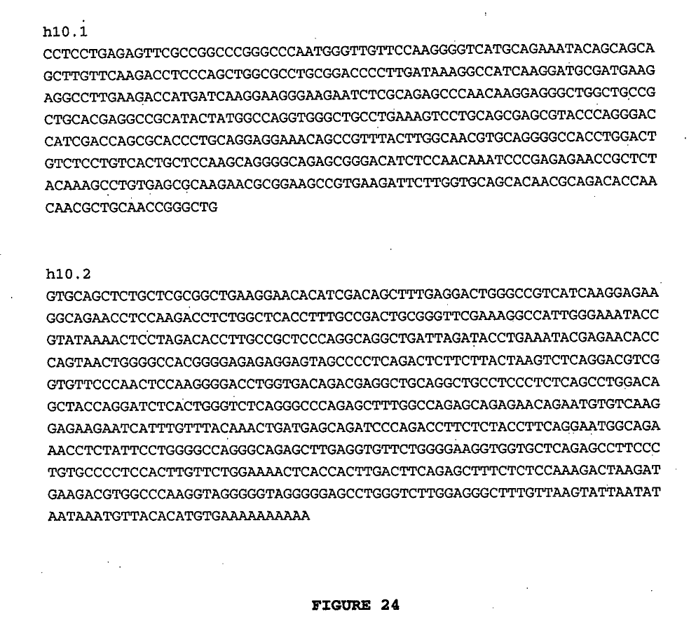

Mouse and human SOCS10 were recognized through searching EST databases using the SOCS box consensus (FIG. 4). Those ESTs derived from mouse and human SOCS10 cDNAs are tabulated below (Table 10.1 and 10.2). Using sequence information derived from mouse ESTs, several oligonucleotides were designed and use to screen, in the conventional manner, a mouse thymus cDNA library. Four cDNA clones (10-9, 10-12, 10-23 and 10-24) encoding mouse SOCS10 were isolated, sequenced in their entirety and shown to overlap with the mouse and human ESTs identified in the database (FIGS. 22 and 23). Analysis of the sequence (FIG. 23) indicates that the mouse SOCS10 cDNA clone is not full length but that it does encode a protein with a SOCS box at its C-terminus, in addition to several ankyrin repeats (FIG. 22). The relationship of the human SOCS10 contigs (h10.1 and h10.2; FIG. 24) derived from analysis of human SOCS10 ESTs (Table 10.2) to the mouse SOCS10 DNA sequence is shown in FIG. 22. Comparison of mouse cDNA clones and ESTs with human ESTs suggests that the 3′ untranslated regions of mouse and human SOCS10 differ significantly. The nucleotide sequence of murine SOCS10 is shown in SEQ ID NO:32 and the nucleotide sequence of SOCS10 human contigs h10.1 and h10.2 are shown in SEQ ID NOS:33 and 34, respectively.

### Example 32

### SOCS11

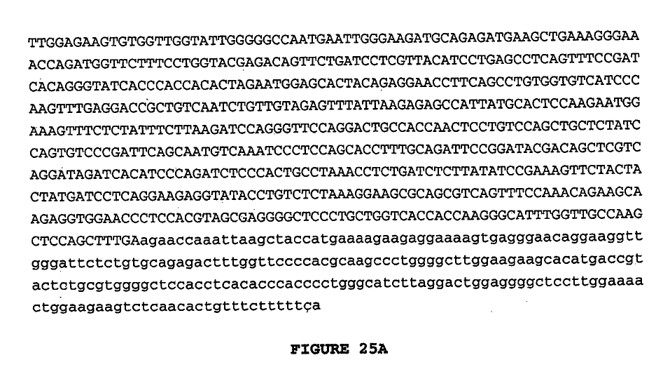

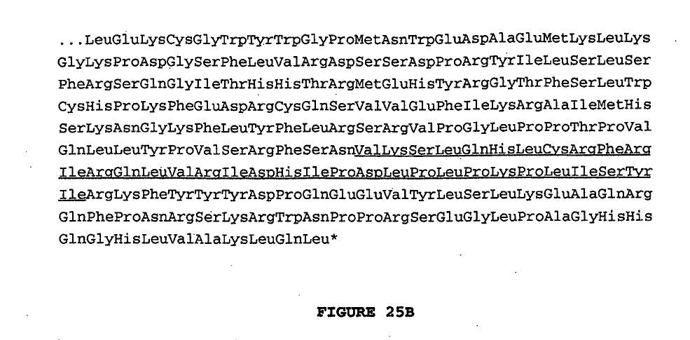

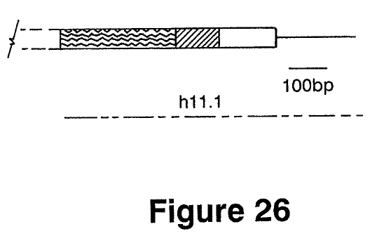

Human SOCS11 were recognized through searching EST databases using the SOCS box consensus (FIG. 4). Those ESTs derived from human SOCS11 cDNAs are tabulated below (Table 11.1 and 11.2). The relationship of the human SOCS11 contigs (h11.1; FIG. 25A, B), derived from analysis ESTs (Table 11.2) to the predicted encoded protein, is shown in FIG. 26. Analysis of the sequence indicates that the human SOCS11 cDNA encodes a protein with a SOCS box at its C-terminus, in addition to an SH2 domain (FIGS. 26 and 25B). The nucleotide sequence and corresponding amino acid sequence of human SOCS11 are represented in SEQ ID NOS:35 and 36, respectively.

### Example 33

### SOCS12

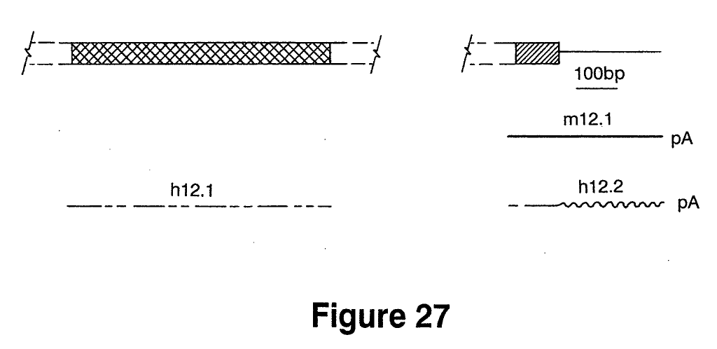

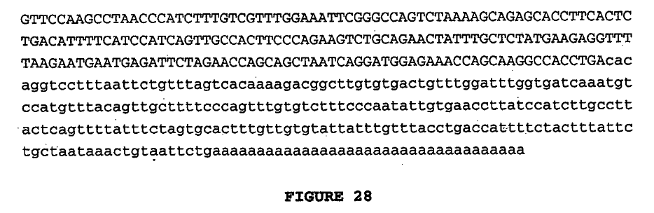

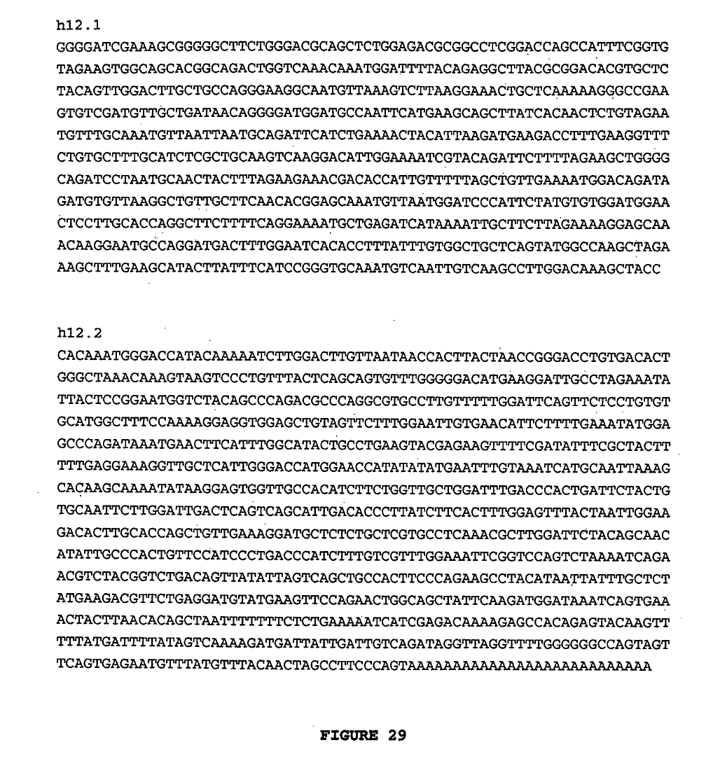

Mouse and human SOCS-12 were recognized through searching EST databases using the SOCS box consensus (FIG. 4). Those ESTs derived from mouse and human SOCS12 cDNAs are tabulated below (Tables 12.1 and 12.2). Using sequence information derived from mouse ESTs, several oligonucleotides were designed and use to screen, in the conventional manner, a mouse thymus cDNA library. Four cDNA clones (10-9, 10-12, 10-23 and 10-24) encoding mouse SOCS12 were isolated, sequenced in their entirety and shown to overlap with the mouse and human ESTs identified in the database (FIGS. 27 and 28). Analysis of the sequence (FIGS. 28 and 29) indicates that the SOCS12 cDNA clone encodes a protein with a SOCS box at its C-terminus, in addition to several ankyrin repeats (FIG. 27). The relationship of the human SOCS12 contigs (h12.1 and h12.2; FIG. 29) derived from analysis of human SOCS12 ESTs (Table 12.2) to the mouse SOCS12 DNA sequence is shown in FIG. 27. Comparison of mouse cDNA clones and ESTs with human ESTs suggests that the 3′ untranslated regions of mouse and human SOCS12 differ significantly. The nucleotide sequence of SOCS12 is shown in SEQ ID NO:37. The nucleotide sequence of human SOCS12 contigs h12.1 and h12.2 are shown in SEQ ID NOS:38 and 39, respectively.

### Example 34

### SOCS13

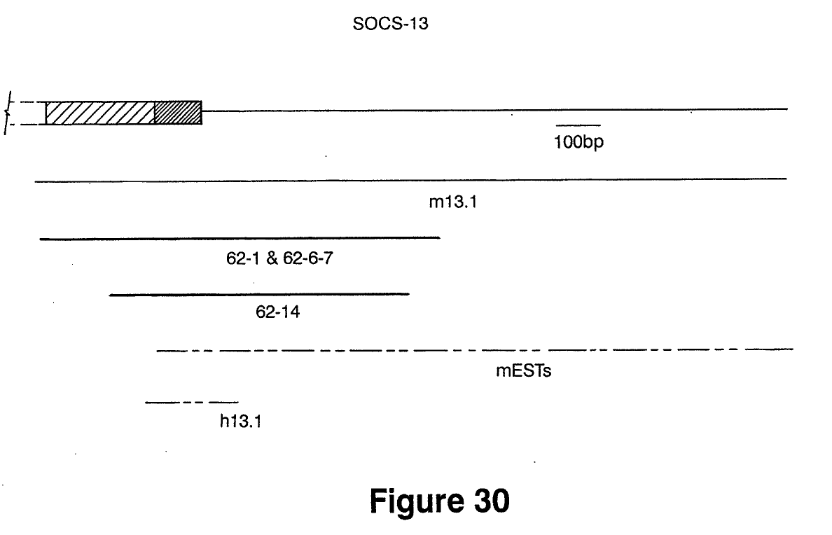

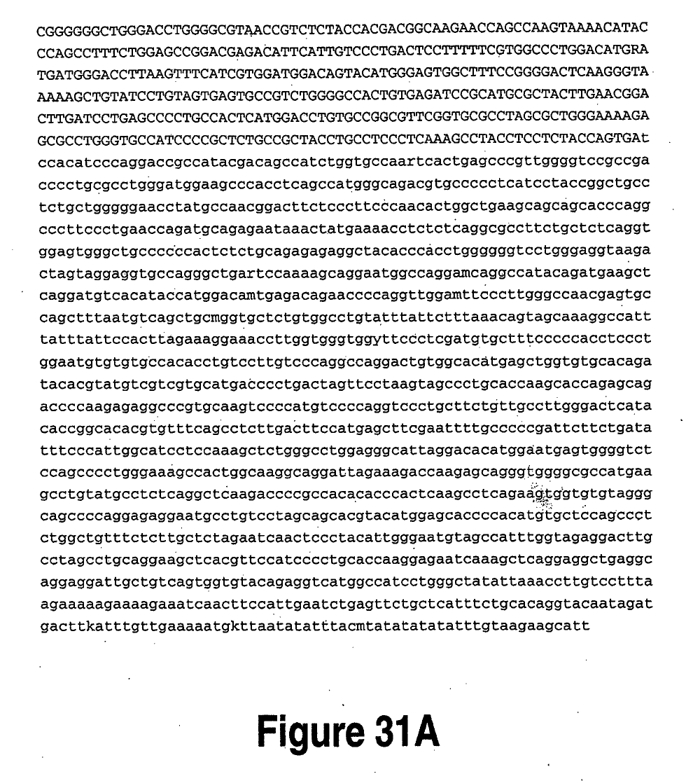

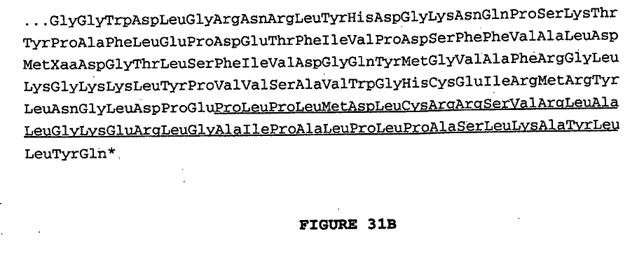

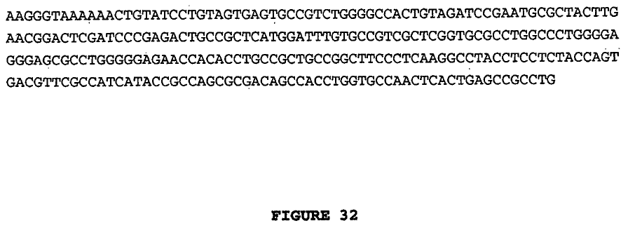

Mouse and human SOCS-13 were recognized through searching EST data bases using the SOCS box consensus (FIG. 4). Those ESTs derived from mouse and human SOCS13 cDNAs are tabulated below (Tables 13.1 and 13.2). Using sequence information derived from mouse ESTs, several oligonucleotides were designed and use to screen, in the conventional manner, a mouse thymus and a mouse embryo cDNA library. Three cDNA clones (62-1, 62-6-7 and 62-14) encoding mouse SOCS13 were isolated, sequenced in their entirety and shown to overlap with the mouse ESTs identified in the database (FIGS. 30 and 31A). Analysis of the sequence (FIG. 31) indicates that the mouse SOCS13 cDNA encodes a protein with a SOCS box at its C-terminus, in addition to a potential WD-40 repeat (FIGS. 30 and 31B). The relationship of the human SOCS13 contigs (h13.1 and h13.2; FIG. 32) derived from analysis of human SOCS13 ESTs (Table 13.2) to the mouse SOCS13 DNA sequence is shown in FIG. 30. The nucleotide sequence and corresponding amino acid sequence of murine SOCS13 and shown in SEQ ID NOS:40 and 41, respectively. The nucleotide sequence of human SOCS13 contig h13.1 shown in SEQ ID NO:42.

Mice lacking SOCS-1 are born and appear outwardly normal. However, they fail to thrive and within two to three weeks are less than half the size of their normal littermates. All SOCS^{-/-} mice die before weaning with profound fatty degeneration of the liver (FIG. 1). Consistent with the SOCS-1 expression pattern outlined above, significant deficiencies in haemopoietic populations, particularly lymphocytes (FIG. 2), are also evident. These experiments highlight the indispensable nature of SOCS-1 action, suggesting that negative regulation of cytokine signalling by this protein is critical in maintaining homeostasis in the liver, as well as in the proper control of the production of specific blood cells.

Analysis of β-galactosidase activity in mice in which one SOCS-1 allele has been replaced with β-gal has revealed expression in most thymocytes, as well as in the spleen and bone marrow, where it appears to be restricted largely to lymphoid populations.

### Example 34A

To explore the physiological role of SOCS-1, the inventors generated mice lacking this gene. SOCS-1 deficient (SOCS-1^{-/-}) mice are born at the expected Mendelian frequency, appear normal for the first week. Between 9 and 21 days of age the mice succumb to an illness characterised by fatty degeneration and monocytic infiltration of the liver, monocytic infiltration of the pancreas and heart and a severe lymphopenia. While the molecular basis of this disease was unclear the most parsimonious hypothesis, given the role of SOCS proteins in negative regulation of signal transduction, is that the SOCS-1^{-/-} mice are hyper-responsive to a cytokine known to have toxic side effects. Strikingly, the phenotype of the SOCS-1^{-/-} mice was similar to that described for neonatal mice injected with interferon gamma from birth.

SOCS-1 mRNA Expression is Induced by IFNα, IFNβ and IFNγ

In order to determine whether the cellular response to IFNα, IFNβ or IFNγ might be regulated by SOCS-1, the inventors examined whether these cytokines induced expression of SOCS-1 mRNA and whether expression of SOCS-1 inhibited the biological effect of interferon's. Northern blot analysis of mRNA from the fibroblast cell lines 2FTGH revealed that expression of SOCS-1, SOCS-2, SOCS-3 and CIS mRNA was low or undetectable in unstimulated cells. Within 15 to 30 minutes of stimulation by IFNα, IFNβ or IFNγ SOCS-1 and to a lesser extent SOCS-3 mRNA were detectable with expression peaking at about 60 minutes. Little or no expression of either SOCS-2 or CIS was observed in response to any of the interferons. A similar pattern of induction of SOCS mRNA was observed upon treatment of the J774 macrophage cell line with IFNγ and, in the liver, following intravenous injection of IFNγ into mice.

### Expression of SOCS-1 and SOCS-3 but not SOCS-2 or CIS Inhibits IFNα, IFNβ or IFNγ Signalling

2FTGH cells were transfected with expression vectors encoding FLAG-tagged versions of SOCS-1, SOCS-2, SOCS-3 and CIS and clones stably expressing these proteins were selected. The capacity of these lines to respond to IFNα, IFNβ or IFNγ was compared with control 2FTGH cells. Cells were infected with virus and incubated with various concentrations of each IFN. Wild type cells and those expressing SOCS-2 and CIS were protected from the effects of virus infection by 300 IU/ml IFNα or IFNγ and 10 IU/ml IFNβ. In contrast, those expressing SOCS-1 and SOCS-3 exhibited reduced sensitivity to the protective effects of all three forms of IFN. In the case of IFNβ SOCS-1 expressing 2FTGH cells were at least 300-fold less sensitive and SOCS-3 expressing 2FTGH cells were 10 to 30-fold less sensitive than unmanipulated counterparts. A similar hypo-responsiveness was observed when the capacity of IFNβ to suppress cell proliferation was assessed.

### Disease in SOCS-1^{-/-} Mice is Predated by Cellular Responses to IFNγ

Three lines of evidence suggested that the pathology observed in SOCS-1 deficient mice might result from a hyper-responsiveness to IFNγ. These were (i) the capacity of IFNγ to stimulate SOCS-1 expression, (ii) the ability of SOCS-1 to inhibit IFNγ signalling when constitutively expressed and (iii) the similarity of the phenotype of SOCS-1 deficient mice and mice injected IFNγ. The inventors therefore examined whether SOCS-1 mice showed evidence of an ongoing response to IFNγ either during the first week of life prior to overt development of disease or during disease onset and progression in the second and third weeks of life.

Phosphorylation of the IFNγRα chain by JAK1 and JAK2 and the consequent activation of STAT1 are key elements in IFNγ signal transduction. In the livers of new born, six-day-old and 14-day-old SOCS-1^{-/-} mice but not SOCS-1^{+/-} or wild type mice tyrosine phosphorylation of the IFNγRα was readily detectable. Likewise, activated STAT1, as measured by EMSA, was also detected in SOCS-1^{-/-} but not littermates, at the three time points examined.

Given the evidence of IFNγ signalling in SOCS-1^{-/-} mice examined the liver for expression of class I and II MHC, iNOS and IRF-1-hallmarks of a biological response to IFNγ. At birth and after 6 and 14 days of life wild type and SOCS-1^{+/-} mice showed a low level of expression of class I MHC and little or no expression of class II MHC, iNOS and IRF-1. In contrast, expression of each of these proteins was elevated in the livers of SOCS-1^{-/-} mice at each time point examined. Class I and II MHC expression was also found to be elevated in many cells of the haemopoietic system, notably thymic and splenic T cells, as well as bone marrow and splenic B cells and monocytes. Expression of markers of a response to IFNγ in the liver and the haemopoietic system occurred before the onset of the overt signs of disease in SOCS-1^{-/-} mice suggesting that it was not a secondary effect of the pathology observed.

### Lack of IFNg Completely Ameliorates Disease in SOCS-1^{-/-} Mice

In order to determine whether a response to IFNγ was the basis of disease development in SOCS-1^{-/-} mice the inventors performed two experiments; (i) injection of mice with neutralising anti-IFNγ antibody and (ii) generation of mice lacking functional SOCS-1 and IFNγ genes.

Litters of mice born following the mating of SOCS-1^{-/+} mice were injected twice weekly from birth with either anti-IFNγ monoclonal antibody or an isotype control antibody. SOCS-1^{-/-} mice injected with control antibody developed disease with the same onset as similar unmanipulated SOCS-1^{-/-}. The disease observed in unmanipulated and control antibody injected SOCS-1^{-/-} mice were also similar and was characterised by fatty degeneration of the liver, monocytic infiltration of several organs including the heart, liver, pancreas and skin, a generalised reduction in the size of the thymus and selective loss of pre-B and mature B cells.

### Example 35

### SOCS14

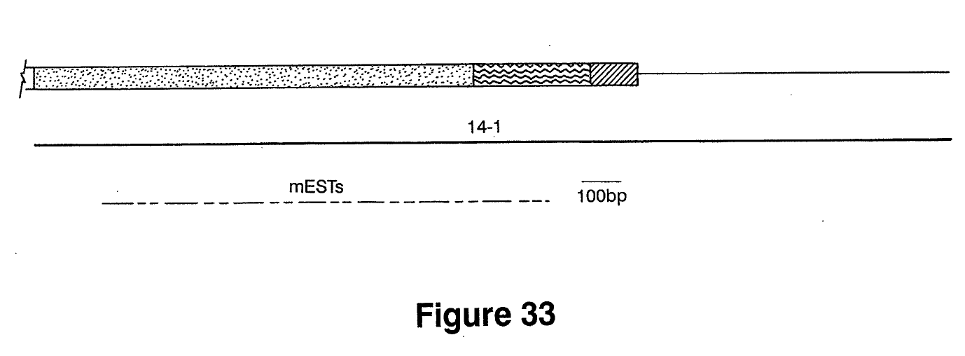

Mouse and human SOCS-14 were recognized through searching EST databases using the SOCS box consensus (FIG. 4). Those ESTs derived from mouse and human SOCS14 cDNAs are tabulated below (Tables 14.1 and 14.2). Using sequence information derived from mouse and human ESTs, several oligonucleotides were designed and use to screen, in the conventional manner, a mouse thymus cDNA library, a mouse genomic DNA library and a human thymus cDNA library cloned into 1-bacteriophage. A single genomic DNA clone (57-2) and (5-3-2) cDNA clone encoding mouse SOCS14 were isolated and sequenced in their entirety and shown to overlap with the mouse ESTs identified in the database (FIGS. 33 and 34A). The entire coding region, in addition to a region of 5′ and 3′ untranslated regions, of mouse SOCS14 appears to be encoded on a single exon (FIG. 33). Analysis of the sequence (FIG. 34) confirms that SOCS14 genomic and cDNA clones encode a protein with a SOCS box at its C-terminus in addition to an SH2 domain (FIGS. 33 and 34B). The relationship of the human SOCS14 contig (h14.1) derived from analysis of cDNA clone 5-94-2 and the human SOCS14 ESTs (Table 14.2) to the mouse SOCS14 DNA sequence is shown in FIG. 33.

The nucleotide sequence and corresponding amino acid sequence of murine SOCS14 are shown in SEQ ID NOS:43 and 44, respectively.

### Example 36

### SOCS15

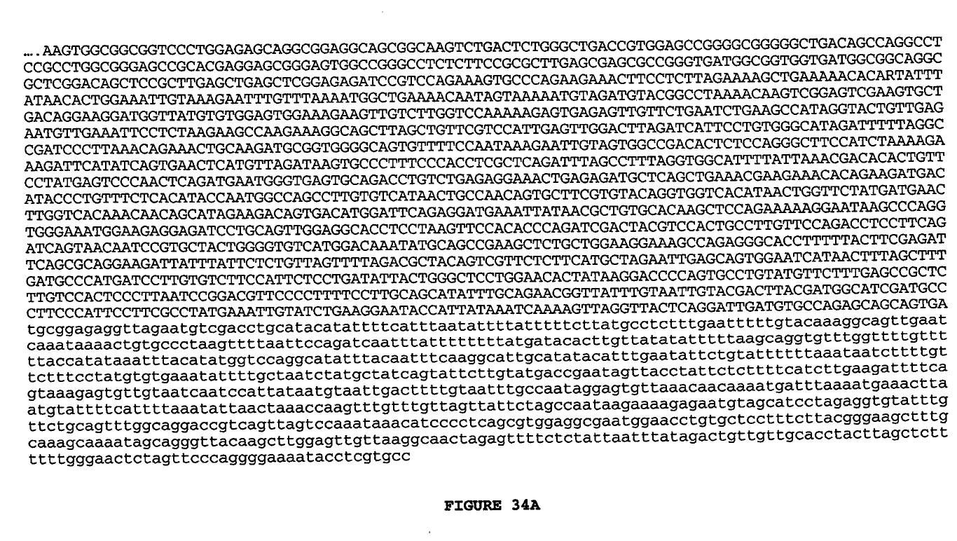

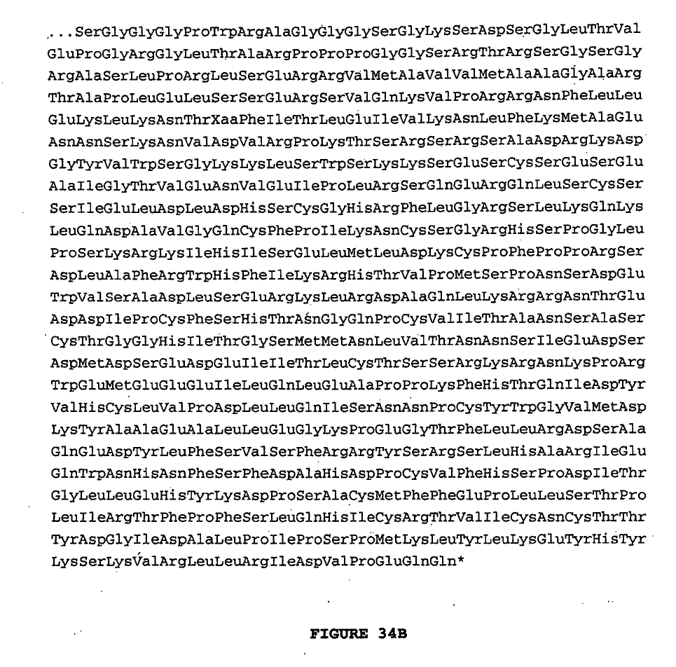

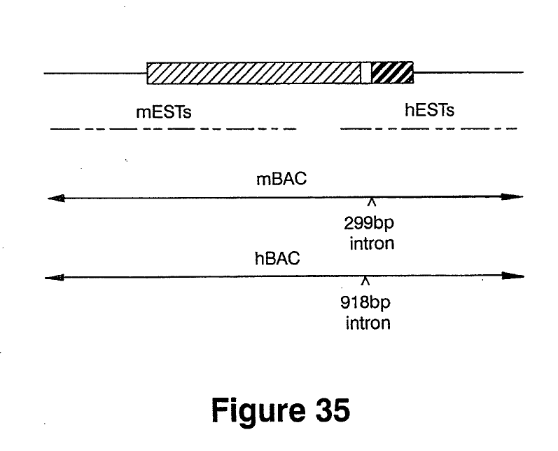

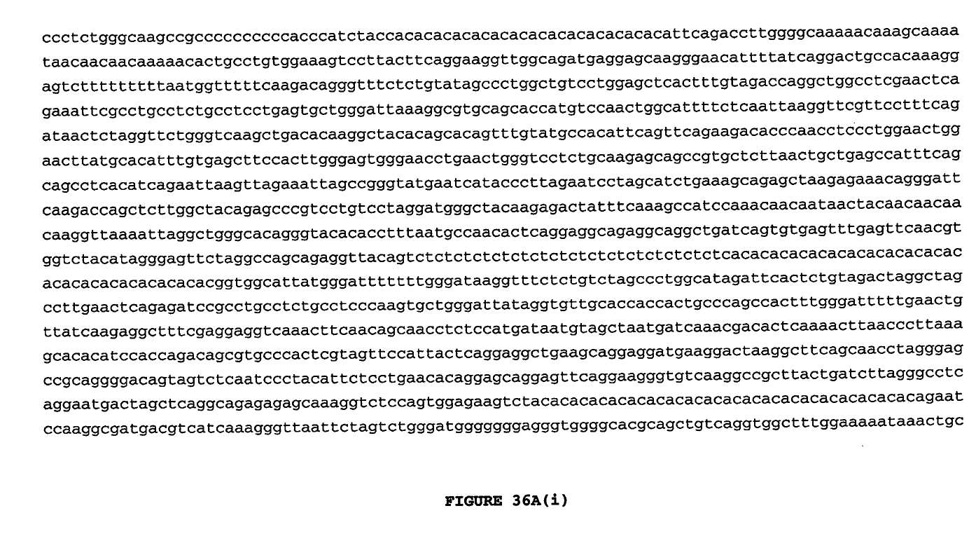

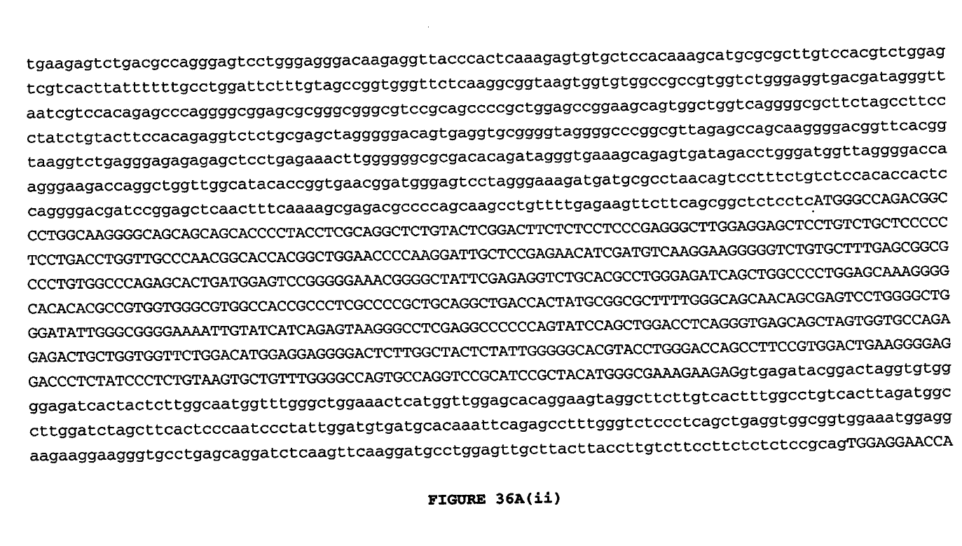

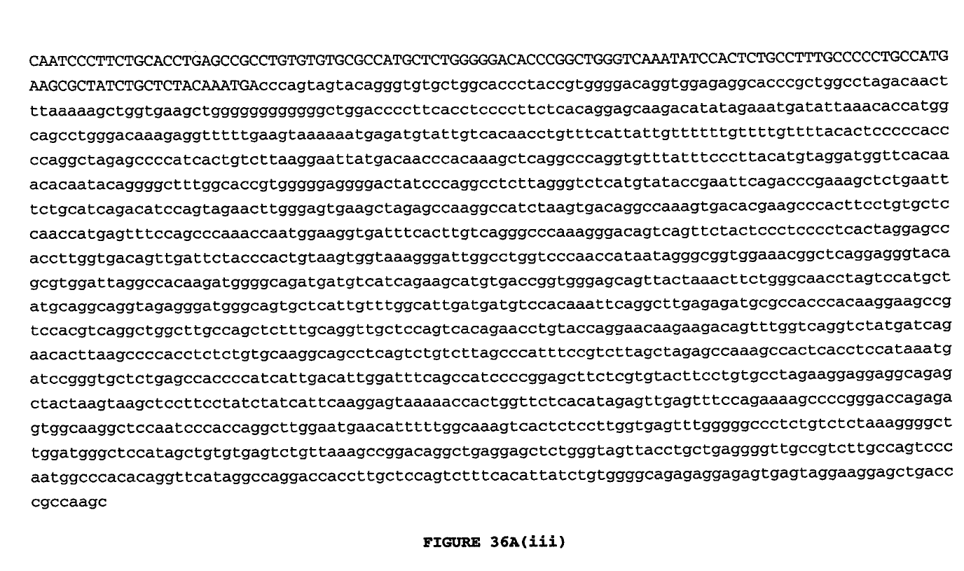

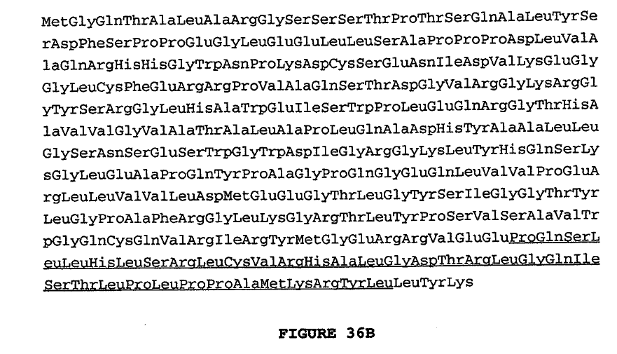

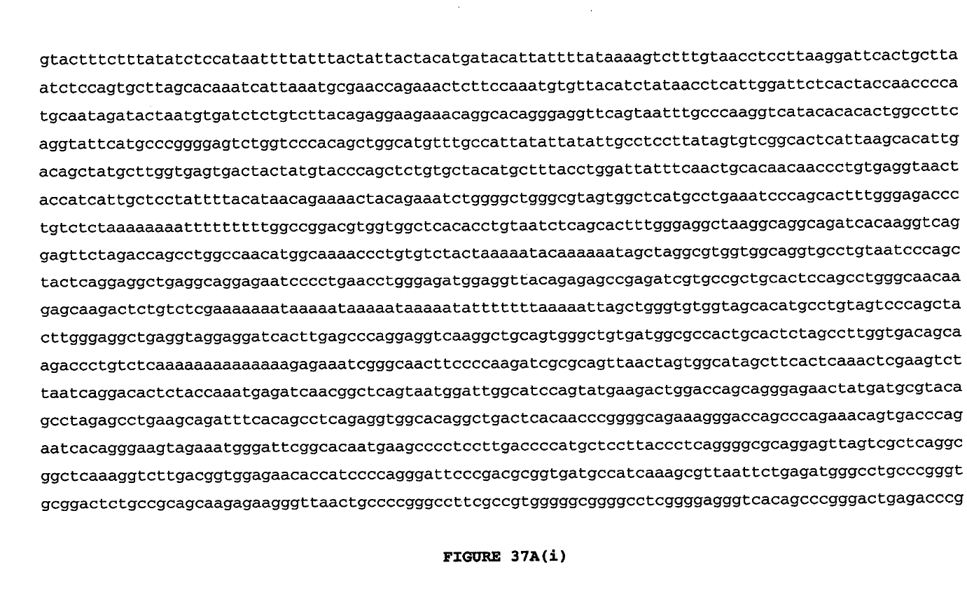

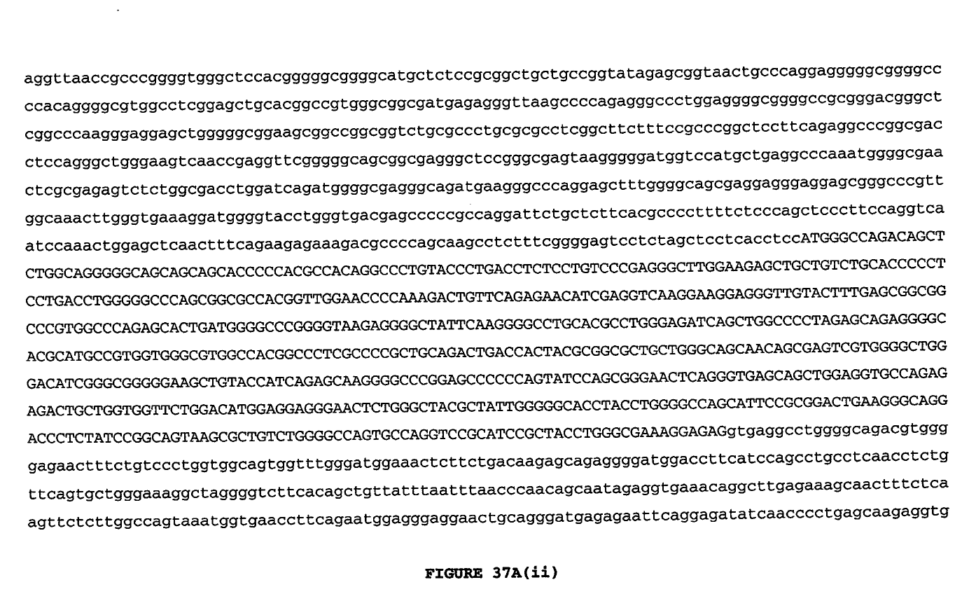

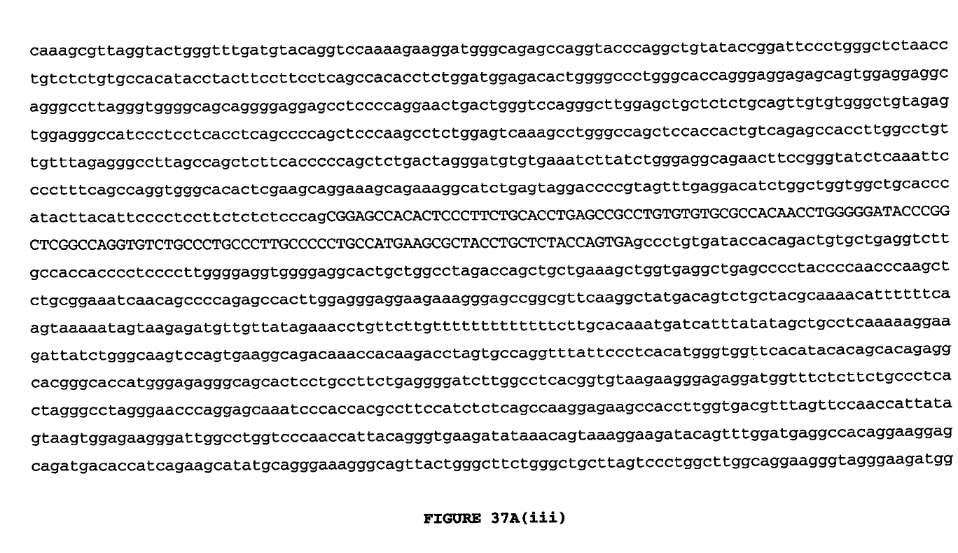

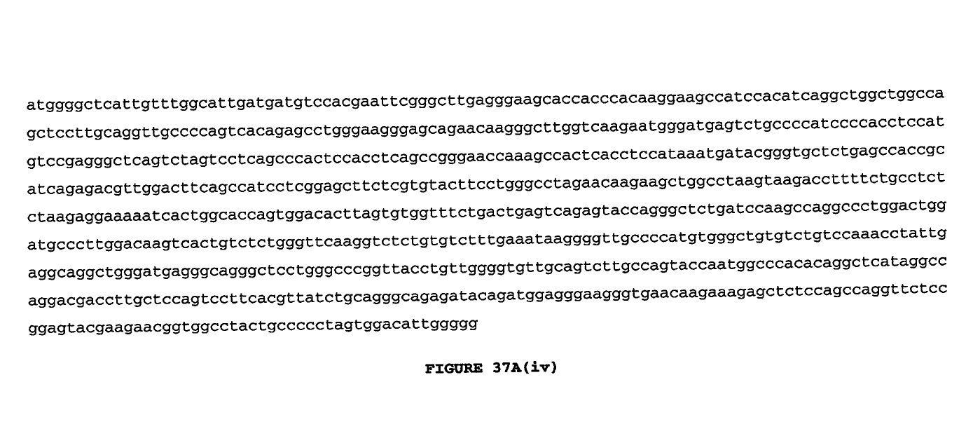

Mouse and human SOCS15 were recognized through searching DNA databases using the SOCS box consensus (FIG. 4). Those ESTs derived from mouse and human SOCS15 cDNAs are tabulated below (Tables 15.1 and 15.2), as are a mouse and human BAC that contain the entire mouse and human SOCS-15 genes. Using sequence information derived from the ESTs and the BACs it is possible to predict the entire amino acid sequence of SOCS15 and as described for the other SOCS genes it is feasible to design specific oligonucleotide probes to allow cDNAs to be isolated. The relationship of the BACs to the ESTs is shown in FIG. 35 and the nucleotide and predicted amino acid sequence of the SOCS-15, derived from the mouse and human BACs is shown in FIGS. 36 and 37. The nucleotide sequence and corresponding amino acid sequence of murine SOCS15 are shown in SEQ ID NOS:46 and 47, respectively. The nucleotide and corresponding amino acid sequence of human SOCS15 are shown in SEQ, ID NOS:48 and 49, respectively.

### Example 37

### SOCS Interaction with JAK2 Kinase

These Examples show interaction between SOCS and JAK2 kinase. Interaction is mediated via the SH2 domain of SOCS1, 2, 3 and CIS. The interaction resulted in inhibition of JAK2 kinase activity by SOCS1.

The following methods are employed:

Immunoprecipitation: Cos 6 cells were transiently transfected by electroporation and cultured for 48 hours. Cells were then lysed on ice in lysis buffer (50 mM Tris/HCL, pH 7.5, 150 mM NaCl, 1% v/v Triton-X-100, 1 mM EDTA, 1 mM Naf, 1 mM Na₃VO₄) with the addition of complete protease inhibitors (Boehringer Mannheim), centrifuged at 4° C. (14,000×g, 10 min) and the supernatant retained for immunoprecipitation. JAK2 proteins were immunoprecipitated using 5 μl anti-JAK2 antibody (UBI). Antigen-antibody complexes were recovered using protein A-Sepharose (30 μl of a 50% slurry).

Western blotting: Immunoprecipitates were analysed by sodium dodecyl sulphate (SDS)-polyacrylamide gel electrophoresis (PAGE) under reducing conditions. Protein was then electrophoretically transferred to nitrocellulose, blocked overnight in 10% w/v skim-milk and washed in PBS/0.1% v/v Tween-20 (Sigma) (wash buffer) prior to incubation with either anti-phosphotyrosine antibody (4G10) (1:5000, UBI), anti-FLAG antibody (1.6 μg/ml) or anti-JAK2 antibody (1:2000, UBI) diluted in wash buffer/1% w/v BSA for 2 hr. Nitrocellulose blots were washed and primary antibody detected with either peroxidase-conjugated sheep anti-rabbit:immunoglobulin (1:5000, Silenus) or peroxidase-conjugated sheep anti-mouse immunoglobulin (1:5000, Silenus) diluted in wash buffer/1% w/v BSA. Blots were washed and antibody binding visualised using the enhanced chemiluminescence (ECL) system (Amersham, UK) according to the manufacturers' instructions.

In-vitro kinase assay: An in vitro kinase assay was performed to assess intrinsic JAK2 kinase catalytic activity. JAK2 protein were immunoprecipitated as described, washed twice in kinase assay buffer (50 mM NaCl, 5 mM MgCl₂, 5 mM MnCl2, 1 mM NaF, 1 mM Na₃VO₄, 10 mM HEPES, pH 7.4) and suspended in an equal volume of kinase buffer containing 0.25 μCi/ml (γ-³²P)-ATP (30 min, room temperature). Excess (γ-³²P)-ATP was removed and the immunoprecipitates analysed by SDS/PAGE under reducing conditions. Gels were subjected to a mild alkaline hydrolysis by treatment with 1 M KOH (55° C., 2 hours) to remove phosphoserine and phosphothreonine. Radioactive bands were visualised with IMAGEQUANT software on a PhosphorImage system (Molecular Dynamics, Sunnyvale, Calif., USA).

### Example 38

### Making SOCS-1 Knockout Constructs

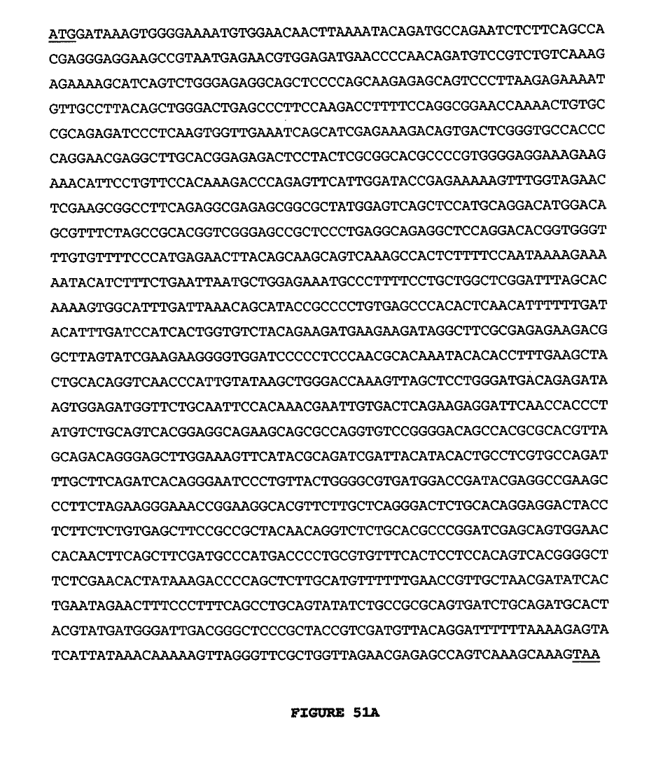

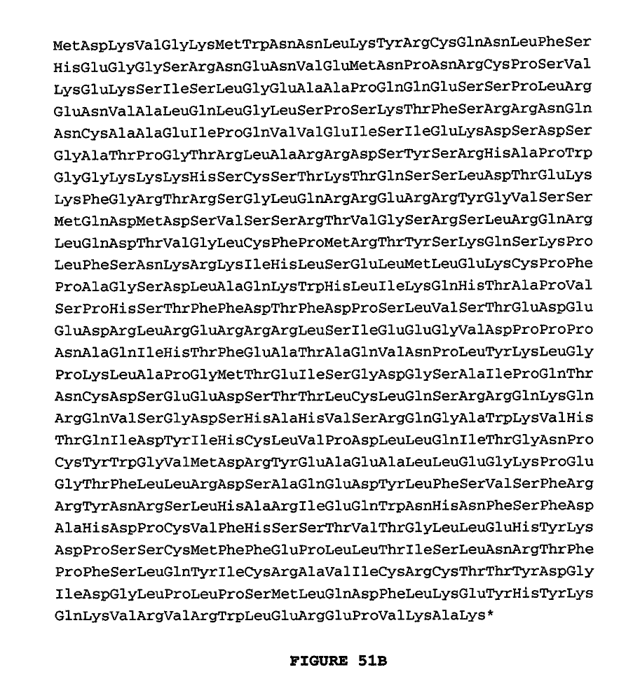

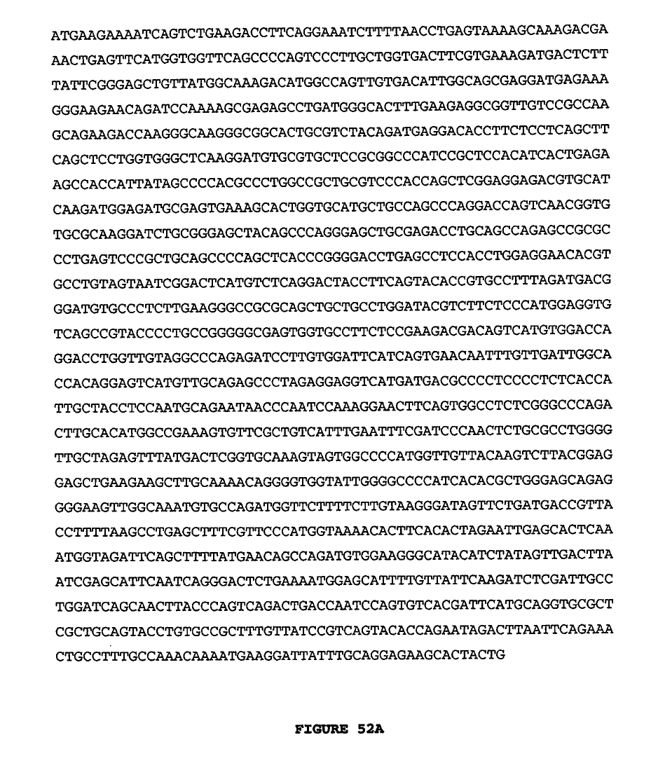

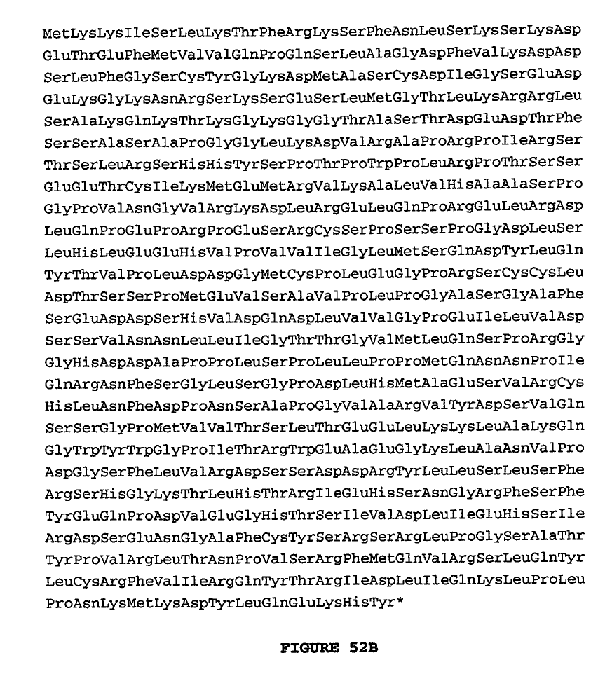

Diagrams of plasmid constructs and knockout constructs are shown in FIGS. 51-53. The genomic SOCS-1 clone 95-11-10 was digested with the restriction enzymes BamH1 and EcoR1 to obtain a 3.6 Kb DNA fragment 3′ of the coding region (SOCS-1 exon), which was used as the 3′ arm in the SOCS-1 knockout vectors. The ends of this fragment were then blunted. This fragment was then ligated into the following vectors:
pBgalpAloxNeo
and pBgalpAloxNeoTK
   which had been linearized at the unique Xho1 site and then blunted. This ligation resulted in the formation of the following vectors:
3′SOCS-1 arm in pBgalpAloxNeo
and 3′SOCS-1 arm in pBgalpAloxNeoTK

The 5′ arm of the SOCS-1 knockout vectors was constructed by using PCR to generate a 2.5 Kb PCR product from the genomic SOCS-1 clone 95-11-10 just 5′ of the SOCS-1 coding region (SOCS-1 exon). The oligo's used to generate this product were:

5′ oligo (sense) (2465)

[SEQ ID NO:49]

AGCT AGA TCT GGA CCC TAC AAT GGC AGC
3′ oligo (antisense) (2466)

[SEQ ID NO:50]

AGCT AG ATC TGC CAT CCT ACT CGA GGG GCC AGC TGG

The PCR product was then digested with the restriction enzyme BglII, to generate BglII ends to the PCR product. This 5′ SOCS-1 PCR product, with BglII, ends was then ligated as follows: 3′SOCS-1 arm in pBgalpAloxNeo and 3′SOCS-1 arm in pBgalpAloxNeoTK, which had been linearized with the unique restriction enzyme BamH1. This resulted in the following vectors being formed:
5′&3′SOCS-1 arms in pBgalpAloxNeo
and 5′&3′SOCS-1 arms in pBgalpAloxNeoTK

These were the final SOCS-1 knockout constructs. Both these constructs lacked the entire SOCS-1 coding region (SOCS-1 EXON), being replaced with portions of the Bgal, B globin polyA, PGK promoter, neomycin and PGK polyA sequences. The 5′&3′SOCS-1 arms in pBgalpAloxNeoTK vector also contained the thymidine kinase gene sequence, between the neomycin and PGK poly A sequences.

The vectors: 5′&3′SOCS-1 arms in pBgalpAloxNeo
and 5′&3′SOCS-1 arms in pBgalpAloxNeoTK were linearized with the unique restriction enzyme Not1 and then transfected into Embryonic stem cells by electroporation. Clones which were resistant to neomycin were selected and analysed by southern blot to determine if they contained the correctly integrated SOCS-1 targeting sequence. In order to determine if correct integration had occurred, genomic DNA from the neomycin resistant clones was digested with the restriction enzyme EcoR1. The digested DNA was then blotted onto nylon filters and probed with a 1.5 Kb EcoR1 /Hind III DNA fragment, which was further 5′ of the 5′arm sequence used in the knockout constructs. The band sizes expected for correct integration were:

Wild type SOCS-1 allele 5.4 Kb

SOCS-1 knockout allele 8.2 Kb in 5′&3′SOCS-1 arms in pBgalpAloxNeo or 11 Kb in 5′&3′SOCS-1 arms in pBgalpAloxNeoTK transformed cells.

### Example 39

### Analysis of SOCS-1 Deletion Mutants

SOCS-1 deletion mutants were generated by PCR to give fragments with Asc 1/Mlu 1 linkers at the N- and C-terminus and subcloned into pEF-FLAG-I (found at http://www.wehi.edu.au/willson vectors) to give N-terminal Flagged proteins. SOCS-1 deletion mutants were therefore constructed in which the N-terminal domain was deleted, retaining amino acids 77-211 (ΔN), the C-terminal domain encompassing the SOCS box was deleted, retaining amino acids 1-169 (ΔC) and both the N-terminal region and the SOCS box were deleted, retaining amino acids 77-169, leaving an intact SH2 domain (ΔN/C). In addition, a construct was created in which the SH2 domain and the SOCS box were both deleted, retaining the N-terminal 81 amino acids (ΔSH2/C). These constructs were transfected into parental M1 cells and with the exception of ΔSH2/C, several stable transfectants were obtained for each construct. Protein expression of the deletion mutants was confirmed by immunoprecipitation and Western blot analysis using anti-Flag antibodies.

These constructs were then assessed for their ability to inhibit IL-6 and LIF signalling in several different assay systems.

### 1. Colony Assays in Soft Agar

Cultures were performed as previously described (Metcalf, 1984). M1 parental cells form large compact colonies in soft agar. When cells are incubated in the presence of IL-6 or LIF the colonies are dispersed with a halo of cells migrating out from the central core. At high concentrations of cytokine, the number of colonies observed is highly diminished, a phenomenon known as clonal suppression. M1 cells which constitutively express SOCS-1 are unable to respond to either L-6 or LIF, as both macrophage differentiation and clonal suppression are inhibited (Starr et al. 1997).

To assess the ability of the SOCS-1 deletion mutants to suppress M1 cell differentiation, cells expressing the various constructs were plated in agar in the presence or absence of increasing concentrations of IL-6 or LIF. M1 cells expressing either SOCS-1 ΔN or ΔN/C protein at equivalent levels to cells expressing full-length SOCS-1 were unable to block IL-6 or LIF-induced differentiation, responding to growth factor in a similar manner to the parental M1 cells (M1-P). These results indicated that the N-terminal region of SOCS-1 was critical for inhibition of M1 macrophage differentiation.

### 2. Inhibition of LIF-Induced Luciferase Activity in 293T Cells

293T is a human fibroblast line expressing endogenous LIF receptors. Briefly, 293T cells were plated into either 24-well plates at 1×105 cells/well or 10 cm dishes at 2×106 cells/dish. The LIF responsive promoter-luciferase reporter gene (APRE-luc) and has been described previously (Masuhara et al. 1997). The positive control vector Sra-b-gal encoding the β-galactosidase gene has also been described (Ogilvy et al. 1998). Plasmids of reporter genes with either vector alone or pEF-FLAG-SOCS constructs were introduced into cells using FuGENE transfection reagent (Boehringer Mannheim) according to the Manufacturers' instructions and harvested after 48 hr. Cells were stimulated with or without 10 ng/ml hLIF overnight prior to lysis with 40 ml Reporter Lysis Buffer (Promega) containing protease inhibitors. Lysates were then assayed for luciferase and β-galactosidase activity.

293T cells were transiently transfected with the LIF-responsive reporter construct, APRE-luc and LIF induction of luciferase activity measured. A clear increase in luciferase activity is observed via JAK activation of Stat3 which dimerizes and in turn binds to the APRE (acute phase response element). The differential ability of the various SOCS proteins to modulate IL-6 and LIF signalling in M1 cells was confirmed by transient expression of the APRE-luc reporter gene with or without Flag-tagged SOCS-1, SOCS-2, SOCS-3, CIS, SOCS-5 and SOCS-6 in 293T cells. SOCS-1 and SOCS-3 completely abolished the LIF-induced activation of luciferase activity, whereas SOCS-2, CIS and SOCS-6 had no effect, and SOCS-5 partially inhibited the LIF response. Transfection efficiency was controlled for by co-expression of a b-galactosidase reporter construct under a constitutive promoter (Sra-b-gal) and luciferase activity normalized against the b-galactosidase results. Similarly, the ability of the SOCS-1 deletion mutant to inhibit M1 differentiation was paralleled by their ability to inhibit luciferase activity when transiently expressed in 293T cells. In addition, expression of SOCS1 ΔSH2/C did not inhibit LIF-induced luciferase activity, suggesting that the N-terminal region alone was insufficient to mediate SOCS-1 inhibition of LIF signalling.

### 3. Inhibition of JAK2 Autophosphorylation in an In Vitro Kinase Assay

To further investigate the function of the SOCS-1 deletion mutants, the inventors examined the ability of the different SOCS proteins to directly inhibit JAK kinase activity. The methods used are outlined below.

Briefly, cell lysates were prepared as previously described (Nicholson et al. 1995). Proteins were immunoprecipitated with either anti-JAK2 antibodies (UBI) or anti-Flag antibody conjugated to Sepharose (M2; Eastman Kodak Company) and proteins separated on 4-15% w/v gradient SDS-PAGE gels. Protein was then electrophoretically transferred to PVDF membranes. Membranes were blocked overnight in 10% w/v skim milk and incubated with primary antibody for 2 hr. Antibody binding was visualized with either peroxidase-conjugated anti-rabbit Ig (Silenus) or peroxidase conjugated anti-mouse IgFc, which specifically recognises the immunoglobulin heavy chain (Jackson Laboratories) and the enhanced chemiluminescence (ECL) system (Amersham). Anti-JAK2 immunoprecipitates were washed and incubated with g-ATP as previously described (Nicholson et al. 1995).

Flag-tagged-JAK2 was transiently expressed in Cos cells with or without the various SOCS-1 deletion mutants. Cells were lysed and JAK2 proteins immunoprecipitated, incubated in kinase buffer containing radiolabelled γ-ATP, and the proteins separated by SDS-PAGE gel (as described above). Incorporation of radiolabelled phosphate into the JAK2 protein (autophosphorylation) was then visualised using a phosphorimager. Co-expression of full length SOCS-1 dramatically inhibited JAK2 autophosphorylation. Three of the SOCS-1 deletion mutants were tested (ΔC, ΔN/C, ΔN) for their ability to functionally inhibit the JAK2 kinase activity. Co-expression of each of these constructs with JAK2 indicated that they were also able to inhibit JAK2 kinase activity to the same degree as full-length SOCS-1. Immunoprecipitation of equal amounts of JAK2 protein was demonstrated by Western blot with anti-Flag antibodies. Expression levels of the various SOCS-1 deletion mutants was determined by immunoprecipitation and Western blot with anti-Flag antibodies. These results indicate that at least in an over-expression system where JAK2 is constitutively active (ie. not a ligand-inducible system), the SH2 domain of SOCS-1 is sufficient to inhibit JAK kinase assay.

JAK2 was transiently expressed in Cos cells with or without the various SOCS-1 deletion mutants. Cells were lysed and JAK2 tyrosine phosphorylation assessed by Western blot with anti-phosphotyrosine antibodies. Inhibition of JAK2 tyrosine phosphorylation correlated with the ability of the various SOCS-1 deletion mutants to inhibit JAK2 kinase activity. In addition, all three of the SOCS-1 mutants tested (ΔC, ΔN/C, ΔN) appeared able to associate with JAK2.

The data obtained with the SOCS-1 deletion mutants indicate that the N-terminal region of SOCS-1 was required for its ability to inhibit LIF and IL-6 signalling, particularly with respect to induction of M1 cell differentiation. In addition, the in vitro kinase data indicates that inhibition of JAK kinase activity is mediated through the SOCS-1 SH2 domain.

### Example 40

### SOCS Chimaeras

In order to further investigate the importance of the different SOCS domains, a series of chimeric proteins were created in which the N-terminal domain of SOCS-1 was replaced with the either the N-terminal domain of SOCS-2, SOCS-3, SOCS-4, SOCS-5 or SOCS-6. A series of chimeric proteins were also created in which the SH2 domain of SOCS-1 was replaced with either the SH2 domain of SOCS-2, SOCS-3, CIS, SOCS-5 or SOCS-6.

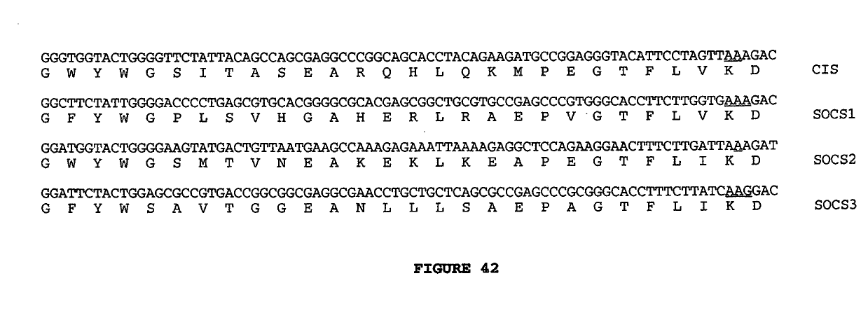

To facilitate the synthesis of chimeric mouse SOCS1 cDNAs an Xho I site was introduced at the boundary between the N-terminal region and SH2 domain of the SOCS1 cDNA (see FIG. 42). Two nucleotide changes were introduced using a PCR-based technique known as splicing by overlap extension (Horton et al. 1989). The PCR fragment, designated mSOCS1 mutXho, was then cloned into the Kpn I and Sac I sites of pBLUESCRIPT SK II(+) [Stratagene]. In order to facilitate cloning of the DNA fragments into the mammalian expression vector pEF-FLAG-I (found at http://www.wehi.edu.au./willson vectors), an in-frame Asc I restriction enzyme site was introduced one amino acid after the predicted translational start site and an Mlu I site was inserted immediately before the stop codon of the mouse SOCS1 cDNA. Since the C>G nucleotide alteration leads to a Δ>E amino acid substitution at position 76, the SOCS1 mutXho cDNA was cloned into the Mlu I site of pEF-FLAG-I and shown to have similar activity to wild-type SOCS-1 in the luciferase assay (FIG. 42).

Hybrid cDNAs, in which the N-terminal region or the SH2 domain of the SOCS1 sequence were replaced with homologous regions of mouse CIS, SOCS2, SOCS3, SOCS5 or SOCS6, were synthesized from PCR generated restriction fragments (N-terminal regions were cloned in as Asc I-Xho I fragments and SH2 domain fragments were clone in as Xho I-Not I fragments). All the hybrid cDNAs were then cloned into the Mlu I site of pEF-FLAG-I in order to express mouse SOCS1 domain swap mutant proteins with an N-terminal FLAG epitope tag. Constructs were sequenced in their entirety before use. The exact specification of sequences present in each SOCS1 domain swap mutant is listed in Table 16.

TABLE 16
CONSTRUCTION OF MOUSE SOCS1
DOMAIN SWAP MUTANT PROTEINS

| Hybrid | Amino Acid Sequence Specifications |
|---|---|
| SOCS1-CNT | CIS(2-80):SOCS1(75-212) |
| SOCS1-2NT | SOCS2(2-46):SOCS1(75-212) |
| SOCS1-3NT | SOCS3(2-44):SOCS1(75-212) |
| SOCS1-5NT | SOCS5(2-379):SOCS1(75-212) |
| SOCS1-6(9)NT | SOCS6(2-380):SOCS1(75-212) |
| SOCS1-CSH2 | SOCS1(2-78):CIS(81-218):SOCS1(172-212) |
| SOCS1-2SH2 | SOCS1(2-78):SOCS2(47-159):SOCS1(172-212) |
| SOCS1-3SH2 | SOCS1(2-78):SOCS3(45-185):SOCS1(172-212) |
| SOCS1-5SH2 | SOCS1(2-78):SOCS5(380-480):SOCS1(172-212) |
| SOCS1-6(9)SH2 | SOCS1(2-78):SOCS6(381-494):SOCS1(172-212) |

Amino acid sequences are designated according to the following example. CIS(2-80):SOCS1 (75-212) (hybrid SOCS1-CNT) denotes that amino acid residues 2 to 80 are derived from N-terminal region of the mCIS sequence and amino acid residues 75 to 212 are derived from mouse SOCS1 sequence.

These chimeric constructs were Flag-tagged, transiently expressed in 293T cells and LIF-induction of luciferase activity assayed. In contrast to wild-type SOCS-1, none of the chimeric proteins were able to inhibit LIF-induction of luciferase activity. Therefore, the N-terminal region of SOCS-1 cannot be functionally replaced by any of the SOCS-2, SOCS-3, CIS, SOCS-4, SOCS-5 and SOCS-6N-terminal domains. Likewise, Although none of the introduced SH2 domains is able to fully replace the SOCS-1-SH2, partial inhibition of LIF-induced luciferase activity was observed with chimeric SOCs proteins, SOCS-1-3SH2, SOCS-1CSH2, and SOCS-1-5SH2.

The data in both M1 cells and 293T cells indicated that whilst the N-terminal region was critical for SOCS-1 function, the SH2 domains of several SOCS proteins was sufficient for some, though not normal, level of activity.

Previous work has shown that mutation of a conserved arginine residue to lysine within SH2 domains results in a non-functional domain. Mouse SOCS1, SOCS2, SOCS3 and CIS cDNAs, in which an R>K amino acid substitution in the SH2 domain was introduced (see FIG. 42), were generated using the PCR-based technique, splicing by overlap extension. To facilitate cloning of the PCR-generated fragments into pEF-FLAG-I, an in-frame Asc I restriction enzyme site was introduced immediately after the predicted translation start site and an Mlu I site was inserted immediately before the translation stop site. Expression construct pEF-FLAG-I/mSOCS1-R105K encodes a mSOCS1 protein with an R>K amino acid substitution at position 105 in the SH2 domain. pEF-FLAG-I/mSOCS2-R73K, pEF-FLAG-I/mSOCS3-R71K, pEF-FLAG-I/mCIS-R107K encode mSOCS2, SOCS3 and CIS proteins with R>K amino acid substitutions at the equivalent position in the SH2 domain, respectively.

To further confirm that the SH2 domain of SOCS-1 was required for activity, point mutations were made in each of the SOCS protein SH2 domains (SOCS-1 to SOCS-3, CIS; FIG. 42) changing the conserved arginine residue to a lysine. These constructs were then transiently expressed in the 293T reporter gene system. SOCS-1 containing a non-functional SH2 domain (SOCS-1-R105K) was unable to inhibit LIF-induced luciferase activity, providing further evidence that the SH2 domain has a critical role in SOCS-1 function. Further, a mutation of the SOCS-3-SH2 domain (SOCS-3-R71K) did not abrogate the ability of SOCS-1 to inhibit LIF signalling. This not only confirms a critical role for the SOCS-1-SH2 domain in LIF signalling, but is the first evidence to suggest that although both SOCS-1 and SOCS-3 are able to inhibit LIF and IL-6 signal transduction, they may do so through entirely different mechanisms.

### Example 41

### Biochemical Analysis of SOCs Action

The inventors sought further evidence as to the molecular site of action of the various SOCS proteins.

M1 parental cells (M1-P) and M1 cells constitutively expressing a Flag-tagged SOCS-1 protein were serum-starved for 2 hrs and stimulated for 0, 5, 10 minutes with 104 U/ml mLIF. Cells were lysed and JAK proteins immunoprecipitated with 5 ml anti-JAK1 antibodies (UBI) and protein-A-Sepharose. Precipitates were washed and proteins separated by SDS-PAGE on a 4-15% w/v gradient gel, prior to analysis by Western blot with anti-phosphotyrosine antibodies. In parental M1 cells JAK1 was clearly phosphorylated in response to LIF. In contrast, constitutive expression of SOCS-1 inhibited JAK1 tyrosine phosphorylation.

High level expression of JAK2 protein in Cos cells results in a constitutively active JAK protein, presumably due to dimerisation and cross-phosphorylation. Flag-tagged JAK2 was, therefore, transiently expressed by electroporation in Cos cells with and without co-expression of Flag-tagged SOCS-1. After 48 hours cells were lysed on ice and JAK2 proteins immunoprecipitated using 5 ml anti-JAK2 antibody (UBI). Immunoprecipitates were washed, divided in two and half the proteins subjected to an in vitro kinase assay as previously described. Proteins were then separated by SDS-PAGE on a 4-15% w/v gradient gel and the gel treated with KOH to remove phosphoserine and phosphothreonine. Incorporation of radiolabelled phosphate was detected using a phosphorimager. The remaining half of the immunoprecipitation was run on a 4-15% w/v gradient gel and analysed by Western blot with anti-JAK2 antibodies (UBI) to demonstrate equal immunoprecipitation.

Lysates from Cos cells expressing either JAK2 or both JAK2 and SOCS-1, were run on a 4-15% w/v gel, electrophoretically transferred to PVDF and analysed with anti-phosphotyrosine antibodies at a 1:5000 dilution (4G10; UBI). A single phosphorylated band corresponding to JAK2 was observed in parental M1 cells, which was not evident in cells expressing SOCS-1. Lysates were re-probed with anti-Flag antibodies to demonstrate equal loading of JAK2 protein. Expression of SOCS-1 was therefore able to inhibit both JAK2 kinase activity or autophosphorylation and tyrosine phosphorylation.

The ability of SOCS family members, SOCS-2, SOCS-3 and CIS to inhibit JAK2 kinase activity was further investigated. Flag-tagged JAK2 was transiently expressed by electroporation, with or without, Flag-tagged-SOCS-1, SOCS-2, SOCS-3 and CIS in Cos cells. After 48 hours cells were lysed on ice and JAK2 proteins immunoprecipitated using 5 ml anti-JAK2 antibody (UBI). Immunoprecipitates were washed and subjected to an in vitro kinase assay as previously described. Proteins were then separated by SDS-PAGE on a 4-15% w/v gradient gel and the gel treated with KOH to remove phosphoserine and phosphothreonine. Incorporation of radiolabelled phosphate was detected using a phosphorimager (FIG. 62b). As had been demonstrated previously, co-expression of SOCS-1 inhibited JAK2 kinase activity. Co-expression of SOCS-2 and CIS also appeared to inhibit JAK2 kinase activity, whilst co-expression of SOCS-3 did not inhibit kinase activity. These results suggests that SOCS-1 and SOCS-3 may have a differential ability to inhibit JAK2 kinase activity.

M1 cell lines stably expressing Flag-tagged SOCS-1, SOCS-2, SOCS-3 or CIS proteins, were lysed and the SOCS proteins immunoprecipitated using anti-Flag antibodies conjugated to sepharose. Immunoprecipitates were washed and the proteins were separated by SDS-PAGE on a 4-15% w/v gradient gel. Proteins were then electrophoretically transferred to PVDF membrane and analysed using anti-Flag antibodies. Expression levels of SOCS-2 (S2) were considerably higher than the other SOCS proteins with the expression levels of SOCS-1 (S1) being the lowest.

Stat3 tyrosine phosphorylation has previously been implicated in IL-6-induced differentiation, both by the use of dominant negative Stat3 constructs and by specific tyrosine mutations within the IL-6 signalling chain, gp130, which block recruitment of Stat3 to the receptor complex. The inventors examined, therefore, LIF-induced Stat3 tyrosine phosphorylation in M1 cells expressing the various SOCS-1, SOCS-2, SOCS-3 and CIS. M1 cell lines stably expressing the various SOCS proteins, were serum-starved for 1.5 hours prior to stimulation with 104 U/ml mLIF for 0, 5 and 10 minutes. Cells were then lysed, Stat3 proteins immunoprecipitated and analyzed by Western blot with antibodies specific to phosphorylated Stat3 (BioLabs). Stat3 was rapidly tyrosine phosphorylated in parental M1 cells in response to LIF. In contrast, Stat3 tyrosine phosphorylation was inhibited in M1 cells expressing SOCS-1 and SOCS-3. In each instance, Stat3 tyrosine phosphorylation correlated inversely with the ability of the expressed SOCS protein to inhibit M1 cell differentiation. This indicates that Stat3 has a critical role in IL-6-induced M1 cell differentiation and further suggests that the ability of SOCS-1 and SOCS-3 to inhibit M1 differentiation may be mediated through inhibition of the JAK-STAT pathway. Western blots were stripped and re-probed with anti-Stat3 antibodies to demonstrate equal loading of Stat3 protein.

### Example 42

### Expression of SOCS-2, SOCS-3 AND CIS in M1 Cells

cDNAs encoding epitope-tagged SOCS-2, SOCS-3 or CIS were generated by subcloning the entire coding region of each gene into the pEF-BOS expression vector, engineered to encode an in-frame FLAG epitope downstream of an initiation methionine. Using electroporation, M1 cells were transfected with 20 mg of linearised expression plasmid and 2 mg of a linearised plasmid in which transcription of a cDNA encoding puromycin N-acetyl transferase was driven from the mouse phosphoglycerokinase promoter. After 48 hours in culture, transfected cells were selected with 20 μg/ml puromycin. Puromycin-resistant cells were screened for the expression of SOCS-2, SOCS-3 or CIS by immunoprecipitation and Western blotting of cell extractors with the M2 anti-FLAG monoclonal antibody.

In order to assay the differentiation of M1 cells in response to cytokines, 300 cells were cultured in 35 mm Petri dishes containing 1 ml of DME supplemented with 20% v/v foetal calf serum (FCS), 0.3% w/v agar and 0.1 ml of serial dilutions of interleukin 6 (IL-6). After 7 days culture at 37° C. in a fully humidified atmosphere, containing 10% v/v CO₂ in air, colonies of M1 cells were counted and classified as differentiated if they were composed of dispersed cells or had a corona of dispersed cells around a tightly packed centre. The total number of colonies in each dish were counted to determine the degree of clonal suppression induced by IL-6.

M1 cells expressing SOCS-2 were slightly hyporesponsive to differentiation induced by IL-6. However, IL-6 was unable to induce clonal suppression in these cells. The level of SOCS-2 expression in M1 cells was 10-fold higher than that for the other SOCS proteins. SOCS-3 expression in M1 cells completely inhibited the ability of IL-6 to induce either clonal suppression or differentiation in agar in response to IL-6, similar to the action of SOCS-1. M1 cells expressing CIS responded to IL-6 in a similar manner to parental M1 cells.

### Example 43

### Knockout of SOCs Genes

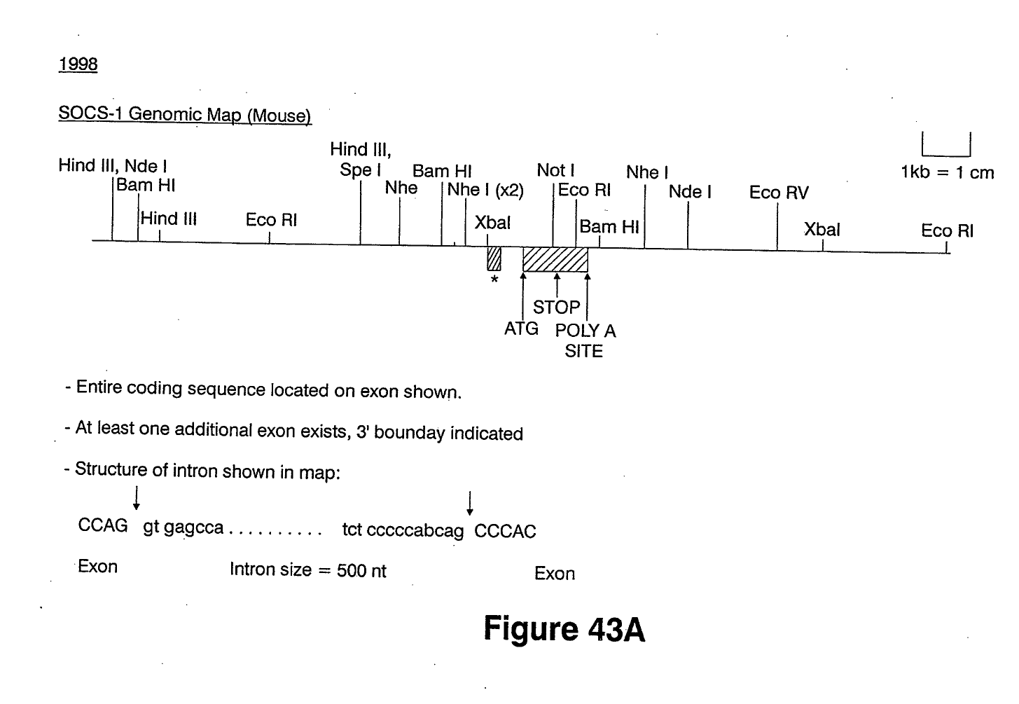

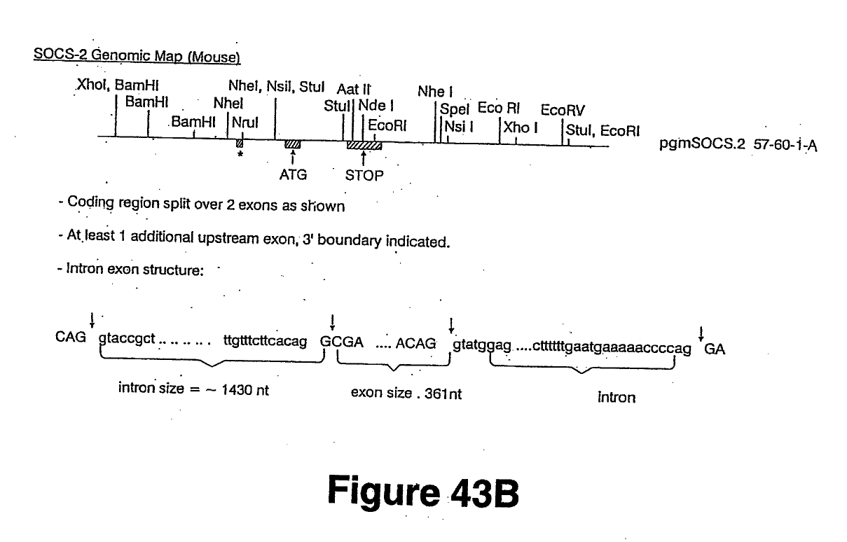

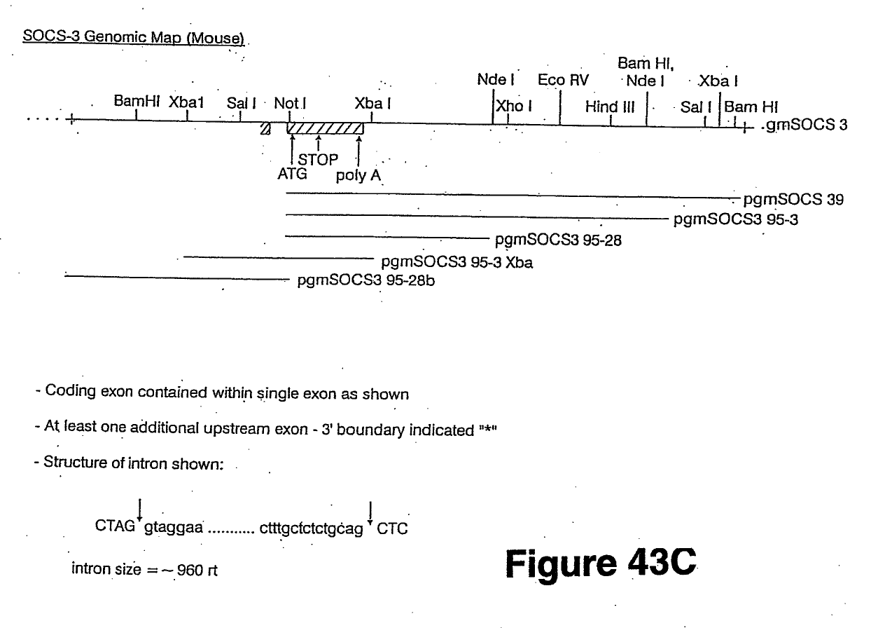

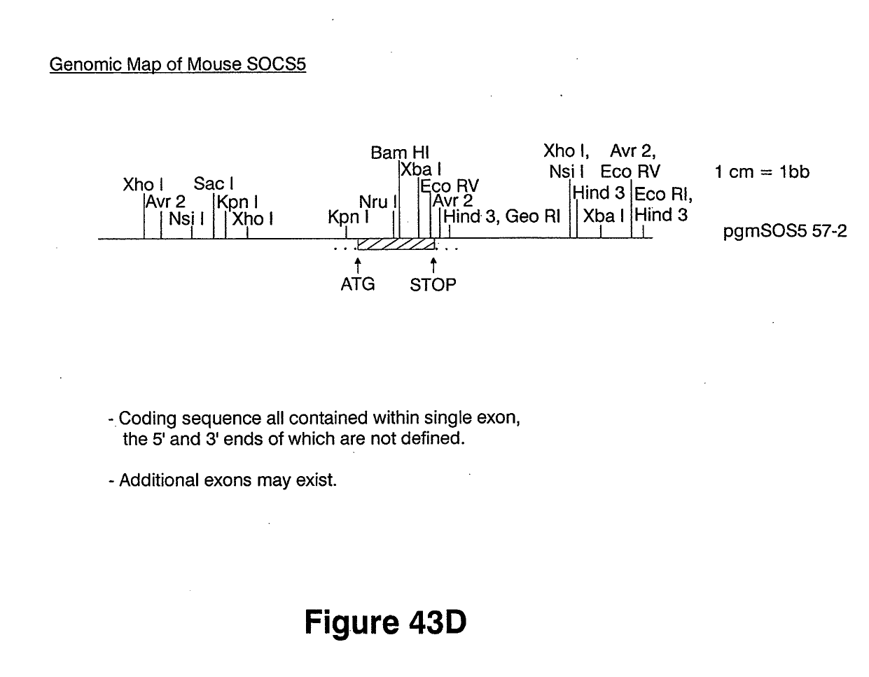

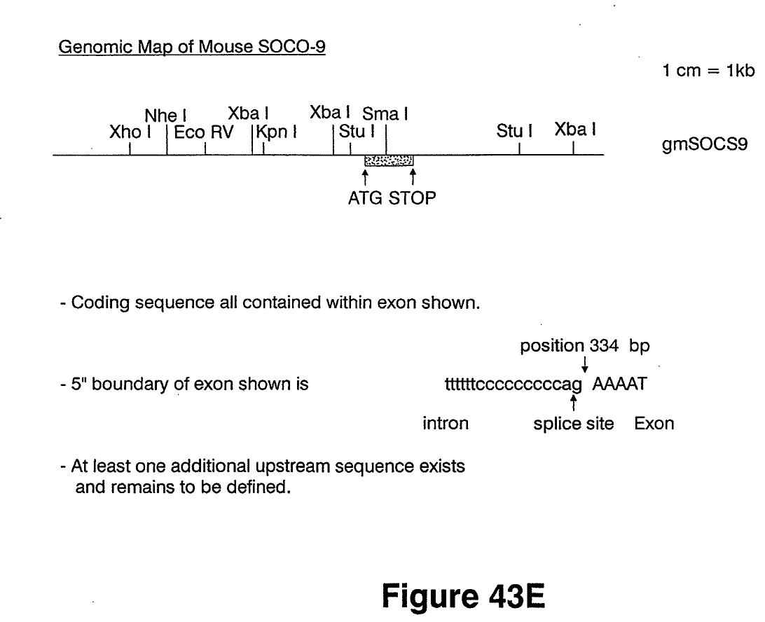

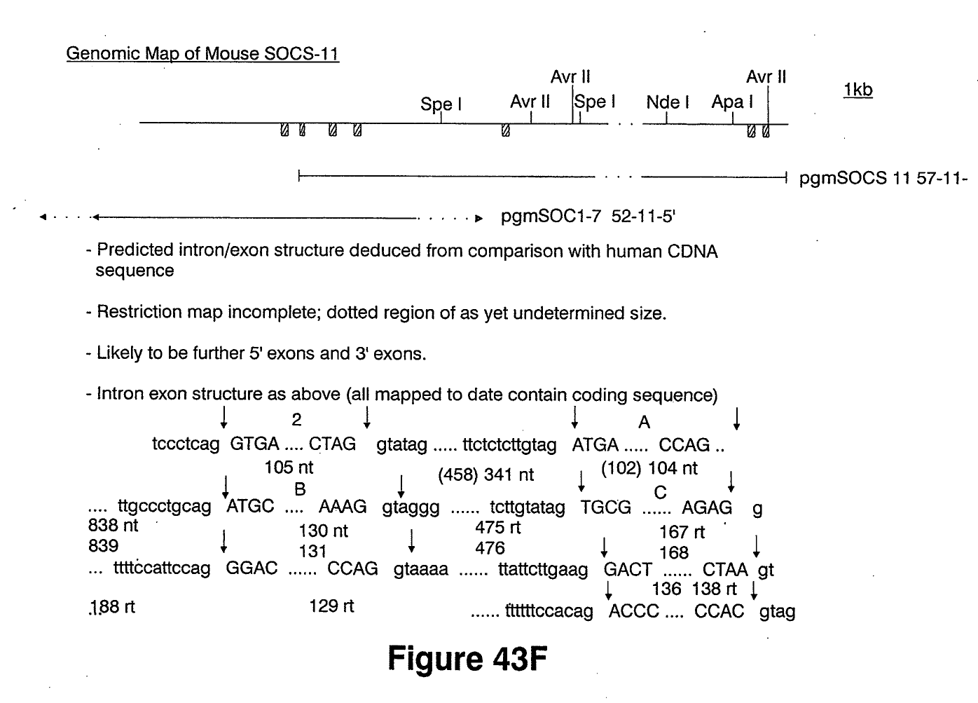

In vitro studies have clearly identified the SOCS protein as key negative regulators of signal transduction. Moreover, injection of cytokines into mice has been shown to result in increased transcription of SOCS genes, implicating these proteins in regulation of cytokine responses in vivo. In order to determine the physiological processes regulated by each SOCS gene the inventors carry out experiments to “knockout” individual SOCS genes in mice. The first step in doing this is to clone genomic DNA encoding each of the SOCS genes. The maps of the genes for mouse SOCS-1, SOCS-2, SOCS-3, SOCS-5, SOCS-9 and SOCS-11 are shown in FIG. 43 A-F).

### Generation and Analysis of SOCs-1 Knockout Mice

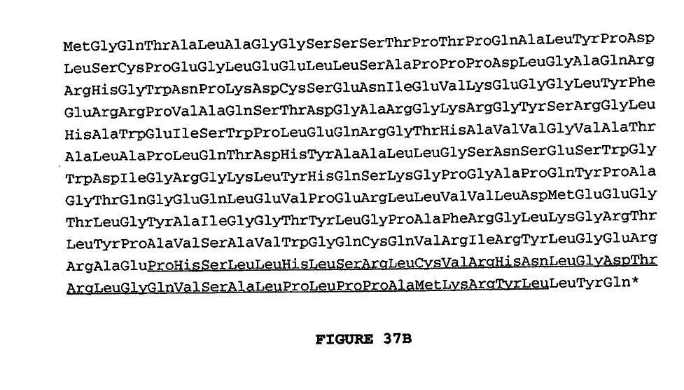

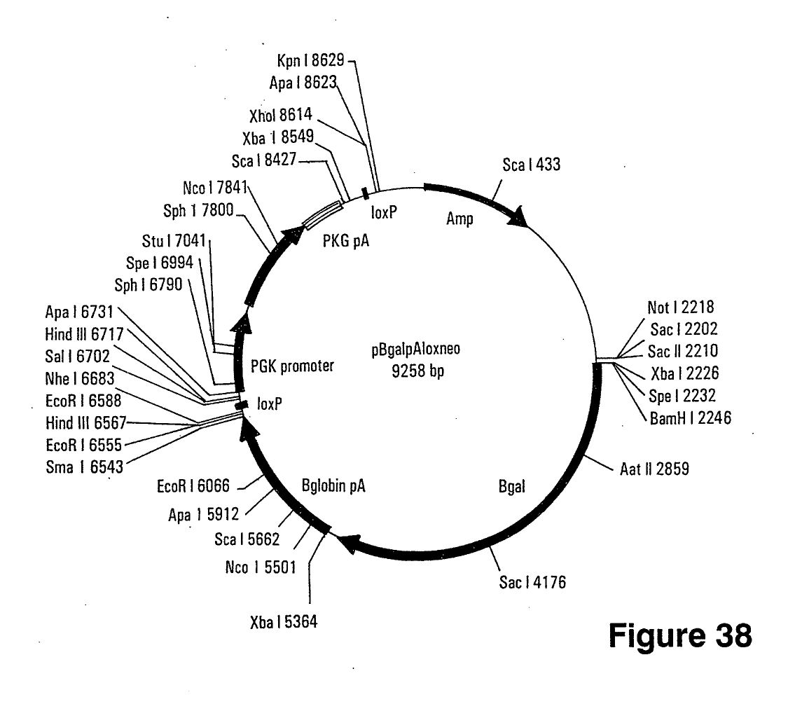

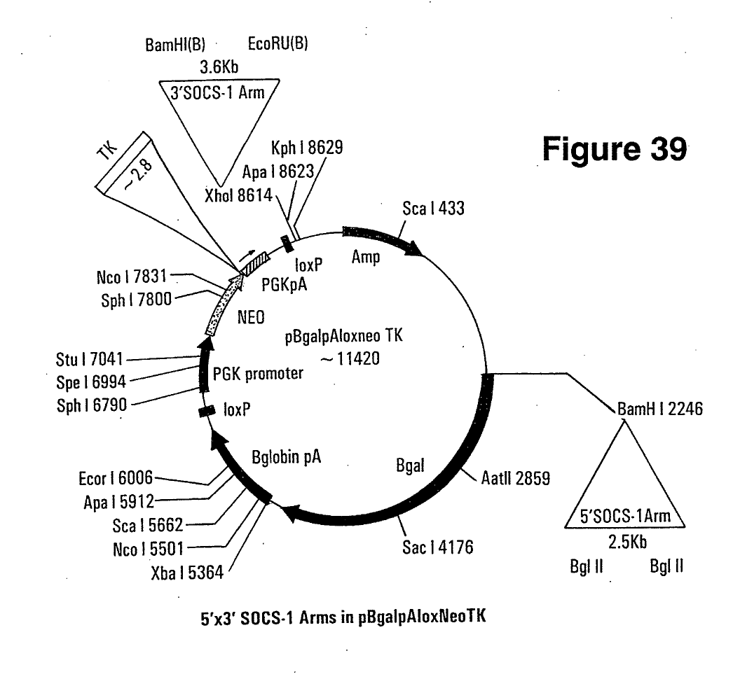

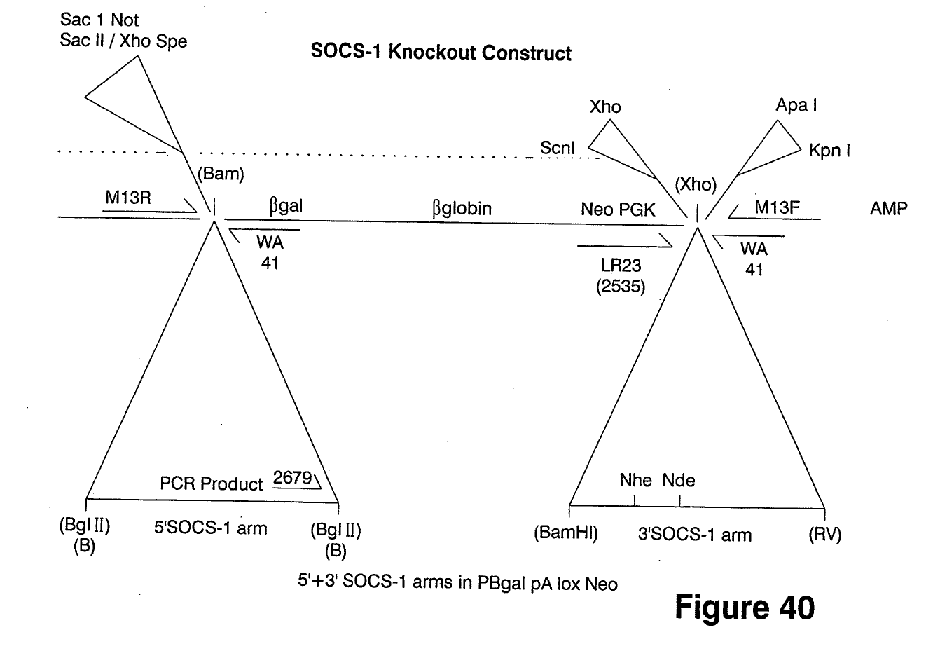

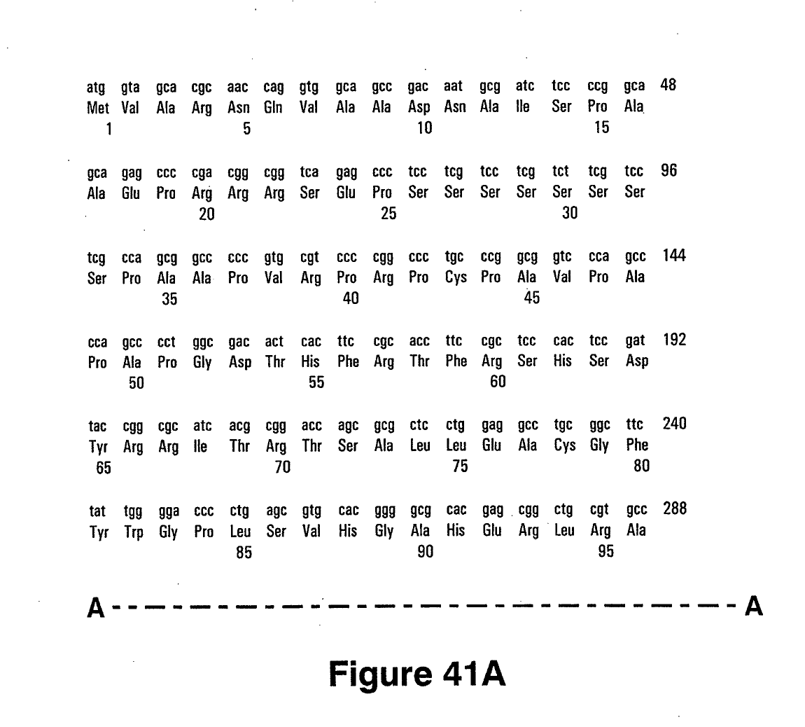

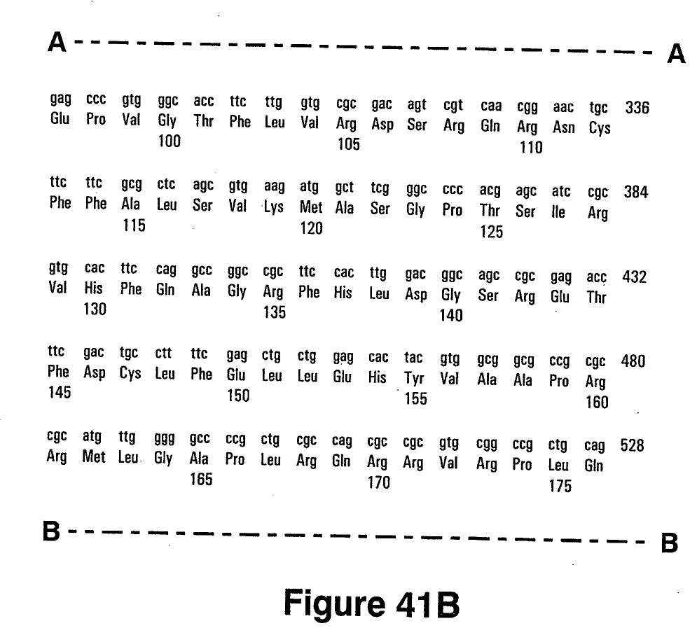

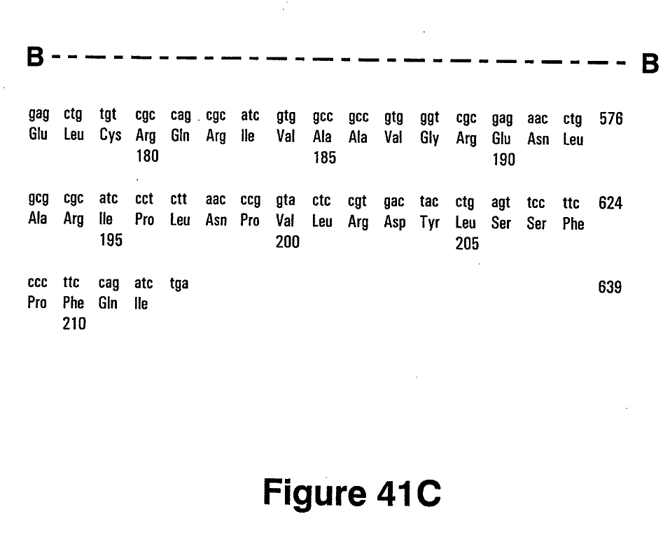

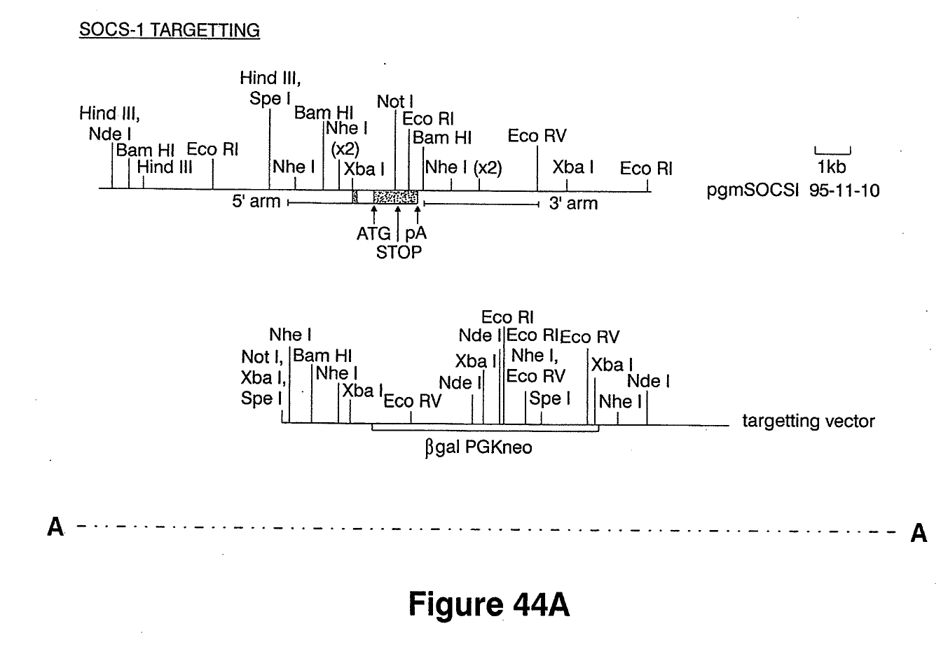

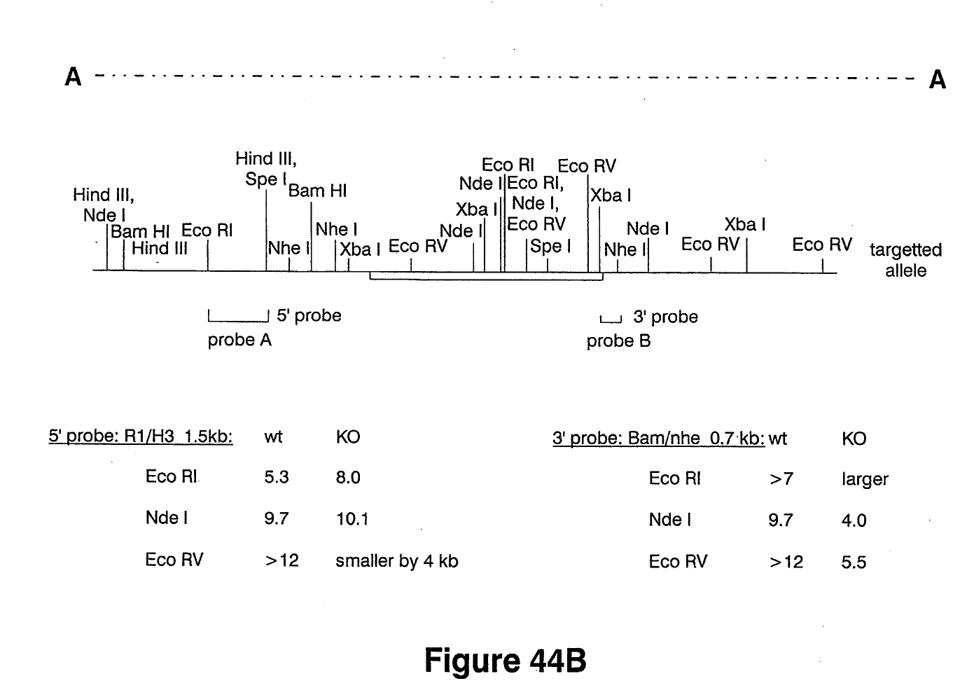

To construct the SOCS1 targeting vector, a 5′ arm extending approximately 2.5 kb from the protein initiation ATG was generated by PCR using specific SOCS1 oligonucleotides and genomic clone pgmSOCS1 95-11-10 as template. This fragment was fused to the ATG of β-galactosidase via the BamHI site in the plasmid vector pβgalpAloxneo (FIG. 38). The 3′ arm, a 3.2 kb BamHI-EcoRV fragment from pgmSOCS1 95-11-10 (FIG. 44) was blunted and ligated into the XhoI (blunted) site of pβgalpAloxneo that already contained the 5′ arm. This targeting vector was linearised with NotI and electroporated into W9.5 embryonic stem cells. After 48 hours, transfected cells were selected in 175 μg/ml G418 and resistant clones picked and expanded after a further 8 days. Clones in which the targeting vector had recombined with the endogenous SOCS1 gene were identified by hybridising EcoRI-digested genomic DNA with 1.5 kb EcoRI-HindIII fragment from pgmSOCS1 95-11-10. This probe (probe A, FIG. 44), which is located 5′ to the SOCS1 sequences in the targeting vector, distinguished between the endogenous (5.3 kb) and targeted (8.0 kb) SOCS1 loci (FIG. 44). The appropriate homologous recombination event was confirmed in ES clones with probe B, a 0.7 kb BamHI-NheI fragment from pgmSOCS1 95-11-10 (FIG. 44) situated 3′ to the SOCS1 gene. Genomic DNA was digested with EcoRI for 16 hrs at 37° C., electrophoresed through 0.8% w/v agarose, transferred to nylon membranes and hybridised to ³²P-labelled probe in a solution containing 0.5M sodium phosphate, 7% w/v SDS, 1 mM EDTA and washed in a solution containing 40 mM sodium phosphate, 1% w/v SDS at 65° C. Hybridising bands were visualised by autoradiography for 16 hours at −70° C. using Kodak XAR-5 film and intensifying screens. A targeted ES cell clones, W9.5SOCS1 1.4-A8 was injected into C57B1/6 blastocysts to generate chimeric mice. Male chimeras were mated with C57B1/6 females to yield SOCS1 heterozygotes which are currently being interbred to produce wild-type (SOCS-1+/+), heterozygous (SOCS+/−) and mutant (SOCS −/−) mice. The genotypes of offspring were determined by Southern Blot analysis of genomic DNA extracted from tail biopsies.

Mice lacking expression of SOCS-1 have been generated by replacing the entire coding region of SOCS-1 with bgal. In both heterozygous mice (SOCS-1+/−) and homozygous knockout mice (SOCS-1−/−), bgal expression should accurately reflect the normal tissue expression of SOCS-1. This experiment was a trial to determine whether βgal expression could be detected in SOCS-1+/− mice. The thymus was specifically chosen for analysis as Northern blots have previously shown high levels of constitutive SOCS-1 expression in this tissue. The procedure for FACSgal involves loading cells with a bgal substrate, fluorescein di-β-D-galactopyranoside (FDG), and allowing time for the bgal to convert this substrate to fluorescein, which can be detected, by FACS analysis.

The steps in this procedure are:

1) Tissues (thymus, spleen femur harvested from:
a) SOCS-1+/− mice
b) SOCS-1+/+ littermates (negative control)
c) ROSA mice (positive control)
   2) Single cell suspension of each tissue obtained by flushing through KDS buffer containing 5% v/v FCS.
   3) RBC lysis.
   4) Cell pellet resuspended in 150 ml KDS/5% v/v FCS.
   5) Hypotonic loading: Warmed cells diluted 1:1 with 2 mM FDG. Incubated at 37° C. for 120 secs.
   6) Cells incubated on ice for ˜3 hr to allow hydrolysis of FDG to fluorescein. 1 mg/ml propidium iodide added to cells prior to FACSCAN analysis.

From this analysis we have shown bgal expression is high (92% of cells) in the thymus of SOCS-1+/− mice, as expected. A smaller percentage of cells from spleen (12%) and bone marrow (22%) were also expressing βgal. The high level expression of SOCS-1 in the thymus accurately reflects the expression of SOCS-1 observed by Northern analysis showing that bgal expression in SOCS-1+/− mice can be used as a marker for SOCS-1 expression. Moreover, since β-galactosidase expression, and indeed any other marker in which is inserted into the SOCS-1 locus, like green fluorescent protein, will be transcribed in response to cytokines these mice are extremely useful reagents for monitoring responses to cytokines in vivo, in addition to being bred to yield mice which lack SOCS-1.

### Generation of SOCS2 Knockout Mice:

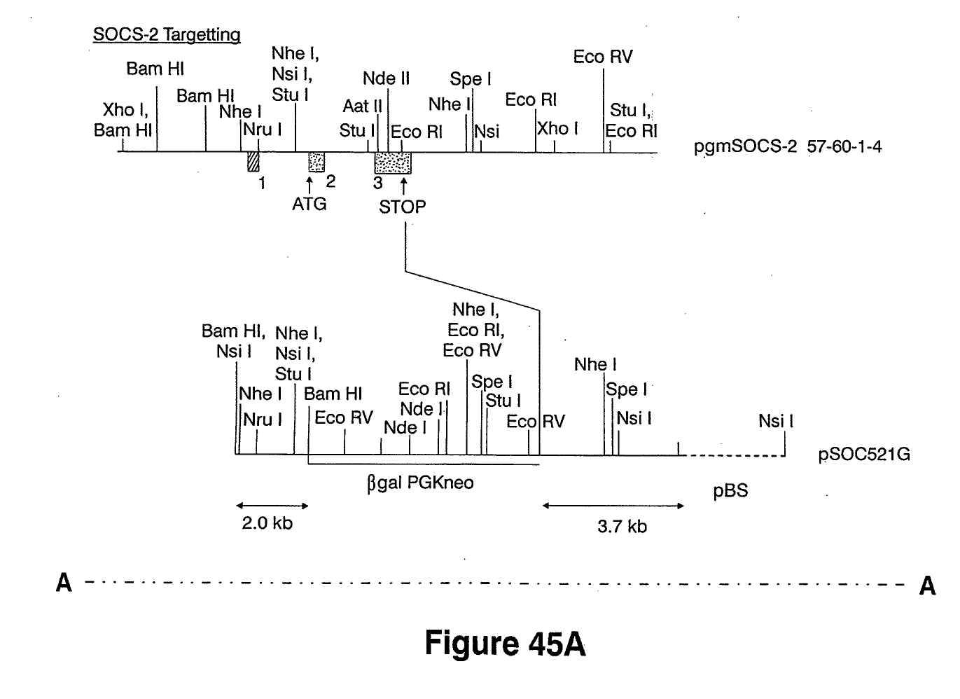

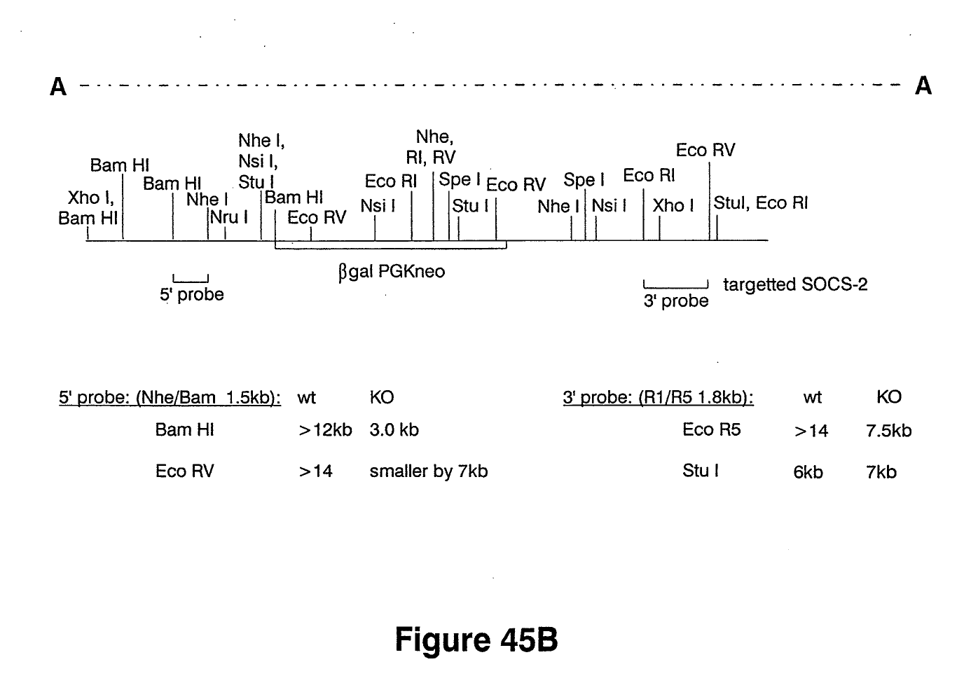

To construct the SOCS2 targeting vector, a 5′ arm extending approximately 2.0 kb from the protein initiation ATG was generated by PCR using specific SOCS2 oligonucleotides and genomic clone pgmSOCS2 57-60-1-45 as template. This fragment was fused to the ATG of β-galactosidase via the BamHI site in the plasmid vector pβgalpAloxneo (FIG. 38). The 3′ arm, a 3.7 kb EcoRI fragment from pgmSOCS2 57-60-1-45 (FIG. 45) was blunted and ligated into the XhoI (blunted) site of pβgalpAloxneo that already contained the 5′ arm. This targeting vector was linearised with NotI and electroporated into BRUCE 4 embryonic stem cells. Transfected cells were selected in G418 and resistant clones picked and expanded. Clones in which the targeting vector had recombined with the endogenous SOCS2 gene were identified by hybridising EcoRV-digested genomic DNA with a 1.8 kb EcoRI-EcoRV fragment from pgmSOCS2 57-60-1-45. This probe (probe A, FIG. 45), which is located 3′ to the SOCS2 sequences in the targeting vector, distinguished between the endogenous (greater than 14 kb) and targeted (7.5 kb) SOCS2 loci (FIG. 45). Several targeted ES cell clones have been identified and are currently being injected into blastocysts to generate chimeric mice.

### Generation of SOCS3 Knockout Mice:

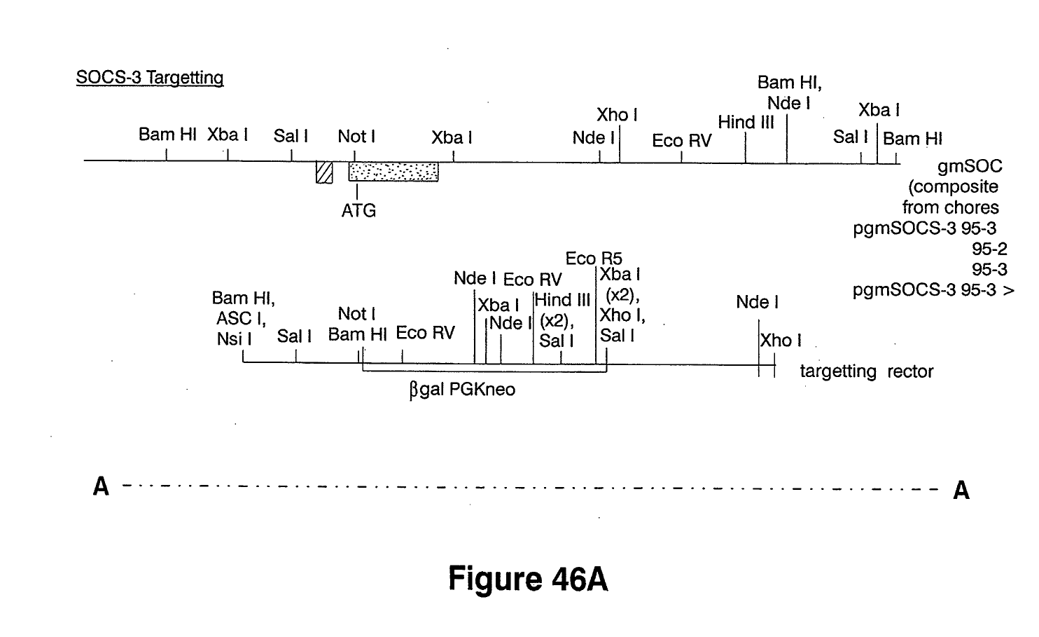

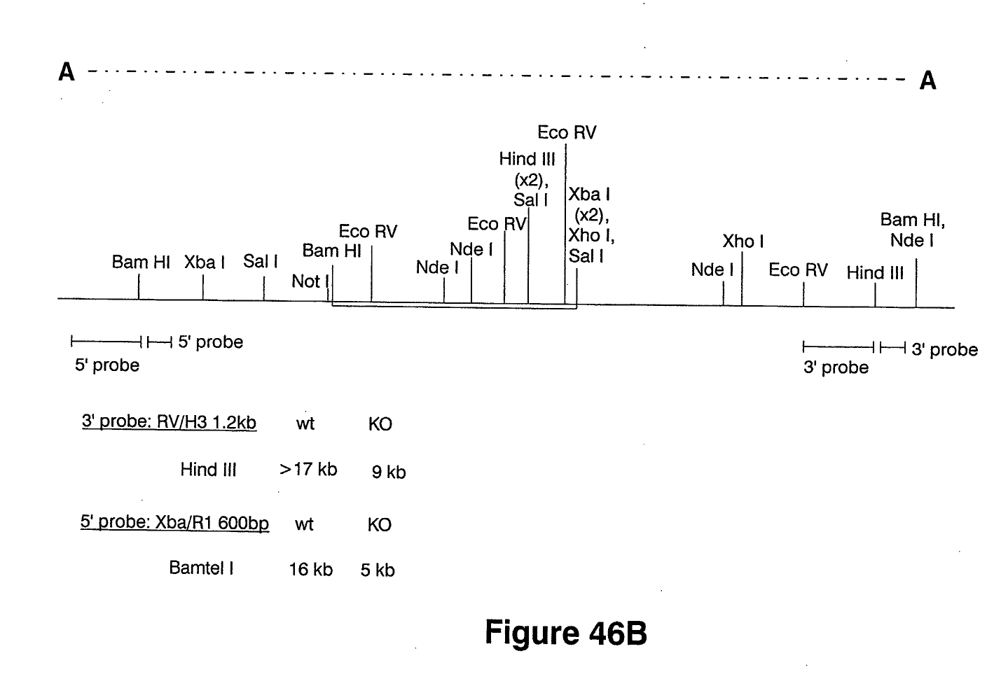

To construct the SOCS3 targeting vector, a 5′ arm extending approximately 3.0 kb from the protein initiation ATG was generated by PCR using specific SOCS3 oligonucleotides and genomic clone pgmSOCS3 95-3 Xha as template. This fragment was fused to the ATG of β-galactosidase via the BamHI site in the plasmid vector pβgalpAloxneo. The 3′ arm is a 4.2 kb XbaI-XhoI fragment from pgmSOCS3 95-3 (FIG. 46). Initially, a 7.4 kb XbaI-HindIII fragment from this genomic clone was ligated into pBluescript, from which the 3′ arm was excised as a XhoI fragment and ligated into the XhoI site of pβgalpAloxneo that already contained the 5′ arm. This targeting vector was linearised with AscI and is currently being electroporated into BRUCE 4 embryonic stem cells.

### Generation of CIS Knockout Mice:

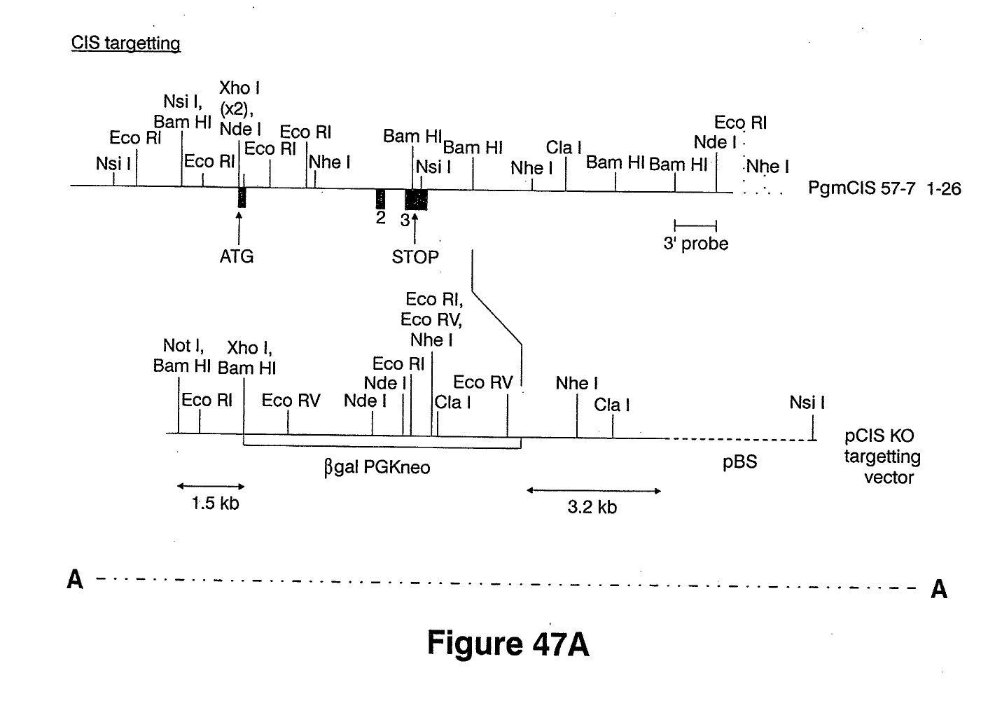

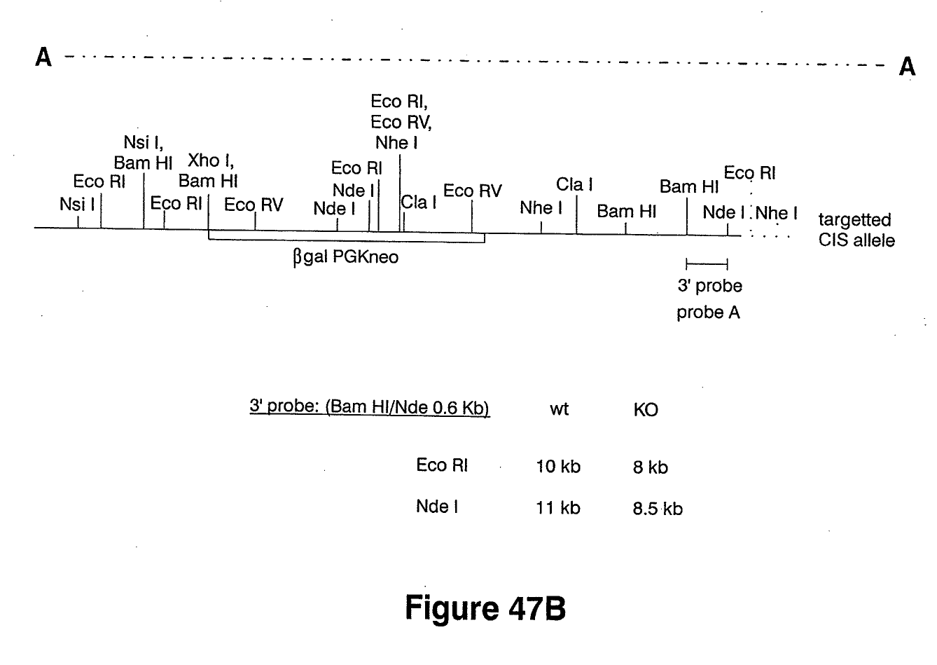

To construct the CIS targeting vector, a 5′ arm extending approximately 1.5 kb from the protein initiation ATG was generated by PCR using specific CIS oligonucleotides and genomic clone pgmCIS 57-7-1-26 as template. This fragment was fused to the ATG of β-galactosidase via the BamHI site in the plasmid vector pβgalpAloxneo (see FIG. 38) into which the 3′ arm, a 3.2 kb BamHI fragment from pgmCIS 57-7-1-26 had already been inserted. The 3′ arm fragment was blunted and ligated into the XhoI (blunted) site of pβgalpAloxneo. The final targeting vector was linearised with NotI and electroporated into BRUCE 4 embryonic stem cells. After selection in G418, resistant clones were picked and expanded. Clones in which the targeting vector have recombined with the endogenous CIS gene are currently being identified by hybridising EcoRI-digested genomic DNA with a 0.8 kb BamHI-NdeI fragment from pgmCIS 57-7-1-26. This probe (probe A, FIG. 47), which is located 3′ to the CIS sequences in the targeting vector, will distinguish between the endogenous (10 kb) and targeted (8 kb) CIS loci (FIG. 47).

### Example 44

### SOCs-1 Fusion Proteins with Green Fluorescent Protein

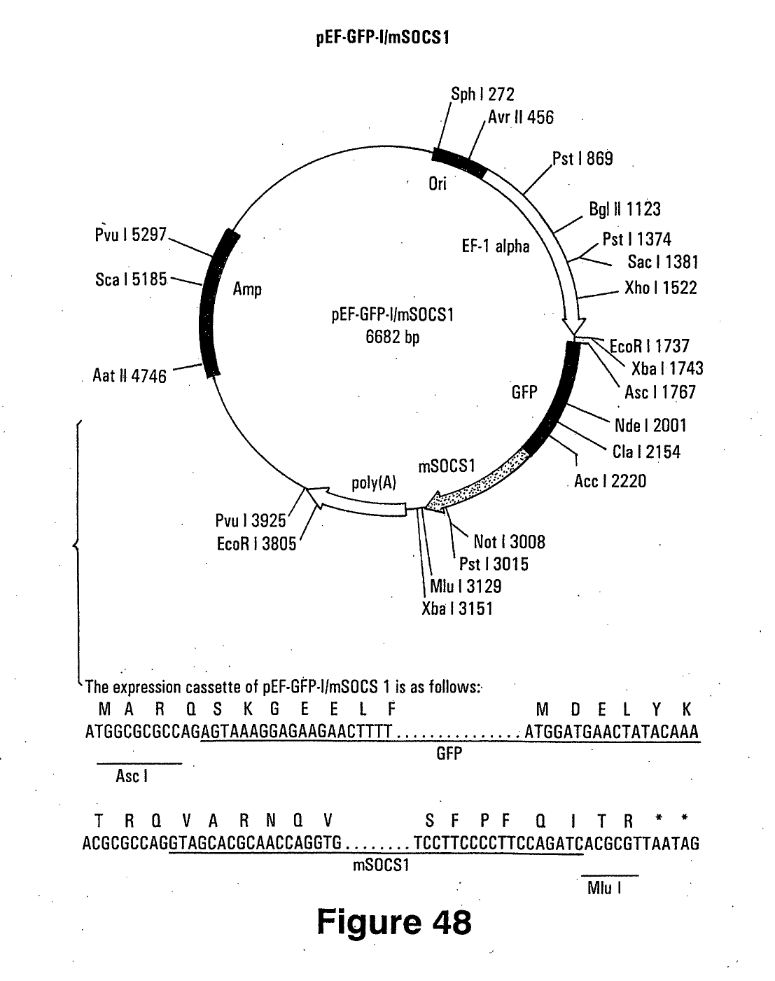

The inventors consider that if the SOCS-1 protein is active as a fusion protein with an easily visualized marker, then this would be a valuable reagent for both monitoring expression and intracellular location of SOCS-1, as well as an extremely useful reagent for monitoring a cells response to cytokines (since production of SOCS-1 is tightly regulated by cytokine). In order to test whether SOCS-1 is active as a fusion protein, the inventors have made certain vectors. Briefly, using the PCR, a derivative of the mouse SOCS1 cDNA was generated that encoded an N-terminal GFP tag (MARQSKGEELFT . . . ELYKTR [SEQ ID NO:51]) preceding the coding region (minus ATG) of mSOCS1 (see FIG. 48A), designated pEF-GFP-I/mSOCS1. Details of mammalian expression vector pEF-GFP-I can be found at http://www.wehi.edu.au./willson vectors.

The activity of these constructs was then tested as follows.

### A. Fluorescence Detection:

Single cell suspensions of M1 cells transfected with EFBOS SOCS1/GFP fusion constructs were washed in balanced salts solution (BSS) supplemented with 2% v/v fetal calf serum (FCS) and resuspended in 50 μl of BSS containing 2% v/v FCS and 1 μg/ml propidium iodide. Analyses were performed on a FACScan cell sorter (Becton-Dickinson) with dead cells excluded by propidium iodide (1 mg/ml) staining. The results showed that M1 cells expressing wild type GFP (M1 GFP 7.1.12), M1 cells expressing SOCS-1 as an N-terminal fusion with GFP (M1 SOCS-1-GFP 5.15) and a C-terminal fusion with GFP (M1 GFP-SOCS-1 6.46) were fluorescent, in contrast to a negative control clone.

### B. Inhibition of M1 Differentiation:

The capacity of M1 cells expressing SOCS1/GFP fusion proteins to differentiate in response to L-6 was assessed in agar cultures. 200 cells in DMEM containing 20% v/v FCS and 0.3% w/v agar were plated in 1 ml cultures in 35 mm Petri dishes stimulated serially diluted concentrations of IL-6. Colony numbers and morphology were scored after 7 days incubation at 37° C. in a fully humidified atmosphere of 10% v/v CO₂ in air. Undifferentiated colonies were compact while colonies composed of dispersed cells or which had a halo of migrating cells around a central core, were scored as differentiated. Importantly, as well as being fluorescent, both SOCS-1/GFP fusion proteins were also able to inhibit IL-6 induced differentiation of M1 cells when stably expressed.

SOCS-1 has previously been shown to inhibit the in vitro kinase activity of JAK2. This experiment examined the ability of SOCS-1-GFP and GFP-SOCS-1 proteins to inhibit the in vitro kinase activity of JAK2. Flag-tagged JAK2 was, therefore, transiently expressed by electroporation in Cos cells with and without co-expression of SOCS-1-GFP and GFP-SOCS-1. After 48 hours cells were lysed on ice and JAK2 proteins immunoprecipitated using 5 ml anti-JAK2 antibody (UBI). Immunoprecipitates were washed, and subjected to an in vitro kinase assay as previously described. Proteins were then separated by SDS-PAGE on a 4-15% w/v gradient gel and the gel treated with KOH to remove phosphoserine and phosphothreonine. Incorporation of radiolabelled phosphate was detected using a phosphorimager. The SOCS-1-GFP and GFP-SOCS-1 chimeric proteins were able to inhibit JAK2 kinase activity to the same extent as wild-type SOCS-1.

### Example 45

### Control of SOCs Gene Transcription by Cytokine

The inventors have shown that socs gene transcription is regulated by a range of cytokines. These studies have been extended by the Northern blot analysis of mRNA from organs of mice injected with a range of cytokine and cells treated with cytokines. SOCS-1 and SOCS-3 expression in ES cells is strictly controlled by LIF, whereas LIF does not appear to tightly regulate either expression of SOCS-2 or CIS. The expression of the SOCS-1, SOCS-2, SOCS-3 and CIS genes in bone marrow, spleen and lung occurs in response to a range of cytokines such as IL-2, IL-4, IL-5, IL-7, IL-9, IL-13, M-CSF, SCF, FL, EPO, TPO, anti-μ and LGH. Furthermore, the regulation of SOCS genes in vivo by clinically important cytokines is highlighted by the injection of GM-CSF into mice and analysis of the bonemarrow, spleen and lung at various times afterward. For SOCS-1, SOCS-2, SOCS-3 and CIS there is evidence of transcriptional control by GM-CSF.

### Example 46

### Cloning of SOCS cDNAs

DNA encoding the entire coding region of SOCS-13 has been constructed from cDNA and genomic DNA clones. Briefly, screening pulled out partial clones from both cDNA and genomic libraries, a full length coding region was generated by overlap PCR using a 5′ genomic fragment and a 3′ cDNa fragment. 5′ oligo to the genomic fragment was made with an Asc1 site and the 3′ oligo to the cDNa fragment was made with an Mlu1 site so that the stitched together coding region could be ligated straight into pEF Bos flag construct.

| | |
|---|---|
| 5′ genomic SSB-1 Oligo No 3342 | |
| AGCT G GCG CGC C AG GGT CAG AAG GTC ACG GGA GGG | [SEQ ID NO:58] |
| Asc 1 G Q K V T G G | |
| 3′ genomic oligo No 3243 | |
| AAG TCC GTT CAA GTA GCG CAT GCG GAT CTC | [SEQ ID NO:52] |
| 5′ cDNA Oligo No 3244 | |
| GAG ATC CGC ATG CGC TAC TTG AAC GGA CTT | [SEQ ID NO:53] |
| E I R M R Y L N G L | [SEQ ID NO:54] |
| 3′ cDNa Oligo No 3245 | |
| AGCT ACG CGT CTG GTA GAG GAG GTA GGC TTT GAG | [SEQ ID NO:55] |
| Mlu1 | |

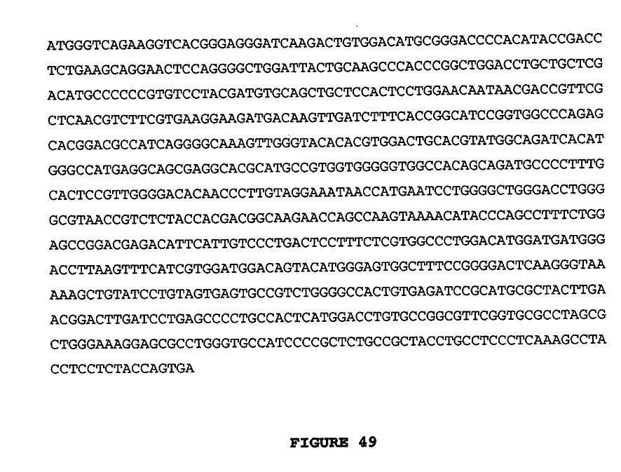

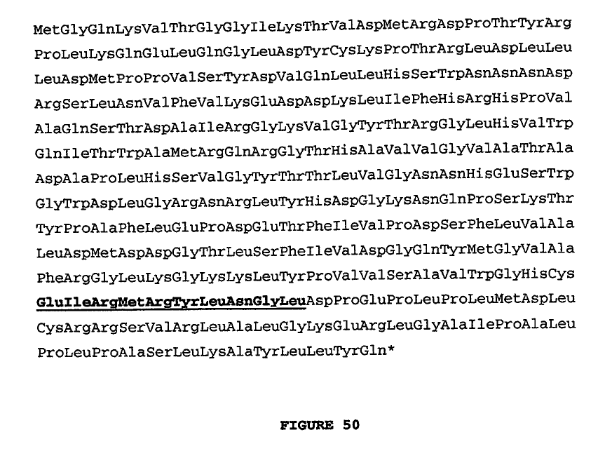

The resulting nucleotide and predicted amino acid sequences are shown in FIGS. 49 and 50.

Similarly complete DNA and predicted amino acid sequences of SOCS-5 and SOCS-9 are shown in FIGS. 51A&B and 52 A&B.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 4.1
Summary of ESTs derived from mouse SOCS-4 cDNAs

| SOCS | Species | EST name | End | EST no | Library source | Contig |
|---|---|---|---|---|---|---|
| SOCS-4 | Mouse | mc65f04 | 5′ | EST0549700 | d13.5-14.5 mouse embryo | m4.1 |
| | | mf42e06 | 5′ | EST0593477 | d13.5-14.5 mouse | m4.1 |
| | | | | | embryo | |
| | | mp10c10 | 5′ | EST0747905 | d 8.5 mouse embryo | m4.1 |
| | | mr81g09 | 5′ | EST0783081 | d13 embryo | m4.1 |
| | | mt19h12 | 5′ | EST0816531 | spleen | m4.1 |

TABLE 4.2
Summary of ESTs derived from human SOCS-4 cDNAs

| SOCS | Species | EST name | End | EST no | Library source | Contig |
|---|---|---|---|---|---|---|
| SOCS-4 | Human | 27b5 | 5′ | EST0534081 | retina | h4.2 |
| | | 30d2 | 5′ | EST0534315 | retina | h4.2 |
| | | J0159F | 5′ | EST0461188 | foetal heart | h4.2 |
| | | J3802F | 5′ | EST0461428 | foetal heart | h4.2 |
| | | EST19523 | 5′ | EST0958884 | retina | h4.2 |
| | | EST81149 | 5′ | EST1011015 | placenta | h4.2 |
| | | EST180909 | 5′ | EST0951375 | Jurkat T-lymphocyte | h4.2 |
| | | EST182619 | 5′ | EST0953220 | Jurkat T- | h4.1 |
| | | | | | lymphocyte | |
| | | ya99h09 | 3′ | EST0103262 | placenta | h4.2 |
| | | ye70c04 | 5′ | EST0172673 | foeatl liver/spleen | h4.2 |
| | | yh53c09 | 5′ | EST0197390 | placenta | h4.2 |
| | | | 3′ | EST0197391 | | h4.2 |
| | | yh77g11 | 5′ | EST0203418 | placenta | h4.2 |
| | | | 3′ | EST0203419 | | h4.1 |
| | | yh87h05 | 5′ | EST0204888 | placenta | h4.1 |
| | | | 3′ | EST0204773 | | h4.1 |
| | | yi45h07 | 5′ | EST0246604 | placenta | h4.2 |
| | | yj04e06 | 5′ | EST0258541 | placenta | h4.1 |
| | | | 3′ | EST0258285 | | h4.1 |
| | | yq12h06 | 5′ | EST0309968 | foetal liver spleen | h4.2 |
| | | yq56a06 | 3′ | EST0346924 | foetal liver spleen | h4.2 |
| | | yq60e02 | 5′ | EST0347259 | foetal liver spleen | h4.2 |
| | | | 3′ | EST0347209 | | h4.2 |
| | | yq92g03 | 5′ | EST0355932 | foetal liver spleen | h4.2 |
| | | | 3′ | EST0355884 | | h4.2 |
| | | yq97h06 | 5′ | EST0357618 | foetal liver spleen | h4.2 |
| | | | 3′ | EST0357416 | | h4.2 |
| | | yr90f01 | 5′ | EST0372402 | foetal liver spleen | h4.2 |
| | | yt69c03 | 5′ | EST0338395 | foetal liver spleen | h4.2 |
| | | | 3′ | EST0338303 | | h4.2 |
| | | yv30a08 | 3′ | EST0458506 | foetal liver spleen | h4.2 |
| | | yv55f07 | 5′ | EST0465391 | foetal liver spleen | h4.2 |
| | | | 3′ | EST0463331 | | h4.2 |
| | | yv57h09 | 5′ | EST0464336 | foetal liver spleen | h4.2 |
| | | | 3′ | EST0458765 | | h4.2 |
| | | yv87h02 | 5′ | EST0388085 | melanocyte | h4.2 |
| | | yv98e11 | 5′ | EST0400679 | melanocyte | h4.2 |
| | | | 3′ | EST0400680 | | h4.2 |
| | | yw68d10 | 5′ | EST0441370 | placenta (8-9 wk) | h4.2 |
| | | yw82a03 | 5′ | EST0463005 | placenta (8-9 wk) | h4.2 |
| | | | 3′ | EST0433678 | | h4.1 |
| | | yx08a07 | 3′ | EST0407016 | melanoocyte | h4.1 |
| | | yx72h06 | 5′ | EST0435158 | melanoocyte | h4.2 |
| | | | 3′ | EST0422871 | melanoocyte | h4.1 |
| | | yx76b09 | 5′ | EST0434011 | melanoocyte | h4.2 |
| | | yy37h08 | 5′ | EST0451704 | melanoocyte | h4.2 |
| | | yy66b02 | 5′ | EST0505446 | multiple sclerosis | h4.2 |
| | | | | | lesion | |
| | | za81f08 | 5′ | EST0511777 | foetal lung | h4.2 |
| | | zb18f07 | 3′ | EST0485315 | foetal lung | h4.1 |
| | | zc06e08 | 5′ | EST0540473 | parathyroid tumor | h4.1 |
| | | | 3′ | EST0540354 | | h4.1 |
| | | zd14g06 | 3′ | EST0564666 | foetal heart | h4.1 |
| | | zd51h12 | 3′ | EST0578099 | foetal heart | h4.1 |
| | | zd52b09 | 5′ | EST0582012 | foetal heart | h4.1 |
| | | | 3′ | EST0581958 | | h4.1 |
| | | ze25g11 | 3′ | EST0679543 | foetal heart | h4.1 |
| | | ze69f02 | 5′ | EST0635563 | retina | h4.2 |
| | | | 3′ | EST0635472 | | h4.1 |
| | | zf54f03 | 5′ | EST0680111 | retina | h4.2 |
| | | zh96e07 | 5′ | EST0616241 | foetal liver spleen | h4.2 |
| | | | 3′ | EST0615745 | | h4.2 |
| | | zv66h12 | 5′ | EST1043265 | 8-9 w foetus | h4.2 |
| | | zs83a08 | 5′ | EST0920072 | germinal centre B | h4.1 |
| | | | | | cell | |
| | | | 3′ | EST0920016 | | h4.1 |
| | | zs83g08 | 5′ | EST0920121 | germinal centre B | h4.1 |
| | | | | | cell | |
| | | | 3′ | EST0920122 | | h4.1 |

TABLE 5.1
Summary of ESTs derived from mouse SOCS-5 cDNAs

| | | | | | Library | |
|---|---|---|---|---|---|---|
| SOCS | Species | EST name | End | EST no | source | Contig |
| SOCS-5 | Mouse | mc55a01 | 5′ | EST0541556 | d13.5-14.5 | m5.1 |
| | | | | | mouse | |
| | | | | | embryo | |
| | | mh98f09 | 5′ | EST0638237 | placenta | m5.1 |
| | | my26h12 | 5′ | EST0859939 | mixed | m5.1 |
| | | | | | organs | |
| | | ve24e06 | 5′ | EST0819106 | heart | m5.1 |

TABLE 5.2
Summary of ESTs derived from human SOCS-5 cDNAs

| SOCS | Species | EST name | End | EST no | Library source | Contig |
|---|---|---|---|---|---|---|
| SOCS-5 | Human | EST15B103 | ? | EST0258029 | adipose tissue | h5.1 |
| | | EST15B105 | ? | EST0258028 | adipose tissue | h5.1 |
| | | EST27530 | 5′ | EST0965892 | cerebellum | h5.1 |
| | | zf50f01 | 5′ | EST0679820 | retina | h5.1 |

TABLE 6.1
Summary of ESTs derived from mouse SOCS-6 cDNAs

| SOCS | Species | EST name | End | EST no | Library source | Contig |
|---|---|---|---|---|---|---|
| SOCS-6 | Mouse | mco4c05 | 5′ | EST0525832 | d19.5 embryo | m6.1 |
| | | md48a03 | 5′ | EST0566730 | d13.5-14.5 embryo | m6.1 |
| | | mf31d03 | 5′ | EST0675970 | d13.5-14.5 embryo | m6.1 |
| | | mh26b07 | 5′ | EST0628752 | d13.5-14.5 placenta | m6.1 |
| | | mh78e11 | 5′ | EST0637608 | d13.5-14.5 placenta | m6.1 |
| | | mh88h09 | 5′ | EST0644383 | d13.5-14.5 placenta | m6.1 |
| | | mh94h07 | 5′ | EST0638078 | d13.5-14.5 placenta | m6.1 |
| | | mi27h04 | 5′ | EST0644252 | d13.5-14.5 embryo | m6.1 |
| | | mj29c05 | 5′ | EST0664093 | d13.5-14.5 embryo | m6.1 |
| | | mp66g04 | 5′ | EST0757905 | thymus | m6.1 |
| | | mw75g03 | 5′ | EST0847938 | liver | m6.1 |
| | | va53b05 | 5′ | EST0901540 | d12.5 embryo | m6.1 |
| | | vb34h02 | 5′ | EST0930132 | lymph node | m6.1 |
| | | vc55d07 | 3′ | EST1057735 | 2 cell embryo | m6.1 |
| | | vc59e05 | 3′ | EST1058201 | 2 cell embryo | m6.1 |
| | | vc67d03 | 3′ | EST1057849 | 2 cell embryo | m6.1 |
| | | vc68d10 | 3′ | EST1058663 | 2 cell embryo | m6.1 |
| | | vc97h01 | 3′ | EST1059343 | 2 cell embryo | m6.1 |
| | | vc99c08 | 3′ | EST1059410 | 2 cell embryo | m6.1 |
| | | vd07h03 | 3′ | EST1058173 | 2 cell embryo | m6.1 |
| | | vd08c01 | 3′ | EST1058275 | 2 cell embryo | m6.1 |
| | | vd09b12 | 3′ | EST1058632 | 2 cell embryo | m6.1 |
| | | vd19b02 | 3′ | EST1059723 | 2 cell embryo | m6.1 |
| | | vd29a04 | 3′ | ? none found | | m6.1 |
| | | vd46d06 | 3′ | ? none found | | m6.1 |

TABLE 6.2
Summary of ESTs derived from human SOCS-5 cDNAs

| SOCS | Species | EST name | End | EST no | Library source | Contig |
|---|---|---|---|---|---|---|
| SOCS-6 | Human | yf61e08 | 5′ | EST0184387 | d73 infant brain | h6.1 |
| | | yf93a09 | 5′ | EST0186084 | d73 infant brain | h6.1 |
| | | yg05f12 | 5′ | EST0191486 | d73 infant brain | h6.1 |
| | | yg41f04 | 5′ | EST0195017 | d73 infant brain | h6.1 |
| | | yg45c02 | 5′ | EST0185308 | d73 infant brain | h6.1 |
| | | yh11f10 | 5′ | EST0236705 | d73 infant brain | h6.1 |
| | | yh13b05 | 5′ | EST0237191 | d73 infant brain | h6.1 |
| | | | 3′ | EST0236958 | | h6.2 |
| | | zc35a12 | 5′ | EST0555518 | senescent | h6.1 |
| | | | | | fibroblasts | |
| | | ze02h08 | 5′ | EST0603826 | foetal heart | h6.1 |
| | | | 3′ | EST0603718 | | h6.2 |
| | | zl09a03 | 5′ | EST0773936 | pregnant uterus | h6.1 |
| | | | 3′ | EST0773892 | | h6.1 |
| | | zl69e10 | 5′ | EST0683363 | colon | h6.1 |
| | | zn39d08 | 5′ | EST0718885 | endothelial cell | h6.1 |
| | | zo39e06 | 5′ | EST0785947 | endothelial cell | h6.1 |

TABLE 7.1
Summary of ESTs derived from mouse SOCS-7 cDNAs

| | | | | | Library | |
|---|---|---|---|---|---|---|
| SOCS | Species | EST name | End | EST no | source | Contig |
| SOCS-7 | Mouse | mj39a01 | 5′ | EST0665627 | d13.5/14.5 | m7.1 |
| | | | | | embryo | |
| | | vi52h07 | 5′ | EST1267404 | d7.5 | m7.1 |
| | | | | | embryo | |

TABLE 7.2
Summary of ESTs derived from human SOCS-5 cDNAs

| SOCS | Species | EST name | End | EST no | Library source | Contig |
|---|---|---|---|---|---|---|
| SOCS-7 | HUMAN | STS WI-30171 | | (G21563) | Chromosome 2 | h7.2 |
| | | EST00939 | 5′ | EST0000906 | hippocampus | h7.1 |
| | | EST12913 | 3′ | EST0944382 | uterus | h7.2 |
| | | yc29b05 | 3′ | EST0128727 | liver | h7.2 |
| | | yp49f10 | 3′ | EST0301914 | retina | h7.2 |
| | | zt10f03 | 5′ | EST0922932 | germinal centre | h7.2 |
| | | | | | B cell | |
| | | | 3′ | EST0921231 | | h7.1 |
| | | zx73g04 | 3′ | EST1102975 | ovarian tumour | h7.1 |

TABLE 8.1
Summary of ESTs derived from mouse SOCS-8 cDNAs

| | | | | | Library | |
|---|---|---|---|---|---|---|
| SOCS | Species | EST name | End | EST no | source | Contig |
| SOCS-8 | Mouse | mj16e09 | r1 | EST0666240 | d13.5/14.5 | m8.1 |
| | | | | | embryo | |
| | | vj27a029 | r1 | EST1155973 | heart | m8.1 |

TABLE 9.1
Summary of ESTs derived from mouse SOCS-9 cDNAs

| | | | | | Library | |
|---|---|---|---|---|---|---|
| SOCS | Species | EST name | End | EST no | source | Contig |
| | Mouse | me65d05 | 5′ | EST0585211 | d | m9.1 |
| | | | | | 13.5/14.5 | |
| | | | | | embryo | |

TABLE 9.2
Summary of ESTs derived from human SOCS-5 cDNAs

| SOCS | Species | EST name | End | EST no | Library source | Contig |
|---|---|---|---|---|---|---|
| SOCS-9 | Human | CSRL-83f2-u | | (B06659) | chromsome 11 | h9.1 |
| | | EST114054 | 5′ | EST0939759 | placenta | h9.1 |
| | | yy06b07 | 3′ | EST0434504 | melanocyte | h9.1 |
| | | yy06g06 | 5′ | EST0443783 | melanocyte | h9.1 |
| | | zr40c09 | 5′ | EST0832461 | melanocyte, heart, | h9.1 |
| | | | | | uterus | |
| | | zr72h01 | 5′ | EST0892025 | melanocyte, heart, | h9.1 |
| | | | | | uterus | |
| | | | 3′ | EST0892026 | | h9.1 |
| | | yx92c08 | 5′ | EST0441160 | melanocyte | h9.1 |
| | | yx93b08 | 5′ | EST0441260 | melanocyte | h9.1 |
| | | hfe0662 | 5′ | EST0889611 | foetal heart | h9.1 |

TABLE 10.1
Summary of ESTs derived from mouse SOCS-10 cDNAs

| SOCS | Species | EST name | End | EST no | Library source | Contig |
|---|---|---|---|---|---|---|
| | Mouse | mb14d12 | 5′ | EST0549887 | d19.5 embryo | m10.1 |
| | | mb40f06 | 5′ | EST0515064 | d19.5 embryo | m10.1 |
| | | mg89b11 | 5′ | EST0630631 | d13.5-14.5 embryo | m10.1 |
| | | mq89e12 | 5′ | EST0776015 | heart | m10.1 |
| | | mp03g12 | 5′ | EST0741991 | heart | m10.1 |
| | | vh53c11 | 5′ | EST1154634 | mammary gland | m10.1 |

TABLE 10.2
Summary of ESTs derived from human SOCS-5 cDNAs

| SOCS | Species | EST name | End | EST no | Library source | Contig |
|---|---|---|---|---|---|---|
| SOCS-10 | Human | aa48h10 | 3′ | EST1135220 | germinal centre B cell | h10.2 |
| | | zp35h01 | 3′ | EST0819137 | muscle | h10.2 |
| | | zp97h12 | 5′ | EST0835442 | muscle | h10.2 |
| | | | 3′ | EST0831211 | | h10.2 |
| | | zq08h01 | 5′ | EST0835907 | muscle | h10.1 |
| | | zr34g05 | 5′ | EST0834251 | melanocyte, heart, | h10.2 |
| | | | | | uterus | |
| | | | 3′ | EST0834440 | | h10.2 |
| | | EST73000 | 5 | EST1004491 | ovary | h10.2 |
| | | HSDHEI005 | ? | EST0013906 | heart | h10.2 |

TABLE 11.1
Summary of ESTs derived from human SOCS-5 cDNAs

| SOCS | Species | EST name | End | EST no | Library source | Contig |
|---|---|---|---|---|---|---|
| SOCS-11 | Human | zt24h06 | r1 | EST0925023 | ovarian tumor | 11.1 |
| | | zr43b02 | r1 | EST0873006 | melanocyte, heart, uterus | 11.1 |
| | | | s1 | EST0872954 | | 11.1 |

TABLE 12.1
Summary of ESTs derived from mouse SOCS-12 cDNAs

| SOCS | Species | EST name | End | EST no | Library source | Contig |
|---|---|---|---|---|---|---|
| SOCS-12 | Mouse | EST03803 | 5′ | EST1054173 | day 7.5 emb | m12.1 |
| | | | | | ectoplacental cone | |
| | | mt18f02 | 5′ | EST0817652 | 3NbMS spleen | m12.1 |
| | | mz60g10 | 5′ | EST0890872 | lymph node | m12.1 |
| | | va05c11 | 5′ | EST0909449 | lymph node | m12.1 |

TABLE 12.2
Summary of ESTs derived from human SOCS-5 cDNAs

| SOCS | Species | EST name | End | EST no | Library source | Contig |
|---|---|---|---|---|---|---|
| SOCS-12 | Human | STS-SHGC-13867 | | | Chromosome 2 | h12.2 |
| | | EST177695 | 5′ | EST0948071 | Jurkat cells | h12.1 |
| | | EST64550 | 5′ | EST0997367 | Jurkat cells | h12.1 |
| | | EST76868 | 5′ | EST1007291 | pineal body | h12.2 |
| | | PMY2369 | 5′ | EST1115998 | KG-1 | h12.1 |
| | | yb38f04 | 5′ | EST0108807 | foetal spleen | h12.1 |
| | | | 3′ | | | h12.2 |
| | | yg74e12 | 5′ | EST0224407 | d73 brain | h12.1 |
| | | yh13g04 | 5′ | EST0237226 | d73 brain | h12.1 |
| | | | 3′ | EST0236992 | | h12.2 |
| | | yh48b06 | 5′ | yh48b06 | placenta | h12.2 |
| | | yh53a05 | 5′ | EST0197282 | placenta | h12.2 |
| | | | 3′ | EST0197486 | | h12.2 |
| | | yn48h09 | 5′ | EST0278258 | brain | h12.2 |
| | | | 3′ | EST0278259 | | h12.2 |
| | | yn90a09 | 3′ | EST0302557 | brain | h12.2 |
| | | yo08f03 | 5′ | EST0301790 | brain | h12.2 |
| | | | 3′ | EST0302059 | | h12.2 |
| | | yo11e01 | 3′ | ? none found | | h12.2 |
| | | yo63b12 | 5′ | EST0303606 | breast | h12.2 |
| | | | 3′ | EST0304085 | | h12.2 |
| | | yq56g02 | 3′ | EST0346935 | foetal liver spleen | h12.1 |
| | | zh57c04 | 3′ | EST0594201 | foetal liver spleen | h12.2 |
| | | zh79h01 | 3′ | EST0598945 | foetal liver spleen | h12.2 |
| | | zh99a11 | 3′ | EST0618570 | foetal liver spleen | h12.2 |
| | | zo92h12 | 5′ | EST0803392 | ovarian cancer | h12.1 |
| | | | 3′ | EST0803393 | | h12.2 |
| | | zs48c01 | 5′ | EST0925714 | germinal centre | h12.1 |
| | | | | | B cell | |
| | | | 3′ | EST0925530 | | h12.2 |
| | | zs45h02 | 3′ | EST0932296 | germinal centre | h12.2 |
| | | | | | B cell | |

TABLE 13.1
Summary of ESTs derived from mouse SOCS-13 cDNAs

| SOCS | Species | EST name | End | EST no | Library source | Contig |
|---|---|---|---|---|---|---|
| SOCS-13 | Mouse | ma39c09 | 5′ | EST0517875 | day 19.5 embryo | m13.1 |
| | | me60c05 | 5′ | EST0584950 | day 13.5/14.5 embryo | m13.1 |
| | | mi78g05 | 5′ | EST0653834 | day 19.5 embryo | m13.1 |
| | | mk10c11 | 5′ | EST0735158 | day 19.5 embryo | m13.1 |
| | | mo48g12 | 5′ | EST0745111 | day 10.5 embryo | m13.1 |
| | | mp94a01 | 5′ | EST0762827 | thymus | m13.1 |
| | | vb57c07 | 5′ | EST1028976 | day 11.5 embryo | m13.1 |
| | | vh07c11 | 5′ | EST1117269 | mammary gland | m13.1 |

TABLE 13.2
Summary of ESTs derived from human SOCS-13 cDNAs

| | | | | | Library | |
|---|---|---|---|---|---|---|
| SOCS | Species | EST name | End | EST no | source | Contig |
| SOCS-13 | Human | EST59161 | 5′ | EST0992726 | infant | h13.1 |
| | | | | | brain | |

TABLE 14.1
Summary of ESTs derived from mouse SOCS-14 cDNAs

| | | | | | Library | |
|---|---|---|---|---|---|---|
| SOCS | Species | EST name | End | EST no | source | Contig |
| SOCS-14 | mouse | mi75e03 | 5′ | EST0651892 | d19.5 | m14.1 |
| | | | | | embryo | |
| | | vd29h11 | 5′ | EST1067080 | 2 cell | m14.1 |
| | | | | | embryo | |
| | | vd53g07 | 5′ | EST1119627 | 2 cell | m14.1 |
| | | | | | embryo | |

TABLE 15.1
Summary of ESTs derived from mouse SOCS-15 cDNAs

| | | | | | Library | |
|---|---|---|---|---|---|---|
| SOCS | Species | EST name | End | EST no | source | Contig |
| SOCS- | Mouse | mh29b05 | 5′ | EST0628834 | placenta | m15.1 |
| 15 | | mh98h09 | 5′ | EST0638243 | placenta | m15.1 |
| | | ml45a02 | 5′ | EST0687171 | testis | m15.1 |
| | | mu43a10 | 5′ | EST851588 | thymus | m15.1 |
| | | my38c09 | 5′ | EST878461 | pooled | m15.1 |
| | | | | | organs | |
| | | vj37h07 | 5′ | EST1174791 | diaphragm | m15.1 |
| | | AC002393 | | | Chromo- | m15.1 |
| | | | | | some 6 | |
| | | | | | BAC | |

TABLE 15.2
Summary of ESTs derived from human SOCS-15 cDNAs

| SOCS | Species | EST name | End | EST no | Library source | Contig |
|---|---|---|---|---|---|---|
| SOCS-15 | Human | EST98889 | 5′ | EST1026568 | thyroid | h15.1 |
| | | ne48bo5 | 3′ | EST1138057 | colon tumour | h15.1 |
| | | yb12h12 | 5′ | EST0098885 | placenta | h15.1 |
| | | | 3′ | EST0098886 | | h15.1 |
| | | HSU47924 | | | Chromosome 12 | h15.1 |
| | | | | | BAC | |

### BIBLIOGRAPHY

Alexander W S, Metcalf D and Dunn A R (1995). Embo Journal 14, 5569-78.
Altschul, S. F. Gish, W. Miller, W. Myers, E. W. & Lipman, D. J. (1990) J. Mol. Biol. 215, 403-10.
Ansari-Lari, M. A., Shen, Y., Munzy, D. M., Lee, W. and Gibbs, R. A. (1997) Genome. Res. 7, 268-280.
Bazan J F (1990). [Review]. Immunology Today 11, 350-4.
Bork, P. (1993) Proteins: Struct. Funct. Genet. 17, 363-374.
Cockwell, L. Y. and Giles, I. G. (1989) Comp. Appl. Biosci. 5, 227-232.
Cutler R L, Liu L, Damen J E and Krystal G (1993). Journal of Biological Chemistry 268, 21463-5.
Darnell J. Jr., Kerr I M and Stark G R (1994). Science 264, 1415-21.
David M, Petricoin E3, Benjamin C, Pine R, Weber M J and Larner A C (1995). Science 269, 1721-3.
David M, Wong L, Flavell R, Thompson S A, Wells A, Lamer A C and Johnson G R (1996). Journal of Biological Chemistry 271, 9185-8.
Dugaiczyk A, Haron J A, Stone E M, Dennison O E, Rothblum K N and Schwartz R J (1983). Biochemistry 22, 1605-13.
Durbin J E, Hackenmiller R, Simon M C and Levy D E (1996). Cell 84, 443-50.
Etzold, T., Ulyanov, A. and Argos, P. (1996) Methods Enzymol. 266, 114-28.
Gearing D P, Nicola N A, Metcalf D, Foote S, Willson T A, Gough N M and Williams L (1989). BioTechnology 7, 1157-1161.
Gupta S, Yan H, Wong L H, Ralph S, Krolewski J and Schindler C (1996). Embo Journal 15, 1075-84.
Hilton D J (1994). An introduction to cytokine receptors, p 8-16 in Guidebook to Cytokines and Their Receptors, Eds: N. A. Nicola. Oxford University Press: Oxford.
Hilton D J, Hilton A A, Raicevic A, Rakar S, Harrison-Smith M, Gough N M, Begley C G,
Metcalf D, Nicola N A and Willson T A (1994). Embo Journal 13, 4765-75.
Hilton D J, Watowich S S, Katz L and Lodish H F (1996). J. Biol. Chem. 271, 4699-4708.
Horton et al. 1989, Gene, 77: 61-68.
Ichikawa Y (1969). Journal of Cellular Physiology 74, 223-34.
Ihle J N, Witthuhn B A, Quelle F W, Yamamoto K and Silvennoinen 0 (1995). Annual Review of Immunology 13, 369-98.
Ihle J N (1995). Nature 377, 591-4.
Kaplan M H, Sun Y L, Hoey T and Grusby M J (1996b). Nature 382, 174-179.
Kaplan M H, Schindler U, Smiley S T and Grusby M J (1996a). Immunity 4, 313-9.
Levy D E and Stark G R (1996). Molecular & Cellular Biology 16, 369-75.
Masuhara, M., Sakamoto, H., Matsumoto, A., Suzuki, R., Yasukawa, H., Mitsui, K., Wakioka, T., Tanimura, S., Sasaki, A., Misawa, H., Yokouchi, M., Ohtsubo, M. & Yoshimura, A. Cloning and characterization of novel CIS family genes (1997) Biochem. Biophys. Res. Comm., 239, 439-446.
Masuhara et al. 1997, Biochem. Biophys. Res. Comm., 239: 439-446.
Meraz M A, White J M, Sheehan K C, Bach E A, Rodig S J, Dighe A S, Kaplan D H, Riley J K, Greenlund A C, Campbell D, Carver-Moore K, DuBois R N, Clark R, Aguet M and Schreiber R D (1996). Cell 84, 431-42.
Metcalf D, Wilson T A, Hilton D J, DiRago L and Mifsud S. (1995) Leukaemia 9, 1556-1564.
Metcalf, D. (1984) Clonal culture of hemopoietic cells: Techniques and applications, pp. 19-72, Elsevier, Amsterdam, The Netherlands.
Mizushima S and Nagata S (1990). Nucleic Acids Research 18, 5322.
Murakami M, Narazaki M, Hibi M, Yawata H, Yasukawa K, Hamaguchi M, Taga T and Kishimoto T (1991). Proc. Natl. Acad. Sci. USA 88, 11349-11353.
Neer, E. J., Schmidt, C. J., Nambudripad, R. and Smith, T. F. (1994) Nature 371, 297-300.
Nicholson et al. 1995, Blood; 86: 3698-3704.
Nicholson, S. E., Novak, U., Ziegler, S. F. & Layton, J. E. Distinct regions of the granulocyte colony-stimulating factor receptor are required for tyrosine phosphorylation of the signaling molecules JAK2, Stat3, and p 42, p44MAPK (1995) Blood, 86, 3698-3704.
Nicola, N V, Viney E, Hilton D J, Roberts B and Wilson T. (1996) Growth Factors 13, 141-149.
Nicola N A (1994). Guidebook to Cytokines and Their Receptors. Oxford University Press: Oxford.
Novak U, Harpur A G, Paradiso L, Kanagasundaram V, Jaworowski A, Wilks A F and Hamilton J A (1995). Blood 86, 2948-56.
Ogilvy, S., Elefanty, A. G., Visvader, J., Bath, M. L., Harris, A. W. and Adams, J. M. (1998) Transcriptional regulation of vav, a gene expressed throughout the hematopoietic compartment. Blood, 91(2), 419-430.
Pearson W R and Lipman D J. (1988) Proc. Natl. Acad. Sci. USA 85, 2444-8.
Pearson W R. (1990) Methods Enzymol. 183, 63-98.
Rayner J R and Gonda T J (1994). Molecular & Cellular Biology 14, 880-7.
Sambrook J, Fritsch E F and Maniatis T (1989). Molecular Cloning, A Laboratory Manual. Cold Spring Harbour Laboratory Press, Cold Spring Harbour USA.
Sato N, Sakamaki K, Terada N, Arai K and Miyajima A (1993). Embo Journal 12, 4181-9.
Shimoda K, van Deursen J, Sangster M Y, Sarawar S R, Carson R T, Tripp R A, Chu C, Quelle F W, Nosaka T, Vignali D A, Doherty P C, Grosveld G, Paul W E and Ihle J N (1996). Nature 380, 630-3.
Shual K, Ziemiecki A, Wilks A F, Harpur A G, Sadowski H B, Gilman M Z and Darnell J E (1993). Nature 366, 580-3.
Sprang S R and Bazan J F (1993). Curr. Opin. Structural Biol. 3, 815-827.
Starr, R., Willson, T. A., Viney, E. M., Murray, L. J. L., Rayner, J. R., Jenkins, B. J., Gonda, T. J., Alexander, W. S., Metcalf, D., Nicola, N. A. & Hilton, D. J. A family of cytokine-inducible inhibitors of signalling (1997) Nature, 386, 917-921.
Starr et al. 1997, Nature, 386: 917-921.
Takeda K, Tanaka T, Shi W, Matsumoto M, Minami M, Kashiwamura S, Nakanishi K, Yoshida N, Kishimoto T and Akira S (1996). Nature 380, 627-30.
Thierfelder W E, Vandeursen J M, Yamamoto K, Tripp R A, Sarawar S R, Carson R T, Sangster M Y, Vignali D D A, Doherty P C, Grosveld G C and Ihle J N (1996). Nature 382, 171-174.
Wakao H, Gouilleux F and Groner B (1994). Embo Journal 13, 2182-91.
Wen Z, Zhong Z and Darnell J. Jr. (1995). Cell 82, 241-50.
Yi T, Mui A L, Krystal G and Ihle J N (1993). Molecular & Cellular Biology 13, 7577-86.
Yoshimura A, Ohkubo T, Kiguchi T, Jenkins N A, Gilbert D J, Copeland N G, Hara T and Miyajima A (1995). Embo Journal 14, 2816-26.

1-16. (canceled)

17. A method of modulating signal transduction in a cell containing a SOCS gene, said method comprising identifying a modulator of SOCS gene expression or SOCS protein activity in a cell and contacting said cell with said identified modulator wherein the modulator binds to SOCS gene or SOCS protein for a time sufficient to modulate signal transduction.

18. (canceled)

19. A method according to claim 17 wherein signal transduction is mediated by a cytokine, a hormone, a microbe or a microbial product, a parasite, an antigen or other effector molecule.

20. A method according to claim 19 wherein the cytokine is one or more of EPO, TPO, G-CSF, GM-CSF, IL-3, IL-2, IL-4, IL-7, IL-13, IL-6, LIF, IL-12, IFN, TNF, IL-1 and/or M-CSF.

21. A method according to claim 20 wherein the cytokine is one or more of IL-6, LIF, OSM, IFN- and/or thrombopoietin.

22. A method according to claim 21 wherein the cytokine is IL-6.

23. A method according to claim 17 wherein the SOCS gene encodes a protein having a SOCS box comprising the amino acid sequence:
(SEQ ID NO: 59)
X₁X₂X₃X₄X₅X₆X₇X₈X₈X₁₀X₁₁X₁₂X₁₃X₁₄X₁₅X₁₆[Xᵢ]ₙX₁₇X₁₈
X₁₉X₂₀X₂₁X₂₂X₂₃[Xj]ₙX₂₄X₂₅X₂₆X₂₇X₂₈
wherein:
X₁ is L, I, V, M, A or P;
X₂ is any amino acid residue;
X₃ is P, T or S;
X₄ is L, I, V, M, A or P;
X₅ is any amino acid;
X₆ is any amino acid;
X₇ is L, I, V, M, A, F, Y or W;
X₈ is C, T or S;
X₉ is R, K or H;
X₁₀ is any amino acid;
X₁₁ is any amino acid;
X₁₂ is L, I, V, M, A or P;
X₁₃ is any amino acid;
X₁₄ is any amino acid;
X₁₅ is any amino acid;
X₁₆ is L, I, V, M, A, P, G, C, T or S;
[Xᵢ]ₙ is a sequence of n amino acids wherein n is from 1 to 50 amino acids and wherein the sequence Xᵢ may comprise the same or different amino acids selected from any amino acid residue;
X₁₇ is L, I, V, M, A or P;
X₁₈ is any amino acid;
X₁₉ is any amino acid;
X₂₀ L, I, V, M, A or P;
X₂₁ is P;
X₂₂ is L, I, V, M, A, P or G;
X₂₃ is P or N;
[Xⱼ]ₙ is a sequence of n amino acids wherein n is from 0 to 50 amino acids and wherein the sequence Xⱼ may comprise the same or different amino acids selected from any amino acid residue;
X₂₄ is L, I, V, M, A or P;
X₂₅ is any amino acid;
X₂₆ is any amino acid;
X₂₇ is Y or F; and
X₂₈ is L, I, V, M, A or P, with the proviso that said protein is not CIS.

24. A method according to claim 23 wherein the SOCS gene comprises a nucleotide sequence selected from the group consisting of SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 16, SEQ ID NO: 17, SEQ ID NO: 20, SEQ ID NO: 22, SEQ ID NO: 23, SEQ ID NO: 24, SEQ ID NO: 26, SEQ ID NO: 27, SEQ ID NO: 28, SEQ ID NO: 30, SEQ ID NO: 31, SEQ ID NO: 32, SEQ ID NO: 33, SEQ ID NO: 34, SEQ ID NO: 35, SEQ ID NO: 37, SEQ ID NO: 38, SEQ ID NO: 39, SEQ ID NO: 40, SEQ ID NO: 42, SEQ ID NO: 43, SEQ ID NO: 45 and SEQ ID NO: 47.

25. A method according to claim 23 wherein the SOCS gene encodes a protein comprising an amino acid sequence substantially as set forth in SEQ ID NO: 4, SEQ ID NO: 6, SEQ ID NO: 8, SEQ ID NO 10, SEQ ID NO: 12, SEQ ID NO: 14, SEQ ID NO: 18, SEQ ID NO: 21, SEQ ID NO: 25, SEQ ID NO: 29, SEQ ID NO: 36, SEQ ID NO: 41, SEQ ID NO: 44, SEQ ID NO: 46 or SEQ ID NO: 48.

26-30. (canceled)

31. The method according to claim 17 wherein the SOCS gene encodes a protein having a SOCS box comprising the amino acid sequence
(SEQ ID NO: 59)
X₁X₂X₃X₄X₅X₆X₇X₈X₈X₁₀X₁₁X₁₂X₁₃X₁₄X₁₅X₁₆[Xᵢ]ₙX₁₇X₁₈
X₁₉X₂₀X₂₁X₂₂X₂₃[Xj]ₙX₂₄X₂₅X₂₆X₂₇X₂₈
wherein: X₁ is L, I, V, M or P;
X₂ is any amino acid residue;
X₃ is P, T or S;
X₄ is L, I, V, M, A or P;
X₅ is any amino acid;
X₆ is any amino acid;
X₇ is L, I, V, M, A, F, Y or W;
X₈ is C, T or S;
X₉ is R, K or H;
X₁₀ is any amino acid;
X₁₁ is any amino acid;
X₁₂ is L, I, V, M, A or P;
X₁₃ is any amino acid;
X₁₄ is any amino acid;
X₁₅ is any amino acid;
X₁₆ is L, I, V, M, A, P, G, C, T or S;
[Xᵢ]ₙ is a sequence of n amino acids wherein n is from 1 to 50 amino acids and wherein the sequence Xᵢ can comprise the same or different amino acids selected from any amino acid residue;
X₁₇ is L, I, V, M, A or P;
X₁₈ is any amino acid;
X₁₉ is any amino acid;
X₂₀ L, I, V, M, A or P;
X₂₁ is P;
X₂₂ is L, I, V, M, A, P or G;
X₂₃ is P or N;
[Xⱼ]ₙ is a sequence of n amino acids wherein n is from 0 to 50 amino acids and wherein the sequence Xⱼ can comprise the same or different amino acids selected from any amino acid residue;
X₂₄ is L, I, V, M, A or P;
X₂₅ is any amino acid;
X₂₆ is any amino acid;
X₂₇ is Y or F; and
X₂₈ is L, I, V, M, A or P.

32. The method of claim 23, wherein said SOCS box comprises a sequence selected from any one of SEQ ID NOs: 64-81.

33. The method of claim 23, wherein said SOCS box comprises a sequence having at least about 70% similarity to any one of SEQ ID NOs: 64-81.

34. The method of claim 31, wherein said SOCS box comprises a sequence selected from any one of SEQ ID NOs: 64-81.

35. The method of claim 31, wherein said SOCS box comprises a sequence having at least about 70% similarity to any one of SEQ ID NOs: 64-81.
